SEQUENCE LISTING (202 sequences)

SEQ ID NO: 1 - protein, 8 aa, synthetic construct
GYTFTSYY

SEQ ID NO: 2 - protein, 8 aa, synthetic construct
GFTFSSYA

SEQ ID NO: 3 - protein, 10 aa, synthetic construct
GDSVSSNSAA

SEQ ID NO: 4 - protein, 8 aa, synthetic construct
INPNSGGT

SEQ ID NO: 5 - protein, 8 aa, synthetic construct
ISGSGGST

SEQ ID NO: 6 - protein, 9 aa, synthetic construct
TYYRSKWYN

SEQ ID NO: 7 - protein, 16 aa, synthetic construct
VRSKGRAARNYYYMDV

SEQ ID NO: 8 - protein, 16 aa, synthetic construct
ARDLYGYRYYYYGMDV

SEQ ID NO: 9 - protein, 19 aa, synthetic construct
ARAYSPSRLRWSRAAAFDI

SEQ ID NO: 10 - protein, 6 aa, synthetic construct
SVRTYY

SEQ ID NO: 11 - protein, 6 aa, synthetic construct
SLRRYF

SEQ ID NO: 12 - protein, 6 aa, synthetic construct
SLRSYY

SEQ ID NO: 13-15 - skipped (3 entries)

SEQ ID NO: 16 - protein, 10 aa, synthetic construct
NSRDSSANPV

SEQ ID NO: 17 - protein, 10 aa, synthetic construct
NSRDRSGTVV

SEQ ID NO: 18 - protein, 12 aa, synthetic construct
SSRDSSGNHLVV

SEQ ID NO: 19 - protein, 123 aa, synthetic construct
QVQLVQSGAEVKKPGASVKVSCKASGYTFTSYYMHWVRQAPGQGLEWMGWINPNSGGTNYAQKFQGRVTITADKSTSTAYMELNSLRAEDTAVYYCVRSKGRAARNYYYMDVWGKGTTVTVSS

SEQ ID NO: 20 - protein, 123 aa, synthetic construct
EVQLVESGGGVVQPGRSLRLSCAASGFTFSSYAMSWVRQAPGKGLEWVSAISGSGGSTYYAGSVKGRFTISRDNSKNTLYLQMNSLRAEDTAVYYCARDLYGYRYYYYGMDVWGQGTMVTVSS

SEQ ID NO: 21 - protein, 129 aa, synthetic construct
QVQLQQSGPGQVKPSQTLSLTCAISGDSVSSNSAAWNWIRQSPSRGLEWLGRTYYRSKWYNDYAVSVKSRITINPDTSKNQFSLQLNSVTPEDTAVYYCARAYSPSRLRWSRAAAFDIWGQGTTVTVSS

SEQ ID NO: 22 - protein, 107 aa, synthetic construct
SSELTQDPAASVALGQTVRITCQGDSVRTYYAGWYQQKPGQAPVLVIYGKNHRPSGIPDRFSGSTSGNTASLTITGVQAEDEADYYCNSRDSSANPVFGGGTKVTVL

SEQ ID NO: 23 - protein, 107 aa, synthetic construct
SSELTQDPAASVALGQTVKITCQGDSLRRYFASWYQQKPGQAPTLVTYGKNRRPSGVPDRLSGSSSGDTASLTITGAQAEDEGDYYCNSRDRSGTVVFGGGTKLTVL

SEQ ID NO: 24 - protein, 109 aa, synthetic construct
SSELTQDPAVSVALGQTVRITCQGDSLRSYYANWYQQKPGQAPILVNYGKNNRPSGIPDRFSGSSSGKTASLTITGAQAEDEADYYCSSRDSSGNHLVVFGGGTQLTVL

SEQ ID NO: 25 - protein, 245 aa, synthetic construct
SSELTQDPAASVALGQTVRITCQGDSVRTYYAGWYQQKPGQAPVLVIYGKNHRPSGIPDRFSGSTSGNTASLTITGVQAEDEADYYCNSRDSSANPVFGGGTKVTVLGGGGSGGGGSGGGGSQVQLVQSGAEVKKPGASVKVSCKASGYTFTSYYMHWVRQAPGQGLEWMGWINPNSGGTNYAQKFQGRVTITADKSTSTAYMELNSLRAEDTAVYYCVRSKGRAARNYYYMDVWGKGTTVTVSS

SEQ ID NO: 26 - protein, 245 aa, synthetic construct
SSELTQDPAASVALGQTVKITCQGDSLRRYFASWYQQKPGQAPTLVTYGKNRRPSGVPDRLSGSSSGDTASLTITGAQAEDEGDYYCNSRDRSGTVVFGGGTKLTVLGGGGSGGGGSGGGGSEVQLVESGGGVVQPGRSLRLSCAASGFTFSSYAMSWVRQAPGKGLEWVSAISGSGGSTYYAGSVKGRFTISRDNSKNTLYLQMNSLRAEDTAVYYCARDLYGYRYYYYGMDVWGQGTMVTVSS

SEQ ID NO: 27 - protein, 253 aa, synthetic construct
SSELTQDPAVSVALGQTVRITCQGDSLRSYYANWYQQKPGQAPILVNYGKNNRPSGIPDRFSGSSSGKTASLTITGAQAEDEADYYCSSRDSSGNHLVVFGGGTQLTVLGGGGSGGGGSGGGGSQVQLQQSGPGQVKPSQTLSLTCAISGDSVSSNSAAWNWIRQSPSRGLEWLGRTYYRSKWYNDYAVSVKSRITINPDTSKNQFSLQLNSVTPEDTAVYYCARAYSPSRLRWSRAAAFDIWGQGTTVTVSS

SEQ ID NO: 28 - protein, 21 aa, synthetic construct
MALPVTALLLPLALLLHAARP

SEQ ID NO: 29 - protein, 20 aa, synthetic construct
METDTLLLWVLLLWVPGSTG

SEQ ID NO: 30 - protein, 22 aa, synthetic construct
MLLLVTSLLLCELPHPAFLLIP

SEQ ID NO: 31 - protein, 21 aa, synthetic construct
MEFGLSWLFLVAILKGVQCSR

SEQ ID NO: 32 - protein, 15 aa, synthetic construct
GGGGSGGGGSGGGGS

SEQ ID NO: 33 - protein, 18 aa, synthetic construct
GSTSGSGKPGSGEGSTKG

SEQ ID NO: 34 - protein, 45 aa, synthetic construct
TTTPAPRPPTPAPTIASQPLSLRPEACRPAAGGAVHTRGLDFACD

SEQ ID NO: 35 - protein, 39 aa, synthetic construct
IEVMYPPPYLDNEKSNGTIIHVKGKHLCPSPLFPGPSKP

SEQ ID NO: 36 - protein, 42 aa, synthetic construct
AAAIEVMYPPPYLDNEKSNGTIIHVKGKHLCPSPLFPGPSKP

SEQ ID NO: 37 - protein, 12 aa, synthetic construct
ESKYGPPCPPCP

SEQ ID NO: 38 - protein, 12 aa, synthetic construct
ESKYGPPCPSCP

SEQ ID NO: 39 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 24 aa, synthetic construct
IYIWAPLAGTCGVLLLSLVITLYC

SEQ ID NO: 41 - protein, 27 aa, synthetic construct
FWVLVVVGGVLACYSLLVTVAFIIFWV

SEQ ID NO: 42 - protein, 28 aa, synthetic construct
MFWVLVVVGGVLACYSLLVTVAFIIFWV

SEQ ID NO: 43 - protein, 42 aa, synthetic construct
KRGRKKLLYIFKQPFMRPVQTTQEEDGCSCRFPEEEEGGCEL

SEQ ID NO: 44 - protein, 41 aa, synthetic construct
RSKRSRLLHSDYMNMTPRRPGPTRKHYQPYAPPRDFAAYRS

SEQ ID NO: 45 - protein, 41 aa, synthetic construct
RSKRSRGGHSDYMNMTPRRPGPTRKHYQPYAPPRDFAAYRS

SEQ ID NO: 46 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 25 aa, synthetic construct
TTTPAPRPPTPAPTIASQPLSLRPE

SEQ ID NO: 49 - protein, 44 aa, synthetic construct
ACRPAAGGAVHTRGLDFACDIYIWAPLAGTCGVLLLSLVITLYC

SEQ ID NO: 50 - protein, 5 aa, synthetic construct
SYWIE

SEQ ID NO: 51 - protein, 5 aa, synthetic construct
DYVIS

SEQ ID NO: 52 - protein, 5 aa, synthetic construct
THWMH

SEQ ID NO: 53 - protein, 5 aa, synthetic construct
GYWMY

SEQ ID NO: 54 - protein, 17 aa, synthetic construct
EILPGSGSTSYNEKFKG

SEQ ID NO: 55 - protein, 17 aa, synthetic construct
EIYPGSGSSYYNEKFKG

SEQ ID NO: 56 - protein, 17 aa, synthetic construct
MINPSDGVTYYAQTFQG

SEQ ID NO: 57 - protein, 17 aa, synthetic construct
AISPGGGSTYYPDSVKG

SEQ ID NO: 58 - protein, 11 aa, synthetic construct
RGYGYDEGFDY

SEQ ID NO: 59 - protein, 8 aa, synthetic construct
PGDLGFAY

SEQ ID NO: 60 - protein, 9 aa, synthetic construct
EYYGEGFDY

SEQ ID NO: 61 - protein, 12 aa, synthetic construct
SLTATHTYEYDY

SEQ ID NO: 62 - protein, 10 aa, synthetic construct
ASQDINSYLS

SEQ ID NO: 63 - protein, 14 aa, synthetic construct
ASQSVDYDGDSYMN

SEQ ID NO: 64 - protein, 11 aa, synthetic construct
RASQGISNYLA

SEQ ID NO: 65 - protein, 7 aa, synthetic construct
RANRLVD

SEQ ID NO: 66 - protein, 7 aa, synthetic construct
AASNLES

SEQ ID NO: 67 - protein, 7 aa, synthetic construct
SASNLQS

SEQ ID NO: 68 - protein, 9 aa, synthetic construct
LQYDEFPPT

SEQ ID NO: 69 - protein, 9 aa, synthetic construct
QQSNKDPFT

SEQ ID NO: 70 - protein, 9 aa, synthetic construct
QQSYSTPLT

SEQ ID NO: 71 - protein, 120 aa, synthetic construct
QVQLQQSGAELMKPGASVKISCKATGYTFSSYWIEWVKQRPGHGLEWIGEILPGSGSTSYNEKFKGKATFTADTSSNTAYMQLSSLTSEDSAVYYCARRGYGYDEGFDYWGQGTTLTVSS

SEQ ID NO: 72 - protein, 117 aa, synthetic construct
QVQLQQSGPELVKPGASVKMSCKASGYTFTDYVISWVRQAPGQGLEWIGEIYPGSGSSYYNEKFKGRATLTADKSSNTAYMQLSSLRSEDSAVYFCARPGDLGFAYWGQGTLVTVSS

SEQ ID NO: 73 - protein, 118 aa, synthetic construct
QVQLVQSGAEVKKPGASVKVSCKASGYSLITHWMHWVRQAPGQGLEWMGMINPSDGVTYYAQTFQGRVTMTRDTSTSTVYMELSSLRSEDTAVYYCAREYYGEGFDYWGQGTLVTVSS

SEQ ID NO: 74 - protein, 121 aa, synthetic construct
EVQLVESGGGLVQSGGSLRLSCAASGFTFSGYWMYWVRQAPGKGLEWVSAISPGGGSTYYPDSVKGRFTISRDNAKNTLYLQMNSLEPEDTALYYCASSLTATHTYEYDYWGQGTQVTVSS

SEQ ID NO: 75 - protein, 108 aa, synthetic construct
DIKMTQSPSSMYASLGERVTITCKASQDINSYLSWFQQKPGKSPKTLIYRANRLVDGVPSRFSGSGSGQDYSLTISSLEYEDMGIYYCLQYDEFPPTFGAGTKLELKR

SEQ ID NO: 76 - protein, 112 aa, synthetic construct
DIVLTQSPSSLAVSLGQRATISCKASQSVDYDGDSYMNWYQQKPGQPPKLLIYAASNLESGIPARFSGSGSGTDFTLTIHPVEEEDAATYYCQQSNKDPFTFGGGTKLELKR

SEQ ID NO: 77 - protein, 108 aa, synthetic construct
DIQMTQSPSSLSASVGDRVTITCRASQGISNYLAWYQQKPGKAPKLLIYSASNLQSGVPSRFSGSGSGTDFTLTISSLQPEDFATYYCQQSYSTPLTFGGGTKVEIKR

SEQ ID NO: 78 - protein, 5 aa, synthetic construct
SYAIS

SEQ ID NO: 79 - protein, 5 aa, synthetic construct
DYYIQ

SEQ ID NO: 80 - protein, 5 aa, synthetic construct
SYYMH

SEQ ID NO: 81 - protein, 5 aa, synthetic construct
NHYMH

SEQ ID NO: 82 - protein, 17 aa, synthetic construct
IIDPSDGNTNYAQNFQG

SEQ ID NO: 83 - protein, 17 aa, synthetic construct
WINPNSGGTSYAQKFQG

SEQ ID NO: 84 - protein, 17 aa, synthetic construct
GFDPEDGETIYAQKFQG

SEQ ID NO: 85 - protein, 17 aa, synthetic construct
WMNPNSGNTGYAQKFQG

SEQ ID NO: 86 - protein, 13 aa, synthetic construct
ERAAAGYYYYMDV

SEQ ID NO: 87 - protein, 10 aa, synthetic construct
EGDYYYGMDA

SEQ ID NO: 88 - protein, 8 aa, synthetic construct
DQGWGMDV

SEQ ID NO: 89 - protein, 9 aa, synthetic construct
SESGSDLDY

SEQ ID NO: 90 - protein, 11 aa, synthetic construct
RASQSISSYLN

SEQ ID NO: 91 - protein, 16 aa, synthetic construct
RSSQSLLHSNGYNYLD

SEQ ID NO: 92 - protein, 11 aa, synthetic construct
RASQSISSYLN

SEQ ID NO: 93 - protein, 11 aa, synthetic construct
RASQTIGNYVN

SEQ ID NO: 94 - protein, 7 aa, synthetic construct
AASSLQS

SEQ ID NO: 95 - protein, 7 aa, synthetic construct
LGSNRAS

SEQ ID NO: 96 - protein, 7 aa, synthetic construct
AASSLQS

SEQ ID NO: 97 - protein, 7 aa, synthetic construct
GASNLHT

SEQ ID NO: 98 - protein, 9 aa, synthetic construct
QQSYSTPLT

SEQ ID NO: 99 - protein, 9 aa, synthetic construct
MQGLQTPHT

SEQ ID NO: 100 - protein, 9 aa, synthetic construct
QQTYSTPYT

SEQ ID NO: 101 - protein, 9 aa, synthetic construct
QQTYSAPLT

SEQ ID NO: 102 - protein, 122 aa, synthetic construct
QVQLVQSGAEVKKPGASVKVSCKASGGTFSSYAISWVRQAPGQGLEWMGIIDPSDGNTNYAQNFQGRVTMTRDTSTSTVYMELSSLRSEDTAVYYCAKERAAAGYYYYMDVWGQGTTVTVSS

SEQ ID NO: 103 - protein, 119 aa, synthetic construct
QVQLVQSGAEVKKPGASVKVSCKASGYTFTDYYIQWVRQAPGQGLEWMGWINPNSGGTSYAQKFQGRVTMTRDTSTSTVYMELSSLRSEDTAVYYCAKEGDYYYGMDAWGQGTMVTVSS

SEQ ID NO: 104 - protein, 117 aa, synthetic construct
QVQLVQSGAEVKKPGASVKVSCKASGYTFTSYYMHWVRQAPGQGLEWMGGFDPEDGETIYAQKFQGRVTMTRDTSTSTVYMELSSLRSEDTAVYYCARDQGWGMDVWGQGTTVTVSS

SEQ ID NO: 105 - protein, 118 aa, synthetic construct
QVQLVQSGAEVKKPGASVKVSCKASGYTFTNHYMHWVRQAPGQGLEWMGWMNPNSGNTGYAQKFQGRVTMTRDTSTSTVYMELSSLRSEDTAVYYCASSESGSDLDYWGQGTLVTVSS

SEQ ID NO: 106 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 107 - protein, 113 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLQTPHTFGQGTKVEIKR

SEQ ID NO: 108 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQTYSTPYTFGQGTKLEIKR

SEQ ID NO: 109 - protein, 108 aa, synthetic construct
DIQMTQSPSSLSASVGDRVTITCRASQTIGNYVNWYQQKPGKAPKLLIYGASNLHTGVPSRFSGSGSGTDFTLTISSLQPEDFATYYCQQTYSAPLTFGGGTKVEIKR

SEQ ID NO: 110 - protein, 546 aa, Henipavirus nipahense
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 111 - protein, 520 aa, Henipavirus nipahense
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 112 - protein, 83 aa, Henipavirus nipahense
ILHYEKLSKIGLVKGVTRKYKIKSNPLTKDIVIKMIPNVSNMSQCTGSVMENYKTRLNGILTPIKGALEIYKNNTHDLVGDVR

SEQ ID NO: 113 - protein, 437 aa, Henipavirus nipahense
LAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 114 - protein, 500 aa, Henipavirus nipahense
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 115 - protein, 417 aa, Henipavirus nipahense
LAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 116 - protein, 500 aa, Henipavirus nipahense
ILHYEKLSKIGLVKGVTRKYKIKSNPLTKDIVIKMIPNVSNMSQCTGSVMENYKTRLNGILTPIKGALEIYKNQTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 117 - protein, 524 aa, Henipavirus nipahense
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 118 - protein, 498 aa, Henipavirus nipahense
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 119 - protein, 415 aa, Henipavirus nipahense
LAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 120 - protein, 602 aa, Henipavirus nipahense
MGPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 121 - protein, 597 aa, Henipavirus nipahense
MGKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 122 - protein, 592 aa, Henipavirus nipahense
MGNTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 123 - protein, 587 aa, Henipavirus nipahense
MGKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 124 - protein, 582 aa, Henipavirus nipahense
MGSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 125 - protein, 577 aa, Henipavirus nipahense
MGSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 126 - protein, 573 aa, Henipavirus nipahense
MGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 127 - protein, 569 aa, Henipavirus nipahense
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 128 - protein, 546 aa, Henipavirus hendraense
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 129 - protein, 604 aa, Henipavirus hendraense
MMADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 130 - protein, 526 aa, Henipavirus nipahense
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 131 - protein, 526 aa, Henipavirus nipahense
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNQTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTGT

SEQ ID NO: 132 - protein, 569 aa, Henipavirus nipahense
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 133 - protein, 498 aa, Henipavirus nipahense; identical to SEQ ID NO: 118

SEQ ID NO: 134 - protein, 557 aa, Henipavirus cedarense
MSNKRTTVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 135 - protein, 545 aa, Henipavirus sp.
MALNKNMFSSLFLGYLLVYATTVQSSIHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 136 - protein, 662 aa, Jeilongvirus sp.
MKKKTDNPTISKRGHNHSRGIKSRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 137 - protein, 36 aa, synthetic construct
  VARIANT 1..36: note = This sequence may encompass 1-6 GGGGGS repeating units
GGGGGSGGGGGSGGGGGSGGGGGSGGGGGSGGGGGS

SEQ ID NO: 138 - protein, 602 aa, Henipavirus nipahense
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 139 - protein, 622 aa, Henipavirus cedarense
MLSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 140 - protein, 632 aa, Jeilongvirus sp.
MPQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 141 - protein, 625 aa, Henipavirus sp.
MATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 142 - protein, 552 aa, Henipavirus nipahense
FNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 143 - protein, 555 aa, Henipavirus hendraense
FNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 144 - protein, 26 aa, synthetic construct
MVVILDKRCYCNLLILILMISECSVG

SEQ ID NO: 145 - protein, 6 aa, synthetic construct
GGGGGS

SEQ ID NO: 146 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 147 - protein, 5 aa, synthetic construct
GGGGS

SEQ ID NO: 148 - protein, 600 aa, Henipavirus nipahense
PAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 149 - protein, 595 aa, Henipavirus nipahense
KVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 150 - protein, 590 aa, Henipavirus nipahense
NTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 151 - protein, 585 aa, Henipavirus nipahense
KGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 152 - protein, 580 aa, Henipavirus nipahense
SKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 153 - protein, 575 aa, Henipavirus nipahense
SYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 154 - protein, 571 aa, Henipavirus nipahense
TMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQC

SEQ ID NO: 155 - protein, 568 aa, Henipavirus nipahense
KKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQCT

SEQ ID NO: 156 - protein, 603 aa, Henipavirus hendraense
MADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 157 - protein, 568 aa, Henipavirus nipahense
KKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 158 - protein, 621 aa, Henipavirus cedarense
LSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 159 - protein, 631 aa, Jeilongvirus sp.
PQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 160 - protein, 624 aa, Henipavirus sp.
ATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 161 - protein, 530 aa, Henipavirus cedarense
DFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 162 - protein, 640 aa, Jeilongvirus sp.
SRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 163 - protein, 520 aa, Henipavirus hendraense
ILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 164 - protein, 519 aa, Henipavirus sp.
IHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 165 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 166 - protein, 15 aa, synthetic construct
TTAASGSSGGSSSGA

SEQ ID NO: 167 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 168 - protein, 305 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 169 - DNA, 972 bp, Homo sapiens
  source 1..972: mol_type = genomic DNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataa

SEQ ID NO: 170 - DNA, 918 bp, Homo sapiens
  source 1..918: mol_type = genomic DNA
atgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactga

SEQ ID NO: 171 - DNA, 969 bp, synthetic construct
  source 1..969: mol_type = other DNA
atgtggcccctggtcgccgccctgttgctgggctcggcatgctgcggatcagctcagctactgtttaataaaacaaaatctgtagaattcacgttttgtaacgacactgtcgtgatcccatgctttgttactaatatggaggcacaaaacaccactgaagtgtacgtgaagtggaaattcaaaggcagagacatttacacctttgacggcgccctcaacaagtccaccgtgcccactgactttagtagcgcaaaaattgaggtcagccaattactaaaaggagatgcctctttgaagatggacaagagcgatgctgtcagccacacagggaactacacttgtgaagtaacagagttaacccgcgaaggtgaaacgatcatcgagctgaagtatcgagtggtgtcctggttttctccgaacgagaatatccttatcgtaattttcccaattttcgctatcctcctgttctggggccagtttggtatcaagacactcaaatatcggtccggtgggatggatgagaagacaattgccctgcttgttgctggactcgtgatcaccgtcatcgtgattgttggggccatccttttcgtcccaggggagtacagcctgaagaatgctacgggcctgggattaattgtgacctctacagggatactcatcctgcttcactactatgtgttcagtaccgcgattggactgacctccttcgtcattgccatattggtgattcaggtgatagcctacatcctcgccgtggttggcctgagtctctgtatcgcggcgtgcatacccatgcatggccctcttctgatttcagggttgagtatcctcgcactagcacagttgctgggactggtttatatgaaatttgtggcctccaaccagaagactatacagcctcctaggaaggctgtagaggagcccctgaatgcattcaaggaatcaaaaggcatgatgaatgatgaa

SEQ ID NO: 172 - protein, 21 aa, synthetic construct
GSGEGRGSLLTCGDVEENPGP

SEQ ID NO: 173 - protein, 22 aa, synthetic construct
GSGATNFSLLKQAGDVEENPGP

SEQ ID NO: 174 - protein, 23 aa, synthetic construct
GSGQCTNYALLKLAGDVESNPGP

SEQ ID NO: 175 - protein, 25 aa, synthetic construct
GSGVKQTLNFDLLKLAGDVESNPGP

SEQ ID NO: 176 - protein, 7 aa, synthetic construct
RRRRGSG

SEQ ID NO: 177 - protein, 7 aa, synthetic construct
RKRRGSG

SEQ ID NO: 178 - protein, 15 aa, synthetic construct
RKRRGSGTPDPWGSG

SEQ ID NO: 179 - skipped

SEQ ID NO: 180 - RNA, 12 nt, synthetic construct
  source 1..12: mol_type = other RNA
gttttagagcta

SEQ ID NO: 181 - skipped

SEQ ID NO: 182 - RNA, 66 nt, synthetic construct
  source 1..66: mol_type = other RNA
tagcaagttaaaataaggctagtccgttatcaacttgaaaaagtggcaccgagtcggtgctttttt

SEQ ID NO: 183 - skipped

SEQ ID NO: 184 - RNA, 17 nt, synthetic construct
  source 1..17: mol_type = other RNA
gtttsagagctatgctg

SEQ ID NO: 185 - skipped

SEQ ID NO: 186 - RNA, 71 nt, synthetic construct
  source 1..71: mol_type = other RNA
cagcatagcaagttsaaataaggctagtccgttatcaacttgaaaaagtggcaccgagtcggtgctttttt

SEQ ID NO: 187 - skipped

SEQ ID NO: 188 - RNA, 14 nt, synthetic construct
  source 1..14: mol_type = other RNA
gttttagtactctg

SEQ ID NO: 189 - skipped

SEQ ID NO: 190 - RNA, 64 nt, synthetic construct
  source 1..64: mol_type = other RNA
cagaatctactaaaacaaggcaaaatgccgtgtttatctcgtcaacttgttggcgagatttttt

SEQ ID NO: 191 - RNA, 88 nt, synthetic construct
  source 1..88: mol_type = other RNA
gtcgtctataggacggcgaggacaacgggaagtgccaatgtgctctttccaagagcaaacaccccgttggcttcaagatgaccgctcg

SEQ ID NO: 192 - skipped

SEQ ID NO: 193 - RNA, 23 nt, synthetic construct
  source 1..23: mol_type = other RNA
cgagcggtctgagaagtggcact

SEQ ID NO: 194 - skipped

SEQ ID NO: 195 - protein, 223 aa, synthetic construct
TTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 196 - protein, 190 aa, synthetic construct
ESKYGPPCPPCPIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 197 - protein, 193 aa, synthetic construct
ESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 198 - protein, 226 aa, synthetic construct
TTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 199 - protein, 220 aa, synthetic construct
AAAIEVMYPPPYLDNEKSNGTIIHVKGKHLCPSPLFPGPSKPIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 200 - protein, 223 aa, synthetic construct
AAAIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 201 - protein, 222 aa, synthetic construct
AAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 202 - protein, 222 aa, synthetic construct
AAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

(19) United States
(12) Patent Application Publication
(10) Pub. No.: US 2025/0177447 A1
(43) Pub. Date: Jun. 5, 2025

(54) GPRC5D-SPECIFIC ANTIBODY CONSTRUCTS AND COMPOSITIONS THEREOF

(71) Applicant: Sana Biotechnology, Inc., Seattle, WA (US)

(72) Inventors: Jeremy M. KINDER, Seattle, WA (US); Adam J. JOHNSON, Seattle, WA (US); Terry J. FRY, Seattle, WA (US)

(21) Appl. No.: 18/945,839

(22) Filed: Nov. 13, 2024

Related U.S. Application Data
(60) Provisional application No. 63/548,515, filed on Nov. 14, 2023.

(51) Int. Cl. A61K 35/17; A61K 40/11; A61K 40/31; A61K 40/42; A61P 35/00; C07K 16/28; C12N 15/86

(52) U.S. Cl. CPC A61K 35/17; A61K 40/11; A61K 40/31; A61K 40/4202; A61P 35/00; C07K 16/28; C12N 15/86; A61K 2239/13; A61K 2239/17; A61K 2239/21; A61K 2239/22; C07K 2317/31; C07K 2317/565; C12N 2740/15022; C12N 2740/15043

(57) ABSTRACT

Disclosed herein are antibodies or antigen binding fragments that specifically bind human GPRC5D. Also disclosed are chimeric antigen receptors and chimeric antigen receptor transgenes comprising an antigen binding domain that specifically binds human GPRC5D and fusion proteins comprising a Henipavirus glycoprotein G and a GPRC5D antibody, or an antigen binding fragment thereof. Viral vectors and other compositions containing the antibodies or antigen binding fragments thereof, chimeric antigen receptors and chimeric antigen receptor transgenes, and fusion proteins are disclosed. The present disclosure additionally relates to cells expressing chimeric antigen receptors, as well as methods of delivering the various antibodies and chimeric antigen receptors and methods of using cells expressing the chimeric antigen receptors.

### CROSS-REFERENCE TO RELATED APPLICATIONS

This application claims priority under 35 U.S.C. § 119(e) to U.S. Provisional Application No. 63/548,515, filed Nov. 14, 2023. The contents of this application are incorporated herein by reference in its entirety

### SEQUENCE LISTING

The instant application contains a Sequence Listing which has been submitted electronically in XML format and is hereby incorporated by reference in its entirety. Said XML copy, created on Nov. 9, 2024, is named 15147_0009-00000_SL, and is 208,896 bytes in size.

### FIELD

The present disclosure relates to antibodies or antigen binding fragments thereof that specifically bind human GPRC5D. Also disclosed are chimeric antigen receptors and chimeric antigen receptor transgenes comprising an antigen binding domain that specifically binds human GPRC5D. Also disclosed are fusion proteins comprising a Henipavirus glycoprotein G and a GPRC5D antibody, or an antigen binding fragment thereof. Viral vectors and other compositions containing the antibodies or antigen binding fragments thereof, chimeric antigen receptors and chimeric antigen receptor transgenes, and fusion proteins are disclosed. The present disclosure additionally relates to cells expressing chimeric antigen receptors, as well as methods of delivering the various antibodies and chimeric antigen receptors and methods of using cells expressing the chimeric antigen receptors.

### INTRODUCTION

G-protein couple receptor family C group 5 member D (GPRC5D) is an orphan G protein-coupled receptor whose function is not well understood. GPRC5D expression has been noted in plasma cells, and expression has also been linked to multiple myeloma.

T lymphocytes are among the prime targets in gene therapy, even more so since chimeric antigen receptor (CAR) T cells have reached the clinic. Genetically modifying T cells with CAR constructs is the most common approach to creating tumor-specific T cells. The use of modified T cells is an emerging cell therapy approach within the area of adoptive cell transfer (ACT). This approach involves collecting T cells from a patient (autologous) or healthy donors (allogeneic), genetically modifying or engineering these T cells, and transferring the modified or engineered T cells into the patient to treat a range of diseases. The use of allogeneic T cells has several advantages over the use of autologous T cells, as the latter suffers from challenges such as a patient having insufficient healthy T cells for harvesting and the patient experiencing disease progression, co-morbidities, or even death in the time it takes to manufacture the engineered T cells. Methods that efficiently produce effective CAR-T cells, including allogeneic CAR-T cells, targeting specific tumor antigens are needed. The present disclosure addresses this need.

There is a significant unmet need for novel CAR-T cells designed to treat B cell malignancies, including multiple myeloma. Of those receiving CAR-T therapies, many do not respond to treatment or relapse. Further, many receiving CAR-T therapies develop humoral immunity against available CAR-T therapeutics. For many patients, current manufacturing methods and capabilities present significant challenges for availability and access of CAR-T therapeutics, including those targeting B-cell malignancies. Thus, novel CAR-T cells for the treatment of patients with B-cell malignancies, like multiple myeloma, through the targeting of GPRC5D and other potential secondary antigens are needed.

### BRIEF SUMMARY

The present disclosure provides an isolated polypeptide that specifically binds G protein-coupled receptor class C group 5 member D (GPRC5D). In some embodiments, the isolated polypeptide comprises certain heavy chain variable regions (VH) and/or certain light chain variable regions (VL). In some embodiments, the isolated polypeptide comprises certain heavy chain complementarity determining regions (HCDR1, HCDR2, and HCDR3) and/or certain light chain complementarity determining regions (LCDR1, LCDR2, and LCDR3).

The present disclosure provides an antibody or antigen binding fragment thereof that specifically binds G protein-coupled receptor class C group 5 member D(GPRC5D). In some embodiments, the antibody or antigen binding fragment thereof comprises certain heavy chain variable regions (VH) and/or certain light chain variable regions (VL). In some embodiments, the antibody or antigen binding fragment thereof comprises certain heavy chain complementarity determining regions (HCDR1, HCDR2, and HCDR3) and/or certain light chain complementarity determining regions (LCDR1, LCDR2, and LCDR3). The disclosure likewise provides for isolated polynucleotides, vectors, and host cells comprising the anti-GPRC5D antibody or antigen binding fragment thereof.

The present disclosure also provides a chimeric antigen receptor (CAR) that specifically binds G protein-coupled receptor class C group 5 member D (GPRC5D). In some embodiments, the CAR comprises at least one of a signal peptide, an extracellular binding domain, a hinge domain, a transmembrane domain, an intracellular costimulatory domain, and/or an intracellular signaling domain. In some embodiments, the CAR extracellular binding domain comprises an antigen binding domain that comprises the antibody or antigen binding fragment thereof disclosed herein. The disclosure likewise provides for isolated polynucleotides, vectors, and host cells comprising the anti-GPRC5D CAR.

The present disclosure also provides a viral vector targeting an immune cell, wherein the vector comprises an antibody or antigen binding fragment thereof that binds to a cell surface molecule on the immune cell and at least one polynucleotide encoding a chimeric antigen receptor (CAR) as disclosed herein. In some embodiments, the antibody or antigen binding fragment thereof binds to CD4 or CD8. In some embodiments, the vector comprises a henipavirus F protein molecule or a biologically active portion thereof. In some embodiments, the vector comprises a henipavirus envelope glycoprotein G (G protein) or a biologically active portion thereof. In some embodiments, the antibody or antigen binding fragment thereof that binds to a cell surface molecule is attached to a membrane-bound protein in the viral vector envelope. In some embodiments, the antibody or antigen binding fragment thereof that binds to a cell surface molecule is attached to a fusogen on the outer surface of the viral vector

The present disclosure also provides a fusion protein comprising a henipavirus envelope glycoprotein G (G protein) or a biologically active portion thereof and an anti-GPRC5D antibody or antigen binding fragment thereof as herein disclosed.

The present disclosure provides a method for selectively modulating the activity of an immune cell, comprising delivery to an immune cell an effective amount of a viral vector comprising a polynucleotide encoding a CAR as disclosed herein. The present disclosure also provides a method for producing a chimeric antigen receptor (CAR) immune cell, comprising delivery to an immune cell an effective amount of a viral vector comprising a polynucleotide encoding a CAR as disclosed herein. In some embodiments, the immune cell is a T cell. In some embodiments the T cell is a primary T cell. In some embodiments, the polynucleotide encoding a CAR as disclosed herein is inserted into a site-specific locus. In some embodiments, the polynucleotide encoding a CAR as disclosed herein is inserted by homology-directed repair. In some embodiments, the immune cell expresses one or more CARs as disclosed herein.

The present disclosure additionally provides an engineered cell, comprising a CAR as herein disclosed and one or more modifications that (i) reduce expression of one or more MHC class I molecules and/or one or more MHC class II molecules, and/or (ii) increase expression of one of more tolerogenic factors, wherein the reduced expression of (i) and the increase expression of (ii) is relative to a cell of the same cell type that does not comprise the modifications.

The present disclosure additionally provides a method of administering to a subject in need thereof an effective amount of the CAR cells disclosed herein. The present disclosure also provides a method for treating a disease in a subject. The present disclosure provides a population of immune cells expressing the CARs disclosed herein. The present disclosure provides a composition of immune cells expressing the CARs disclosed herein. The present disclosure also provides a pharmaceutical composition of immune cells expressing the CARs disclosed herein. The present disclosure provides the use of the cells or the method disclosed herein for the treatment of a disease. In some embodiments, the disease is cancer. In some embodiments, the cancer is a hematologic malignancy. In some embodiments, the cancer is a solid malignancy.

### BRIEF DESCRIPTION OF DRAWINGS

FIGS. 1A-1B depict the cytolytic activity of the disclosed chimeric antigen receptors in MM.1S (1A) and RPMI8226 (1B) cells.

FIGS. 2A-2F show the effector cytokine production of disclosed chimeric antigen receptors compared against a benchmark chimeric antigen receptor.

FIGS. 3A-3D depict in vitro cytotoxicity and expansion kinetics using GPRC5D-directed CAR-T cells in two different tumor cell lines.

FIGS. 4A-4D depict the in vitro cytotoxicity of GPRC5D-directed CAR-T cells against two different tumor cell lines.

FIG. 5A depicts in vitro control of tumor cell growth using GPRC5D-directed CAR-T cells.

FIGS. 5B-5G depict in vivo tumor growth as measured by flux.

FIG. 6 depicts the component make up of various CAR constructs.

FIGS. 7A-7B depicts the cytolytic activity of CAR T cells generated from apheresis sample 1 that express the disclosed chimeric antigen receptors. Cytolytic activity is accessed for MM.1S cells (7A) and NCI-H929 (7B) cells.

FIGS. 8A-8B depicts the cytolytic activity of CAR T cells generated from apheresis sample 2 that express the disclosed chimeric antigen receptors. Cytolytic activity is accessed for MM.1S cells (8A) and NCI-H929 (8B) cells.

FIG. 9 depicts the experimental design of a serial tumor challenge using CAR T cells that express the disclosed chimeric antigen receptors.

FIGS. 10A-10E depict in vitro expansion kinetics using GPRC5D-directed CAR-T cells generated from apheresis sample 1. The effector:target ratio was 1:1 (10A), 1:2 (10B), 1:4 (10C), 1:8 (10D); or 1:16 (10E).

FIGS. 11A-11E depict in vitro expansion kinetics using GPRC5D-directed CAR-T cells generated from apheresis sample 2. The effector:target ratio was 1:1 (11A), 1:2 (11B), 1:4 (11C), 1:8 (11D); or 1:16 (11E).

FIGS. 12A-12E depict in vitro cytotoxicity kinetics using GPRC5D-directed CAR-T cells generated from apheresis sample 1. The effector:target ratio was 1:1 (12A), 1:2 (12B), 1:4 (12C), 1:8 (12D); or 1:16 (12E).

FIGS. 13A-13E depict in vitro cytotoxicity kinetics using GPRC5D-directed CAR-T cells generated from apheresis sample 2. The effector:target ratio was 1:1 (13A), 1:2 (13B), 1:4 (13C), 1:8 (13D); or 1:16 (13E).

FIGS. 14A-14D show the GM-CSF production of disclosed chimeric antigen receptors. FIG. 14A shows GM-CSF production for CAR-T cells generated from apheresis sample 1, E:T ratio 1:4. FIG. 14B shows GM-CSF production for CAR-T cells generated from apheresis sample 1, E:T ratio 1:8. FIG. 14C shows GM-CSF production for CAR-T cells generated from apheresis sample 2, E:T ratio 1:4. FIG. 14D shows GM-CSF production for CAR-T cells generated from apheresis sample 2, E:T ratio 1:8.

FIGS. 15A-15D show the Granzyme B production of disclosed chimeric antigen receptors. FIG. 15A shows Granzyme B production for CAR-T cells generated from apheresis sample 1, E:T ratio 1:4. FIG. 15B shows Granzyme B production for CAR-T cells generated from apheresis sample 1, E:T ratio 1:8. FIG. 15C shows Granzyme B production for CAR-T cells generated from apheresis sample 2, E:T ratio 1:4. FIG. 15D shows Granzyme B production for CAR-T cells generated from apheresis sample 2, E:T ratio 1:8.

FIGS. 16A-16D show the IFN-γ production of disclosed chimeric antigen receptors. FIG. 16A shows IFN-γ production for CAR-T cells generated from apheresis sample 1, E:T ratio 1:4. FIG. 16B shows IFN-γ production for CAR-T cells generated from apheresis sample 1, E:T ratio 1:8. FIG. 16C shows IFN-γ production for CAR-T cells generated from apheresis sample 2, E:T ratio 1:4. FIG. 16D shows IFN-γ production for CAR-T cells generated from apheresis sample 2, E:T ratio 1:8.

FIGS. 17A-17D show the IL-2 production of disclosed chimeric antigen receptors. FIG. 17A shows IL-2 production for CAR-T cells generated from apheresis sample 1, E:T ratio 1:4. FIG. 17B shows IL-2 production for CAR-T cells generated from apheresis sample 1, E:T ratio 1:8. FIG. 17C shows IL-2 production for CAR-T cells generated from apheresis sample 2, E:T ratio 1:4. FIG. 17D shows TL-2 production for CAR-T cells generated from apheresis sample 2, E:T ratio 1:8.

FIGS. 18A-18D show the TNF-α production of disclosed chimeric antigen receptors. FIG. 18A shows TNF-α production for CAR-T cells generated from apheresis sample 1, E:T ratio 1:4. FIG. 18B shows TNF-α production for CAR-T cells generated from apheresis sample 1, E:T ratio 1:8. FIG. 18C shows TNF-α production for CAR-T cells generated from apheresis sample 2, E:T ratio 1:4. FIG. 18D shows TNF-α production for CAR-T cells generated from apheresis sample 2, E:T ratio 1:8.

FIGS. 19A-19D show the IL-5 production of disclosed chimeric antigen receptors. FIG. 19A shows IL-5 production for CAR-T cells generated from apheresis sample 1, E:T ratio 1:4. FIG. 19B shows IL-5 production for CAR-T cells generated from apheresis sample 1, E:T ratio 1:8. FIG. 19C shows IL-5 production for CAR-T cells generated from apheresis sample 2, E:T ratio 1:4. FIG. 19D shows IL-5 production for CAR-T cells generated from apheresis sample 2, E:T ratio 1:8.

FIGS. 20A-20D show the IL-17a production of disclosed chimeric antigen receptors. FIG. 20A shows IL-17a production for CAR-T cells generated from apheresis sample 1, E:T ratio 1:4. FIG. 20B shows IL-17a production for CAR-T cells generated from apheresis sample 1, E:T ratio 1:8. FIG. 20C shows IL-17a production for CAR-T cells generated from apheresis sample 2, E:T ratio 1:4. FIG. 20D shows IL-17a production for CAR-T cells generated from apheresis sample 2, E:T ratio 1:8.

### DETAILED DESCRIPTION

Unless defined otherwise, all terms of art, notations, and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. Unless defined otherwise, all technical and scientific terms, acronyms, and abbreviations used herein have the same meaning as commonly understood by one of ordinary skill in the art to which the disclosure pertains. Unless indicated otherwise, abbreviations and symbols for chemical and biochemical names is per IUPAC-IUB nomenclature. Unless indicated otherwise, all numerical ranges are inclusive of the values defining the range as well as all integer values in-between.

As used herein, the articles “a” and “an” refer to one or to more than one (i.e., to at least one) of the grammatical object of the article. By way of example, “an element” means one element or more than one element.

As used herein, the term “about” will be understood by persons of ordinary skill in the art and will vary to some extent on the context in which it is used. In some embodiments, the term “about” when referring to a measurable value such as an amount, a temporal duration, and the like, is meant to encompass art-accepted variations based on standard errors in making such measurements. In some embodiments, the term “about” when referring to such values, is meant to encompass variations of 20% or ±10%, more preferably +5%, even more preferably +1%, and still more preferably ±0.1% from the specified value, as such variations are appropriate to perform the disclosed methods.

As used herein, “GPRC5D” or “G protein-coupled receptor class C group 5 member D” refers to a transmembrane glycoprotein which is expressed on cells of B cell lineage.

As used herein, “CD4” or “cluster of differentiation 4” refers to a transmembrane glycoprotein which is a specific marker for a subclass of T cells (which includes helper T cells). The CD4 protein acts as a co-receptor together with the T cell receptor (TCR) to recognize antigen presentation by NMC class II cells. CD4 plays a role in the development of T cells and activation of mature T cells.

As used herein, “CD8” or “cluster of differentiation 8” refers to a transmembrane glycoprotein which is a specific marker for a subclass of T cells (which includes cytotoxic T cells). CD8 assembles as either a heterodimer of the CD8 alpha (“CD8α” or “CD8A”) and CD8 beta (“CD8β” or “CD8B”) subunits (“CD8αβ” or “CD8AB”), or a CD8 alpha homodimer (“CD8aa” or “CD8AA”). The assembled dimeric CD8 complex acts as a co-receptor together with the T cell receptor (TCR) to recognize antigen presentation by NMC class I cells. CD8 plays a role in the development of T cells and activation of mature T cells.

As used herein, “affinity” refers to the strength of the sum total of noncovalent interactions between a single binding site of a molecule (e.g., an antibody) and its binding partner (e.g., an antigen). The affinity of a molecule for its partner can generally be represented by the equilibrium dissociation constant (K_{D}) (or its inverse equilibrium association constant, K_{A}). Affinity can be measured by common methods known in the art, including those described herein. See, for example, Pope M. E., Soste M. V., Eyford B. A., Anderson N. L., Pearson T. W., (2009) J. Immunol. Methods. 341(1-2):86-96 and methods described therein.

As used herein, “antibody” is meant in a broad sense and includes immunoglobulin molecules including monoclonal antibodies including murine, human, humanized and chimeric antibodies, antibody fragments, bispecific or multispecific antibodies formed from at least two intact antibodies or antibody fragments, dimeric, tetrameric or multimeric antibodies, single chain antibodies, and any other modified configuration of the immunoglobulin molecule that comprises an antigen recognition site of the required specificity.

Immunoglobulins can be assigned to five major classes, namely IgA, IgD, IgE, IgG, and IgM, depending on the heavy chain constant domain amino acid sequence. IgA and IgG are further sub-classified to IgA1, IgA2, IgG1, IgG2, IgG3, and IgG4. Antibody light chains of any vertebrate species can be assigned to one of two clearly distinct types, namely kappa (κ) and lambda (A), based on the amino acid sequences of their constant domains.

The term “antigen” refers to an immunogenic molecule that provokes an immune response. This immune response involves antibody production, activation of specific immunologically competent cells, or both. An antigen is, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can also be produced by cells that have been modified or genetically engineered to express an antigen.

As used herein, “antigen binding fragment” or “antibody fragment” refers to a portion of an immunoglobulin molecule that retains the heavy chain and/or the light chain antigen binding site, such as a heavy chain complementarity determining regions (HCDR) 1 (HCDR1), 2 (HCDR2), and 3 (HCDR3), a light chain complementarity determining regions (LCDR) 1 (LCDR1), 2 (LCDR2), and 3 (LCDR3), a heavy chain variable region (VH), or a light chain variable region (VL). Antibody fragments include a Fab fragment (a monovalent fragment consisting of the VL or the VH); a F(ab) 2 fragment (a bivalent fragment comprising two Fab fragments linked by a disulfide bridge at the hinge region); a Fd fragment consisting of the VH and CH1 domains; a Fv fragment consisting of the VL and VH domains of a single arm of an antibody; a dAb fragment, which consists of a VH domain; and a variable domain (VHH) from, e.g., human or camelid origin. VH and VL domains are engineered and linked together via a synthetic linker to form various types of single chain antibody designs in which the VH/VL domains pair intramolecularly, or intermolecularly in those embodiments in which the VH and VL domains are expressed by separate single chain antibody constructs, to form a monovalent antigen binding site, such as a single-chain Fv (scFv) or diabody. These antibody fragments are obtained using well known techniques and the fragments are characterized in the same manner as are intact antibodies.

An antibody variable region consists of a “framework” region interrupted by three “antigen binding sites.” The antigen binding sites are defined using various terms, including, for example (i) “Complementarity Determining Regions” (CDRs), three in the VH (HCDR1, HCDR2, HCDR3) and three in the VL (LCDR1, LCDR2, LCDR3) (Wu and Kabat, JExpMed 132:211-50, 1970; Kabat et al., Sequences of Proteins of Immunological Interest, 5th Ed. Public Health Service, National Institutes of Health, Bethesda, Md., 1991), and (ii) “Hypervariable regions,” “HVR,” or “HV,” three in the VH (H1, H2, H3) and three in the VL (L1, L2, L3) (Chothia and LeskMolBiol 196:901-17, 1987). Other terms include “IMGT-CDRs” (Lefranc et al., Dev Comparat Immunol 27:55-77, 2003) and “Specificity Determining Residue Usage” (SDRU) (Almagro Mol Recognit, 17:132-43, 2004). The International ImMunoGeneTics (IMGT) database (http://www_imgt org) provides a standardized numbering and definition of antigen-binding sites. The correspondence between CDRs, HVs, and IMGT delineations is described in Lefranc et al., Dev Comparat Immunol 27:55-77, 2003.

The term “framework,” or “FR” or “framework sequence” refers to the remaining sequences of a variable region other than those sequences defined to be antigen binding sites. Because the antigen binding site can be defined by various terms as described above, the exact amino acid sequence of a framework depends on how the antigen-binding site was defined.

A “binding domain,” also referred to as a “binding region,” refers to an antibody or portion thereof that possesses the ability to specifically and non-covalently associate, unite, or combine with a target. A binding domain includes any naturally occurring, synthetic, semi-synthetic, or recombinantly produced binding partner for a biological molecule, a molecular complex, or other target of interest. Exemplary binding domains include receptor ectodomains, ligands, scFvs, disulfide linked Fvs, sdAbs, VHH antibodies, Fab fragments, Fab′ fragments, F(ab′)2 fragments, diabodies, or other synthetic polypeptides selected for their specific ability to bind to a biological molecule, a molecular complex, or other target of interest.

The term “CDR” denotes a complementarity determining region as defined by at least one manner of identification to one of skill in the art. The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Martin et al., “Modeling antibody hypervariable loops: a combined algorithm,” PNAS, 1989, 86(23):9268-9272, (“AbM” numbering scheme).

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

In some embodiments, CDRs can be defined in accordance with any of the Chothia numbering schemes, the Kabat numbering scheme, the IMGT numbering scheme, a combination of Kabat, IMGT, and Chothia, the AbM definition, and/or the contact definition. A sdAb variable domain comprises three CDRs, designated CDR1, CDR2, and CDR3. Table 1 lists exemplary position boundaries of CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-H1 located before CDR-H1, FR-H2 located between CDR-H1 and CDR-H2, FR-H3 located between CDR-H2 and CDR-H3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes. For example, where it is stated that a particular CDR (e.g., a CDR-H3) contains the amino acid sequence of a corresponding CDR in a given sdAb amino acid sequence, it is understood that such a CDR has a sequence of the corresponding CDR (e.g., CDR-H3) within the sdAb, as defined by any of the aforementioned schemes. It is understood that any antibody, such as a sdAb, includes CDRs and such are identified according to any of the other aforementioned numbering schemes or other numbering schemes known to a skilled artisan.

As used herein, “Fv” refers to the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) may have the ability to recognize and bind an antigen, although at a lower affinity than the entire binding site.

As used herein, “single-chain Fv” or “scFv” antibody fragments comprise the VH and VL domains of an antibody, wherein these domains are present in a single polypeptide chain. Preferably, the Fv polypeptide further comprises a polypeptide linker between the VH and VL domains which enables the scFv to form the desired structure for antigen binding. For a review of scFv see Pluckthun in The Pharmacology of Monoclonal Antibodies, vol. 113, Rosenburg and Moore eds., Springer-Verlag, New York, pp. 269-315 (1994).

As used herein, “VHH” or “VHH antibodies” refer to single domain antibodies that consist of the variable region of a heavy chain of an IgG antibody. For example, the terms “VHH” and “VHH antibody” can refer to the antigen binding domain of a heavy chain IgG (hcIgG) molecule produced by a Camelidae family mammal (e.g., llamas, camels, and alpacas).

As used herein, the term “specifically binds” to a target molecule, such as an antigen, means that a binding molecule, such as a single domain antibody (sdAb), reacts or associates more frequently, more rapidly, with greater duration, and/or with greater affinity with a particular target molecule than it does with alternative molecules. A binding molecule, such as a sdAb or scFv, “specifically binds” to a target molecule if it binds with greater affinity, avidity, more readily, and/or with greater duration than it binds to other molecules. It is understood that a binding molecule, such as a sdAb or scFv, that specifically binds to a first target may or may not specifically bind to a second target. As such, “specific binding” does not necessarily require (although it can include) exclusive binding.

As used herein, the term “cell surface molecule” means a molecule that is present on the outer surface of a cell. In some embodiments, the cell surface molecule is an antigen, as herein defined and disclosed. In some embodiments, the cell surface molecule is, for example, a peptide, glycopeptide, polypeptide, glycopolypeptide, polynucleotide, polysaccharide, lipid, or the like that is not immunogenic.

As used herein, “percent (%) amino acid sequence identity” and “homology” with respect to a peptide, polypeptide or antibody sequence are used interchangeably and are defined as the percentage of amino acid residues in a candidate sequence that are identical with the amino acid residues in another peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN (DNASTAR) software. Those skilled in the art can determine appropriate parameters for measuring alignment, including any algorithms needed to achieve maximal alignment over the full length of the sequences being compared.

An amino acid substitution may include but is not limited to the replacement of one amino acid in a polypeptide with another amino acid. Exemplary substitutions are shown in Table 2. Amino acid substitutions are introduced into an antibody of interest and the products screened for a desired activity, for example, retained/improved binding.

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class. The term, “corresponding to” with reference to nucleotide or amino acid positions of a sequence, such as set forth in the Sequence Listing, refers to nucleotides or amino acid positions identified upon alignment with a target sequence based on structural sequence alignment or using a standard alignment algorithm, such as the GAP algorithm. For example, corresponding residues of a similar sequence (e.g., fragment or species variant) can be determined by alignment to a reference sequence by structural alignment methods. By aligning the sequences, one skilled in the art can identify corresponding residues, for example, using conserved and identical amino acid residues as guides.

The term “construct” refers to any polynucleotide that contains a recombinant nucleic acid molecule. A construct is present in a vector (e.g., a bacterial vector, a viral vector) or is integrated into a genome. A “vector” is a nucleic acid molecule that is capable of introducing a specific nucleic acid sequence into a cell or into another nucleic acid sequence, or as a means of transporting another nucleic acid molecule. Vectors are, for example, plasmids, cosmids, viruses, an RNA vector, or a linear or circular DNA or RNA molecule that may include chromosomal, non-chromosomal, semi-synthetic, or synthetic nucleic acid molecules. Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, “polypeptide” refers to a polymer comprising amino acids that are linked together. In some embodiments, a polypeptide is a linear polymer of nucleic acids in a chain. In some embodiments, a polypeptide is a polymer of nucleic acids that is folded into a structure or shape.

The term “hypoimmunogenicity,” “hypoimmunogeneic,” “hypoimmunogenic,” “hypoimmunity,” or “hypoimmune” is used interchangeably to describe a cell being less prone to immune rejection by a subject into which such cell is transplanted. For example, relative to an unaltered or unmodified wild-type cell, such a hypoimmunogenic cell is about 2.5%, 5%, 10%, 20%, 30%, 40%, 50%, 60%, 70%, 80%, 90%, 95%, 97.5%, 99% or more less prone to immune rejection by a subject into which such cell is transplanted. In some examples described herein, genome editing technologies are used to modulate the expression of MHC I and/or MHC II genes, and thus, to generate a hypoimmunogenic cell. In other examples described herein, a tolerogenic factor is introduced into a cell and when expressed can modulate or affect the ability of the cell to be recognized by host immune system and thus confer hypoimmunogenicity. Hypoimmunogenicity of a cell is determined by evaluating the cell's ability to elicit adaptive and innate immune responses. Such immune response can be measured using assays recognized by those skilled in the art, for example, by measuring the effect of a hypoimmunogenic cell on T cell proliferation, T cell activation, T cell killing, NK cell proliferation, NK cell activation, and macrophage activity. Hypoimmunogenic cells may undergo decreased killing by T cells and/or NK cells upon administration to a subject or show decreased macrophage engulfment compared to an unmodified or wildtype cell. In some embodiments, a hypoimmunogenic cell elicits a reduced or diminished immune response in a recipient subject compared to a corresponding unmodified wild-type cell. In some embodiments, a hypoimmunogenic cell is nonimmunogenic or fails to elicit an immune response in a recipient subject.

The term “isolated” as used herein refers to a molecule that has been separated from at least some of the components with which it is typically found in nature or produced. For example, a polypeptide is referred to as “isolated” when it is separated from at least some of the components of the cell in which it was produced. When a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced. Thus, a DNA polynucleotide that is contained in a vector inside a host cell is referred to as “isolated.”

As used herein, “lipid particle” refers to any biological or synthetic particle that contains a bilayer of amphipathic lipids enclosing a lumen or cavity. Typically, a lipid particle does not contain a nucleus. Examples of lipid particles include nanoparticles, viral-derived particles, or cell-derived particles. Such lipid particles include, but are not limited to, viral particles (e.g., lentiviral particles), virus-like particles, viral vectors (e.g., lentiviral vectors), exosomes, enucleated cells, vesicles (e.g., microvesicles, membrane vesicles, extracellular membrane vesicles, plasma membrane vesicles, and giant plasma membrane vesicles), apoptotic bodies, mitoparticles, pyrenocytes, or lysosomes. In some embodiments, a lipid particle is a fusosome. In some embodiments, the lipid particle is not a platelet.

As used herein a “biologically active portion,” such as with reference to a protein such as a G protein or an F protein, refers to a portion of the protein that exhibits or retains an activity or property of the full-length of the protein. For example, a biologically active portion of an F protein retains fusogenic activity in conjunction with the G protein when each are embedded in a lipid bilayer. A biologically active portion of the G protein retains fusogenic activity in conjunction with an F protein when each is embedded in a lipid bilayer. In some embodiments, the retained activity includes 10%-150% or more of the activity of a full-length or wild-type F protein or G protein. Examples of biologically active portions of F and G proteins include truncations of the cytoplasmic domain, e.g., truncations of up to 1, 2, 3, 4, 5, 6, 7, 8 9, 10, 11, 12, 13, 14, 15, 20, 22, 25, 30, 33, 34, 35, or more contiguous amino acids, see e.g. Khetawat and Broder 2010 Virology Journal 7:312; Witting et al. 2013 Gene Therapy 20:997-1005; published international; patent application No. WO/2013/148327.

As used herein, “G protein” refers to a henipavirus envelope attachment glycoprotein G or biologically active portion thereof “F protein” refers to a henipavirus fusion protein F or biologically active portion thereof. In some embodiments, the F and G proteins are from a Hendra (HeV) or a Nipah (NiV) virus, and are a wild-type protein or are a variant thereof that exhibits reduced binding for the native binding partner. The F (fusion) and G (attachment) glycoproteins mediate cellular entry of Nipah virus. The G protein initiates infection by binding to the cellular surface receptor ephrin-B2 (EphB2) or EphB3. The subsequent release of the viral genome into the cytoplasm is mediated by the action of the F protein, which induces the fusion of the viral envelope with cellular membranes. In some embodiments, the efficiency of transduction of targeted lipid particles is improved by engineering hyperfusogenic mutations in one or both of the F protein (such as NiV-F) and G protein (such as NiV-G).

As used herein, “fusosome” refers to a particle containing a bilayer of amphipathic lipids enclosing a lumen or cavity and a fusogen that interacts with the amphipathic lipid bilayer. In some embodiments, the fusosome comprises a nucleic acid. In some embodiments, the fusosome is a membrane enclosed preparation. In some embodiments, the fusosome is derived from a source cell. As used herein, “fusosome composition” refers to a composition comprising one or more fusosomes.

As used herein, “fusogen” refers to an agent or molecule that creates an interaction between two membrane enclosed lumens. In embodiments, the fusogen facilitates fusion of the membranes. In other embodiments, the fusogen creates a connection, e.g., a pore, between two lumens (e.g., a lumen of a retroviral vector and a cytoplasm of a target cell). In some embodiments, the fusogen comprises a complex of two or more proteins, e.g., wherein neither protein has fusogenic activity alone. In some embodiments, the fusogen comprises a targeting domain.

As used herein, a “re-targeted fusogen” refers to a fusogen that comprises a targeting moiety having a sequence that is not part of the naturally-occurring form of the fusogen. In embodiments, the fusogen comprises a different targeting moiety relative to the targeting moiety in the naturally-occurring form of the fusogen. In embodiments, the naturally occurring form of the fusogen lacks a targeting domain, and the re-targeted fusogen comprises a targeting moiety that is absent from the naturally occurring form of the fusogen. In embodiments, the fusogen is modified to comprise a targeting moiety. In embodiments, the fusogen comprises one or more sequence alterations outside of the targeting moiety relative to the naturally occurring form of the fusogen, e.g., in a transmembrane domain, fusogenically active domain, or cytoplasmic domain.

As used herein, a “targeted envelope protein” refers to a polypeptide that contains a henipavirus G protein (G protein) attached to a single domain antibody (sdAb) variable domain, such as a VL or VH sdAb, a scFv, a nanobody, a camelid VHH domain, a shark IgNAR, or fragments thereof, that target a molecule on a desired cell type. In some such embodiments, the attachment is directly or indirectly via a linker, such as a peptide linker. The “targeted envelope protein” may also be referred to as a “fusion protein” comprising the G protein and antibodies or antigen binding fragments of the disclosure in which the antibody or antigen binding fragment is fused to the C-terminus of the G protein or a biologically active portion thereof.

As used herein, a “targeted lipid particle” refers to a lipid particle that contains a targeted envelope protein embedded in the lipid bilayer, e.g., a targeted envelope protein targeting CD4 or CD8. Such targeted lipid particles are any lipid particle as herein disclosed, e.g., a viral particle, a virus-like particle, a nanoparticle, a vesicle, an exosome, a dendrimer, a lentivirus, a viral vector, an enucleated cell, a microvesicle, a membrane vesicle, an extracellular membrane vesicle, a plasma membrane vesicle, a giant plasma membrane vesicle, an apoptotic body, a mitoparticle, a pyrenocyte, a lysosome, another membrane enclosed vesicle, or a lentiviral vector, a viral based particle, a virus like particle (VLP), or a cell derived particle.

As used herein, a “retroviral nucleic acid” refers to a nucleic acid containing at least the minimal sequence requirements for packaging into a retrovirus or retroviral vector, alone or in combination with a helper cell, helper virus, or helper plasmid. In some embodiments, the retroviral nucleic acid further comprises or encodes an exogenous agent, a positive target cell-specific regulatory element, a non-target cell-specific regulatory element, or a negative TCSRE. In some embodiments, the retroviral nucleic acid comprises one or more of (e.g., all of) a 5′ LTR (e.g., to promote integration), U3 (e.g., to activate viral genomic RNA transcription), R (e.g., a Tat-binding region), U5, a 3′ LTR (e.g., to promote integration), a packaging site (e.g., psi (Y)), and RRE (e.g., to bind to Rev and promote nuclear export). The retroviral nucleic acid can comprise RNA (e.g., when part of a virion) or DNA (e.g., when being introduced into a source cell or after reverse transcription in a recipient cell). In some embodiments, the retroviral nucleic acid is packaged using a helper cell, helper virus, or helper plasmid which comprises one or more of (e.g., all of) gag, pol, and env.

As used herein, a “target cell” refers to a cell of a type to which it is desired that a targeted lipid particle delivers an exogenous agent. In embodiments, a target cell is a cell of a specific tissue type or class, e.g., an immune effector cell, e.g., a T cell. In some embodiments, a target cell is a diseased cell, e.g., a cancer cell. In some embodiments, the fusogen, e.g., a re-targeted fusogen, leads to preferential delivery of the exogenous agent to a target cell compared to a non-target cell.

As used herein a “non-target cell” refers to a cell of a type to which it is not desired that a targeted lipid particle delivers an exogenous agent. In some embodiments, a non-target cell is a cell of a specific tissue type or class. In some embodiments, a non-target cell is a non-diseased cell, e.g., a non-cancerous cell. In some embodiments, the fusogen, e.g., a re-targeted fusogen, leads to lower delivery of the exogenous agent to a non-target cell compared to a target cell.

The term “effective amount” as used herein means an amount of a pharmaceutical composition which is sufficient to significantly and positively modify the symptoms and/or conditions to be treated (e.g., provide a positive clinical response). The effective amount of the targeted lipid particles of the disclosure for use in a pharmaceutical composition will vary with the particular condition being treated, the severity of the condition, the duration of treatment, the nature of concurrent therapy, the particular lipid particle) being employed, the particular pharmaceutically-acceptable excipient(s) and/or carrier(s) utilized, and like factors with the knowledge and expertise of the attending physician.

An “exogenous agent” as used herein with reference to a targeted lipid particle, refers to an agent that is neither comprised by nor encoded in the corresponding wild-type virus or fusogen made from a corresponding wild-type source cell. In some embodiments, the exogenous agent does not naturally exist, such as a protein or nucleic acid that has a sequence that is altered (e.g., by insertion, deletion, or substitution) relative to a naturally occurring protein. In some embodiments, the exogenous agent does not naturally exist in the source cell. In some embodiments, the exogenous agent exists naturally in the source cell but is exogenous to the virus. In some embodiments, the exogenous agent does not naturally exist in the recipient cell. In some embodiments, the exogenous agent exists naturally in the recipient cell, but is not present at a desired level or at a desired time. In some embodiments, the exogenous agent comprises DNA, RNA, or protein.

As used herein, the term “operably linked” refers to the association of two or more nucleic acid molecules on a single nucleic acid fragment so that the function of one is affected by the other.

As used herein, “nucleic acid” or “polynucleotide” refers to a polymeric compound including covalently linked nucleotides comprising natural subunits (e.g., purine or pyrimidine bases). In some embodiments, a polynucleotide comprises a transgene. Purine bases include adenine and guanine, and pyrimidine bases include uracil, thymine, and cytosine. Nucleic acid molecules include ribonucleic acid (RNA) and deoxyribonucleic acid (DNA), which includes cDNA, genomic DNA, and synthetic DNA, either of which are single- or double-stranded. A nucleic acid molecule encoding an amino acid sequence includes all nucleotide sequences that encode the same amino acid sequence.

As used herein, a “transgene” refers to genetic material that has been transferred to a cell (e.g., a host cell). A transgene comprises nucleic acids, and is, in some embodiments, incorporated into a cell through any of the methods disclosed herein.

As used herein, a “promoter” refers to a cis-regulatory DNA sequence that, when operably linked to a gene coding sequence, drives transcription of the gene. The promoter may comprise one or more transcription factor binding sites. In some embodiments, a promoter works in concert with one or more enhancers which are distal to the gene.

The term “safe harbor locus” refers to a gene locus that allows safe expression of a transgene or an exogenous gene. Safe harbors or genomic safe harbors are sites in the genome able to accommodate the integration of new genetic material in a manner that permits the newly inserted genetic elements to: (i) function predictably and (ii) do not cause alterations of the host genome posing a risk to the host cell or organism. Exemplary “safe harbor” loci include a CCR5 gene, a CXCR4 gene, a PPP1R12C (also known as AAVS1) gene, an albumin gene, and a Rosa gene.

The term “safety switch” refers to a system for controlling the expression of a gene or protein of interest that, when downregulated or upregulated, leads to clearance or death of the cell, e.g., through recognition by the host's immune system. A safety switch is designed to be or include an exogenous molecule administered to prevent or mitigate an adverse clinical event. A safety switch is engineered by regulating the expression on the DNA, RNA and protein levels. A safety switch may include a protein or molecule that allows for the control of cellular activity in response to an adverse event. In some embodiments, a safety switch refers to an agent (e.g., protein, molecule, etc.) that binds a specific cell and targets it for cell death or elimination. In some instances, the safety switch is a blockade agent that binds a target protein on the surface of a cell, which in turn, triggers an immune response. In one embodiment, the safety switch is a “kill switch” that is expressed in an inactive state and is fatal to a cell expressing the safety switch upon activation of the switch by a selective, externally provided agent. In one embodiment, the safety switch gene is cis-acting in relation to the gene of interest in a construct. Activation of the safety switch causes the cell to kill solely itself or itself and neighboring cells through apoptosis or necrosis.

The term “tolerogenic factor” as used herein includes hypoimmunity factors, complement inhibitors, and other factors that modulate or affect (e.g., reduce) the ability of a cell to be recognized by the immune system of a host or recipient subject upon administration, transplantation, or engraftment. Tolerogenic factors include but are not limited to CD16, CD24, CD35, CD39, CD46, CD47, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-L1, Serpinb9, CCl21, Mfge8, A20/TNFAIP3, CCL21, CD16 Fc receptor, CD27, CR1, DUX4, H2-M3 (HLA-G), HLA-F, IL15-RF, MANF, IL-39, and B2M-HLA-E.

As used herein, a composition refers to any mixture of two or more products, substances, or compounds, including cells. It includes a solution, a suspension, liquid, powder, a paste, aqueous, non-aqueous, or any combination thereof.

As used herein, the term “pharmaceutically acceptable” refers to a material, such as a carrier or diluent, which does not abrogate the biological activity or properties of a therapeutic compound, and is relatively nontoxic, i.e., the material is administered to an individual without causing undesirable biological effects or interacting in a deleterious manner with any of the components of the composition in which it is contained.

As used herein, the term “pharmaceutical composition” refers to a mixture of at least one targeted lipid particle of the disclosure with other chemical components, such as carriers, stabilizers, diluents, dispersing agents, suspending agents, thickening agents, and/or excipients. The pharmaceutical composition facilitates administration of the targeted lipid particle to an organism. Multiple techniques of administering targeted lipid particles of the disclosure exist in the art including, but not limited to, intravenous, oral, aerosol, parenteral, ophthalmic, pulmonary, and topical administration.

A “disease” or “disorder” as used herein refers to a condition in which treatment is needed and/or desired.

As used herein, the terms “treat,” “treating,” or “treatment” refer to ameliorating a disease or disorder, e.g., slowing or arresting or reducing the development of the disease or disorder or reducing at least one of the clinical symptoms thereof. For purposes of this disclosure, ameliorating a disease or disorder includes obtaining a beneficial or desired clinical result that includes, but is not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total).

The terms “individual” and “subject” are used interchangeably herein to refer to an animal; for example, a mammal. The terms include human and veterinary animals. In some embodiments, methods of treating animals, including, but not limited to, humans, rodents, simians, felines, canines, equines, bovines, porcines, ovines, caprines, mammalian laboratory animals, mammalian farm animals, mammalian sport animals, and mammalian pets, are provided. The animal is male or female and is any suitable age, including infant, juvenile, adolescent, adult, and geriatric. In some examples, an “individual” or “subject” refers to an animal in need of treatment for a disease or disorder. In some embodiments, the animal to receive the treatment is a “patient,” designating the fact that the animal has been identified as having a disorder of relevance to the treatment, or being at adequate risk of contracting the disorder. In some embodiments, the animal is a human, such as a human patient.

The terms “treat,” “treating,” and “treatment” as used herein with regard to cancer refers to alleviating the cancer partially or entirely; preventing the cancer; decreasing the likelihood of occurrence or recurrence of the cancer; slowing the progression or development of the cancer; eliminating, reducing, or slowing the development of one or more symptoms associated with the cancer; or increasing progression-free or overall survival of the cancer. For example, “treating” may refer to preventing or slowing the existing cancer from growing larger; preventing or slowing the formation or metastasis of cancer; and/or slowing the development of certain symptoms of the cancer. In some embodiments, the term “treat,” “treating,” or “treatment” means that the subject has a reduced number or size of cancer cells comparing to a subject without being administered with the treatment. In some embodiments, the term “treat,” “treating,” or “treatment” means that one or more symptoms of the cancer are alleviated in a subject receiving the treatment as disclosed and described herein comparing to a subject who does not receive such treatment.

All publications, patents, and patent applications cited in this specification are incorporated herein by reference to the same extent as if each individual publication, patent, or patent application were specifically and individually indicated to be incorporated by reference. Furthermore, each cited publication, patent, or patent application is incorporated herein by reference to disclose and describe the subject matter in connection with which the publications are cited. The citation of any publication is for its disclosure prior to the filing date and should not be construed as an admission that the technology described herein is not entitled to antedate such publication by virtue of prior technology. Further, the dates of publication provided might be different from the actual publication dates, which may need to be independently confirmed.

Before the technology is further described, it is to be understood that this technology is not limited to particular embodiments described, as such may, of course, vary. It is also to be understood that the terminology used herein is for the purpose of describing particular embodiments only, and is not intended to be limiting, since the scope of the present disclosure will be limited only by the appended claims. It should also be understood that the headers used herein are not limiting and are merely intended to orient the reader, but the subject matter generally applies to the technology disclosed herein.

### GPRC5D-Specific Polypeptides

Described herein are novel polypeptides that specifically target and bind human GPRC5D. In some embodiments, the polypeptides may cross-react with cynomolgus (or “cyno”) or M. nemestrina GPRC5D. In some embodiments, the polypeptides are antibodies or antigen binding fragments thereof. The present disclosure also provides polynucleotides encoding the polypeptides, vectors, and host cells, and methods of using the polypeptides thereof. In some embodiments, e.g., the polypeptides are fused to henipavirus glycoprotein G for targeted binding and transduction to cells. In some embodiments, the polypeptide comprises an antigen binding region that specifically binds GPRC5D.

Sequences for exemplary polypeptides of the disclosure comprising antigen binding regions using the Kabat numbering scheme are shown in Tables 3-4 below. In some embodiments, the antigen binding regions comprises one or more heavy chain complementarity determining regions (HCDRs). In some embodiments, the antigen binding regions comprises one or more light chain complementarity determining regions (LCDRs). In some embodiments, the antigen binding regions comprise a heavy chain variable region (VH). In some embodiments, the antigen binding regions comprise a light chain variable region (VL). Sequences for exemplary HCDRs of the disclosure are shown in Table 3. Sequences for exemplary LCDRs of the disclosure are shown in Table 4.

The sequences for the disclosed VH and VL domains are provided in Tables 5-6.

In some embodiments, a polypeptide capable of binding GPRC5D is disclosed. In some embodiments, the polypeptide comprises a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3). In some embodiments, the HCDR1, HCDR2, and HCDR3 comprise amino acid sequences of any one of the SEQ ID NOs recited in Table 3, and the LCDR1, LCDR2, and LCDR3 comprise amino acid sequences of any one of the SEQ ID NOs recited in Table 4. In some embodiments, the heavy chain variable region (VH) comprises an amino acid sequence of any one of SEQ ID NOs: 19-21 (Table 5) and the light chain variable region (VL) comprises an amino acid sequence of any one of SEQ ID NOs: 22-24 (Table 6).

In some embodiments, the polypeptide comprises an amino acid sequence with at least 80%, 85%, 90%, 95%, 96%, 97%, 98%, 99%, or 100% identity to a sequence selected from SEQ ID NOs: 19-21.

In some embodiments, the polypeptide comprises an amino acid sequence with at least 80%, 85%, 90%, 95%, 96%, 97%, 98%, 99%, or 100% identity to a sequence selected from SEQ ID NOs: 22-24.

In some embodiments, the polypeptide comprises an amino acid sequence with at least 80%, 85%, 90%, 95%, 96%, 97%, 98%, 99%, or 100% identity to a sequence selected from SEQ ID NOs: 19-21 and an amino acid sequence with at least 80%, 85%, 90%, 95%, 96%, 97%, 98%, 99%, or 100% identity to a sequence selected from SEQ ID NOs: 22-24.

In some embodiments, the polypeptide comprises the amino acid sequence of SEQ ID NOs: 1, 4, 7, 10, 13, and 16.

In some embodiments, the polypeptide comprises the amino acid sequence of SEQ ID NOs: 2, 5, 8, 11, 14, and 17.

In some embodiments, the polypeptide comprises the amino acid sequence of SEQ ID NOs: 3, 6, 9, 12, 15, and 18.

In some embodiments, the polypeptide is an antibody or antigen binding fragment thereof as disclosed herein.

Polypeptides whose amino acid sequences differ insubstantially from those shown in Tables 3-6 are encompassed within the scope of the disclosure. Typically, this involves one or more conservative amino acid substitutions with an amino acid having similar charge, hydrophobic, or stereo chemical characteristics in the antigen-binding site or in the framework without adversely altering the properties of the polypeptide. Conservative substitutions may also be made to improve polypeptide properties, for example stability or affinity. 1, 2, 3, 4, 5, 6, 7, 8, 9, 10, 11, 12, 13, 14, or 15 amino acid substitutions are made to the amino acid sequence. For example, a “conservative amino acid substitution” may involve a substitution of a native amino acid residue with a nonnative residue such that there is little or no effect on the polarity or charge of the amino acid residue at that position. Desired amino acid substitutions are determined by those skilled in the art at the time such substitutions are desired. For example, amino acid substitutions are used to identify important residues of the molecule sequence, or to increase or decrease the affinity of the molecules described herein. The following eight groups contain amino acids that are conservative amino acid substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

In some embodiments, the polypeptide binds to human GPRC5D. In some embodiments, the polypeptide is an antibody or antigen binding fragment binding that specifically binds GPRC5D as disclosed herein.

In some embodiments, the polypeptide binds to human GPRC5D with an affinity constant (K_{D}) of between about 1 nM and about 900 nM. In some embodiments, the K_{D} to human GPRC5D is between about 5 nM about 500 nM, about 6 nM to about 10 nM, about 11 nM to about 20 nM, about 25 nM to about 40 nM, about 40 nM to about 60 nM, about 70 nM to about 90 nM, about 100 nM to about 120 nM, about 125 nM to about 140 nM, about 145 nM to about 160 nM, about 170 nM and to about 200 nM, about 210 nM to about 250 nM, about 260 nM to about 300 nM, about 310 nM to about 350 nM, about 360 nM to about 400 nM, about 410 nM to about 450 nM, and about 460 nM to about 500 nM. In some embodiments, the polypeptide binds to human GPRC5D with an affinity constant (K_{D}) of 500 nM, 400 nM, 300 nM, 200 nM, 100 nM, 90 nM, 80 nM, 70 nM, 60 nM, 50 nM, 40 nM, 30 nM, 20 nM, or 10 nM or lower. In some embodiments, the polypeptide binds to human GPRC5D and cynomolgus, M. mulatta (rhesus monkey), or M. nemestrina GPRC5D with comparable binding affinity (K_{D}).

In some embodiments, the polypeptide binds to cynomolgus, M. mulatta (rhesus monkey), or N. nemestrina GPRC5D. In some embodiments, the polypeptide binds to mouse, dog, pig, etc., GPRC5D. In some embodiments, the K_{D} to cynomolgus or M. nemestrina GPRC5D is between about 5 nM about 500 nM, about 6 nM to about 10 nM, about 11 nM to about 20 nM, about 25 nM to about 40 nM, about 40 nM to about 60 nM, about 70 nM to about 90 nM, about 100 nM to about 120 nM, about 125 nM to about 140 nM, about 145 nM to about 160 nM, about 170 nM and to about 200 nM, about 210 nM to about 250 nM, about 260 nM to about 300 nM, about 310 nM to about 350 nM, about 360 nM to about 400 nM, about 410 nM to about 450 nM, and about 460 nM to about 500 nM. In some embodiments, the polypeptide binds to cynomolgus or M. nemestrina GPRC5D with an affinity constant (K_{D}) of 500 nM, 400 nM, 300 nM, 200 nM, 100 nM, 90 nM, 80 nM, 70 nM, 60 nM, 50 nM, 40 nM, 30 nM, 20 nM, or 10 nM or lower.

A polypeptide that specifically binds GPRC5D refers to a polypeptide that preferentially binds to GPRC5D, respectively, over other antigen targets. As used herein, the term is interchangeable with an “anti-GPRC5D” polypeptide or an “polypeptide that binds GPRC5D.” In some embodiments, the polypeptide capable of binding to GPRC5D can do so with higher affinity for that antigen than others. In some embodiments, polypeptide capable of binding GPRC5D can bind to that antigen with a K_{D} of at least about 10⁻¹, 10⁻², 10⁻³, 10⁻⁴, 10⁻⁵, 10⁻⁶, 10⁻⁷, 10⁻⁸, 10⁻⁹, 10⁻¹⁰, 10⁻¹¹, 10⁻¹² or greater (or any value in between), e.g., as measured by surface plasmon resonance or other methods known to those skilled in the art.

In some embodiments, the polypeptide is bispecific. In some embodiments, the bispecific polypeptide comprises an antigen binding region that specifically binds GPRC5D as herein disclosed and an antigen binding region that specifically binds CD3, BCMA, CD19, 4-1BB, IL-6, NKG2D, Fc-gamma-RIIIA (CD16), APRIL, CD38, TACI, Fc-gamma-RIIIA (CD16) and NKG2D, CD3 and serum albumin, or CD47 and TAC. In some embodiments, the antigen binding region comprises an antibody or antibody binding fragment thereof.

In some embodiments, the polypeptide is conjugated. In some embodiments, the polypeptide is a polypeptide-drug conjugate, wherein the polypeptide that specifically binds GPRC5D as herein disclosed is conjugated to a therapeutic agent or diagnostic agent. In some embodiments, the polypeptide is conjugated to a tag for detection. In some embodiments, the polypeptide is conjugated to a conjugate that enhances polypeptide stability. In some embodiments, the polypeptide is conjugated to a cleavable linker, wherein the linker allows for another molecule to be conjugated to the polypeptide. In some embodiments, the polypeptide is conjugated to a nanoparticle.

Some embodiments of the disclosure are an isolated polynucleotide encoding any of the polypeptides of the disclosure. Certain exemplary polynucleotides are disclosed herein, however, other polynucleotides which, given the degeneracy of the genetic code or codon preferences in a given expression system, encode the polypeptides of the disclosure are also within the scope of the disclosure. The polynucleotide sequences encoding an antigen binding region thereof of the polypeptide of the disclosure are operably linked to one or more regulatory elements, such as a promoter and enhancer, that allow expression of the nucleotide sequence in the intended host cell. In some embodiments, the polynucleotide is a cDNA.

Some embodiments of the disclosure are a vector comprising the polynucleotide of the disclosure. In some embodiments, such vectors are plasmid vectors, viral vectors, vectors for baculovirus expression, transposon-based vectors, or any other vector suitable for introduction of the polynucleotide of the disclosure into a given organism or genetic background by any means. In some embodiments, the vector is polycistronic. For example, polynucleotides encoding light and heavy chain variable regions of the polypeptide of the disclosure, optionally linked to constant regions, are inserted into expression vectors. The light and heavy chains are cloned in the same or different expression vectors. The DNA segments encoding immunoglobulin chains are operably linked to control sequences in the expression vector(s) that ensure the expression of immunoglobulin polypeptides. Such control sequences include signal sequences, promoters (e.g., naturally associated or heterologous promoters), enhancer elements, and transcription termination sequences, and are chosen to be compatible with the host cell chosen to express the polypeptide. Once the vector has been incorporated into the appropriate host, the host is maintained under conditions suitable for high level expression of the polypeptides encoded by the incorporated polynucleotides.

Suitable expression vectors are typically replicable in the host organisms either as episomes or as an integral part of the host chromosomal DNA. Commonly, expression vectors contain selection markers such as ampicillin-resistance, hygromycin-resistance, tetracycline resistance, kanamycin resistance, or neomycin resistance to permit detection of those cells transformed with the desired DNA sequences. Suitable vectors, promoter, and enhancer elements are known in the art; many are commercially available for generating subject recombinant constructs.

Some embodiments of the disclosure are a host cell comprising the vector of the disclosure. The term “host cell” refers to a cell into which a vector has been introduced. It is understood that the term host cell is intended to refer not only to the particular subject cell but to the progeny of such a cell. Because certain modifications may occur in succeeding generations due to either mutation or environmental influences, such progeny may not be identical to the parent cell, but are still included within the scope of the term “host cell” as used herein. Such host cells include eukaryotic cells, prokaryotic cells, plant cells, or archaeal cells. Escherichia coli, bacilli, such as Bacillus subtilis, and other enterobacteriaceae, such as Salmonella, Serratia, and various Pseudomonas species are examples of prokaryotic host cells. Other microbes, such as yeast, are also useful for expression. Saccharomyces (e.g., S. cerevisiae) and Pichia are examples of suitable yeast host cells. Exemplary eukaryotic cells include cells of mammalian, insect, avian, or other animal origins.

### GPRC5D-Specific Antibodies

Described herein are novel antibodies and antigen binding fragments thereof that specifically target and bind human GPRC5D. In some embodiments, the antibodies or antigen binding fragments thereof may cross-react with cynomolgus (or “cyno”) or M. nemestrina GPRC5D. In some embodiments, the antibodies or antigen binding fragments thereof are single-chain variable fragments (scFvs) composed of the antigen-binding domains derived from the heavy (VH) and the light (VL) chains of the IgG molecule and connected via a linker domain. In some embodiments, the antibodies or antigen binding fragments are single domain antibodies (sdAbs) composed of the antigen-binding domain derived from a heavy (VH) or light (VL) chain of the IgG molecule. In some embodiments, the antibodies or antigen binding fragments thereof are VHHs that correspond to the VH of the IgG molecule. The present disclosure also provides polynucleotides encoding the antibodies and fragments thereof, vectors, and host cells, and methods of using the antibodies or antigen binding fragments thereof. In some embodiments, e.g., the antibodies or antigen binding fragments thereof are fused to henipavirus glycoprotein G for targeted binding and transduction to cells.

Sequences for exemplary antibodies and antigen binding fragments of the disclosure using the Kabat numbering scheme are shown in Tables 3-4 below. Sequences for exemplary HCDRs of the disclosure are shown in Table 3. Sequences for exemplary LCDRs of the disclosure are shown in Table 4. The sequences for the disclosed VH and VL domains are provided in Tables 5-6. The full GPRC5D binder sequences of the variant GPRC5D scFvs and VHHs of the disclosure are shown in Table 7.

In some embodiments, an antibody or antigen binding fragment thereof capable of binding GPRC5D is disclosed, comprising a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3). In some embodiments, the HCDR1, HCDR2, and HCDR3 comprise amino acid sequences of any one of the SEQ ID NOs recited in Table 3, and the LCDR1, LCDR2, and LCDR3 comprise amino acid sequences of any one of the SEQ ID NOs recited in Table 4. In some embodiments, the heavy chain variable region (VH) comprises an amino acid sequence of any one of SEQ ID NOs: 19-21 (Table 5) and the light chain variable region (VL) comprises an amino acid sequence of any one of SEQ ID NOs: 22-24 (Table 6).

In some embodiments, the antibody or antigen binding fragment thereof comprises a VH having an amino acid sequence with at least 80%, 85%, 90%, 95%, 96%, 97%, 98%, 99%, or 100% identity to a sequence selected from SEQ ID NOs: 19-21.

In some embodiments, the antibody or antigen binding fragment thereof comprises a VL having an amino acid sequence with at least 80%, 85%, 90%, 95%, 96%, 97%, 98%, 99%, or 100% identity to a sequence selected from SEQ ID NOs: 22-24.

In some embodiments, the antibody or antigen binding fragment comprises a VH having an amino acid sequence with at least 80%, 85%, 90%, 95%, 96%, 97%, 98%, 99%, or 100% identity to a sequence selected from SEQ ID NOs: 19-21 and a VL having an amino acid sequence with at least 80%, 85%, 90%, 95%, 96%, 97%, 98%, 99%, or 100% identity to a sequence selected from SEQ ID NOs: 22-24.

In some embodiments, the antibody or antigen binding fragment thereof comprises the HCDR1, HCDR2, HCDR3, LCDR1, LCDR2, and LCDR3 of SEQ ID NOs: 1, 4, 7, 10, 13, and 16, respectively.

In some embodiments, the antibody or antigen binding fragment thereof comprises the HCDR1, HCDR2, HCDR3, LCDR1, LCDR2, and LCDR3 of SEQ ID NOs: 2, 5, 8, 11, 14, and 17, respectively.

In some embodiments, the antibody or antigen binding fragment thereof comprises the HCDR1, HCDR2, HCDR3, LCDR1, LCDR2, and LCDR3 of SEQ ID NOs: 3, 6, 9, 12, 15, and 18, respectively.

In some embodiments, the single domain antibody is human or humanized. In some embodiments, the single domain antibody or portion thereof is naturally occurring. In some embodiments, the single domain antibody or portion thereof is synthetic.

In some embodiments, the single domain antibodies are antibodies whose complementary determining regions are part of a single domain polypeptide. In some embodiments, the single domain antibody is a heavy chain only antibody variable domain. In some embodiments, the single domain antibody does not include light chains.

In various embodiments, any of the antibodies or antigen binding fragments described herein can comprise a heavy chain constant region and a light chain constant region. In some embodiments, the heavy chain constant region is an IgG, IgM, IgA, IgD, or IgE isotype, or a derivative or fragment thereof that retains at least one effector function of the intact heavy chain. In some embodiments, the heavy chain constant region is a human IgG isotype. In some embodiments, the heavy chain constant region is a human IgG1 or human IgG4 isotype. In some embodiments, the heavy chain constant region is a human IgG1 isotype. In some embodiments, the light chain constant region is a human kappa light chain or lambda light chain or a derivative or fragment thereof that retains at least one effector function of the intact light chain. In some embodiments, the light chain constant region is a human kappa light chain.

In various embodiments, any of the disclosed antibodies or antigen binding fragments are a rodent antibody or antigen binding fragment thereof, a chimeric antibody or an antigen binding fragment thereof, a CDR-grafted antibody or an antigen binding fragment thereof, or a humanized antibody or an antigen binding fragment thereof. In some embodiments, any of the disclosed antibodies or antigen binding fragments comprises human or human-derived heavy and light chain variable regions, including human frameworks or human frameworks with one or more backmutations. In various embodiments, any of the disclosed antibodies or antigen binding fragments are a Fab, Fab′, F(ab′)2, Fd, scFv, (scFv)2, scFv-Fc, VHH, or Fv fragment.

Antibodies whose heavy chain CDR, light chain CDR, VH, or VL amino acid sequences differ insubstantially from those shown in Tables 3-6 are encompassed within the scope of the disclosure. Typically, this involves one or more conservative amino acid substitutions with an amino acid having similar charge, hydrophobic, or stereo chemical characteristics in the antigen-binding site or in the framework without adversely altering the properties of the antibody. Conservative substitutions may also be made to improve antibody properties, for example stability or affinity. 1, 2, 3, 4, 5, 6, 7, 8, 9, 10, 11, 12, 13, 14, or 15 amino acid substitutions are made to the VH or VL sequence. For example, a “conservative amino acid substitution” may involve a substitution of a native amino acid residue with a nonnative residue such that there is little or no effect on the polarity or charge of the amino acid residue at that position. Desired amino acid substitutions are determined by those skilled in the art at the time such substitutions are desired. For example, amino acid substitutions are used to identify important residues of the molecule sequence, or to increase or decrease the affinity of the molecules described herein. The following eight groups contain amino acids that are conservative amino acid substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

In some embodiments, the antibody or antigen binding fragment thereof binds to human GPRC5D. In some embodiments, the antibody or antigen binding fragment binding GPRC5D is a single-chain variable fragment (scFv). In embodiments involving a single polypeptide containing both a heavy chain variable region and a light chain variable region, both orientations of these variable regions are contemplated. In some embodiments, the heavy chain variable region is on the N-terminal side of the light chain variable region, which means the heavy chain variable region is closer to the N-terminus of the polypeptide. In other embodiments, the light chain variable region is on the N-terminal side of the heavy chain variable region, which means the light chain variable region is closer to the N-terminus of the polypeptide than the heavy chain variable region.

In some embodiments, the scFv binding proteins comprise a linker. In some embodiments, the linker is between the heavy chain variable region (VH) and the light chain variable region (VL) (or vice versa). In some embodiments, the linker comprises the amino acid sequence of GS, GGS, GGGS, GGGGS (SEQ ID NO: 147), GGGGGS (SEQ ID NO: 145), any one of SEQ ID NOs: 32-33, 165-166, or combinations thereof. Substitutions to introduce new disulfide bonds are also within the scope of the disclosure, e.g., by making substitutions G44C in the VH FR 2 and G100C in the VL FR4.

In some embodiments, the anti-GPRC5D antibody or antigen binding fragment binds to human GPRC5D with an affinity constant (K_{D}) of between about 1 nM and about 900 nM. In some embodiments, the K_{D} to human GPRC5D is between about 5 nM about 500 nM, about 6 nM to about 10 nM, about 11 nM to about 20 nM, about 25 nM to about 40 nM, about 40 nM to about 60 nM, about 70 nM to about 90 nM, about 100 nM to about 120 nM, about 125 nM to about 140 nM, about 145 nM to about 160 nM, about 170 nM and to about 200 nM, about 210 nM to about 250 nM, about 260 nM to about 300 nM, about 310 nM to about 350 nM, about 360 nM to about 400 nM, about 410 nM to about 450 nM, and about 460 nM to about 500 nM. In some embodiments, the anti-GPRC5D antibody or antigen binding fragment binds to human GPRC5D with an affinity constant (K_{D}) of 500 nM, 400 nM, 300 nM, 200 nM, 100 nM, 90 nM, 80 nM, 70 nM, 60 nM, 50 nM, 40 nM, 30 nM, 20 nM, or 10 nM or lower. In some embodiments, the anti-GPRC5D antibody or antigen binding fragment binds to human GPRC5D and cynomolgus, M. mulatta (rhesus monkey), or M. nemestrina GPRC5D with comparable binding affinity (K_{D}).

In some embodiments, the anti-GPRCSD antibody or antigen binding fragment binds to cynomolgus, M. mulatta (rhesus monkey), or N. nemestrina GPRCSD. In some embodiments, the anti-GPRCSD antibody or antigen binding binds to mouse, dog, pig, etc., GPRCSD. In some embodiments, the K_{D} to cynomolgus or M. nemestrina GPRCSD is between about 5 nM about 500 nM, about 6 nM to about 10 nM, about 11 nM to about 20 nM, about 25 nM to about 40 nM, about 40 nM to about 60 nM, about 70 nM to about 90 nM, about 100 nM to about 120 nM, about 125 nM to about 140 nM, about 145 nM to about 160 nM, about 170 nM and to about 200 nM, about 210 nM to about 250 nM, about 260 nM to about 300 nM, about 310 nM to about 350 nM, about 360 nM to about 400 nM, about 410 nM to about 450 nM, and about 460 nM to about 500 nM. In some embodiments, the anti-GPRC5D antibody or antigen binding fragment binds to cynomolgus or M. nemestrina GPRC5D with an affinity constant (K_{D}) of 500 nM, 400 nM, 300 nM, 200 nM, 100 nM, 90 nM, 80 nM, 70 nM, 60 nM, 50 nM, 40 nM, 30 nM, 20 nM, or 10 nM or lower.

An antibody or antigen binding fragment thereof that specifically binds GPRC5D refers to an antibody or binding fragment that preferentially binds to GPRC5D, respectively, over other antigen targets. As used herein, the term is interchangeable with an “anti-GPRC5D” antibody or an “antibody that binds GPRC5D.” In some embodiments, the antibody or binding fragment capable of binding to GPRC5D can do so with higher affinity for that antigen than others. In some embodiments, the antibody or binding fragment capable of binding GPRC5D can bind to that antigen with a K_{D} of at least about 10⁻¹, 10⁻², 10⁻³, 10⁻⁴, 10⁻⁵, 10⁻⁶, 10⁻⁷ 10⁻⁸, 10⁻⁹, 10⁻¹⁰, 10⁻¹¹, 10⁻¹² or greater (or any value in between), e.g., as measured by surface plasmon resonance or other methods known to those skilled in the art.

In some embodiments, the antibody or antigen binding fragment thereof is bispecific. In some embodiments, the bispecific antibody or antigen binding fragment comprises an antibody or antigen binding fragment thereof that specifically binds GPRC5D as herein disclosed and an antigen or antibody binding fragment thereof that specifically binds CD3, BCMA, CD19, 4-1BB, IL-6, NKG2D, Fc-gamma-RIIIA (CD16), APRIL, CD38, TACI, Fc-gamma-RIIIA (CD16) and NKG2D, CD3 and serum albumin, or CD47 and TACI.

In some embodiments, the antibody or antigen binding fragment thereof is conjugated. In some embodiments, the antibody or antigen-binding fragment thereof is an antibody-drug conjugate, wherein the antibody or antigen binding fragment thereof that specifically binds GPRC5D as herein disclosed is conjugated to a therapeutic agent or diagnostic agent. In some embodiments, the antibody or antigen binding fragment thereof is conjugated to a tag for detection. In some embodiments, the antibody or antigen binding fragment thereof is conjugated to a conjugate that enhances antibody or antigen binding fragment thereof stability. In some embodiments, the antibody or antigen binding fragment thereof is conjugated to a cleavable linker, wherein the linker allows for another molecule to be conjugated to the antibody or antigen binding fragment thereof. In some embodiments, the antibody or antigen binding fragment thereof is conjugated to a nanoparticle.

Some embodiments of the disclosure are an isolated polynucleotide encoding any of the antibody heavy chain variable regions or the antibody light chain variable regions of the disclosure. Certain exemplary polynucleotides are disclosed herein, however, other polynucleotides which, given the degeneracy of the genetic code or codon preferences in a given expression system, encode the antibodies or antigen binding fragments thereof of the disclosure are also within the scope of the disclosure. The polynucleotide sequences encoding a VH or a VL or a fragment thereof of the antibody or antigen binding fragments thereof of the disclosure are operably linked to one or more regulatory elements, such as a promoter and enhancer, that allow expression of the nucleotide sequence in the intended host cell. In some embodiments, the polynucleotide is a cDNA.

Some embodiments of the disclosure are a vector comprising the polynucleotide of the disclosure. In some embodiments, such vectors are plasmid vectors, viral vectors, vectors for baculovirus expression, transposon-based vectors, or any other vector suitable for introduction of the polynucleotide of the disclosure into a given organism or genetic background by any means. In some embodiments, the vector is polycistronic. For example, polynucleotides encoding light and heavy chain variable regions of the antibodies of the disclosure, optionally linked to constant regions, are inserted into expression vectors. The light and heavy chains are cloned in the same or different expression vectors. The DNA segments encoding immunoglobulin chains are operably linked to control sequences in the expression vector(s) that ensure the expression of immunoglobulin polypeptides. Such control sequences include signal sequences, promoters (e.g., naturally associated or heterologous promoters), enhancer elements, and transcription termination sequences, and are chosen to be compatible with the host cell chosen to express the antibody. In some embodiments, the polycistronic vector comprises one or more tolerogenic factor, safety switch, additional antibodies or antigen binding fragments thereof, or other regulatory elements as disclosed herein. Once the vector has been incorporated into the appropriate host, the host is maintained under conditions suitable for high level expression of the proteins encoded by the incorporated polynucleotides.

Suitable expression vectors are typically replicable in the host organisms either as episomes or as an integral part of the host chromosomal DNA. Commonly, expression vectors contain selection markers such as ampicillin-resistance, hygromycin-resistance, tetracycline resistance, kanamycin resistance, or neomycin resistance to permit detection of those cells transformed with the desired DNA sequences. Suitable vectors, promoter, and enhancer elements are known in the art; many are commercially available for generating subject recombinant constructs.

Some embodiments of the disclosure are a host cell comprising the vector of the disclosure. The term “host cell” refers to a cell into which a vector has been introduced. It is understood that the term host cell is intended to refer not only to the particular subject cell but to the progeny of such a cell. Because certain modifications may occur in succeeding generations due to either mutation or environmental influences, such progeny may not be identical to the parent cell, but are still included within the scope of the term “host cell” as used herein. Such host cells include eukaryotic cells, prokaryotic cells, plant cells, or archaeal cells. Escherichia coli, bacilli, such as Bacillus subtilis, and other enterobacteriaceae, such as Salmonella, Serratia, and various Pseudomonas species are examples of prokaryotic host cells. Other microbes, such as yeast, are also useful for expression. Saccharomyces (e.g., S. cerevisiae) and Pichia are examples of suitable yeast host cells. Exemplary eukaryotic cells include cells of mammalian, insect, avian, or other animal origins.

### GPRC5D Chimeric Antigen Receptor

In some embodiments the provided disclosure relates to chimeric receptors, such as a chimeric antigen receptor (CAR), that contain one or more domains that combine an antigen- or ligand-binding domain (e.g., antibody or antigen binding fragment thereof) that provides specificity for a desired antigen (e.g., tumor antigen) with intracellular signaling domains. In some embodiments, the intracellular signaling domain is a stimulating or an activating intracellular domain portion, such as a T cell stimulating or activating domain, providing a primary activation signal or a primary signal. In some embodiments, the intracellular signaling domain contains or additionally contains a costimulatory signaling domain to facilitate effector functions. In some embodiments, chimeric receptors when genetically engineered into immune cells can modulate T cell activity, and, in some embodiments, can modulate T cell differentiation or homeostasis, thereby resulting in genetically engineered cells with improved longevity, survival and/or persistence in vivo, such as for use in adoptive cell therapy methods.

In some embodiments, the chimeric antigen receptor includes an extracellular portion containing an antibody or antigen binding fragment thereof that comprises an antigen-binding domain. In some aspects, the chimeric antigen receptor includes an extracellular portion containing the antibody or antigen binding fragment thereof comprising an antigen-binding domain and an intracellular signaling domain. In some embodiments, the antibody or antigen binding fragment thereof includes an scFv.

In some embodiments, the antigen targeted by the antigen-binding domain is GPRC5D. In some aspects, the antigen-binding domain of the recombinant receptor, e.g., CAR, binds, such as specifically binds or specifically recognizes, a GPRC5D, such as a human GPRC5D. In some embodiments, the antibody or antigen binding fragment thereof comprises a VH and a VL derived from an antibody or an antibody fragment specific to GPRC5D as disclosed herein. In some embodiments, the antibody or antigen binding fragment thereof is a human antibody, e.g., as described in U.S. Patent Publication No. US 2016/0152723.

In some embodiments, the CAR is a GPRC5D CAR (“GPRC5D-CAR”). In some of these embodiments, a polycistronic vector comprises an expression cassette that contains a nucleotide sequence encoding a GPRC5D CAR or another CAR disclosed herein. GPRC5D is an orphan G protein-coupled receptor family member that is expressed on plasma cells. The expression of GPRC5D has been linked to multiple myeloma. In some embodiments, the GPRC5D CAR may comprise a signal peptide, an extracellular binding domain that specifically binds GPRC5D, a hinge domain, a transmembrane domain, an intracellular costimulatory domain, and/or an intracellular signaling domain in tandem.

In some embodiments, the GPRC5D specific CAR includes an antibody or antigen binding fragment thereof, a transmembrane domain, a co-stimulatory signaling domain, and a signaling domain. In some embodiments, the antibody or antigen binding fragment thereof is an anti-GPRC5D single-chain antibody fragment (scFv) or single-domain antibody fragment (sdAb). Table 7 provides several non-limiting exemplary sequences of full-length GPRC5D scFv and sdAb sequences. In some embodiments, the GPRC5D specific CAR includes an anti-GPRC5D single-chain antibody fragment (scFv) or single-domain antibody fragment (sdAb), a transmembrane domain such as one derived from human CD8α, a 4-1BB (CD137) co-stimulatory signaling domain, and a CD3ζ signaling domain. In some embodiments, the CAR is bispecific and specifically binds human GPRC5D and another tumor antigen selected from CD5, CD19, CD20, CD22, CD23, CD30, CD33, CD38, CD70, CD123, CD138, GPRC5D, LeY, NKG2D, WT1, GD2, HER2, EGFR, EGFRvIII, B7H3, PSMA, PSCA, CAIX, CD171, CEA, CSPG4, EPHA2, FAP, FRα, IL-13Rα, Mesothelin, MUC1, MUC16, ROR1, C-Met, CD133, Ep-CAM, GPC3, HPV16, IL13Ra2, MAGEA3, MAGEA4, MART1, NY-ESO, VEGFR2, α-Folate, CD24, CD44v7/8, EGP-2, EGP-40, erb-B2, erb-B, FBP, Fetal acetylcholine e receptor, GD2, GD3, HMW-MAA, IL-11Rα, KDR, Lewis Y, L1-cell adhesion molecule, MADE-A1, Oncofetal antigen (h5T4), TAG-72, CD19/22, Syndecan 1, or BCMA. In some embodiments, the bispecific CAR includes an anti-GPRC5D scFv, and a scFv that specifically binds one of CD5, CD19, CD20, CD22, CD23, CD30, CD33, CD38, CD70, CD123, CD138, GPRC5D, LeY, NKG2D, WT1, GD2, HER2, EGFR, EGFRvIII, B7H3, PSMA, PSCA, CAIX, CD171, CEA, CSPG4, EPHA2, FAP, FRα, IL-13Rα, Mesothelin, MUC1, MUC16, ROR1, C-Met, CD133, Ep-CAM, GPC3, HPV16, IL13Ra2, MAGEA3, MAGEA4, MART1, NY-ESO, VEGFR2, α-Folate, CD24, CD44v7/8, EGP-2, EGP-40, erb-B2, erb-B, FBP, Fetal acetylcholine e receptor, GD2, GD3, HMW-MAA, IL-11Rα, KDR, Lewis Y, L1-cell adhesion molecule, MADE-A1, Oncofetal antigen (h5T4), TAG-72, CD19/22, Syndecan 1, or BCMA, a transmembrane domain, a co-stimulatory signaling domain, and a signaling domain. In some embodiments, the bispecific CAR includes an anti-GPRC5D scFv, and a scFv that specifically binds one of CD5, CD19, CD20, CD22, CD23, CD30, CD33, CD38, CD70, CD123, CD138, GPRC5D, LeY, NKG2D, WT1, GD2, HER2, EGFR, EGFRvIII, B7H3, PSMA, PSCA, CAIX, CD171, CEA, CSPG4, EPHA2, FAP, FRα, IL-13Rα, Mesothelin, MUC1, MUC16, ROR1, C-Met, CD133, Ep-CAM, GPC3, HPV16, IL13Ra2, MAGEA3, MAGEA4, MART1, NY-ESO, VEGFR2, α-Folate, CD24, CD44v7/8, EGP-2, EGP-40, erb-B2, erb-B, FBP, Fetal acetylcholine e receptor, GD2, GD3, HMW-MAA, IL-11Rα, KDR, Lewis Y, L1-cell adhesion molecule, MADE-A1, Oncofetal antigen (h5T4), TAG-72, CD19/22, Syndecan 1, or BCMA, a transmembrane domain such as one derived from human CD8α, a 4-1BB (CD137) co-stimulatory signaling domain, and a CD3ζ signaling domain.

In some embodiments, the signal peptide of the GPRC5D CAR comprises a CD8a signal peptide. In some embodiments, the CD8a signal peptide comprises or consists of an amino acid sequence set forth in SEQ ID NO: 28 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 28. In some embodiments, the signal peptide comprises an IgK signal peptide. In some embodiments, the IgK signal peptide comprises or consists of an amino acid sequence set forth in SEQ ID NO: 29 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:29. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO: 30 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 30. In some embodiments, the signal peptide comprises a Immunoglobulin heavy chain signal peptide. In some embodiments, the Immunoglobulin heavy chain signal peptide comprises or consists of an amino acid sequence set forth in SEQ ID NO: 31 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 31. Table 8 provides several non-limiting examples of sequences of exemplary signal peptides.

In some embodiments, the extracellular binding domain of the GPRC5D CAR is specific to GPRC5D, for example, human GPRC5D. The extracellular binding domain of the GPRC5D CAR is codon-optimized for expression in a host cell or to have variant sequences to increase functions of the extracellular binding domain.

In some embodiments, the extracellular binding domain comprises an immunogenically active portion of an immunoglobulin molecule, for example, an scFv. In some embodiments, the extracellular binding domain of the GPRC5D CAR is derived from an antibody specific to GPRC5D, including, for example, any one of the antibodies or antigen binding fragments thereof herein disclosed, and telquetamab. In any of these embodiments, the extracellular binding domain of the GPRC5D CAR can comprise or consist of the V_{H}, the V_{L}, and/or one or more CDRs of any of the antibodies or antigen binding fragments thereof disclosed herein.

In some embodiments, the extracellular binding domain of the GPRC5D CAR comprises an scFv. The scFv may comprise the heavy chain variable region (V_{H}) and the light chain variable region (V_{L}) connected by a (G4S)3 linker or by a Whitlow linker, the amino acid sequences of which set forth in SEQ ID NO: 32 and 33, respectively, set forth in Table 9. In some embodiments, the GPRC5D-specific extracellular binding domain comprises or consists of an amino acid sequence set forth in SEQ ID NO: 25, 26, or 27, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 25, 26, or 27, set forth in Table 7. In some embodiments, the GPRC5D-specific extracellular binding domain may comprise one or more heavy chain CDRs having amino acid sequences set forth in Table 3 and one or more light chain CDRs having amino acid sequences set forth in Table 4. In some embodiments, the GPRC5D-specific extracellular binding domain may comprise a heavy chain having amino acid sequences set forth in Table 5. In some embodiments, the GPRC5D-specific extracellular binding domain may comprise a light chain having amino acid sequences set forth in Table 6. In any of these embodiments, the GPRC5D-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In any of these embodiments, the GPRCSD-specific scFv may comprise one or more heavy chains (VH) comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In any of these embodiments, the GPRCSD-specific scFv may comprise one or more light chains (VL) comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the GPRC5D CAR comprises or consists of the one or more CDRs as described herein.

In some embodiments, the extracellular binding domain of the GPRC5D CAR comprises single variable fragments of a heavy chain (VH) that can bind to an epitopes of GPRC5D.

In some embodiments, the hinge domain of the GPRC5D CAR comprises a CD8a hinge domain, for example, a human CD8a hinge domain. In some embodiments, the CD8a hinge domain comprises or consists of an amino acid sequence set forth in SEQ ID NO: 34 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 34. In some embodiments, the hinge domain comprises a CD28 hinge domain, for example, a human CD28 hinge domain. In some embodiments, the CD28 hinge domain comprises or consists of an amino acid sequence set forth in SEQ ID NO: 35 or 36, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 35 or 36. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO: 37 or SEQ ID NO: 38, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 37 or SEQ ID NO: 38. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO: 39 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 39. Non-limiting exemplary sequences of hinge domains are set forth in Table 10.

In some embodiments, the transmembrane domain comprises one selected from a group that includes a transmembrane region of TCRα, TCRβ, TCRζ, CD3ε, CD3γ, CD3δ, CD3ζ, CD4, CDS, CD8α, CD8β, CD9, CD16, CD28, CD45, CD22, CD33, CD34, CD37, CD40, CD40L/CD154, CD45, CD64, CD80, CD86, OX40/CD134, 4-1BB/CD137, CD154, FcεRIγ, VEGFR2, FAS, FGFR2B, and functional variant thereof.

In some embodiments, the transmembrane domain of the GPRC5D CAR comprises a CD8a transmembrane domain, for example, a human CD8a transmembrane domain. In some embodiments, the CD8a transmembrane domain comprises or consists of an amino acid sequence set forth in SEQ ID NO: 40 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 40.

In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO: 41 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 41. In some embodiments, the CD28 transmembrane domain comprises or consists of an amino acid sequence set forth in SEQ ID NO: 42 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 42. Non-limiting exemplary sequences of transmembrane domains are set forth in Table 11.

In some embodiments, the signaling domain(s) of the CAR comprises a costimulatory domain(s). For instance, a signaling domain can contain a costimulatory domain. Or, a signaling domain can contain one or more costimulatory domains. In some embodiments, the signaling domain comprises a costimulatory domain. In other embodiments, the signaling domains comprise costimulatory domains. In some embodiments, when the CAR comprises two or more costimulatory domains, two costimulatory domains are not the same. In some embodiments, the costimulatory domains comprise two costimulatory domains that are not the same. In some embodiments, the costimulatory domain enhances cytokine production, CAR-T cell proliferation, and/or CAR-T cell persistence during T cell activation. In some embodiments, the costimulatory domains enhance cytokine production, CAR-T cell proliferation, and/or CAR-T cell persistence during T cell activation.

In some embodiments, the intracellular costimulatory domain of the GPRC5D CAR comprises a 4-1BB costimulatory domain, for example, a human 4-1BB costimulatory domain. In some embodiments, the 4-1BB costimulatory domain comprises or consists of an amino acid sequence set forth in SEQ ID NO: 43 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 43. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO: 44 or 45, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 44 or 45. In some embodiments, the CD3ζ signaling domain of SEQ ID NO:46 may have a mutation, e.g., a glutamine (Q) to lysine (K) mutation, at amino acid position 14 (see SEQ ID NO:47). Non-limiting exemplary sequences of intracellular costimulatory and/or signaling domains are set forth in Table 12.

In some embodiments, the intracellular signaling domain of the GPRC5D CAR comprises a CD3 zeta (ζ) signaling domain, for example, a human CD3ζ signaling domain. In some embodiments, the CD3ζ signaling domain comprises or consists of an amino acid sequence set forth in SEQ ID NO: 46 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 46.

In some embodiments, the CAR comprises an amino acid sequence set for in SEQ ID NOs: 195, 196, 197, 198, 199, 200, 201, or 202, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NOs: 195, 196, 197, 198, 199, 200, 201, or 202. In some embodiments, the CAR comprises a binding domain comprising an amino acid sequence set forth in SEQ ID NOs: 25, 26, or 27, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NOs: 25, 26, or 27.

In some embodiments, the polycistronic vector comprises an expression cassette that contains a nucleotide sequence encoding a GPRC5D CAR, including, for example, a GPRC5D CAR comprising any of the GPRCSD-specific extracellular binding domains as described, the CD8a hinge domain of SEQ ID NO: 48, the CD8a transmembrane domain of SEQ ID NO: 40, the 4-1BB costimulatory domain of SEQ ID NO: 43, the CD3ζ signaling domain of SEQ ID NO: 46, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the GPRC5D CAR may additionally comprise a signal peptide (e.g., a CD8a signal peptide) as described.

In some embodiments, the polycistronic vector comprises an expression cassette that contains a nucleotide sequence encoding a GPRC5D CAR, including, for example, a GPRC5D CAR comprising any of the GPRC5D-specific extracellular binding domains as described, the CD8a hinge domain of SEQ ID NO: 34, the CD8a transmembrane domain of SEQ ID NO: 40, the CD28 costimulatory domain of SEQ ID NO: 44, the CD3ζ signaling domain of SEQ ID NO: 46, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the GPRCSD CAR may additionally comprise a signal peptide as described.

In some embodiments, the antibody portion of the recombinant receptor, e.g., CAR, further includes spacer between the transmembrane domain and extracellular antigen binding domain. In some embodiments, the spacer includes at least a portion of an immunoglobulin constant region, such as a hinge region, e.g., an IgG4 hinge region, and/or a CH1/CL and/or Fc region. In some embodiments, the constant region or portion is of a human IgG, such as IgG4 or IgGl. In some aspects, the portion of the constant region serves as a spacer region between the antigen-recognition component, e.g., scFv, and transmembrane domain. The spacer is of a length that provides for increased responsiveness of the cell following antigen binding, as compared to in the absence of the spacer. Exemplary spacers include, but are not limited to, those described in Hudecek et al. (2013) Clin. Cancer Res., 19:3153, WO2014031687, U.S. Pat. No. 8,822,647 or published app. No. US 2014/0271635. In some embodiments, the constant region or portion is of a human IgG, such as IgG4 or IgGl.

In some embodiments, the antigen receptor comprises an intracellular domain linked directly or indirectly to the extracellular domain. In some embodiments, the chimeric antigen receptor includes a transmembrane domain linking the extracellular domain and the intracellular signaling domain. In some embodiments, the intracellular signaling domain comprises an ITAM.

For example, in some aspects, the antigen recognition domain (e.g., extracellular domain) generally is linked to one or more intracellular signaling components, such as signaling components that mimic activation through an antigen receptor complex, such as a TCR complex, in the case of a CAR, and/or signal via another cell surface receptor. In some embodiments, the chimeric receptor comprises a transmembrane domain linked or fused between the extracellular domain (e.g., scFv) and intracellular signaling domain. Thus, in some embodiments, the antigen-binding component (e.g., antibody) is linked to one or more transmembrane and intracellular signaling domains.

In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e., comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s). In some aspects, the transmembrane domain contains a transmembrane portion of CD28.

In some embodiments, the extracellular domain and transmembrane domain are linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

The receptor, e.g., the CAR, generally includes at least one intracellular signaling component or components. In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the intracellular component is or includes a CD3-zeta intracellular signaling domain. In some embodiments, the intracellular component is or includes a signaling domain from Fc receptor gamma chain. In some embodiments, the receptor, e.g., CAR, includes the intracellular signaling domain and further includes a portion, such as a transmembrane domain and/or hinge portion, of one or more additional molecules such as CD8, CD4, CD25, or CD16.

For example, in some aspects, the CAR or other chimeric receptor is a chimeric molecule of CD3-zeta (CD3-z) or Fc receptor g and a portion of one of CD8, CD4, CD25 or CD16.

In some embodiments, upon ligation of the CAR or other chimeric receptor, the cytoplasmic domain or intracellular signaling domain of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

In some embodiments, the chimeric antigen receptor contains an intracellular domain of a T cell costimulatory molecule. In some embodiments, the CAR includes a signaling domain and/or transmembrane portion of a costimulatory receptor, such as CD28, 4-1BB, 0X40, DAP10, and ICOS. In some aspects, the same CAR includes both the activating and costimulatory components. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In some embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments the intracellular signaling domain includes intracellular components of a 4-1BB signaling domain and a CD3-zeta signaling domain. In some embodiments, the intracellular signaling domain includes intracellular components of a CD28 signaling domain and a CD3zeta signaling domain.

In some embodiments, the CAR comprises an extracellular antigen binding domain (e.g., antibody or antibody fragment, such as an scFv) that binds to an antigen (e.g., tumor antigen), a spacer (e.g., containing a hinge domain, such as any as described herein), a transmembrane domain (e.g., any as described herein), and an intracellular signaling domain (e.g., any intracellular signaling domain, such as a primary signaling domain or costimulatory signaling domain as described herein). In some embodiments, the intracellular signaling domain is or includes a primary cytoplasmic signaling domain. In some embodiments, the intracellular signaling domain additionally includes an intracellular signaling domain of a costimulatory molecule (e.g., a costimulatory domain). Non-limiting examples of exemplary components of a CAR are described in Table 13. In provided aspects, the sequences of each component in a CAR include any combination listed in Table 13.

In some embodiments, the antigen receptor further includes a marker and/or cells expressing the CAR or other antigen receptor further includes a surrogate marker, such as a cell surface marker, which is used to confirm transduction or engineering of the cell to express the receptor. In some aspects, the marker includes all or part (e.g., truncated form) of CD34, a NGFR, or epidermal growth factor receptor, such as truncated version of such a cell surface receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, is any as disclosed in published patent application No. WO2014031687. For example, the marker is a truncated EGFR (tEGFR) that is, optionally, linked to a linker sequence, such as a T2A cleavable linker sequence.

In some embodiments, the marker is a molecule, e.g., cell surface protein, not naturally found on T cells or not naturally found on the surface of T cells, or a portion thereof. In some embodiments, the molecule is a non-self molecule, e.g., non-self protein, i.e., one that is not recognized as “self by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker is a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some embodiments, CARs are referred to as first, second, third generation, and/or fourth generation CARs. In some embodiments, the CAR disclosed herein is selected from a group including: (a) a first generation CAR comprising an antigen binding domain, a transmembrane domain, and a signaling domain; (b) a second generation CAR comprising an antigen binding domain, a transmembrane domain, and at least two signaling domains; (c) a third generation CAR comprising an antigen binding domain, a transmembrane domain, and at least three signaling domains; and (d) a fourth generation CAR comprising an antigen binding domain, a transmembrane domain, three or four signaling domains, and a domain which upon successful signaling of the CAR induces expression of a cytokine gene.

As described herein, a fourth generation CAR can contain an antigen binding domain, a transmembrane domain, three or four signaling domains, and a domain which upon successful signaling of the CAR induces expression of a cytokine gene. In some instances, the cytokine gene is an endogenous or exogenous cytokine gene of the hypoimmunogenic cells. In some embodiments, the cytokine gene encodes a pro-inflammatory cytokine. In some embodiments, the pro-inflammatory cytokine is selected from a group that includes IL-1, IL-2, IL-9, IL-12, IL-18, TNF, IFN-gamma, and a functional fragment thereof. In some embodiments, the domain which upon successful signaling of the CAR induces expression of the cytokine gene comprises a transcription factor or functional domain or fragment thereof.

In some embodiments, the CAR contains an antibody, e.g., an antibody fragment, as disclosed herein, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, as disclosed herein, a transmembrane domain that is or contains a transmembrane portion of CD28 or a functional variant thereof, and an intracellular signaling domain containing a signaling portion of a 4-IBB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g., an IgG4 hinge, such as a hinge-only spacer.

In some aspects, the spacer contains only a hinge region of an IgG, such as only a hinge of IgG4 or IgG. In other embodiments, the spacer is or contains an Ig hinge, e.g., an IgG4-derived hinge, optionally linked to a CH2 and/or CH3 domains. In some embodiments, the spacer is an Ig hinge, e.g., an IgG4 hinge, linked to CH2 and CH3 domains. In some embodiments, the spacer is an Ig hinge, e.g., an IgG4 hinge, linked to a CH3 domain only. In some embodiments, the spacer is or comprises a glycine-serine rich sequence or other flexible linker such as known flexible linkers.

For example, in some embodiments, the CAR includes an antibody such as an antibody fragment, including scFvs and sdAbs as disclosed herein, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv or sdAb as disclosed herein, a spacer such as any of the Ig-hinge containing spacers, a CD28-derived transmembrane domain, a 4-1BB-derived intracellular signaling domain, and a CD3 zeta-derived signaling domain.

The recombinant receptors, such as CARs, expressed by the cells administered to the subject generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells express a CAR that specifically binds to an antigen expressed by a cell or tissue of the disease or condition or associated with the disease or condition.

Some embodiments of the disclosure are an isolated polynucleotide encoding any of the CARs or CAR components of the disclosure. Certain exemplary polynucleotides are disclosed herein, however, other polynucleotides which, given the degeneracy of the genetic code or codon preferences in a given expression system, encode the antibodies or antigen binding fragments thereof of the disclosure are also within the scope of the disclosure. The polynucleotide sequences encoding the CARs or CAR components thereof of the disclosure are operably linked to one or more regulatory elements, such as a promoter and enhancer, that allow expression of the nucleotide sequence in the intended host cell. The polynucleotide is a cDNA.

Some embodiments of the disclosure are a vector comprising the polynucleotide of the disclosure. In some embodiments, such vectors are plasmid vectors, viral vectors, vectors for baculovirus expression, transposon-based vectors, or any other vector suitable for introduction of the polynucleotide of the disclosure into a given organism or genetic background by any means. For example, polynucleotides encoding light and heavy chain variable regions of the antibodies of the disclosure, optionally linked to constant regions, are inserted into expression vectors. The light and heavy chains are cloned in the same or different expression vectors. In some embodiments, the DNA segments encoding immunoglobulin chains are operably linked to control sequences in the expression vector(s) that ensure the expression of immunoglobulin polypeptides. Such control sequences include signal sequences, promoters (e.g., naturally associated or heterologous promoters), enhancer elements, and transcription termination sequences, and are chosen to be compatible with the host cell chosen to express the antibody. Once the vector has been incorporated into the appropriate host, the host is maintained under conditions suitable for high level expression of the proteins encoded by the incorporated polynucleotides.

Suitable expression vectors are typically replicable in the host organisms either as episomes or as an integral part of the host chromosomal DNA. Commonly, expression vectors contain selection markers such as ampicillin-resistance, hygromycin-resistance, tetracycline resistance, kanamycin resistance, or neomycin resistance to permit detection of those cells transformed with the desired DNA sequences. Suitable vectors, promoter, and enhancer elements are known in the art; many are commercially available for generating subject recombinant constructs.

Some embodiments of the disclosure are a method of producing a CAR, comprising delivering a polynucleotide encoding a CAR as herein described, or a vector comprising a polynucleotide encoding a CAR as herein described to a host cell. In some embodiments, the method of delivery of the polynucleotide or the vector is any method for delivery of nucleic acids known to those skilled in the art, and include, but are not limited to, transfection, transduction, electroporation, and transformation.

Some embodiments of the disclosure are a host cell comprising the vector of the disclosure. The term “host cell” refers to a cell into which a vector has been introduced. It is understood that the term host cell is intended to refer not only to the particular subject cell but to the progeny of such a cell. Because certain modifications may occur in succeeding generations due to either mutation or environmental influences, such progeny may not be identical to the parent cell, but are still included within the scope of the term “host cell” as used herein. Such host cells include eukaryotic cells, prokaryotic cells, plant cells, or archaeal cells. Escherichia coli, bacilli, such as Bacillus subtilis, and other enterobacteriaceae, such as Salmonella, Serratia, and various Pseudomonas species are examples of prokaryotic host cells. Other microbes, such as yeast, are also useful for expression. Saccharomyces (e.g., S. cerevisiae) and Pichia are examples of suitable yeast host cells. Exemplary eukaryotic cells are of mammalian, insect, avian, or other animal origins.

### Vector for Delivering a CAR

Also provided herein are targeted lipid particles (e.g., vectors) that comprise a targeting antibody or antigen binding fragment thereof for delivery of the targeted lipid particle to a target cell and an exogenous agent. In some embodiments, the targeted lipid particle comprises a henipavirus F protein molecule or a biologically active portion thereof. In some embodiments the targeted lipid particle comprises a henipavirus G protein molecule or a biologically active portion thereof. In some embodiments, the targeted lipid particle comprises a henipavirus F protein molecule or biologically active portion thereof and a henipavirus G protein molecule or biologically active portion thereof.

In some embodiments, the targeting antibody or antigen binding fragment thereof is attached on a membrane-bound protein of the targeted lipid particle. In other embodiments, the targeting antibody or antigen binding fragment thereof is attached to a fusogen on the outer surface of the targeted lipid particle. In some embodiments the targeting antibody or antigen binding fragment thereof is attached to the henipavirus G protein or a biologically active portion thereof.

In some embodiments, the target cell is an immune cell. In some embodiments, the immune cell is a NK cell, a T cell, a macrophage, or a monocyte. In some embodiments, the immune cell is a T cell. In some embodiments, the T cell is a CD3+ T cell, a CD4+ T cell, a CDS+ T cell, a naive T cell, a regulatory T (Treg) cell, a non-regulatory T cell, a Th1 cell, a Th2 cell, a Th9 cell, a Th17 cell, a T-follicular helper (Tfh) cell, a cytotoxic T lymphocyte (CTL), an effector T (Teff) cell, a central memory T cell, an effector memory T cell, an effector memory T cell expressing CD45RA (TEMRA cell), a tissue-resident memory (Trm) cell, a virtual memory T cell, an innate memory T cell, a memory stem cell (Tse), or a TS T cell. In some embodiments, the T cell is a cytotoxic T cell, a helper T cell, a memory T cell, a regulatory T cell, or a tumor infiltrating lymphocyte. In some embodiments, the T cell is a CD4+ T cell. In other embodiments, the T cell is a CD8+ T cell.

### A. Lipid Bilayer

In some embodiments, the targeted lipid particle includes a naturally derived bilayer of amphipathic lipids that encloses a lumen or cavity. In some embodiments, the targeted lipid particle comprises a lipid bilayer as the outermost surface. In some embodiments, the lipid bilayer encloses a lumen. In some embodiments, the lumen is aqueous. In some embodiments, the lumen is in contact with the hydrophilic head groups on the interior of the lipid bilayer. In some embodiments, the lumen is a cytosol. In some embodiments, the cytosol contains cellular components present in a source cell. In some embodiments, the cytosol does not contain cellular components present in a source cell. In some embodiments, the lumen is a cavity. In some embodiments, the cavity contains an aqueous environment. In some embodiments, the cavity does not contain an aqueous environment.

In some aspects, the lipid bilayer is derived from a source cell during a process to produce a lipid-containing particle. In some embodiments, the lipid bilayer includes membrane components of the cell from which the lipid bilayer is produced, e.g., phospholipids, membrane proteins, etc. In some embodiments, the lipid bilayer includes a cytosol that includes components found in the cell from which the lipid bilayer is produced, e.g., solutes, proteins, nucleic acids, etc., but not all of the components of a cell, e.g., it lacks a nucleus. In some embodiments, the lipid bilayer is considered to be exosome-like. The lipid particle may vary in size, and in some instances have a diameter ranging from 30 and 300 nm, such as from 30 and 150 nm, and including from 40 to 100 nm.

In some embodiments, the lipid bilayer is a viral envelope. In some embodiments, the viral envelope is obtained from a source cell. In some embodiments, the viral envelope is obtained by the viral capsid from the source cell plasma membrane. In some embodiments, the lipid bilayer is obtained from a membrane other than the plasma membrane of a host cell. In some embodiments, the viral envelope lipid bilayer is embedded with viral proteins, including viral glycoproteins.

In other aspects, the lipid bilayer includes synthetic lipid complex. In some embodiments, the synthetic lipid complex is a liposome. In some embodiments, the lipid particle is a vesicular structure characterized by a phospholipid bilayer membrane and an inner aqueous medium. In some embodiments, the lipid bilayer has multiple lipid layers separated by aqueous medium. In some embodiments, the lipid bilayer forms spontaneously when phospholipids are suspended in an excess of aqueous solution. In some examples, the lipid components undergo self-rearrangement before the formation of closed structures and entrap water and dissolved solutes between the lipid bilayers.

In some embodiments, a targeted envelope protein and fusogen, such as any described above including any that are exogenous or overexpressed relative to the source cell, is disposed in the lipid bilayer.

In some embodiments, the targeted lipid particle comprises several different types of lipids. In some embodiments, the lipids are amphipathic lipids. In some embodiments, the amphipathic lipids are phospholipids. In some embodiments, the phospholipids comprise phosphatidylcholine, phosphatidylethanolamine, phosphatidylinositol, and phosphatidylserine. In some embodiments, the lipids comprise phospholipids such as phosphocholines and phosphoinositols. In some embodiments, the lipids comprise DMPC, DOPC, and DSPC.

In some embodiments, the bilayer is comprised of one or more lipids of the same or different type. In some embodiments, the source cell comprises a cell selected from CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

### B. Targeting Antibody

In some aspects, the targeted lipid particles (e.g., vectors) comprise a targeting antibody or antigen binding fragment thereof for delivery of the targeted lipid particle to a target cell.

In some embodiments, the targeting antibody or antigen binding fragment thereof is attached on a membrane-bound protein of the targeted lipid particle. In other embodiments, the targeting antibody or antigen binding fragment thereof is attached to a fusogen on the outer surface of the targeted lipid particle. In some embodiments the targeting antibody or antigen binding fragment thereof is attached to a henipavirus G protein or a biologically active portion thereof. In some embodiments, the C-terminus of the targeting antibody or antigen binding fragment thereof is attached to the C-terminus of a G protein or biologically active portion thereof. In some embodiments, the N-terminus end of the targeting antibody or antigen binding fragment thereof is exposed on the exterior surface of the lipid bilayer. In some embodiments, the N-terminus end of the targeting antibody or antigen binding fragment thereof binds to a cell surface molecule of a target cell. In some embodiments, the targeting antibody or antigen binding fragment thereof specifically binds to a cell surface molecule present on a target cell. In some embodiments, the cell surface molecule is a protein, glycan, lipid, or low molecular weight molecule.

In some embodiments, the cell surface molecule of a target cell is an antigen or portion thereof. In some embodiments, the targeting antibody or antigen binding fragment thereof is an antibody having a single monomeric domain antigen binding/recognition domain that is able to bind selectively to a specific antigen. In some embodiments, the single domain antibody binds an antigen present on a target cell. In some embodiments, the cell surface molecule is CD4 or CD8.

Exemplary cells include immune effector cells, peripheral blood mononuclear cells (PBMC) such as lymphocytes (T cells, B cells, natural killer cells) and monocytes, granulocytes (neutrophils, basophils, eosinophils), macrophages, dendritic cells, cytotoxic T lymphocytes, polymorphonuclear cells (also known as PMN, PML, or PMNL), stem cells, embryonic stem cells, neural stem cells, mesenchymal stem cells (MSCs), hematopoietic stem cells (HSCs), human myogenic stem cells, muscle-derived stem cells (MuStem), embryonic stem cells (ES or ESCs), limbal epithelial stem cells, cardio-myogenic stem cells, cardiomyocytes, progenitor cells, allogenic cells, resident cardiac cells, induced pluripotent stem cells (iPS), adipose-derived or phenotypic modified stem or progenitor cells, CD133+ cells, aldehyde dehydrogenase-positive cells (ALDH+), umbilical cord blood (UCB) cells, peripheral blood stem cells (PBSCs), neurons, neural progenitor cells, pancreatic beta cells, glial cells, or hepatocytes.

In some embodiments, the target cell is a cell of a target tissue. In some embodiments, the target tissue is liver, lungs, heart, spleen, pancreas, gastrointestinal tract, kidney, testes, ovaries, brain, reproductive organs, central nervous system, peripheral nervous system, skeletal muscle, endothelium, inner ear, or eye.

In some embodiments, the target cell is a muscle cell (e.g., skeletal muscle cell), kidney cell, liver cell (e.g., hepatocyte), or a cardiac cell (e.g., cardiomyocyte). In some embodiments, the target cell is a cardiac cell, e.g., a cardiomyocyte (e.g., a quiescent cardiomyocyte), a hepatoblast (e.g., a bile duct hepatoblast), an epithelial cell, a T cell (e.g., a naive T cell), a macrophage (e.g., a tumor infiltrating macrophage), or a fibroblast (e.g., a cardiac fibroblast).

In some embodiments, the target cell is a tumor-infiltrating lymphocyte, a T cell, a neoplastic or tumor cell, a virus-infected cell, a stem cell, a central nervous system (CNS) cell, a hematopoietic stem cell (HSC), a liver cell or a fully differentiated cell. In some embodiments, the target cell is a CD3+ T cell, a CD4+ T cell, a CD8+ T cell, a hepatocyte, a hematopoietic stem cell, a CD34+ hematopoietic stem cell, a CD105+ hematopoietic stem cell, a CD 117+ hematopoietic stem cell, a CD105+ endothelial cell, a B cell, a CD20+B cell, a CD19+B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacytoid dendritic cell, a CD11c+ cell, a CD11b+ cell, a splenocyte, a B cell, a hepatocyte, an endothelial cell, or a non-cancerous cell.

i. CD4 Antibody

In some embodiments, the targeting antibody or antigen binding fragment thereof that specifically target and bind CD4 for delivery of the targeted lipid particle to a cell expressing CD4. In some embodiments, the antibodies or antigen binding fragments thereof may cross-react with cynomolgus (or “cyno”) or M. nemestrina CD4. In some embodiments, the antibodies or antigen binding fragments thereof are single-chain variable fragments (scFvs) composed of the antigen-binding domains derived from the heavy (VH) and the light (VL) chains of the IgG molecule and connected via a linker domain. In some embodiments, the antibodies or antigen binding fragments thereof are VHHs that correspond to the VH of the IgG molecule. The present disclosure also provides polynucleotides encoding the antibodies and fragments thereof, vectors, and host cells, and methods of using the antibodies or antigen binding fragments thereof. In some embodiments, e.g., the antibodies or antigen binding fragments thereof are fused to henipavirus glycoprotein G for targeted binding and transduction to cells.

Sequences for exemplary antibodies and antigen binding fragments of the disclosure using the Kabat numbering scheme are shown in Tables 14-15 below. Sequences for exemplary HCDRs of the disclosure are shown in Table 14. Sequences for exemplary LCDRs of the disclosure are shown in Table 15. Additional suitable sequences of antibodies or antigen binding fragments thereof that specifically bind CD4 are disclosed, for example, in U.S. Provisional Application No. 63/326,269 and U.S. Provisional Application No. 63/341,681, which are hereby incorporated by reference in their entirety.

The sequences for the disclosed VH and VL domains are provided in Tables 16-17.

In some embodiments, an antibody or antigen binding fragment thereof capable of binding CD4 is disclosed, comprising a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3). In some embodiments, the HCDR1, HCDR2, and HCDR3 comprise amino acid sequences of any one of the SEQ ID NOs recited in Table 14 and the LCDR1, LCDR2, and LCDR3 comprise amino acid sequences of any one of the SEQ ID NOs recited in Table 15. In some embodiments, the heavy chain variable region (VH) comprises an amino acid sequence of any one of SEQ ID NOs: 71-74 (Table 16) and the light chain variable region (VL) comprises an amino acid sequence of any one of SEQ ID NOs: 75-77 (Table 17).

In some embodiments, the antibody or antigen binding fragment thereof comprises a VH having an amino acid sequence with at least 80%, 85%, 90%, 95%, 96%, 97%, 98%, 99%, or 100% identity to a sequence selected from SEQ ID NOs: 71-74.

In some embodiments, the antibody or antigen binding fragment thereof comprises a VL having an amino acid sequence with at least 80%, 85%, 90%, 95%, 96%, 97%, 98%, 99%, or 100% identity to a sequence selected from SEQ ID NOs: 75-77.

In some embodiments, the antibody or antigen binding fragment comprises a VH having an amino acid sequence with at least 80%, 85%, 90%, 95%, 96%, 97%, 98%, 99%, or 100% identity to a sequence selected from SEQ ID NOs: 71-74 and a VL having an amino acid sequence with at least 80%, 85%, 90%, 95%, 96%, 97%, 98%, 99%, or 100% identity to a sequence selected from SEQ ID NOs: 75-77.

In some embodiments, the antibody or antigen binding fragment thereof comprises the HCDR1, HCDR2, HCDR3, LCDR1, LCDR2, and LCDR3 of SEQ ID NOs: 50, 54, 58, 62, 65, and 68, respectively.

In some embodiments, the antibody or antigen binding fragment thereof comprises the HCDR1, HCDR2, HCDR3, LCDR1, LCDR2, and LCDR3 of SEQ ID NOs: 51, 55, 59, 63, 66, and 69, respectively.

In some embodiments, the antibody or antigen binding fragment thereof comprises the HCDR1, HCDR2, HCDR3, LCDR1, LCDR2, and LCDR3 of SEQ ID NOs: 52, 56, 60, 64, 67, and 70, respectively.

In some embodiments, the antibody or antigen binding fragment thereof comprises the HCDR1, HCDR2, and HCDR3 of SEQ ID NOs: 53, 57, and 61, respectively.

In some embodiments, the single domain antibody is human or humanized. In some embodiments, the single domain antibody or portion thereof is naturally occurring. In some embodiments, the single domain antibody or portion thereof is synthetic.

In some embodiments, the single domain antibodies are antibodies whose complementary determining regions are part of a single domain polypeptide. In some embodiments, the single domain antibody is a heavy chain only antibody variable domain. In some embodiments, the single domain antibody does not include light chains.

In various embodiments, any of the antibodies or antigen binding fragments described herein can comprise a heavy chain constant region and a light chain constant region. In some embodiments, the heavy chain constant region is an IgG, IgM, IgA, IgD, or IgE isotype, or a derivative or fragment thereof that retains at least one effector function of the intact heavy chain. In some embodiments, the heavy chain constant region is a human IgG isotype. In some embodiments, the heavy chain constant region is a human IgG1 or human IgG4 isotype. In some embodiments, the heavy chain constant region is a human IgG1 isotype. In some embodiments, the light chain constant region is a human kappa light chain or lambda light chain or a derivative or fragment thereof that retains at least one effector function of the intact light chain. In some embodiments, the light chain constant region is a human kappa light chain.

In various embodiments, any of the disclosed antibodies or antigen binding fragments are a rodent antibody or antigen binding fragment thereof, a chimeric antibody or an antigen binding fragment thereof, a CDR-grafted antibody or an antigen binding fragment thereof, or a humanized antibody or an antigen binding fragment thereof. In some embodiments, any of the disclosed antibodies or antigen binding fragments comprises human or human-derived heavy and light chain variable regions, including human frameworks or human frameworks with one or more backmutations. In various embodiments, any of the disclosed antibodies or antigen binding fragments are a Fab, Fab′, F(ab′)2, Fd, scFv, (scFv)2, scFv-Fc, VHH, or Fv fragment.

Antibodies whose heavy chain CDR, light chain CDR, VH, or VL amino acid sequences differ insubstantially from those shown in Tables 14-17 are encompassed within the scope of the disclosure. Typically, this involves one or more conservative amino acid substitutions with an amino acid having similar charge, hydrophobic, or stereo chemical characteristics in the antigen-binding site or in the framework without adversely altering the properties of the antibody. Conservative substitutions may also be made to improve antibody properties, for example stability or affinity. 1, 2, 3, 4, 5, 6, 7, 8, 9, 10, 11, 12, 13, 14, or 15 amino acid substitutions are made to the VH or VL sequence. For example, a “conservative amino acid substitution” may involve a substitution of a native amino acid residue with a nonnative residue such that there is little or no effect on the polarity or charge of the amino acid residue at that position. Desired amino acid substitutions are determined by those skilled in the art at the time such substitutions are desired. For example, amino acid substitutions are used to identify important residues of the molecule sequence, or to increase or decrease the affinity of the molecules described herein. The following eight groups contain amino acids that are conservative amino acid substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

In some embodiments, the antibody or antigen binding fragment thereof binds to human CD4. In some embodiments, the antibody or antigen binding fragment binding CD4 is a single-chain variable fragment. In embodiments involving a single polypeptide containing both a heavy chain variable region and a light chain variable region, both orientations of these variable regions are contemplated. In some embodiments, the heavy chain variable region is on the N-terminal side of the light chain variable region, which means the heavy chain variable region is closer to the N-terminus of the polypeptide. In other embodiments, the light chain variable region is on the N-terminal side of the heavy chain variable region, which means the light chain variable region is closer to the N-terminus of the polypeptide than the heavy chain variable region.

In some embodiments, the scFv binding proteins comprise a linker. In some embodiments, the linker is between the heavy chain variable region (VH) and the light chain variable region (VL) (or vice versa). In some embodiments, the linker comprises the amino acid sequence of GS, GGS, GGGS, GGGGS (SEQ ID NO: 147), GGGGGS (SEQ ID NO: 145), any one of SEQ ID NOs: 32-33, 165-166, or combinations thereof. Substitutions to introduce new disulfide bonds are also within the scope of the disclosure, e.g., by making substitutions G44C in the VH FR 2 and G100C in the VL FR4.

In some embodiments, the anti-CD4 antibody or antigen binding fragment binds to human CD4 with an affinity constant (K_{D}) of between about 1 nM and about 900 nM. In some embodiments, the K_{D} to human CD4 is between about 5 nM about 500 nM, about 6 nM to about 10 nM, about 11 nM to about 20 nM, about 25 nM to about 40 nM, about 40 nM to about 60 nM, about 70 nM to about 90 nM, about 100 nM to about 120 nM, about 125 nM to about 140 nM, about 145 nM to about 160 nM, about 170 nM and to about 200 nM, about 210 nM to about 250 nM, about 260 nM to about 300 nM, about 310 nM to about 350 nM, about 360 nM to about 400 nM, about 410 nM to about 450 nM, and about 460 nM to about 500 nM. In some embodiments, the anti-CD4 antibody or antigen binding fragment binds to human CD4 with an affinity constant (K_{D}) of 500 nM, 400 nM, 300 nM, 200 nM, 100 nM, 50 nM, 20 nM, or 10 nM or lower. In some embodiments, the anti-CD4 antibody or antigen binding fragment binds to human CD4 and cynomolgus, M. mulatta (rhesus monkey), or M. nemestrina CD4 with comparable binding affinity (K_{D}).

In some embodiments, the anti-CD4 antibody or antigen binding fragment binds to cynomolgus, M. mulatta (rhesus monkey), or N. nemestrina CD4. In some embodiments, the anti-CD4 antibody or antigen binding binds to mouse, dog, pig, etc., CD4. In some embodiments, the K_{D} to cynomolgus or M. nemestrina CD4 is between about 5 nM about 500 nM, about 6 nM to about 10 nM, about 11 nM to about 20 nM, about 25 nM to about 40 nM, about 40 nM to about 60 nM, about 70 nM to about 90 nM, about 100 nM to about 120 nM, about 125 nM to about 140 nM, about 145 nM to about 160 nM, about 170 nM and to about 200 nM, about 210 nM to about 250 nM, about 260 nM to about 300 nM, about 310 nM to about 350 nM, about 360 nM to about 400 nM, about 410 nM to about 450 nM, and about 460 nM to about 500 nM. In some embodiments, the anti-CD4 antibody or antigen binding fragment binds to cynomolgus or M. nemestrina CD4 with an affinity constant (K_{D}) of 500 nM, 400 nM, 300 nM, 200 nM, 100 nM, 50 nM, 20 nM, or 10 nM or lower.

An antibody or antigen binding fragment thereof that specifically binds CD4 refers to an antibody or binding fragment that preferentially binds to CD4 over other antigen targets. As used herein, the term is interchangeable with an “anti-CD4” antibody or an “antibody that binds CD4.” In some embodiments, the antibody or binding fragment capable of binding to CD4 can do so with higher affinity for that antigen than others. In some embodiments, the antibody or binding fragment capable of binding CD4 can bind to that antigen with a K_{D} of at least about 10⁻¹, 10⁻², 10⁻³, 10⁻⁴, 10⁻⁵, 10⁻⁶, 10⁻⁷, 10⁻⁸, 10⁻⁹, 10⁻¹⁰, 10⁻¹¹, 10⁻¹² or greater (or any value in between), e.g., as measured by surface plasmon resonance or other methods known to those skilled in the art.

ii. CD8 Antibody

In some embodiments, the targeting antibody or antigen binding fragment thereof that specifically target and bind CD8a or CD8β for delivery of the targeted lipid particle to a cell expressing CD8. In some embodiments, the antibodies or antigen binding fragments thereof may cross-react with cynomolgus (or “cyno”) or M. nemestrina CD8. In some embodiments, the antibodies or antigen binding fragments thereof are single-chain variable fragments (scFvs) composed of the antigen-binding domains derived from the heavy (VH) and the light (VL) chains of the IgG molecule and connected via a linker domain. In some embodiments, the antibodies or antigen binding fragments thereof are VHHs that correspond to the VH of the IgG molecule. The present disclosure also provides polynucleotides encoding the antibodies and fragments thereof, vectors, and host cells, and methods of using the antibodies or antigen binding fragments thereof. In some embodiments, e.g., the antibodies or antigen binding fragments thereof are fused to henipavirus glycoprotein G for targeted binding and transduction to cells.

Sequences for exemplary antibodies and antigen binding fragments of the disclosure using the Kabat numbering scheme are shown in Tables 18-19 below. Sequences for exemplary HCDRs of the disclosure are shown in Table 18. Sequences for exemplary LCDRs of the disclosure are shown in Table 19. Additional suitable sequences of antibodies or antigen binding fragments thereof that specifically bind CD8 are disclosed, for example, in PCT Application Publication No. WO2022/216915, which is hereby incorporated by reference in its entirety.

The sequences for the disclosed VH and VL domains are provided in Tables 20-21.

In some embodiments, an antibody or antigen binding fragment thereof capable of binding CD8α or CD8β is disclosed, comprising a heavy chain variable region and a light chain variable region, wherein the heavy chain variable region comprises three heavy chain complementarity determining regions (HCDR1, HCDR2, and HCDR3), and the light chain variable region comprises three light chain complementarity determining regions (LCDR1, LCDR2, and LCDR3). In some embodiments, the HCDR1, HCDR2, and HCDR3 comprise amino acid sequences of any one of the SEQ ID NOs recited in Table 18, and the LCDR1, LCDR2, and LCDR3 comprise amino acid sequences of any one of the SEQ ID NOs recited in Table 19. In some embodiments, the heavy chain variable region (VH) comprises an amino acid sequence of any one of SEQ ID NOs: 102-105 (Table 20) and the light chain variable region (VL) comprises an amino acid sequence of any one of SEQ ID NOs: 106-109 (Table 21).

In some embodiments, the antibody or antigen binding fragment thereof comprises a VH having an amino acid sequence with at least 80%, 85%, 90%, 95%, 96%, 97%, 98%, 99%, or 100% identity to a sequence selected from SEQ ID NOs: 102-105.

In some embodiments, the antibody or antigen binding fragment thereof comprises a VL having an amino acid sequence with at least 80%, 85%, 90%, 95%, 96%, 97%, 98%, 99%, or 100% identity to a sequence selected from SEQ ID NOs: 106-109.

In some embodiments, the antibody or antigen binding fragment comprises a VH having an amino acid sequence with at least 80%, 85%, 90%, 95%, 96%, 97%, 98%, 99%, or 100% identity to a sequence selected from SEQ ID NOs: 102-105 and a VL having an amino acid sequence with at least 80%, 85%, 90%, 95%, 96%, 97%, 98%, 99%, or 100% identity to a sequence selected from SEQ ID NOs: 106-109.

In some embodiments, the antibody or antigen binding fragment thereof comprises the HCDR1, HCDR2, HCDR3, LCDR1, LCDR2, and LCDR3 of SEQ ID NOs: 78, 82, 86, 90, 94, and 98, respectively.

In some embodiments, the antibody or antigen binding fragment thereof comprises the HCDR1, HCDR2, HCDR3, LCDR1, LCDR2, and LCDR3 of SEQ ID NOs: 79, 83, 87, 91, 95, and 99, respectively.

In some embodiments, the antibody or antigen binding fragment thereof comprises the HCDR1, HCDR2, HCDR3, LCDR1, LCDR2, and LCDR3 of SEQ ID NOs: 80, 84, 88, 92, 11960, and 100, respectively.

In some embodiments, the antibody or antigen binding fragment thereof comprises the HCDR1, HCDR2, HCDR3, LCDR1, LCDR2, and LCDR3 of SEQ ID NOs: 81, 85, 89, 93, 97, and 101, respectively.

In some embodiments, the single domain antibody is human or humanized. In some embodiments, the single domain antibody or portion thereof is naturally occurring. In some embodiments, the single domain antibody or portion thereof is synthetic.

In some embodiments, the single domain antibodies are antibodies whose complementary determining regions are part of a single domain polypeptide. In some embodiments, the single domain antibody is a heavy chain only antibody variable domain. In some embodiments, the single domain antibody does not include light chains.

In various embodiments, any of the antibodies or antigen binding fragments described herein can comprise a heavy chain constant region and a light chain constant region. In some embodiments, the heavy chain constant region is an IgG, IgM, IgA, IgD, or IgE isotype, or a derivative or fragment thereof that retains at least one effector function of the intact heavy chain. In some embodiments, the heavy chain constant region is a human IgG isotype. In some embodiments, the heavy chain constant region is a human IgG1 or human IgG4 isotype. In some embodiments, the heavy chain constant region is a human IgG1 isotype. In some embodiments, the light chain constant region is a human kappa light chain or lambda light chain or a derivative or fragment thereof that retains at least one effector function of the intact light chain. In some embodiments, the light chain constant region is a human kappa light chain.

In various embodiments, any of the disclosed antibodies or antigen binding fragments are a rodent antibody or antigen binding fragment thereof, a chimeric antibody or an antigen binding fragment thereof, a CDR-grafted antibody or an antigen binding fragment thereof, or a humanized antibody or an antigen binding fragment thereof. In some embodiments, any of the disclosed antibodies or antigen binding fragments comprises human or human-derived heavy and light chain variable regions, including human frameworks or human frameworks with one or more backmutations. In various embodiments, any of the disclosed antibodies or antigen binding fragments are a Fab, Fab′, F(ab′)2, Fd, scFv, (scFv)2, scFv-Fc, VHH, or Fv fragment.

Antibodies whose heavy chain CDR, light chain CDR, VH, or VL amino acid sequences differ insubstantially from those shown in Tables 18-21 are encompassed within the scope of the disclosure. Typically, this involves one or more conservative amino acid substitutions with an amino acid having similar charge, hydrophobic, or stereo chemical characteristics in the antigen-binding site or in the framework without adversely altering the properties of the antibody. Conservative substitutions may also be made to improve antibody properties, for example stability or affinity. 1, 2, 3, 4, 5, 6, 7, 8, 9, 10, 11, 12, 13, 14, or 15 amino acid substitutions are made to the VH or VL sequence. For example, a “conservative amino acid substitution” may involve a substitution of a native amino acid residue with a nonnative residue such that there is little or no effect on the polarity or charge of the amino acid residue at that position. Desired amino acid substitutions are determined by those skilled in the art at the time such substitutions are desired. For example, amino acid substitutions are used to identify important residues of the molecule sequence, or to increase or decrease the affinity of the molecules described herein. The following eight groups contain amino acids that are conservative amino acid substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

In some embodiments, the antibody or antigen binding fragment thereof binds to human CD8α or CD8β. In some embodiments, the antibody or antigen binding fragment thereof binds to a human CD8α homodimer composed of two a chains. In some embodiments, the antibody or antigen binding fragment thereof binds to a human CD8 heterodimer composed of one a chain and one β chain.

In some embodiments, the antibody or antigen binding fragment binding CD8 is a single-chain variable fragment. In embodiments involving a single polypeptide containing both a heavy chain variable region and a light chain variable region, both orientations of these variable regions are contemplated. In some embodiments, the heavy chain variable region is on the N-terminal side of the light chain variable region, which means the heavy chain variable region is closer to the N-terminus of the polypeptide. In other embodiments, the light chain variable region is on the N-terminal side of the heavy chain variable region, which means the light chain variable region is closer to the N-terminus of the polypeptide than the heavy chain variable region.

In some embodiments, the scFv binding proteins comprise a linker. In some embodiments, the linker is between the heavy chain variable region (VH) and the light chain variable region (VL) (or vice versa). In some embodiments, the linker comprises the amino acid sequence of GS, GGS, GGGS, GGGGS (SEQ ID NO: 147), GGGGGS (SEQ ID NO: 145), any one of SEQ ID NOs: 32-33, 165-166, or combinations thereof. Substitutions to introduce new disulfide bonds are also within the scope of the disclosure, e.g., by making substitutions G44C in the VH FR 2 and G100C in the VL FR4.

In some embodiments, the anti-CD8 antibody or antigen binding fragment binds to human CD8 with an affinity constant (K_{D}) of between about 1 nM and about 900 nM. In some embodiments, the K_{D} to human CD8 is between about 5 nM about 500 nM, about 6 nM to about 10 nM, about 11 nM to about 20 nM, about 25 nM to about 40 nM, about 40 nM to about 60 nM, about 70 nM to about 90 nM, about 100 nM to about 120 nM, about 125 nM to about 140 nM, about 145 nM to about 160 nM, about 170 nM and to about 200 nM, about 210 nM to about 250 nM, about 260 nM to about 300 nM, about 310 nM to about 350 nM, about 360 nM to about 400 nM, about 410 nM to about 450 nM, and about 460 nM to about 500 nM. In some embodiments, the anti-CD8 antibody or antigen binding fragment binds to human CD8 with an affinity constant (K_{D}) of 500 nM, 400 nM, 300 nM, 200 nM, 100 nM, 50 nM, 20 nM, or 10 nM or lower. In some embodiments, the anti-CD8 antibody or antigen binding fragment binds to human CD8 and cynomolgus, M. mulatta (rhesus monkey), or M. nemestrina CD8 with comparable binding affinity (K_{D}).

In some embodiments, the anti-CD8 antibody or antigen binding fragment binds to cynomolgus, M. mulatta (rhesus monkey), or N. nemestrina CD8. In some embodiments, the anti-CD8 antibody or antigen binding binds to mouse, dog, pig, etc., CD8. In some embodiments, the K_{D} to cynomolgus or M. nemestrina CD8 is between about 5 nM about 500 nM, about 6 nM to about 10 nM, about 11 nM to about 20 nM, about 25 nM to about 40 nM, about 40 nM to about 60 nM, about 70 nM to about 90 nM, about 100 nM to about 120 nM, about 125 nM to about 140 nM, about 145 nM to about 160 nM, about 170 nM and to about 200 nM, about 210 nM to about 250 nM, about 260 nM to about 300 nM, about 310 nM to about 350 nM, about 360 nM to about 400 nM, about 410 nM to about 450 nM, and about 460 nM to about 500 nM. In some embodiments, the anti-CD8 antibody or antigen binding fragment binds to cynomolgus or M. nemestrina CD8 with an affinity constant (K_{D}) of 500 nM, 400 nM, 300 nM, 200 nM, 100 nM, 50 nM, 20 nM, or 10 nM or lower.

An antibody or antigen binding fragment thereof that specifically binds CD8a or CD8β refers to an antibody or binding fragment that preferentially binds to CD8a or CD8β, respectively, over other antigen targets. As used herein, the term is interchangeable with an “anti-CD8” antibody or an “antibody that binds CD8.” In some embodiments, the antibody or binding fragment capable of binding to CD8α or CD8β can do so with higher affinity for that antigen than others. In some embodiments, the antibody or binding fragment capable of binding CD8α or CD8β can bind to that antigen with a K_{D} of at least about 10⁻¹, 10⁻², 10⁻³, 10⁻⁴, 10⁻⁵, 10⁻⁶, 10⁻⁷, 10⁻⁸, 10⁻⁹, 10⁻¹⁰, 10⁻¹¹, 10⁻¹² or greater (or any value in between), e.g., as measured by surface plasmon resonance or other methods known to those skilled in the art.

### C. Exogenous Agent

In some embodiments, the targeted vector further comprises an agent that is exogenous relative to the source cell (also referred to herein as a “cargo” or “payload”). In some embodiments, the exogenous agent is a small molecule, a protein, or a nucleic acid (e.g., a DNA, a chromosome (e.g., a human artificial chromosome), an RNA, e.g., an mRNA or miRNA). In some embodiments, the exogenous agent or cargo encodes a cytosolic protein. In some embodiments the exogenous agent or cargo comprises or encodes a membrane protein. In some embodiments, the exogenous agent or cargo comprises a therapeutic agent. In some embodiments, the therapeutic agent is chosen from one or more of a protein, e.g., an enzyme, a transmembrane protein, a receptor, an antibody; a nucleic acid, e.g., DNA, a chromosome (e.g., a human artificial chromosome), RNA, mRNA, siRNA, miRNA; or a small molecule.

In some embodiments, the exogenous agent is present in at least, or no more than, 10, 20, 50, 100, 200, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the targeted lipid particle has an altered, e.g., increased or decreased level of one or more endogenous molecules, e.g., protein or nucleic acid (e.g., in some embodiments, endogenous relative to the source cell, and in some embodiments, endogenous relative to the target cell), e.g., due to treatment of the source cell, e.g., mammalian source cell with a siRNA or gene editing enzyme. In some embodiments, the endogenous molecule is present in at least, or no more than, 10, 20, 50, 100, 200, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the endogenous molecule (e.g., an RNA or protein) is present at a concentration of at least 1, 2, 3, 4, 5, 10, 20, 50, 100, 500, 10³, 5.0×10³, 10⁴, 5.0×10⁴, 105, 5.0×10⁵, 10⁶, 5.0×10⁶, 1.0×10⁷, 5.0×10⁷, or 1.0×10⁸, greater than its concentration in the source cell. In some embodiments, the endogenous molecule (e.g., an RNA or protein) is present at a concentration of at least 1, 2, 3, 4, 5, 10, 20, 50, 100, 500, 10³, 5.0×10³, 10⁴, 5.0×10⁴, 105, 5.0×10⁵, 10⁶, 5.0×10⁶, 1.0×10⁷, 5.0×10⁷, or 1.0×10⁸ less than its concentration in the source cell.

In some embodiments, the targeted lipid particle (e.g., targeted vector) delivers to a target cell at least 10%, 20%, 30%, 40%, 50%, 60%, 70%, 80%, 90%, 95%, 96%, 97%, 98%, or 99% of the cargo (e.g., a therapeutic agent, e.g., an exogenous therapeutic agent) comprised by the targeted lipid particle. In some embodiments, the targeted lipid particle that fuses with the target cell(s) delivers to the target cell an average of at least 10%, 20%, 30%, 40%, 50%, 60%, 70%, 80%, 90%, 95%, 96%, 97%, 98%, or 99% of the cargo (e.g., a therapeutic agent, e.g., an exogenous therapeutic agent) comprised by the targeted lipid particle that fuses with the target cell(s). In some embodiments, the targeted lipid particle composition delivers to a target tissue at least 10%, 20%, 30%, 40%, 50%, 60%, 70%, 80%, 90%, 95%, 96%, 97%, 98%, or 99% of the cargo (e.g., a therapeutic agent, e.g., an exogenous therapeutic agent) comprised by the targeted lipid particle composition.

In some embodiments, the exogenous agent or cargo is not expressed naturally in the cell from which the targeted lipid particle is derived. In some embodiments, the exogenous agent or cargo is expressed naturally in the cell from which the vector is derived. In some embodiments, the exogenous agent or cargo is loaded into the targeted lipid particle via expression in the cell from which the vector is derived (e.g., expression from DNA or mRNA introduced via transfection, transduction, or electroporation). In some embodiments, the exogenous agent or cargo is expressed from DNA integrated into the genome or maintained episosomally. In some embodiments, expression of the exogenous agent or cargo is constitutive. In some embodiments, expression of the exogenous agent or cargo is induced. In some embodiments, expression of the exogenous agent or cargo is induced immediately prior to generating the targeted lipid particle. In some embodiments, expression of the exogenous agent or cargo is induced at the same time as expression of the fusogen.

In some embodiments, the exogenous agent or cargo is loaded into the targeted lipid particle via electroporation into the targeted lipid particle itself or into the cell from which the targeted lipid particle is derived. In some embodiments, the exogenous agent or cargo is loaded into the targeted lipid particle via transfection (e.g., of a DNA or mRNA encoding the cargo) into the targeted lipid particle itself or into the cell from which the targeted lipid particle is derived.

In some embodiments, the exogenous agent or cargo may include one or more nucleic acid sequences, one or more polypeptides, a combination of nucleic acid sequences and/or polypeptides, one or more organelles, and any combination thereof. In some embodiments, the exogenous agent or cargo may include one or more cellular components. In some embodiments, the exogenous agent or cargo includes one or more cytosolic and/or nuclear components.

In some embodiments, the exogenous agent or cargo includes a nucleic acid, e.g., DNA, nDNA (nuclear DNA), mtDNA (mitochondrial DNA), protein coding DNA, gene, transgene, operon, chromosome, genome, transposon, retrotransposon, viral genome, vector, polycistronic vector, intron, exon, modified DNA, mRNA (messenger RNA), tRNA (transfer RNA), modified RNA, microRNA, siRNA (small interfering RNA), tmRNA (transfer messenger RNA), rRNA (ribosomal RNA), mtRNA (mitochondrial RNA), snRNA (small nuclear RNA), small nucleolar RNA (snoRNA), SmY RNA (mRNA trans-splicing RNA), gRNA (guide RNA), TERC (telomerase RNA component), aRNA (antisense RNA), cis-NAT (Cis-natural antisense transcript), CRISPR RNA (crRNA), lncRNA (long noncoding RNA), piRNA (piwi-interacting RNA), shRNA (short hairpin RNA), tasiRNA (trans-acting siRNA), eRNA (enhancer RNA), satellite RNA, pcRNA (protein coding RNA), dsRNA (double stranded RNA), RNAi (interfering RNA), circRNA (circular RNA), reprograming RNAs, aptamers, and any combination thereof. In some embodiments, the nucleic acid is a wild-type nucleic acid. In some embodiments, the nucleic acid is a mutant nucleic acid. In some embodiments the nucleic acid is a fusion or chimera of multiple nucleic acid sequences.

In some embodiments, the exogenous agent or cargo may include a nucleic acid. For example, the exogenous agent or cargo may comprise RNA to enhance expression of an endogenous protein, or a siRNA or miRNA that inhibits protein expression of an endogenous protein. For example, the endogenous protein may modulate structure or function in the target cells. In some embodiments, the cargo may include a nucleic acid encoding an engineered protein that modulates structure or function in the target cells. In some embodiments, the exogenous agent or cargo is a nucleic acid that targets a transcriptional activator that modulate structure or function in the target cells.

In some embodiments, the exogenous agent or cargo includes a polypeptide, e.g., enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides, metabolic polypeptides, kinases, transferases, hydrolases, lyases, isomer ases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, transposase polypeptides, DNA integration polypeptides, targeted endonucleases (e.g., Zinc-finger nucleases, transcription-activator-like nucleases (TALENs), cas9 and homologs thereof), recombinases, and any combination thereof. In some embodiments the protein targets a protein in the cell for degradation. In some embodiments the protein targets a protein in the cell for degradation by localizing the protein to the proteasome. In some embodiments, the protein is a wild-type protein. In some embodiments, the protein is a mutant protein. In some embodiments the protein is a fusion or chimeric protein.

In some embodiments, the exogenous agent or cargo includes a small molecule, e.g., ions (e.g., Ca²⁺, C1-, Fe²⁺), carbohydrates, lipids, reactive oxygen species, reactive nitrogen species, isoprenoids, signaling molecules, heme, polypeptide cofactors, electron accepting compounds, electron donating compounds, metabolites, ligands, and any combination thereof. In some embodiments the small molecule is a pharmaceutical that interacts with a target in the cell. In some embodiments the small molecule targets a protein in the cell for degradation. In some embodiments the small molecule targets a protein in the cell for degradation by localizing the protein to the proteasome. In some embodiments that small molecule is a proteolysis targeting chimera molecule (PROTAC).

In some embodiments, the exogenous agent or cargo includes a mixture of proteins, nucleic acids, or metabolites, e.g., multiple polypeptides, multiple nucleic acids, multiple small molecules; combinations of nucleic acids, polypeptides, and small molecules; ribonucleoprotein complexes (e.g., Cas9-gRNA complex); multiple transcription factors, multiple epigenetic factors, reprogramming factors (e.g., Oct4, Sox2, cMyc, and Klf4); multiple regulatory RNAs; and any combination thereof.

In some embodiments, the exogenous agent or cargo includes one or more organelles, e.g., chondrisomes, mitochondria, lysosomes, nucleus, cell membrane, cytoplasm, endoplasmic reticulum, ribosomes, vacuoles, endosomes, spliceosomes, polymerases, capsids, acrosome, autophagosome, centriole, glycosome, glyoxysome, hydrogenosome, melanosome, mitosome, myofibril, cnidocyst, peroxisome, proteasome, vesicle, stress granule, networks of organelles, and any combination thereof.

In some embodiments, the exogenous agent encodes a therapeutic agent or a diagnostic agent. In some embodiments, the therapeutic agent is a chimeric antigen receptor (CAR). In some embodiments, the CAR specifically binds GPRC5D. In some embodiments the CAR is bispecific and specifically binds GPRC5D and specifically binds one of CD5, CD19, CD20, CD22, CD23, CD30, CD33, CD38, CD70, CD123, CD138, GPRC5D, LeY, NKG2D, WT1, GD2, HER2, EGFR, EGFRvIII, B7H3, PSMA, PSCA, CAIX, CD171, CEA, CSPG4, EPHA2, FAP, FRα, IL-13Rα, Mesothelin, MUC1, MUC16, ROR1, C-Met, CD133, Ep-CAM, GPC3, HPV16, IL13Ra2, MAGEA3, MAGEA4, MART1, NY-ESO, VEGFR2, α-Folate, CD24, CD44v7/8, EGP-2, EGP-40, erb-B2, erb-B, FBP, Fetal acetylcholine e receptor, GD2, GD3, HMW-MAA, IL-11Rα, KDR, Lewis Y, L1-cell adhesion molecule, MADE-A1, Oncofetal antigen (h5T4), TAG-72, CD19/22, Syndecan 1, or BCMA. In some embodiments, the CAR is engineered to comprise an intracellular signaling domain of the T cell antigen receptor complex zeta chain (e.g., CD3 zeta). In some embodiments, the intracellular domain is selected from a CD137 (4-1BB) signaling domain, a CD28 signaling domain, and a CD3zeta signaling domain.

### D. G Protein

Also provided herein are fusion proteins comprising an envelope glycoprotein G, H, and/or an F protein of the Paramyxoviridae family and a targeting antibody or antigen binding fragment thereof herein disclosed that are exposed on the surface on a lipid particle or viral vector. In some embodiments, the targeting antibody or antigen binding fragment thereof disclosed herein is fused to an envelope glycoprotein G, H, and/or an F protein of the Paramyxoviridae family. In some embodiments the fusogen contains a Nipah virus protein F, a measles virus F protein, a tupaia paramyxovirus F protein, a paramyxovirus F protein, a Hendra virus F protein, a Henipavirus F protein, a Morbilivirus F protein, a respirovirus F protein, a Sendai virus F protein, a rubulavirus F protein, or an avulavirus F protein. In some embodiments, the lipid particle contains a henipavirus envelope attachment glycoprotein G (G protein) or a biologically active portion thereof and/or a henipavirus envelope fusion glycoprotein F (F protein) or a biologically active portion thereof.

In some embodiments, the fusogen is glycoprotein GP64 of baculovirus, or glycoprotein GP64 variant E45K/T259A.

In some embodiments, the fusogen is a hemagglutinin-neuraminidase (HN) and/or fusion (F) protein (F/HN) from a respiratory paramyxovirus. In some embodiments, the respiratory paramyxovirus is a Sendai virus. The HN and F glycoproteins of Sendai viruses function to attach to sialic acids via the HN protein, and to mediate cell fusion for entry into cells via the F protein. In some embodiments, the fusogen is a F and/or HN protein from the murine parainfluenza virus type 1 (see e.g., U.S. Pat. No. 10,704,061).

In some embodiments, the lipid particle (e.g., viral vector) is pseudotyped with viral glycoproteins as described herein such as a NiV-F and/or NiV-G protein.

In some embodiments, the viral vector further comprises a vector-surface targeting moiety which specifically binds to a target ligand. In some embodiments, the vector-surface targeting moiety is a polypeptide. In some embodiments, a nucleic acid encoding the Paramyxovirus envelope protein (e.g., G protein) is modified with a targeting moiety to specifically bind to a target molecule on a target cell. In some embodiments, the targeting moiety is any targeting protein, including but not necessarily limited to antibodies and antigen binding fragments thereof as herein disclosed.

It has been reported that the henipavirus F proteins from various species exhibit compatibility with G proteins from other species to trigger fusion (Brandel-Tretheway et al. Journal of Virology. 2019. 93(13):e00577-19). In some aspects of the provided lipid particles (e.g., lentiviral vectors), the F protein is heterologous to the G protein, i.e., the F and G proteins or biologically active portions thereof are from different henipavirus species. For example, in some embodiments the G protein is from Hendra virus and the F protein is a NiV-F as described. In other aspects, the F and/or G protein are chimeric F and/or G protein containing regions of F and/or G proteins from different species of Henipavirus. In some embodiments, replacing a portion of the F protein with amino acids from a heterologous sequence of Henipavirus results in fusion to the G protein with the heterologous sequence. (Brandel-Tretheway et al. 2019). In some embodiments, the chimeric F and/or G protein contains an extracellular domain from one henipavirus species and a transmembrane and/or cytoplasmic domain from a different henipavirus species. For example, in some embodiments the F protein contains an extracellular domain of Hendra virus and a transmembrane/cytoplasmic domain of Nipah virus.

In some embodiments, the fusion protein contains a henipavirus envelope attachment glycoprotein G (G protein) or a biologically active portion thereof and a single domain antibody (sdAb) variable domain or a single chain variable fragment (scFv). In some embodiments, the sdAb variable domain or scFv is linked directly or indirectly to the G protein. In some embodiments, the sdAb variable domain or scFv is linked to the C-terminus (C-terminal amino acid) of the G protein or the biologically active portion thereof. In some embodiments, the linkage is via a peptide linker, such as a flexible peptide linker. Table 22 provides a list of non-limiting examples of G proteins.

In some embodiments the G protein is a Henipavirus G protein or a biologically active portion thereof. In some embodiments, the Henipavirus G protein is a Hendra (HeV) virus G protein, a Nipah (NiV) virus G-protein (NiV-G), a Cedar (CedPV) virus G-protein, a Mojiang virus G-protein, a bat Paramyxovirus G-protein, or a biologically active portion thereof. Non-limiting examples of G proteins include those corresponding to SEQ ID NOs: 129, 128, 129, 130, and 131.

In some embodiments, the attachment G proteins are type II transmembrane glycoproteins containing an N-terminal cytoplasmic tail (e.g., corresponding to amino acids 1-49 of SEQ ID NO: 120), a transmembrane domain (e.g., corresponding to amino acids 50-70 of SEQ ID NO: 120), and an extracellular domain containing an extracellular stalk (e.g., corresponding to amino acids 71-187 of SEQ ID NO: 120), and a globular head (corresponding to amino acids 188-602 of SEQ ID NO: 120). In such embodiments, the N-terminal cytoplasmic domain is within the inner lumen of the lipid bilayer and the C-terminal portion is the extracellular domain that is exposed on the outside of the lipid bilayer. Regions of the stalk in the C-terminal region (e.g., corresponding to amino acids 159-167 of NiV-G) have been shown to be involved in interactions with F protein and triggering of F protein fusion (Liu et al. 2015 J of Virology 89:1838). In wild-type G protein, the globular head mediates receptor binding to henipavirus entry receptors ephrin B2 and ephrin B3, but is dispensable for membrane fusion (Brandel-Tretheway et al. Journal of Virology. 2019. 93(13)e00577-19). In some embodiments herein, tropism of the G protein is altered by linkage of the G protein or biologically active fragment thereof (e.g., cytoplasmic truncation) to a sdAb variable domain. Binding of the G protein to a binding partner can trigger fusion mediated by a compatible F protein or a biologically active portion thereof. G protein sequences disclosed herein are predominantly disclosed as expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

G glycoproteins are highly conserved among henipavirus species. For example, the G proteins of NiV and HeV viruses share 79% amino acid identity. Studies have shown a high degree of compatibility among G proteins with F proteins of different species as demonstrated by heterotypic fusion activation (Brandel-Tretheway et al. Journal of Virology. 2019). As described further below, in some embodiments, a targeted lipid particle contains heterologous G and F proteins from different species.

In some embodiments, the G protein has a sequence set forth in any of SEQ ID NOs: 120, 129, 138, 139, 140, 141, 148, 156, or 158-160, or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOs: 120, 129, 138, 139, 140, 141, 148, 156, or 158-160. In some embodiments, the G protein or functionally active variant or biologically active portion is a protein that retains fusogenic activity in conjunction with a Henipavirus F protein, such as an F protein (e.g., NiV-F or HeV-F). Fusogenic activity includes the activity of the G protein in conjunction with a Henipavirus F protein to promote or facilitate fusion of two membrane lumens, such as the lumen of the targeted lipid particle having embedded in its lipid bilayer a henipavirus F and G protein, and a cytoplasm of a target cell, e.g., a cell that contains a surface receptor or molecule that is recognized or bound by the targeted lipid particle. In some embodiments, the F protein and G protein are from the same Henipavirus species (e.g., NiV-G and NiV-F). In some embodiments, the F protein and G protein are from different Henipavirus species (e.g., NiV-G and HeV-F).

In some embodiments, the G protein has the sequence of amino acids set forth in SEQ ID NOs: 120, 129, 138, 139, 140, 141, 148, 156, or 158-160, or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to any one of SEQ ID NOs: 120, 129, 138, 139, 140, 141, 148, 156, or 158-160 and retains fusogenic activity in conjunction with a Henipavirus F protein (e.g., NiV-F or HeV-F). In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to any one of SEQ ID NOs: 120, 129, 138, 139, 140, 141, 148, 156, or 158-160 and retains fusogenic activity in conjunction with a Henipavirus F protein (e.g., NiV-F or HeV-F).

Reference to retaining fusogenic activity includes activity (in conjunction with a Henipavirus F protein) that is at or about 10% to at or about 150% or more of the level or degree of binding of the corresponding wild-type G protein, such as set forth in any one of SEQ ID NOs: 120, 129, 138, 139, 140, 141, 148, 156, or 158-160, such as at least or at least about 10% of the level or degree of fusogenic activity of the corresponding wild-type G protein, such as at least or at least about 15% of the level or degree of fusogenic activity of the corresponding wild-type G protein, such as at least or at least about 20% of the level or degree of fusogenic activity of the corresponding wild-type G protein, such as at least or at least about 25% of the level or degree of fusogenic activity of the corresponding wild-type G protein, such as at least or at least about 30% of the level or degree of fusogenic activity of the corresponding wild-type G protein, such as at least or at least about 35% of the level or degree of fusogenic activity of the corresponding wild-type G protein, such as at least or at least about 40% of the level or degree of fusogenic activity of the corresponding wild-type G protein, such as at least or at least about 45% of the level or degree of fusogenic activity of the corresponding wild-type G protein, such as at least or at least about 50% of the level or degree of fusogenic activity of the corresponding wild-type G protein, such as at least or at least about 55% of the level or degree of fusogenic activity of the corresponding wild-type G protein, such as at least or at least about 60% of the level or degree of fusogenic activity of the corresponding wild-type G protein, such as at least or at least about 65% of the level or degree of fusogenic activity of the corresponding wild-type G protein, such as at least or at least about 70% of the level or degree of fusogenic activity of the corresponding wild-type G protein, such as at least or at least about 75% of the level or degree of fusogenic activity of the corresponding wild-type G protein, such as at least or at least about 80% of the level or degree of fusogenic activity of the corresponding wild-type G protein, such as at least or at least about 85% of the level or degree of fusogenic activity of the corresponding wild-type G protein, such as at least or at least about 90% of the level or degree of fusogenic activity of the corresponding wild-type G protein, such as at least or at least about 95% of the level or degree of fusogenic activity of the corresponding wild-type G protein, such as at least or at least about 100% of the level or degree of fusogenic activity of the corresponding wild-type G protein, or such as at least or at least about 120% of the level or degree of fusogenic activity of the corresponding wild-type G protein.

In some embodiments, the G protein is a mutant G protein that is a functionally active variant or biologically active portion containing one or more amino acid mutations, such as one or more amino acid insertions, deletions, substitutions, or truncations. In some embodiments, the mutations described herein relate to amino acid insertions, deletions, substitutions, or truncations of amino acids compared to a reference G protein sequence. In some embodiments, the reference G protein sequence is the wild-type sequence of a G protein or a biologically active portion thereof. In some embodiments, the functionally active variant or the biologically active portion thereof is a mutant of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein, or biologically active portions thereof. In some embodiments, the wild-type G protein has the sequence set forth in any one of SEQ ID NOs: 120, 129, 138, 139, 140, 141, 148, 156, or 158-160.

In some embodiments, the G protein is a mutant G protein that is a biologically active portion that is an N-terminally and/or C-terminally truncated fragment of a wild-type_Hendra (HeV) virus G protein, a wild-type Nipah (NiV) virus G-protein (NiV-G), a wild-type Cedar (CedPV) virus G-protein, a wild-type Mojiang virus G-protein, or a wild-type bat Paramyxovirus G-protein. In some embodiments, the truncation is an N-terminal truncation of all or a portion of the cytoplasmic domain. In some embodiments, the mutant G protein is a biologically active portion that is truncated and lacks up to 49 contiguous amino acid residues at or near the N-terminus of the wild-type G protein, such as a wild-type G protein set forth in any one of SEQ ID NOs: 120, 129, 138, 139, 140, 141, 148, 156, or 158-160. In some embodiments, the mutant G protein is truncated and lacks up to 49 contiguous amino acids, such as up to 49, 48, 47, 46, 45, 44, 43, 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acid(s) at the N-terminus of the wild-type G protein.

In some embodiments, the G protein is a wild-type Nipah virus G (NiV-G) protein or a Hendra virus G protein, or is a functionally active variant or biologically active portion thereof. In some embodiments, the G protein is a NiV-G protein that has the sequence set forth in SEQ ID NO:120, SEQ ID NO:138, or SEQ ID NO:148, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:120, SEQ ID NO:138, or SEQ ID NO:148.

In some embodiments, the G protein is a mutant NiV-G protein that is a biologically active portion of a wild-type NiV-G. In some embodiments, the biologically active portion is an N-terminally truncated fragment. In some embodiments, the mutant NiV-G protein is truncated and lacks up to 5 contiguous amino acid residues at or near the N-terminus of the wild-type NiV-G protein (SEQ ID NO:120, SEQ ID NO:138, or SEQ ID NO:148), up to 6 contiguous amino acid residues at or near the N-terminus of the wild-type NiV-G protein (SEQ ID NO:120, SEQ ID NO:138, or SEQ ID NO:148), up to 7 contiguous amino acid residues at or near the N-terminus of the wild-type NiV-G protein (SEQ ID NO: 120, SEQ ID NO:138, or SEQ ID NO:148), up to 8 contiguous amino acid residues at or near the N-terminus of the wild-type NiV-G protein (SEQ ID NO:120, SEQ ID NO:138, or SEQ ID NO:148), up to 9 contiguous amino acid residues at or near the N-terminus of the wild-type NiV-G protein (SEQ ID NO:120, SEQ ID NO:138, or SEQ ID NO:148), up to 10 contiguous amino acid residues at or near the N-terminus of the wild-type NiV-G protein (SEQ ID NO:120, SEQ ID NO:138, or SEQ ID NO:148), up to 11 contiguous amino acid residues at or near the N-terminus of the wild-type NiV-G protein (SEQ ID NO:134, SEQ ID NO:152, or SEQ ID NO:162), up to 12 contiguous amino acid residues at or near the N-terminus of the wild-type NiV-G protein (SEQ ID NO:120, SEQ ID NO:138, or SEQ ID NO:148), up to 13 contiguous amino acid residues at or near the N-terminus of the wild-type NiV-G protein (SEQ ID NO:120, SEQ ID NO:138, or SEQ ID NO:148), up to 14 contiguous amino acid residues at or near the N-terminus of the wild-type NiV-G protein (SEQ ID NO: 120, SEQ ID NO:138, or SEQ ID NO:148), up to 15 contiguous amino acid residues at or near the N-terminus of the wild-type NiV-G protein (SEQ ID NO:120, SEQ ID NO:138, or SEQ ID NO:148), up to 16 contiguous amino acid residues at or near the N-terminus of the wild-type NiV-G protein (SEQ ID NO:120, SEQ ID NO:138, or SEQ ID NO:148), up to 17 contiguous amino acid residues at or near the N-terminus of the wild-type NiV-G protein (SEQ ID NO: 120, SEQ ID NO:138, or SEQ ID NO:148), up to 18 contiguous amino acid residues at or near the N-terminus of the wild-type NiV-G protein (SEQ ID NO:120, SEQ ID NO:138, or SEQ ID NO:148), up to 19 contiguous amino acid residues at or near the N-terminus of the wild-type NiV-G protein (SEQ ID NO:120, SEQ ID NO:138, or SEQ ID NO:148), up to 20 contiguous amino acid residues at or near the N-terminus of the wild-type NiV-G protein (SEQ ID NO: 120, SEQ ID NO:138, or SEQ ID NO:148), up to 21 contiguous amino acid residues at or near the N-terminus of the wild-type NiV-G protein (SEQ ID NO:120, SEQ ID NO:138, or SEQ ID NO:148), up to 22 contiguous amino acid residues at or near the N-terminus of the wild-type NiV-G protein (SEQ ID NO:120, SEQ ID NO:138, or SEQ ID NO:148), up to 23 contiguous amino acid residues at or near the N-terminus of the wild-type NiV-G protein (SEQ ID NO: 120, SEQ ID NO:138, or SEQ ID NO:148), up to 24 contiguous amino acid residues at or near the N-terminus of the wild-type NiV-G protein (SEQ ID NO:120, SEQ ID NO:138, or SEQ ID NO:148), up to 25 contiguous amino acid residues at or near the N-terminus of the wild-type NiV-G protein (SEQ ID NO:120, SEQ ID NO:138, or SEQ ID NO:148), up to 26 contiguous amino acid residues at or near the N-terminus of the wild-type NiV-G protein (SEQ ID NO: 120, SEQ ID NO:138, or SEQ ID NO:148), up to 27 contiguous amino acid residues at or near the N-terminus of the wild-type NiV-G protein (SEQ ID NO:120, SEQ ID NO:138, or SEQ ID NO:148), up to 28 contiguous amino acid residues at or near the N-terminus of the wild-type NiV-G protein (SEQ ID NO:120, SEQ ID NO:138, or SEQ ID NO:148), up to 29 contiguous amino acid residues at or near the N-terminus of the wild-type NiV-G protein (SEQ ID NO: 120, SEQ ID NO:138, or SEQ ID NO:148), up to 30 contiguous amino acid residues at or near the N-terminus of the wild-type NiV-G protein (SEQ ID NO:120, SEQ ID NO:138, or SEQ ID NO:148), up to 31 contiguous amino acid residues at or near the N-terminus of the wild-type NiV-G protein (SEQ ID NO:120, SEQ ID NO:138, or SEQ ID NO:148), up to 32 contiguous amino acid residues at or near the N-terminus of the wild-type NiV-G protein (SEQ ID NO: 120, SEQ ID NO:138, or SEQ ID NO:148), up to 33 contiguous amino acid residues at or near the N-terminus of the wild-type NiV-G protein (SEQ ID NO:120, SEQ ID NO:138, or SEQ ID NO:148), up to 34 contiguous amino acid residues at or near the N-terminus of the wild-type NiV-G protein (SEQ ID NO:120, SEQ ID NO:138, or SEQ ID NO:148), up to 35 contiguous amino acid residues at or near the N-terminus of the wild-type NiV-G protein (SEQ ID NO: 120, SEQ ID NO:138, or SEQ ID NO:148), up to 36 contiguous amino acid residues at or near the N-terminus of the wild-type NiV-G protein (SEQ ID NO:120, SEQ ID NO:138, or SEQ ID NO:148), up to 37 contiguous amino acid residues at or near the N-terminus of the wild-type NiV-G protein (SEQ ID NO:120, SEQ ID NO:138, or SEQ ID NO:148), up to 38 contiguous amino acid residues at or near the N-terminus of the wild-type NiV-G protein (SEQ ID NO: 120, SEQ ID NO:138, or SEQ ID NO:148), up to 39 contiguous amino acid residues at or near the N-terminus of the wild-type NiV-G protein (SEQ ID NO:120, SEQ ID NO:138, or SEQ ID NO:148), up to 40 contiguous amino acid residues at or near the N-terminus of the wild-type NiV-G protein (SEQ ID NO:120, SEQ ID NO:138, or SEQ ID NO:148), up to 41 contiguous amino acid residues at or near the N-terminus of the wild-type NiV-G protein (SEQ ID NO: 120, SEQ ID NO:138, or SEQ ID NO:148), up to 42 contiguous amino acid residues at or near the N-terminus of the wild-type NiV-G protein (SEQ ID NO:120, SEQ ID NO:138, or SEQ ID NO:148), up to 43 contiguous amino acid residues at or near the N-terminus of the wild-type NiV-G protein (SEQ ID NO:120, SEQ ID NO:138, or SEQ ID NO:148), up to 44 contiguous amino acid residues at or near the N-terminus of the wild-type NiV-G protein (SEQ ID NO: 120, SEQ ID NO:138, or SEQ ID NO:148), or up to 45 contiguous amino acid residues at or near the N-terminus of the wild-type NiV-G protein (SEQ ID NO:120, SEQ ID NO:138, or SEQ ID NO:148).

In some embodiments, the NiV-G protein is a biologically active portion that does not contain a cytoplasmic domain. In some embodiments, the NiV-G protein without the cytoplasmic domain is encoded by SEQ ID NO:142.

In some embodiments, the mutant NiV-G protein comprises a sequence set forth in any of SEQ ID NOs: 121-126, 149-154, 132, 142, or 157, or is a functional variant thereof that has an amino acid sequence having at least at or about 80%, at least at or about 81%, at least at or about 82%, at least at or about 83%, at least at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NOs: 121-126, 149-154, 132, 142, or 157.

In some embodiments, the mutant NiV-G protein has a 5 amino acid truncation at or near the N-terminus of the wild-type NiV-G protein (SEQ ID NO:120, SEQ ID NO:138, or SEQ ID NO:148), such as set forth in SEQ ID NO:121 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:121, or as set forth in SEQ ID NO:149 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:149 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:149.

In some embodiments, the mutant NiV-G protein has a 10 amino acid truncation at or near the N-terminus of the wild-type NiV-G protein (SEQ ID NO:120, SEQ ID NO:138, or SEQ ID NO:148), such as set forth in SEQ ID NO:122 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:122, or such as set forth in SEQ ID NO:150 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:150.

In some embodiments, the mutant NiV-G protein has a 15 amino acid truncation at or near the N-terminus of the wild-type NiV-G protein (SEQ ID NO:120, SEQ ID NO:138, or SEQ ID NO:148), such as set forth in SEQ ID NO: 123 or a functional variant thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:123, or such as set forth in SEQ ID NO:151 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:151.

In some embodiments, the mutant NiV-G protein has a 20 amino acid truncation at or near the N-terminus of the wild-type NiV-G protein (SEQ ID NO:120, SEQ ID NO:138, or SEQ ID NO:148) such as set forth in SEQ ID NO:124, or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least or about 96%, at least at or about 97%, at least at or about 98%, or at least at or about 99% sequence identity to SEQ ID NO:124, or such as set forth in SEQ ID NO:152 or a functional variant thereof having at least at or about 80%, at least at or about 81%, at least at or about 82%, at least at or about 83%, at least or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:152.

In some embodiments, the mutant NiV-G protein has a 25 amino acid truncation at or near the N-terminus of the wild-type NiV-G protein (SEQ ID NO:120, SEQ ID NO:138, or SEQ ID NO:148), such as set forth in SEQ ID NO: 125 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least or about 96%, at least at or about 97%, at least at or about 98%, or at least at or about 99% sequence identity to SEQ ID NO:125, or such as set forth in SEQ ID NO:153 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:153.

In some embodiments, the mutant NiV-G protein has a 30 amino acid truncation at or near the N-terminus of the wild-type NiV-G protein (SEQ ID NO:120, SEQ ID NO:138, or SEQ ID NO:148), such as set forth in SEQ ID NO: 126 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:126, or such as set forth in SEQ ID NO:154 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:154.

In some embodiments, the mutant NiV-G protein has a 33 amino acid truncation at or near the N-terminus of the wild-type NiV-G protein (SEQ ID NO:120, SEQ ID NO:138, or SEQ ID NO:148) or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:132, or such as set forth in SEQ ID NO:155 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:155. [0293]n some embodiments, the mutant NiV-G protein has a 34 amino acid truncation at or near the N-terminus of the wild-type NiV-G protein (SEQ ID NO:120, SEQ ID NO:138, or SEQ ID NO:148), such as set forth in SEQ ID NO: 132 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:132, or such as set forth in SEQ ID NO:155 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:155.

In some embodiments, the NiV-G protein has a 34 amino acid truncation at or near the N-terminus of the wild-type NiV-G protein (SEQ ID NO: 134, SEQ ID NO: 152, or SEQ ID NO: 162) and one or more amino acid substitutions corresponding to amino acid substitutions selected from E501A, W504A, Q530A, and E533A with reference to the numbering set forth in SEQ ID NO: 152.

In some embodiments, the mutant NiV-G protein lacks the N-terminal cytoplasmic domain of the wild-type NiV-G protein (SEQ ID NO:120, SEQ ID NO:138, or SEQ ID NO:148), such as set forth in SEQ ID NO: 142 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO: 142.

In some embodiments, the mutant G protein is a mutant HeV-G protein that has the sequence set forth in SEQ ID NO: 129 or 156, or is a functional variant or biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least or about 96%, at least at or about 97%, at least at or about 98%, or at least at or about 99% sequence identity to SEQ ID NO:129 or 156.

In some embodiments, the G protein is a mutant HeV-G protein that is a biologically active portion of a wild-type HeV-G. In some embodiments, the biologically active portion is an N-terminally truncated fragment. In some embodiments, the mutant HeV-G protein is truncated and lacks up to 5 contiguous amino acid residues at or near the N-terminus of the wild-type HeV-G protein (SEQ ID NO: 129 or 156), up to 6 contiguous amino acid residues at or near the N-terminus of the wild-type HeV-G protein (SEQ ID NO:129 or 156), up to 7 contiguous amino acid residues at or near the N-terminus of the wild-type HeV-G protein (SEQ ID NO:129 or 156), up to 8 contiguous amino acid residues at or near the N-terminus of the wild-type HeV-G protein (SEQ ID NO:129 or 156), up to 9 contiguous amino acid residues at or near the N-terminus of the wild-type HeV-G protein (SEQ ID NO:129 or 156), up to 10 contiguous amino acid residues at or near the N-terminus of the wild-type HeV-G protein (SEQ ID NO:129 or 156), up to 11 contiguous amino acid residues at or near the N-terminus of the wild-type HeV-G protein (SEQ ID NO:129 or 156), up to 12 contiguous amino acid residues at or near the N-terminus of the wild-type HeV-G protein (SEQ ID NO:129 or 156), up to 13 contiguous amino acid residues at or near the N-terminus of the wild-type HeV-G protein (SEQ ID NO:129 or 156), up to 14 contiguous amino acid residues at or near the N-terminus of the wild-type HeV-G protein (SEQ ID NO:129 or 156), up to 15 contiguous amino acid residues at or near the N-terminus of the wild-type HeV-G protein (SEQ ID NO:129 or 156), up to 16 contiguous amino acid residues at or near the N-terminus of the wild-type HeV-G protein (SEQ ID NO:129 or 156), up to 17 contiguous amino acid residues at or near the N-terminus of the wild-type HeV-G protein (SEQ ID NO:129 or 156), up to 18 contiguous amino acid residues at or near the N-terminus of the wild-type HeV-G protein (SEQ ID NO:129 or 156), up to 19 contiguous amino acid residues at or near the N-terminus of the wild-type HeV-G protein (SEQ ID NO:129 or 156), up to 20 contiguous amino acid residues at or near the N-terminus of the wild-type HeV-G protein (SEQ ID NO:129 or 156), up to 21 contiguous amino acid residues at or near the N-terminus of the wild-type HeV-G protein (SEQ ID NO:129 or 156), up to 22 contiguous amino acid residues at or near the N-terminus of the wild-type HeV-G protein (SEQ ID NO:129 or 156), up to 23 contiguous amino acid residues at or near the N-terminus of the wild-type HeV-G protein (SEQ ID NO:129 or 156), up to 24 contiguous amino acid residues at or near the N-terminus of the wild-type HeV-G protein (SEQ ID NO:129 or 156), up to 25 contiguous amino acid residues at or near the N-terminus of the wild-type HeV-G protein (SEQ ID NO:129 or 156), up to 26 contiguous amino acid residues at or near the N-terminus of the wild-type HeV-G protein (SEQ ID NO:129 or 156), up to 27 contiguous amino acid residues at or near the N-terminus of the wild-type HeV-G protein (SEQ ID NO:129 or 156), up to 28 contiguous amino acid residues at or near the N-terminus of the wild-type HeV-G protein (SEQ ID NO:129 or 156), up to 29 contiguous amino acid residues at or near the N-terminus of the wild-type HeV-G protein (SEQ ID NO:129 or 156), up to 30 contiguous amino acid residues at or near the N-terminus of the wild-type HeV-G protein (SEQ ID NO:129 or 159), up to 31 contiguous amino acid residues at or near the N-terminus of the wild-type HeV-G protein (SEQ ID NO:129 or 159), up to 32 contiguous amino acid residues at or near the N-terminus of the wild-type HeV-G protein (SEQ ID NO:129 or 159), up to 33 contiguous amino acid residues at or near the N-terminus of the wild-type HeV-G protein (SEQ ID NO:129 or 159), up to 34 contiguous amino acid residues at or near the N-terminus of the wild-type HeV-G protein (SEQ ID NO:129 or 159), up to 35 contiguous amino acid residues at or near the N-terminus of the wild-type HeV-G protein (SEQ ID NO:129 or 159), up to 36 contiguous amino acid residues at or near the N-terminus of the wild-type HeV-G protein (SEQ ID NO:129 or 159), up to 37 contiguous amino acid residues at or near the N-terminus of the wild-type HeV-G protein (SEQ ID NO:129 or 159), up to 38 contiguous amino acid residues at or near the N-terminus of the wild-type HeV-G protein (SEQ ID NO:129 or 159), up to 39 contiguous amino acid residues at or near the N-terminus of the wild-type HeV-G protein (SEQ ID NO:129 or 159), up to 40 contiguous amino acid residues at or near the N-terminus of the wild-type HeV-G protein (SEQ ID NO:129 or 159), up to 41 contiguous amino acid residues at or near the N-terminus of the wild-type HeV-G protein (SEQ ID NO:129 or 159), up to 42 contiguous amino acid residues at or near the N-terminus of the wild-type HeV-G protein (SEQ ID NO:129 or 159), up to 43 contiguous amino acid residues at or near the N-terminus of the wild-type HeV-G protein (SEQ ID NO:129 or 159), up to 44 contiguous amino acid residues at or near the N-terminus of the wild-type HeV-G protein (SEQ ID NO:129 or 159), or up to 45 contiguous amino acid residues at or near the N-terminus of the wild-type HeV-G protein (SEQ ID NO:129 or 159).

In some embodiments, the HeV-G protein is a biologically active portion that does not contain a cytoplasmic domain. In some embodiments, the mutant HeV-G protein lacks the N-terminal cytoplasmic domain of the wild-type HeV-G protein (SEQ ID NO:129 or 159), such as set forth in SEQ ID NO: 143 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO: 143.

In some embodiments, the G protein or the functionally active variant or biologically active portion thereof binds to Ephrin B2 or Ephrin B3. In some aspects, the G protein has the sequence of amino acids set forth in any one of SEQ ID NO:120, SEQ ID NO:129, SEQ ID NO:138, SEQ ID NO:139, SEQ ID NO: 148, SEQ ID NO:140, or SEQ ID NO:141, or is a functionally active variant thereof or a biologically active portion thereof that is able to bind to Ephrin B2 or Ephrin B3. In some embodiments, the functionally active variant or biologically active portion has an amino acid sequence having at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, and retains binding to Ephrin B2 or B3.

Reference to retaining binding to Ephrin B2 or B3 includes binding that is at least or at least about 5% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 10% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 15% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO: 140, or SEQ ID NO:141, or a functionally active variant or biologically active portion thereof, 20% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 25% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion, 30% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 35% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 40% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 45% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 50% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO: 140, or SEQ ID NO:141, or a functionally active variant or biologically active portion thereof, 55% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 60% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 65% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 70% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, such as at least or at least about 75% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, such as at least or at least about 80% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, such as at least or at least about 85% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, such as at least or at least about 90% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, or such as at least or at least about 95% of the level or degree of binding of the corresponding wild-type protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof.

In some embodiments, the G protein is NiV-G or a functionally active variant or biologically active portion thereof and binds to Ephrin B2 or Ephrin B3. In some aspects, the NiV-G has the sequence of amino acids set forth in SEQ ID NO:120, SEQ ID NO:138, or SEQ ID NO:148, or is a functionally active variant thereof or a biologically active portion thereof that is able to bind to Ephrin B2 or Ephrin B3. In some embodiments, the functionally active variant or biologically active portion has an amino acid sequence having at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:120, SEQ ID NO:138, or SEQ ID NO:148 and retains binding to Ephrin B2 or B3. Exemplary biologically active portions include N-terminally truncated variants lacking all or a portion of the cytoplasmic domain, e.g., 1 or more, such as 1 to 49 contiguous N-terminal amino acid residues, e.g., set forth in any one of SEQ ID NOS: 121-126, 142, and 149-154.

Reference to retaining binding to Ephrin B2 or B3 includes binding that is at least or at least about 5% of the level or degree of binding of the corresponding wild-type NiV-G, such as set forth in SEQ ID NO: 120, SEQ ID NO:138, or SEQ ID NO:148, 10% of the level or degree of binding of the corresponding wild-type NiV-G, such as set forth in SEQ ID NO:120, SEQ ID NO:138, or SEQ ID NO:148, 15% of the level or degree of binding of the corresponding wild-type NiV-G, such as set forth in SEQ ID NO:120, SEQ ID NO:138, or SEQ ID NO:148, 20% of the level or degree of binding of the corresponding wild-type NiV-G, such as set forth in SEQ ID NO:120, SEQ ID NO:138, or SEQ ID NO:148, 25% of the level or degree of binding of the corresponding wild-type NiV-G, such as set forth in SEQ ID NO:120, SEQ ID NO:138, or SEQ ID NO:148, 30% of the level or degree of binding of the corresponding wild-type NiV-G, such as set forth in SEQ ID NO:120, SEQ ID NO:138, or SEQ ID NO:148, 35% of the level or degree of binding of the corresponding wild-type NiV-G, such as set forth in SEQ ID NO:120, SEQ ID NO:138, or SEQ ID NO:148, 40% of the level or degree of binding of the corresponding wild-type NiV-G, such as set forth in SEQ ID NO:120, SEQ ID NO:138, or SEQ ID NO:148, 45% of the level or degree of binding of the corresponding wild-type NiV-G, such as set forth in SEQ ID NO:120, SEQ ID NO:138, or SEQ ID NO:148, 50% of the level or degree of binding of the corresponding wild-type NiV-G, such as set forth in SEQ ID NO:120, SEQ ID NO:138, or SEQ ID NO:148, 55% of the level or degree of binding of the corresponding wild-type NiV-G, such as set forth in SEQ ID NO:120, SEQ ID NO:138, or SEQ ID NO:148, 60% of the level or degree of binding of the corresponding wild-type NiV-G, such as set forth in SEQ ID NO:120, SEQ ID NO:138, or SEQ ID NO:148, 65% of the level or degree of binding of the corresponding wild-type NiV-G, such as set forth in SEQ ID NO:120, SEQ ID NO:138, or SEQ ID NO:148, 70% of the level or degree of binding of the corresponding wild-type NiV-G, such as set forth in SEQ ID NO:120, SEQ ID NO:138, or SEQ ID NO:148, such as at least or at least about 75% of the level or degree of binding of the corresponding wild-type NiV-G, such as set forth in SEQ ID NO:120, SEQ ID NO:138, or SEQ ID NO:148, such as at least or at least about 80% of the level or degree of binding of the corresponding wild-type NIV-G, such as set forth in SEQ ID NO:120, SEQ ID NO:138, or SEQ ID NO:148, such as at least or at least about 85% of the level or degree of binding of the corresponding wild-type NiV-G, such as set forth in SEQ ID NO:120, SEQ ID NO:138, or SEQ ID NO:148, such as at least or at least about 90% of the level or degree of binding of the corresponding wild-type NiV-G, such as set forth in SEQ ID NO:120, SEQ ID NO:138, or SEQ ID NO:148, or such as at least or at least about 95% of the level or degree of binding of the corresponding wild-type NiV-G, such as set forth in SEQ ID NO:120, SEQ ID NO:138, or SEQ ID NO:148.

In some embodiments, the G protein is HeV-G or a functionally active variant or biologically active portion thereof and binds to Ephrin B2 or Ephrin B3. In some aspects, the HeV-G has the sequence of amino acids set forth in SEQ ID NO:129 or 159, or is a functionally active variant thereof or a biologically active portion thereof that is able to bind to Ephrin B2 or Ephrin B3. In some embodiments, the functionally active variant or biologically active portion has an amino acid sequence having at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:129 or 159 and retains binding to Ephrin B2 or B3. Exemplary biologically active portions include N-terminally truncated variants lacking all or a portion of the cytoplasmic domain, e.g., 1 or more, such as 1 to 49 contiguous N-terminal amino acid residues, e.g., set forth in any one of SEQ ID NO:143.

Reference to retaining binding to Ephrin B2 or B3 includes binding that is at least or at least about 5% of the level or degree of binding of the corresponding wild-type HeV-G, such as set forth in SEQ ID NO: 129 or 159, 10% of the level or degree of binding of the corresponding wild-type HeV-G, such as set forth in SEQ ID NO:129 or 159, 15% of the level or degree of binding of the corresponding wild-type HeV-G, such as set forth in SEQ ID NO:129 or 159, 20% of the level or degree of binding of the corresponding wild-type HeV-G, such as set forth in SEQ ID NO:129 or 159, 25% of the level or degree of binding of the corresponding wild-type HeV-G, such as set forth in SEQ ID NO: 129 or 159, 30% of the level or degree of binding of the corresponding wild-type HeV-G, such as set forth in SEQ ID NO:129 or 159, 35% of the level or degree of binding of the corresponding wild-type HeV-G, such as set forth in SEQ ID NO:129 or 159, 40% of the level or degree of binding of the corresponding wild-type HeV-G, such as set forth in SEQ ID NO:129 or 159, 45% of the level or degree of binding of the corresponding wild-type HeV-G, such as set forth in SEQ ID NO:129 or 159, 50% of the level or degree of binding of the corresponding wild-type HeV-G, such as set forth in SEQ ID NO:129 or 159, 55% of the level or degree of binding of the corresponding wild-type HeV-G, such as set forth in SEQ ID NO:129 or 159, 60% of the level or degree of binding of the corresponding wild-type HeV-G, such as set forth in SEQ ID NO: 129 or 159, 65% of the level or degree of binding of the corresponding wild-type HeV-G, such as set forth in SEQ ID NO:129 or 159, 70% of the level or degree of binding of the corresponding wild-type HeV-G, such as set forth in SEQ ID NO:129 or 159, such as at least or at least about 75% of the level or degree of binding of the corresponding wild-type HeV-G, such as set forth in SEQ ID NO:129 or 159, such as at least or at least about 80% of the level or degree of binding of the corresponding wild-type NIV-G, such as set forth in SEQ ID NO:129 or 159, such as at least or at least about 85% of the level or degree of binding of the corresponding wild-type HeV-G, such as set forth in SEQ ID NO:129 or 159, such as at least or at least about 90% of the level or degree of binding of the corresponding wild-type HeV-G, such as set forth in SEQ ID NO: 129 or 159, or such as at least or at least about 95% of the level or degree of binding of the corresponding wild-type HeV-G, such as set forth in SEQ ID NO:129 or 159.

In some embodiments, the G protein or the biologically thereof is a mutant G protein that exhibits reduced binding for the native binding partner of a wild-type G protein. In some embodiments, the mutant G protein or the biologically active portion thereof is a mutant of wild-type Niv-G and exhibits reduced binding to one or both of the native binding partners Ephrin B2 or Ephrin B3. In some embodiments, the mutant G-protein or the biologically active portion, such as a mutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the mutations described herein can improve transduction efficiency. In some embodiments, the mutations described herein allow for specific targeting of other desired cell types that are not Ephrin B2 or Ephrin B3. In some embodiments, the mutations described herein result in at least the partial inability to bind at least one natural receptor, such as to reduce the binding to at least one of Ephrin B2 or Ephrin B3. In some embodiments, the mutations described herein interfere with natural receptor recognition.

In some embodiments, the mutant NiV-G protein or the biologically active portion thereof is truncated and lacks up to 5 contiguous amino acid residues at or near the N-terminus of the wild-type NiV-G protein (SEQ ID NO:138), 6 contiguous amino acid residues at or near the N-terminus of the wild-type NiV-G protein (SEQ ID NO:138), 7 contiguous amino acid residues at or near the N-terminus of the wild-type NiV-G protein (SEQ ID NO:138), 8 contiguous amino acid residues at or near the N-terminus of the wild-type NiV-G protein (SEQ ID NO:138), 9 contiguous amino acid residues at or near the N-terminus of the wild-type NiV-G protein (SEQ ID NO:138), 10 contiguous amino acid residues at or near the N-terminus of the wild-type NiV-G protein (SEQ ID NO:138), 11 contiguous amino acid residues at or near the N-terminus of the wild-type NiV-G protein (SEQ ID NO:138), 12 contiguous amino acid residues at or near the N-terminus of the wild-type NiV-G protein (SEQ ID NO:138), 13 contiguous amino acid residues at or near the N-terminus of the wild-type NiV-G protein (SEQ ID NO:138), 14 contiguous amino acid residues at or near the N-terminus of the wild-type NiV-G protein (SEQ ID NO:138), 15 contiguous amino acid residues at or near the N-terminus of the wild-type NiV-G protein (SEQ ID NO:138), 16 contiguous amino acid residues at or near the N-terminus of the wild-type NiV-G protein (SEQ ID NO: 138), 17 contiguous amino acid residues at or near the N-terminus of the wild-type NiV-G protein (SEQ ID NO:138), 18 contiguous amino acid residues at or near the N-terminus of the wild-type NiV-G protein (SEQ ID NO:38), 19 contiguous amino acid residues at or near the N-terminus of the wild-type NiV-G protein (SEQ ID NO:138), 20 contiguous amino acid residues at or near the N-terminus of the wild-type NiV-G protein (SEQ ID NO:138), 21 contiguous amino acid residues at or near the N-terminus of the wild-type NiV-G protein (SEQ ID NO:138), 22 contiguous amino acid residues at or near the N-terminus of the wild-type NiV-G protein (SEQ ID NO:138), 23 contiguous amino acid residues at or near the N-terminus of the wild-type NiV-G protein (SEQ ID NO:138), 24 contiguous amino acid residues at or near the N-terminus of the wild-type NiV-G protein (SEQ ID NO:138), 25 contiguous amino acid residues at or near the N-terminus of the wild-type NiV-G protein (SEQ ID NO:138), 26 contiguous amino acid residues at or near the N-terminus of the wild-type NiV-G protein (SEQ ID NO:138), 27 contiguous amino acid residues at or near the N-terminus of the wild-type NiV-G protein (SEQ ID NO: 138), 28 contiguous amino acid residues at or near the N-terminus of the wild-type NiV-G protein (SEQ ID NO:138), 29 contiguous amino acid residues at or near the N-terminus of the wild-type NiV-G protein (SEQ ID NO:138), 30 contiguous amino acid residues at or near the N-terminus of the wild-type NiV-G protein (SEQ ID NO:138), 31 contiguous amino acid residues at or near the N-terminus of the wild-type NiV-G protein (SEQ ID NO:138), 32 contiguous amino acid residues at or near the N-terminus of the wild-type NiV-G protein (SEQ ID NO:138), 33 contiguous amino acid residues at or near the N-terminus of the wild-type NiV-G protein (SEQ ID NO:138), 34 contiguous amino acid residues at or near the N-terminus of the wild-type NiV-G protein (SEQ ID NO:138), 35 contiguous amino acid residues at or near the N-terminus of the wild-type NiV-G protein (SEQ ID NO:138), 36 contiguous amino acid residues at or near the N-terminus of the wild-type NiV-G protein (SEQ ID NO:138), 37 contiguous amino acid residues at or near the N-terminus of the wild-type NiV-G protein (SEQ ID NO:138), 38 contiguous amino acid residues at or near the N-terminus of the wild-type NiV-G protein (SEQ ID NO:138), 39 contiguous amino acid residues at or near the N-terminus of the wild-type NiV-G protein (SEQ ID NO:138), or 40 contiguous amino acid residues at or near the N-terminus of the wild-type NiV-G protein (SEQ ID NO:138).

In some embodiments, the G protein contains one or more amino acid substitutions in a residue that is involved in the interaction with one or both of Ephrin B2 and Ephrin B3. In some embodiments, the amino acid substitutions correspond to mutations E501A, W504A, Q530A, and E533A with reference to numbering set forth in SEQ ID NO: 138.

In some embodiments, the G protein is a mutant G protein containing one or more amino acid substitutions selected from the group consisting of E501A, W504A, Q530A, and E533A with reference to numbering set forth in SEQ ID NO:138. In some embodiments, the G protein is a mutant G protein that contains one or more amino acid substitutions selected from the group consisting of E501A, W504A, Q530A, and E533A with reference to SEQ ID NO:138 or a biologically active portion thereof containing an N-terminal truncation. In some embodiments, the G protein is a mutant G protein that contains one or more amino acid substitutions selected from the group consisting of E501A, W504A, Q530A, and E533A in combination with any one of the N-terminal truncations disclosed above with reference to SEQ ID NO:138 or a biologically active portion thereof. In some embodiments, any of the mutant G proteins described above contains one, two, three, or all four amino acids selected from the group consisting of E501A, W504A, Q530A, and E533A with reference to numbering set forth in SEQ ID NO:138, in all pairwise and triple combinations thereof.

In some embodiments, the mutant NiV-G protein has the amino acid sequence set forth in SEQ ID NO:127 or 155 or an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO: 141 or 169. In some embodiments, the G protein has the sequence of amino acids set forth in SEQ ID NO:127 or 155.

In some embodiments, the targeted envelope protein contains a G protein or a functionally active variant or biologically active portion thereof and a targeting antibody or antigen binding fragment thereof, in which the targeted envelope protein exhibits increased binding for another molecule that is different from the native binding partner of a wild-type G protein. In some embodiments, the targeting antibody or antigen binding fragment thereof is a single domain antibody (sdAb) or a scFv. In some embodiments, the other molecule is a protein expressed on the surface of a desired target cell. In some embodiments, the increased binding to the other molecule is increased by greater than at or about 25%, at or about 30%, at or about 40%, at or about 50%, at or about 60%, at or about 70%, at or about 80%, at or about 90%, or at or about 100%. In some embodiments, the binding confers re-targeted binding compared to the binding of a wild-type G protein in which a new or different binding activity is conferred.

In some embodiments, the C-terminus of the targeting antibody or antigen binding fragment thereof is attached to the C-terminus of the G protein or biologically active portion thereof. In some embodiments, the N-terminus end of the targeting antibody or antigen binding fragment thereof is exposed on the exterior surface of the lipid bilayer. In some embodiments, the N-terminus end of the targeting antibody or antigen binding fragment thereof binds to a cell surface molecule of a target cell. In some embodiments, the targeting antibody or antigen binding fragment thereof specifically binds to a cell surface molecule present on a target cell. In some embodiments, the cell surface molecule is a protein, glycan, lipid, or low molecular weight molecule.

In some embodiments, the cell surface molecule of a target cell is an antigen or portion thereof. In some embodiments, the targeting antibody or antigen binding fragment thereof is an antibody having a single monomeric domain antigen binding/recognition domain that is able to bind selectively to a specific antigen. In some embodiments, the single domain antibody binds an antigen present on a target cell.

Exemplary cells include immune effector cells, peripheral blood mononuclear cells (PBMC) such as lymphocytes (T cells, B cells, natural killer cells) and monocytes, granulocytes (neutrophils, basophils, eosinophils), macrophages, dendritic cells, cytotoxic T lymphocytes, polymorphonuclear cells (also known as PMN, PML, or PMNL), stem cells, embryonic stem cells, neural stem cells, mesenchymal stem cells (MSCs), hematopoietic stem cells (HSCs), human myogenic stem cells, muscle-derived stem cells (MuStem), embryonic stem cells (ES or ESCs), limbal epithelial stem cells, cardio-myogenic stem cells, cardiomyocytes, progenitor cells, allogenic cells, resident cardiac cells, induced pluripotent stem cells (iPS), adipose-derived or phenotypic modified stem or progenitor cells, CD133+ cells, aldehyde dehydrogenase-positive cells (ALDH+), umbilical cord blood (UCB) cells, peripheral blood stem cells (PBSCs), neurons, neural progenitor cells, pancreatic beta cells, glial cells, or hepatocytes.

In some embodiments, the target cell is a cell of a target tissue. In some embodiments, the target tissue is liver, lungs, heart, spleen, pancreas, gastrointestinal tract, kidney, testes, ovaries, brain, reproductive organs, central nervous system, peripheral nervous system, skeletal muscle, endothelium, inner ear, or eye.

In some embodiments, the target cell is a muscle cell (e.g., skeletal muscle cell), kidney cell, liver cell (e.g., hepatocyte), or a cardiac cell (e.g., cardiomyocyte). In some embodiments, the target cell is a cardiac cell, e.g., a cardiomyocyte (e.g., a quiescent cardiomyocyte), a hepatoblast (e.g., a bile duct hepatoblast), an epithelial cell, a T cell (e.g., a naive T cell), a macrophage (e.g., a tumor infiltrating macrophage), or a fibroblast (e.g., a cardiac fibroblast).

In some embodiments, the target cell is a tumor-infiltrating lymphocyte, a T cell, a neoplastic or tumor cell, a virus-infected cell, a stem cell, a central nervous system (CNS) cell, a hematopoietic stem cell (HSC), a liver cell or a fully differentiated cell. In some embodiments, the target cell is a CD3+ T cell, a CD4+ T cell, a CD8+ T cell, a hepatocyte, a hematopoietic stem cell, a CD34+ hematopoietic stem cell, a CD105+ hematopoietic stem cell, a CD 117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacytoid dendritic cell, a CD11c+ cell, a CD11b+ cell, a splenocyte, a B cell, a hepatocyte, an endothelial cell, or a non-cancerous cell. In some embodiments, the cell surface molecule is any one of CD8.

In some embodiments, the G protein or functionally active variant or biologically active portion thereof is linked directly to the sdAb variable domain (e.g., a VHH) or scFv. In some embodiments, the targeted envelope protein is a fusion protein that has the following structure: (N′-single domain antibody-C′)-(C′-G protein-N′). In some embodiments, the targeted envelope protein is a fusion protein that has the following structure: (N′-scFv-C′)-(C′-G protein-N′).

In some embodiments, the G protein or functionally active variant or biologically active portion thereof is linked indirectly via a linker to the sdAb variable domain or scFv. In some embodiments, the linker is a peptide linker. In some embodiments, the linker is a chemical linker.

In some embodiments, the linker is a peptide linker and the targeted envelope protein is a fusion protein containing the G protein or functionally active variant or biologically active portion thereof linked via a peptide linker to the sdAb variable domain or scFv. In some embodiments, the targeted envelope protein is a fusion protein that has the following structure: (N′-single domain antibody-C′)-Linker-(C′-G protein-N′). In some embodiments, the targeted envelope protein is a fusion protein that has the following structure: (N′-scFv-C′)-Linker-(C′-G protein-N′). In some embodiments, the peptide linker is up to 65 amino acids in length. In some embodiments, the peptide linker comprises from or from about 2 to 65 amino acids, 2 to 60 amino acids, 2 to 56 amino acids, 2 to 52 amino acids, 2 to 48 amino acids, 2 to 44 amino acids, 2 to 40 amino acids, 2 to 36 amino acids, 2 to 32 amino acids, 2 to 28 amino acids, 2 to 24 amino acids, 2 to 20 amino acids, 2 to 18 amino acids, 2 to 14 amino acids, 2 to 12 amino acids, 2 to 10 amino acids, 2 to 8 amino acids, 2 to 6 amino acids, 6 to 65 amino acids, 6 to 60 amino acids, 6 to 56 amino acids, 6 to 52 amino acids, 6 to 48 amino acids, 6 to 44 amino acids, 6 to 40 amino acids, 6 to 36 amino acids, 6 to 32 amino acids, 6 to 28 amino acids, 6 to 24 amino acids, 6 to 20 amino acids, 6 to 18 amino acids, 6 to 14 amino acids, 6 to 12 amino acids, 6 to 10 amino acids, 6 to 8 amino acids, 8 to 65 amino acids, 8 to 60 amino acids, 8 to 56 amino acids, 8 to 52 amino acids, 8 to 48 amino acids, 8 to 44 amino acids, 8 to 40 amino acids, 8 to 36 amino acids, 8 to 32 amino acids, 8 to 28 amino acids, 8 to 24 amino acids, 8 to 20 amino acids, 8 to 18 amino acids, 8 to 14 amino acids, 8 to 12 amino acids, 8 to 10 amino acids, 10 to 65 amino acids, 10 to 60 amino acids, 10 to 56 amino acids, 10 to 52 amino acids, 10 to 48 amino acids, 10 to 44 amino acids, 10 to 40 amino acids, 10 to 36 amino acids, 10 to 32 amino acids, 10 to 28 amino acids, 10 to 24 amino acids, 10 to 20 amino acids, 10 to 18 amino acids, 10 to 14 amino acids, 10 to 12 amino acids, 12 to 65 amino acids, 12 to 60 amino acids, 12 to 56 amino acids, 12 to 52 amino acids, 12 to 48 amino acids, 12 to 44 amino acids, 12 to 40 amino acids, 12 to 36 amino acids, 12 to 32 amino acids, 12 to 28 amino acids, 12 to 24 amino acids, 12 to 20 amino acids, 12 to 18 amino acids, 12 to 14 amino acids, 14 to 65 amino acids, 14 to 60 amino acids, 14 to 56 amino acids, 14 to 52 amino acids, 14 to 48 amino acids, 14 to 44 amino acids, 14 to 40 amino acids, 14 to 36 amino acids, 14 to 32 amino acids, 14 to 28 amino acids, 14 to 24 amino acids, 14 to 20 amino acids, 14 to 18 amino acids, 18 to 65 amino acids, 18 to 60 amino acids, 18 to 56 amino acids, 18 to 52 amino acids, 18 to 48 amino acids, 18 to 44 amino acids, 18 to 40 amino acids, 18 to 36 amino acids, 18 to 32 amino acids, 18 to 28 amino acids, 18 to 24 amino acids, 18 to 20 amino acids, 20 to 65 amino acids, 20 to 60 amino acids, 20 to 56 amino acids, 20 to 52 amino acids, 20 to 48 amino acids, 20 to 44 amino acids, 20 to 40 amino acids, 20 to 36 amino acids, 20 to 32 amino acids, 20 to 28 amino acids, 20 to 26 amino acids, 20 to 24 amino acids, 24 to 65 amino acids, 24 to 60 amino acids, 24 to 56 amino acids, 24 to 52 amino acids, 24 to 48 amino acids, 24 to 44 amino acids, 24 to 40 amino acids, 24 to 36 amino acids, 24 to 32 amino acids, 24 to 30 amino acids, 24 to 28 amino acids, 28 to 65 amino acids, 28 to 60 amino acids, 28 to 56 amino acids, 28 to 52 amino acids, 28 to 48 amino acids, 28 to 44 amino acids, 28 to 40 amino acids, 28 to 36 amino acids, 28 to 34 amino acids, 28 to 32 amino acids, 32 to 65 amino acids, 32 to 60 amino acids, 32 to 56 amino acids, 32 to 52 amino acids, 32 to 48 amino acids, 32 to 44 amino acids, 32 to 40 amino acids, 32 to 38 amino acids, 32 to 36 amino acids, 36 to 65 amino acids, 36 to 60 amino acids, 36 to 56 amino acids, 36 to 52 amino acids, 36 to 48 amino acids, 36 to 44 amino acids, 36 to 40 amino acids, 40 to 65 amino acids, 40 to 60 amino acids, 40 to 56 amino acids, 40 to 52 amino acids, 40 to 48 amino acids, 40 to 44 amino acids, 44 to 65 amino acids, 44 to 60 amino acids, 44 to 56 amino acids, 44 to 52 amino acids, 44 to 48 amino acids, 48 to 65 amino acids, 48 to 60 amino acids, 48 to 56 amino acids, 48 to 52 amino acids, 50 to 65 amino acids, 50 to 60 amino acids, 50 to 56 amino acids, 50 to 52 amino acids, 54 to 65 amino acids, 54 to 60 amino acids, 54 to 56 amino acids, 58 to 65 amino acids, 58 to 60 amino acids, or 60 to 65 amino acids. In some embodiments, the peptide linker is a polypeptide that is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 amino acids in length.

In some embodiments, the linker is a flexible peptide linker. In some such embodiments, the linker is 1-20 amino acids, such as 1-20 amino acids comprising glycine. In some embodiments, the linker is 1-20 amino acids, such as 1-20 amino acids comprising glycine and serine. In some embodiments, the linker is a flexible peptide linker containing amino acids Glycine and Serine, referred to as GS-linkers. In some embodiments, the peptide linker includes the sequences GS, GGS, GGGGS (SEQ ID NO:147), GGGGGS (SEQ ID NO:145) or combinations thereof. In some embodiments, the polypeptide linker has the sequence (GGS)n, wherein n is 1 to 10. In some embodiments, the polypeptide linker has the sequence (GGGGS)n, (SEQ ID NO:146) wherein n is 1 to 10. In some embodiments, the polypeptide linker has the sequence (GGGGGS)n (SEQ ID NO: 137), wherein n is 1 to 6.

Also provided herein are polynucleotides comprising a nucleic acid sequence encoding a targeted envelope protein. In some embodiments, the polynucleotides comprise a nucleic acid sequence encoding a G protein or biologically active portion thereof. In some embodiments, the polynucleotides further comprise a nucleic acid sequence encoding a single domain antibody (sdAb) variable domain or scFv or biologically active portion thereof. The polynucleotides may include a sequence of nucleotides encoding any of the targeted envelope proteins described above. In some embodiments, the polynucleotide is a synthetic nucleic acid. Also provided are expression vectors containing any of the provided polynucleotides.

In some embodiments, expression of natural or synthetic nucleic acids is achieved by operably linking a nucleic acid encoding the gene of interest to a promoter and incorporating the construct into an expression vector. In some embodiments, vectors are suitable for replication and integration in eukaryotes. In some embodiments, cloning vectors contain transcription and translation terminators, initiation sequences, and promoters useful for expression of the desired nucleic acid sequence. In some of any embodiments, a plasmid comprises a promoter suitable for expression in a cell.

In some embodiments, the polynucleotides contain at least one promoter that is operatively linked to control expression of the targeted envelope protein containing the G protein and the single domain antibody (sdAb) variable domain or scFv. For expression of the targeted envelope protein, at least one module in each promoter functions to position the start site for RNA synthesis. The best-known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

In some embodiments, additional promoter elements, e.g., enhancers, regulate the frequency of transcriptional initiation. In some embodiments, additional promoter elements are located in the region 30-110 bp upstream of the start site, although a number of promoters have been shown to contain functional elements downstream of the start site as well. In some embodiments, spacing between promoter elements frequently is flexible, so that promoter function is preserved when elements are inverted or moved relative to one another. In some embodiments, such as with the thymidine kinase (tk) promoter, the spacing between promoter elements is increased to 50 bp apart before activity begins to decline. In some embodiments, depending on the promoter, individual elements can function either cooperatively or independently to activate transcription.

In some embodiments, a promoter is one naturally associated with a gene or polynucleotide sequence, as is obtained by isolating the 5′ non-coding sequences located upstream of the coding segment and/or exon. In some embodiments, such a promoter is referred to as “endogenous.” In some embodiments, an enhancer is one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences are produced using recombinant cloning and/or nucleic acid amplification technology, including PCR, in connection with the compositions disclosed herein.

In some embodiments, a suitable promoter is the immediate early cytomegalovirus (CMV) promoter sequence. In some embodiments, the promoter sequence is a strong constitutive promoter sequence capable of driving high levels of expression of any polynucleotide sequence operatively linked thereto. In some embodiments, a suitable promoter is Elongation Growth Factor-la (EF-l a). In some embodiments, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

In some embodiments, the promoter is an inducible promoter. In some embodiments, the inducible promoter provides a molecular switch capable of turning on expression of the polynucleotide sequence to which it is operatively linked when such expression is desired, or turning off the expression when expression is not desired. In some embodiments, inducible promoters comprise a metallothionine promoter, a glucocorticoid promoter, a progesterone promoter, and a tetracycline promoter.

In some embodiments, exogenously controlled inducible promoters are used to regulate expression of the G protein and single domain antibody (sdAb) variable domain or scFv. For example, radiation-inducible promoters, heat-inducible promoters, and/or drug-inducible promoters are used to selectively drive transgene expression in, for example, targeted regions. In such embodiments, the location, duration, and level of transgene expression are regulated by the administration of the exogenous source of induction.

In some embodiments, expression of the targeted envelope protein containing a G protein and single domain antibody (sdAb) variable domain or scFv is regulated using a drug-inducible promoter. For example, in some embodiments, the promoter, enhancer, or transactivator comprises a Lac operator sequence, a tetracycline operator sequence, a galactose operator sequence, a doxycycline operator sequence, a rapamycin operator sequence, a tamoxifen operator sequence, or a hormone-responsive operator sequence, or an analog thereof. In some instances, the inducible promoter comprises a tetracycline response element (TRE). In some embodiments, the inducible promoter comprises an estrogen response element (ERE), which can activate gene expression in the presence of tamoxifen. In some instances, a drug-inducible element, such as a TRE, is combined with a selected promoter to enhance transcription in the presence of drug, such as doxycycline. In some embodiments, the drug-inducible promoter is a small molecule-inducible promoter.

In some embodiments, any of the provided polynucleotides are modified to remove CpG motifs and/or to optimize codons for translation in a particular species, such as human, canine, feline, equine, ovine, bovine, etc. species. In some embodiments, the polynucleotides are optimized for human codon usage (i.e., human codon-optimized). In some embodiments, the polynucleotides are modified to remove CpG motifs. In other embodiments, the provided polynucleotides are modified to remove CpG motifs and are codon-optimized, such as human codon-optimized. Methods of codon optimization and CpG motif detection and modification are well-known. Typically, polynucleotide optimization enhances transgene expression, increases transgene stability and preserves the amino acid sequence of the encoded polypeptide.

In order to assess the expression of the targeted envelope protein, the expression vector to be introduced into a cell can also contain either a selectable marker gene or a reporter gene or both to facilitate identification and selection of expressing particles, e.g., viral particles. In other embodiments, the selectable marker is carried on a separate piece of DNA and used in a co-transfection procedure. In some embodiments, both selectable markers and reporter genes are flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000, FEBS Lett. 479:79-82). Suitable expression systems are well known and may be prepared using well known techniques or obtained commercially. In some embodiments, internal deletion constructs are generated using unique internal restriction sites or by partial digestion of non-unique restriction sites. Constructs may then be transfected into cells that display high levels of the desired polynucleotide and/or polypeptide expression. In general, the construct with the minimal 5′ flanking region showing the highest level of expression of reporter gene is identified as the promoter. In some embodiments, such promoter regions are linked to a reporter gene and used to evaluate agents for the ability to modulate promoter-driven transcription.

### E. F Protein

In some embodiments, the targeted lipid particle comprises one or more fusogens, e.g., henipavirus F proteins. In some embodiments, the targeted lipid particle contains an exogenous or overexpressed fusogen. In some embodiments, the fusogen is disposed in the lipid bilayer. In some embodiments, the fusogen facilitates the fusion of the targeted particle's lipid bilayer to a membrane. In some embodiments, the membrane is a plasma cell membrane.

In some embodiments, fusogens comprise protein based, lipid based, and chemical based fusogens. In some embodiments, the targeted lipid particle comprises a first fusogen comprising a protein fusogen and a second fusogen comprising a lipid fusogen or chemical fusogen. In some embodiments, the fusogen binds a fusogen binding partner on a target cell surface.

In some embodiments, the fusogen comprises a protein with a hydrophobic fusion peptide domain. In some embodiments, the fusogen comprises a henipavirus F protein molecule or biologically active portion thereof. In some embodiments, the Henipavirus F protein is a Hendra (Hev) virus F protein, a Nipah (NiV) virus F-protein, a Cedar (CedPV) virus F protein, a Mojiang virus F protein, a bat Paramyxovirus F protein, or a biologically active portion thereof. Table 22 provides a list of non-limiting examples of F proteins.

In some embodiments, the N-terminal hydrophobic fusion peptide domain of the F protein molecule or biologically active portion thereof is exposed on the outside of a lipid bilayer.

F proteins of henipaviruses are encoded as F0 precursors containing a signal peptide (e.g., corresponding to amino acid residues 1-26 of SEQ ID NO: 110). Following cleavage of the signal peptide, the mature F0 (e.g., gSEQ ID NO: 111) is transported to the cell surface, then endocytosed and cleaved by cathepsin L (e.g., between amino acids 109-110 of SEQ ID NO: 110) into the mature fusogenic subunits F1 (e.g., corresponding to amino acids 110-546 of SEQ ID NO:110; set forth in SEQ ID NO:113) and F2 (e.g., corresponding to amino acid residues 27-109 of SEQ ID NO:110; set forth in SEQ ID NO: 112). The F1 and F2 subunits are associated by a disulfide bond and recycled back to the cell surface. The F1 subunit contains the fusion peptide domain located at the N terminus of the F1 subunit (e.g., corresponding to amino acids 110-129 of SEQ ID NO:110) where it is able to insert into a cell membrane to drive fusion. In some embodiments, fusion activity is blocked by association of the F protein with G protein, until G engages with a target molecule resulting in its disassociation from F and exposure of the fusion peptide to mediate membrane fusion.

Among different henipavirus species, the sequence and activity of the F protein is highly conserved. For examples, the F protein of NiV and HeV viruses share 89% amino acid sequence identity. Further, in some embodiments, the henipavirus F proteins exhibit compatibility with G proteins from other species to trigger fusion (Brandel-Tretheway et al. Journal of Virology. 2019. 93(13):e00577-19). In some aspects of the provided targeted lipid particle, the F protein is heterologous to the G protein, i.e., the F and G protein or biologically active portions thereof are from different henipavirus species. For example, the F protein is from Hendra virus and the G protein is from Nipah virus. In other aspects, the F protein is a chimeric F protein containing regions of F proteins from different species of Henipavirus. In some embodiments, switching a region of amino acid residues of the F protein from one species of Henipavirus to another can result in fusion to the G protein of the species comprising the amino acid insertion. (Brandel-Tretheway et al. 2019). In some embodiments, the chimeric F protein contains an extracellular domain from one henipavirus species and a transmembrane and/or cytoplasmic domain from a different henipavirus species. For example, the F protein may contain an extracellular domain of Hendra virus and a transmembrane/cytoplasmic domain of Nipah virus. F protein sequences disclosed herein are predominantly disclosed as expressed sequences including an N-terminal signal sequence. Such N-terminal signal sequences are commonly cleaved co- or post-translationally, thus the mature protein sequences for all F protein sequences disclosed herein are also contemplated as lacking the N-terminal signal sequence.

In some embodiments, the F protein is encoded by a nucleotide sequence that encodes the sequence set forth by any one of SEQ ID NOs: 110, 111, 128, 134-136, or 161-164, or is a functionally active variant or a biologically active portion thereof that has a sequence that is at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOS: 110, 111, 128, 134-136, or 161-164. In some embodiments, the F protein or the functionally active variant or biologically active portion thereof retains fusogenic activity in conjunction with a Henipavirus G protein, such as a G protein set forth herein. Fusogenic activity includes the activity of the F protein in conjunction with a Henipavirus G protein to promote or facilitate fusion of two membrane lumens, such as the lumen of the targeted lipid particle having embedded in its lipid bilayer a henipavirus F and G protein, and a cytoplasm of a target cell, e.g., a cell that contains a surface receptor or molecule that is recognized or bound by the targeted envelope protein. In some embodiments, the F protein and G protein are from the same Henipavirus species (e.g., NiV-G and NiV-F). In some embodiments, the F protein and G protein are from different Henipavirus species (e.g., NiV-G and HeV-F). In some embodiments, the F protein of the functionally active variant or biologically active portion retains the cleavage site cleaved by cathepsin L (e.g., corresponding to the cleavage site between amino acids 109-110 of SEQ ID NO:110).

In some embodiments, the F protein has the sequence of amino acids set forth in SEQ ID NO:110, SEQ ID NO:111, SEQ ID NO:128, SEQ ID NO:134, SEQ ID NO:135, SEQ ID NO:136, SEQ ID NO:161, SEQ ID NO:162, SEQ ID NO:163, or SEQ ID NO:164 or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:110, SEQ ID NO:111, SEQ ID NO:128, SEQ ID NO:134, SEQ ID NO:135, SEQ ID NO:136, SEQ ID NO:161, SEQ ID NO:162, SEQ ID NO:163, or SEQ ID NO:164 and retains fusogenic activity in conjunction with a Henipavirus G protein (e.g., NiV-G or HeV-G). In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:110, SEQ ID NO:111, SEQ ID NO:128, SEQ ID NO:134, SEQ ID NO:135, SEQ ID NO:136, SEQ ID NO:161, SEQ ID NO:162, SEQ ID NO:163, or SEQ ID NO:164 and retains fusogenic activity in conjunction with a Henipavirus G protein (e.g., NiV-G or HeV-G).

Reference to retaining fusogenic activity includes activity (in conjunction with a Henipavirus G protein) that is at or about 10% to at or about 150% or more of the level or degree of binding of the corresponding wild-type F protein, such as set forth in SEQ ID NO:110, SEQ ID NO:111, SEQ ID NO:128, SEQ ID NO:134, SEQ ID NO:135, SEQ ID NO:136, SEQ ID NO:161, SEQ ID NO:162, SEQ ID NO:163, or SEQ ID NO:164, such as at least or at least about 10% of the level or degree of fusogenic activity of the corresponding wild-type F protein, such as at least or at least about 15% of the level or degree of fusogenic activity of the corresponding wild-type F protein, such as at least or at least about 20% of the level or degree of fusogenic activity of the corresponding wild-type F protein, such as at least or at least about 25% of the level or degree of fusogenic activity of the corresponding wild-type F protein, such as at least or at least about 30% of the level or degree of fusogenic activity of the corresponding wild-type F protein, such as at least or at least about 35% of the level or degree of fusogenic activity of the corresponding wild-type F protein, such as at least or at least about 40% of the level or degree of fusogenic activity of the corresponding wild-type F protein, such as at least or at least about 45% of the level or degree of fusogenic activity of the corresponding wild-type F protein, such as at least or at least about 50% of the level or degree of fusogenic activity of the corresponding wild-type F protein, such as at least or at least about 55% of the level or degree of fusogenic activity of the corresponding wild-type f protein, such as at least or at least about 60% of the level or degree of fusogenic activity of the corresponding wild-type F protein, such as at least or at least about 65% of the level or degree of fusogenic activity of the corresponding wild-type F protein, such as at least or at least about 70% of the level or degree of fusogenic activity of the corresponding wild-type F protein, such as at least or at least about 75% of the level or degree of fusogenic activity of the corresponding wild-type F protein, such as at least or at least about 80% of the level or degree of fusogenic activity of the corresponding wild-type F protein, such as at least or at least about 85% of the level or degree of fusogenic activity of the corresponding wild-type F protein, such as at least or at least about 90% of the level or degree of fusogenic activity of the corresponding wild-type F protein, such as at least or at least about 95% of the level or degree of fusogenic activity of the corresponding wild-type F protein, such as at least or at least about 100% of the level or degree of fusogenic activity of the corresponding wild-type F protein, or such as at least or at least about 120% of the level or degree of fusogenic activity of the corresponding wild-type F protein.

In some embodiments, the F protein is a mutant F protein that is a functionally active fragment or a biologically active portion containing one or more amino acid mutations, such as one or more amino acid insertions, deletions, substitutions, or truncations. In some embodiments, the mutations described herein relate to amino acid insertions, deletions, substitutions, or truncations of amino acids compared to a reference F protein sequence. In some embodiments, the reference F protein sequence is the wild-type sequence of an F protein or a biologically active portion thereof. In some embodiments, the mutant F protein or the biologically active portion thereof is a mutant of a wild-type Hendra (Hev) virus F protein, a Nipah (NiV) virus F-protein, a Cedar (CedPV) virus F protein, a Mojiang virus F protein, or a bat Paramyxovirus F protein. In some embodiments, the wild-type F protein is encoded by a sequence of nucleotides that encodes any one of SEQ ID NO: 110, 111, 128, 134-136, or 161-164.

In some embodiments, the mutant F protein is a biologically active portion of a wild-type F protein that is an N-terminally and/or C-terminally truncated fragment. In some embodiments, the mutant F protein or the biologically active portion of a wild-type F protein thereof comprises one or more amino acid substitutions. In some embodiments, the mutations described herein can improve transduction efficiency. In some embodiments, the mutations described herein can increase fusogenic capacity. Exemplary mutations include any as described, see e.g., Khetawat and Broder 2010 Virology Journal 7:312; Witting et al. 2013 Gene Therapy 20:997-1005; published international; patent application No. WO/2013/148327.

In some embodiments, the mutant F protein is a biologically active portion that is truncated and lacks up to 20 contiguous amino acid residues at or near the C-terminus of the wild-type F protein, such as a wild-type F protein encoded by a sequence of nucleotides encoding the F protein set forth in any one of SEQ ID NOS: 110, 111, 128, or 134-136. In some embodiments, the mutant F protein is truncated and lacks up to 19 contiguous amino acids, such as up to 18, 17, 16, 15, 14, 13, 12, 11, 10, 9, 8, 7, 6, 5, 4, 3, 2, or 1 contiguous amino acid(s) at the C-terminus of the wild-type F protein.

In some embodiments, the F protein or the functionally active variant or biologically active portion thereof comprises an F1 subunit or a fusogenic portion thereof. In some embodiments, the F1 subunit is a proteolytically cleaved portion of the F0 precursor. In some embodiments, the F0 precursor is inactive. In some embodiments, the cleavage of the F0 precursor forms a disulfide-linked F1+F2 heterodimer. In some embodiments, the cleavage exposes the fusion peptide and produces a mature F protein. In some embodiments, the cleavage occurs at or around a single basic residue. In some embodiments, the cleavage occurs at Arginine 109 of NiV-F protein. In some embodiments, cleavage occurs at Lysine 109 of the Hendra virus F protein.

In some embodiments, the F protein is a wild-type Nipah virus F (NiV-F) protein or is a functionally active variant or biologically active portion thereof. In some embodiments, the F0 precursor is encoded by a sequence of nucleotides encoding the sequence set forth in SEQ ID NO:110. The encoding nucleic acid can encode a signal peptide sequence that has the sequence MVVILDKRCY CNLLILILMI SECSVG (SEQ ID NO:144) or another signal peptide sequence. In some embodiments, the F protein has the sequence set forth in SEQ ID NO:111. In some examples, the F protein is cleaved into an F1 subunit comprising the sequence set forth in SEQ ID NO:113 and an F2 subunit comprising the sequence set forth in SEQ ID NO:112.

In some embodiments, the F protein is a NiV-F protein that is encoded by a sequence of nucleotides encoding the sequence set forth in SEQ ID NO:110, or is a functionally active variant or biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO: 110. In some embodiments, the NiV-F-protein has the sequence of set forth in SEQ ID NO:111, or is a functionally active variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:111. In some embodiments, the F protein or the functionally active variant or biologically active portion thereof retains the cleavage site cleaved by cathepsin L (e.g., corresponding to the cleavage site between amino acids 109-110 of SEQ ID NO:110).

In some embodiments, the F protein or the functionally active variant or the biologically active portion thereof includes an F1 subunit that has the sequence set forth in SEQ ID NO:113, or an amino acid sequence having, at least at or about 80%, at least at or about 81%, at least at or about 82%, at least at or about 83%, at least at or about 84%, at least at or about 85%, at least at or about 86%, at least at or about 87%, at least at or about 88%, at least at or about 89% at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:113.

In some embodiments, the F protein or the functionally active variant or biologically active portion thereof includes an F2 subunit that has the sequence set forth in SEQ ID NO:112, or an amino acid sequence having, at least at or about 80%, at least at or about 81%, at least at or about 82%, at least at or about 83%, at least at or about 84%, at least at or about 85%, at least at or about 86%, at least at or about 87%, at least at or about 88%, at least at or about 89% at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:112.

In some embodiments, the F protein is a mutant NiV-F protein that is a biologically active portion thereof that is truncated and lacks up to 20 contiguous amino acid residues at or near the C-terminus of the wild-type NiV-F protein (e.g., set forth SEQ ID NO:111). In some embodiments, the mutant NiV-F protein comprises an amino acid sequence set forth in SEQ ID NO:114. In some embodiments, the mutant NiV-F protein has a sequence that has at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:114. In some embodiments, the mutant F protein contains an F1 protein that has the sequence set forth in SEQ ID NO:115. In some embodiments, the mutant F protein has a sequence that has at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:115.

In some embodiments, the F protein is a mutant NiV-F protein that is a biologically active portion thereof that comprises a 20 amino acid truncation at or near the C-terminus of the wild-type NiV-F protein (SEQ ID NO:111); and a point mutation on an N-linked glycosylation site. In some embodiments, the mutant NiV-F protein comprises an amino acid sequence set forth in SEQ ID NO:116. In some embodiments, the mutant

Methods of Generating Targeted Lipid Particles Derived from Virus

Provided herein are targeted lipid particles that are derived from virus, such as viral particles or virus-like particles, including those derived from retroviruses or lentiviruses. In some embodiments, the targeted lipid particle's bilayer of amphipathic lipids is or comprises the viral envelope. In some embodiments, the targeted lipid particle's bilayer of amphipathic lipids is or comprises lipids derived from a producer cell. In some embodiments, the viral envelope may comprise a fusogen, e.g., a fusogen that is endogenous to the virus or a pseudotyped fusogen. In some embodiments, the targeted lipid particles' lumen or cavity comprises a viral nucleic acid, e.g., a retroviral nucleic acid, e.g., a lentiviral nucleic acid. In some embodiments, the viral nucleic acid is a viral genome. In some embodiments, the targeted lipid particle further comprises one or more viral non-structural proteins, e.g., in its cavity or lumen. In some embodiments, the targeted lipid particles is or comprises a virus-like particle (VLP). In some embodiments, the VLP does not comprise an envelope. In some embodiments, the VLP comprises an envelope.

In some embodiments, the viral particle or virus-like particle, such as a retrovirus or retrovirus-like particle, comprises one or more of a Gag polyprotein, polymerase (e.g., Pol), integrase (IN, e.g., a functional or non-functional variant), protease (PR), and a fusogen. In some embodiments, the targeted lipid particle further comprises Rev. In some embodiments, one or more of the aforesaid proteins are encoded in the retroviral genome, and in some embodiments, one or more of the aforesaid proteins are provided in trans, e.g., by a helper cell, helper virus, or helper plasmid. In some embodiments, the targeted lipid particle nucleic acid (e.g., retroviral nucleic acid) comprises one or more of the following nucleic acid sequences: 5′ LTR (e.g., comprising U5 and lacking a functional U3 domain), Psi packaging element (Psi), Central polypurine tract (cPPT) Promoter operatively linked to the payload gene, payload gene (optionally comprising an intron before the open reading frame), Poly A tail sequence, WPRE, and 3′ LTR (e.g., comprising U5 and lacking a functional U3). In some embodiments the targeted lipid particle nucleic acid further comprises one or more insulator elements. In some embodiments, the recognition sites are situated between the poly A tail sequence and the WPRE.

In some embodiments, the targeted lipid particle comprises supramolecular complexes formed by viral proteins that self-assemble into capsids. In some embodiments, the targeted lipid particle is a viral particle or virus-like particle derived from viral capsids. In some embodiments, the targeted lipid particle is a viral particle or virus-like particle derived from viral nucleocapsids. In some embodiments, the targeted lipid particle comprises nucleocapsid-derived proteins that retain the property of packaging nucleic acids. In some embodiments, the viral particles or virus-like particles comprises only viral structural glycoproteins. In some embodiments, the targeted lipid particle does not contain a viral genome.

In some embodiments, the targeted lipid particle packages nucleic acids from host cells during the expression process. In some embodiments, the nucleic acids do not encode any genes involved in virus replication. In some embodiments, the targeted lipid particle is a virus-like particle, e.g., retrovirus-like particle such as a lentivirus-like particle, that is replication defective.

In some embodiments, the targeted lipid particle is a viral particle that is morphologically indistinguishable from the wild-type infectious virus. In some embodiments, the viral particle presents the entire viral proteome as an antigen. In some embodiments, the viral particle presents only a portion of the proteome as an antigen.

In some embodiments, the viral particle or virus-like particle is produced utilizing proteins (e.g., envelope proteins) from a virus within the Paramyxoviridae family. In some embodiments, the Paramyxoviridae family comprises members within the Henipavirus genus. In some embodiments, the Henipavirus is or comprises a Hendra (HeV) or a Nipah (NiV) virus. In some embodiments, the viral particles or virus-like particles incorporate a targeted envelope protein and fusogen.

In some embodiments, viral particles or virus-like particles are produced in multiple cell culture systems including bacteria, mammalian cell lines, insect cell lines, yeast, and plant cells.

Suitable cell lines which are used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, 211A cells, and cyno and Macaca nemestrina cell lines. In embodiments, the packaging cells are 293 cells, 293T cells, or A549 cells.

In some embodiments, a source cell line includes a cell line which is capable of producing recombinant retroviral particles, comprising a producer cell line and a transfer vector construct comprising a packaging signal. Methods of preparing viral stock solutions are illustrated by, e.g., Y. Soneoka et al. (1995) Nucl. Acids Res. 23:628-633, and N. R. Landau et al. (1992) J. Virol. 66:5110-5113, which are incorporated herein by reference.

In some embodiments, the assembly of a viral particle or virus-like particle is initiated by binding of the core protein to a unique encapsidation sequence within the viral genome (e.g., UTR with stem-loop structure). In some embodiments, the interaction of the core with the encapsidation sequence facilitates oligomerization.

In some embodiments, the targeted lipid particle is a virus-like particle which comprises a sequence that is devoid of or lacking viral RNA. In some embodiments, such particles are the result of removing or eliminating the viral RNA from the sequence. In some embodiments, this is achieved by using an endogenous packaging signal binding site on Gag. In some embodiments, the endogenous packaging signal binding site is on Pol. In some embodiments, the RNA which is to be delivered will contain a cognate packaging signal. In some embodiments, a heterologous binding domain (which is heterologous to Gag) located on the RNA to be delivered, and a cognate binding site located on Gag or Pol, are used to ensure packaging of the RNA to be delivered. In some embodiments, the heterologous sequence is non-viral or it could be viral, in which case it is derived from the same virus or a different virus. In some embodiments, the vector particles could be used to deliver therapeutic RNA, in which case functional integrase and/or reverse transcriptase is not required. In some embodiments, the vector particles could also be used to deliver a therapeutic gene of interest, in which case Pol is typically included. In some embodiments, the retroviral nucleic acid comprises one or more of (e.g., all of): a 5′ promoter (e.g., to control expression of the entire packaged RNA), a 5′ LTR (e.g., that includes R (polyadenylation tail signal) and/or U5 which includes a primer activation signal), a primer binding site, a Psi packaging signal, a RRE element for nuclear export, a promoter directly upstream of the transgene to control transgene expression, a transgene (or other exogenous agent element), a polypurine tract, and a 3′ LTR (e.g., that includes a mutated U3, a R, and U5). In some embodiments, the retroviral nucleic acid further comprises one or more of a cPPT, a WPRE, and/or an insulator element.

A retrovirus typically replicates by reverse transcription of its genomic RNA into a linear double-stranded DNA copy and subsequently covalently integrates its genomic DNA into a host genome. Illustrative retroviruses suitable for use in some embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV), Rous Sarcoma Virus (RSV), and other lentiviruses.

In some embodiments the retrovirus is a Gammaretrovirus. In some embodiments the retrovirus is an Epsilonretrovirus. In some embodiments the retrovirus is an Alpharetrovirus. In some embodiments the retrovirus is a Betaretrovirus. In some embodiments the retrovirus is a Deltaretrovirus. In some embodiments the retrovirus is a Lentivirus. In some embodiments the retrovirus is a Spumaretrovirus. In some embodiments the retrovirus is an endogenous retrovirus.

Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, HIV based vector backbones (i.e., HIV cis-acting sequence elements) are used.

In some embodiments, a vector herein is a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

In some embodiments, a viral vector comprises a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). In some embodiments, a viral vector comprises e.g., a virus or viral particle capable of transferring a nucleic acid into a cell, or the transferred nucleic acid (e.g., as naked DNA). In some embodiments, a viral vectors and transfer plasmids comprise structural and/or functional genetic elements that are primarily derived from a virus. A retroviral vector can comprise a viral vector or plasmid containing structural and functional genetic elements, or portions thereof, that are primarily derived from a retrovirus. A lentiviral vector can comprise a viral vector or plasmid containing structural and functional genetic elements, or portions thereof, including LTRs that are primarily derived from a lentivirus.

In embodiments, a lentiviral vector (e.g., lentiviral expression vector) may comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements are present in RNA form in lentiviral particles and are present in DNA form in DNA plasmids.

In some embodiments, in the vectors described herein at least part of one or more protein coding regions that contribute to or are essential for replication are absent compared to the corresponding wild-type virus. In some embodiments, the viral vector is replication-defective. In some embodiments, the vector is capable of transducing a target non-dividing host cell and/or integrating its genome into a host genome.

In some embodiments, different cells differ in their usage of particular codons. In some embodiments, this codon bias corresponds to a bias in the relative abundance of particular tRNAs in the cell type. In some embodiments, by altering the codons in the sequence so that they are tailored to match with the relative abundance of corresponding tRNAs, it is possible to increase expression. In some embodiments, it is possible to decrease expression by deliberately choosing codons for which the corresponding tRNAs are known to be rare in the particular cell type. In some embodiments, an additional degree of translational control is available. An additional description of codon optimization is found, e.g., in WO 99/41397, which is herein incorporated by reference in its entirety.

Conventional techniques for generating retrovirus vectors (and, in particular, lentivirus vectors) with or without the use of packaging/helper vectors are known to those skilled in the art and are used to generate targeted lipid particles according to the present disclosure. (See, e.g., Derse and Newbold 1993 Virology 194:530-6; Maury et al. 1994 Virology 200:632-42; Wanisch et al. 2009. Mol Ther. 1798:1316-1332; Martarano et al. 1994 J. Virol. 68:3102-11; Naldini et al., (1996a, 1996b, and 1998); Zufferey et al., 1999, J. Virol., 73:2886; Huang et al., Mol. Cell. Biol., 5:3864; Liu et al., 1995, Genes Dev., 9:1766; Cullen et al., 1991. J. Virol. 65: 1053; and Cullen et al., 1991. Cell 58: 423; Dull et al., 1998, U.S. Pat. Nos. 6,013,516; and 5,994,136; PCT patent applications WO 99/15683, WO 98/17815, WO 99/32646, and WO 01/79518). Conventional techniques relating to packaging vectors and producer cells known in the art may also be used according to the present disclosure. (See, e.g., Yao et al, 1998; Jones et al, 2005.)

Provided herein are targeted lipid particles that comprise a naturally derived membrane. In some embodiments, the naturally derived membrane comprises membrane vesicles prepared from cells or tissues. In some embodiments, the targeted lipid particle comprises a vesicle that is obtainable from a cell. In some embodiments, the targeted lipid particle comprises a microvesicle, an exosome, a membrane enclosed body, an apoptotic body (from apoptotic cells), a particle (which is derived from e.g., platelets), an ectosome (derivable from, e.g., neutrophiles and monocytes in serum), a prostatosome (obtainable from prostate cancer cells), or a cardiosome (derivable from cardiac cells).

In some embodiments, the source cell is an endothelial cell, a fibroblast, a blood cell (e.g., a macrophage, a neutrophil, a granulocyte, a leukocyte), a stem cell (e.g., a mesenchymal stem cell, an umbilical cord stem cell, bone marrow stem cell, a hematopoietic stem cell, an induced pluripotent stem cell e.g., an induced pluripotent stem cell derived from a subject's cells), an embryonic stem cell (e.g., a stem cell from embryonic yolk sac, placenta, umbilical cord, fetal skin, adolescent skin, blood, bone marrow, adipose tissue, erythropoietic tissue, hematopoietic tissue), a myoblast, a parenchymal cell (e.g., hepatocyte), an alveolar cell, a neuron (e.g., a retinal neuronal cell), a precursor cell (e.g., a retinal precursor cell, a myeloblast, myeloid precursor cells, a thymocyte, a meiocyte, a megakaryoblast, a promegakaryoblast, a melanoblast, a lymphoblast, a bone marrow precursor cell, a normoblast, or an angioblast), a progenitor cell (e.g., a cardiac progenitor cell, a satellite cell, a radial glial cell, a bone marrow stromal cell, a pancreatic progenitor cell, an endothelial progenitor cell, a blast cell), or an immortalized cell (e.g., HeLa, HEK293, HFF-1, MRC-5, WI-38, IMR 90, IMR 91, PER.C6, HT-1080, or BJ cell). In some embodiments, the source cell is other than a 293 cell, HEK cell, human endothelial cell, or a human epithelial cell, monocyte, macrophage, dendritic cell, or stem cell.

In some embodiments, the targeted lipid particle has a density of <1, 1-1.1, 1.05-1.15, 1.1-1.2, 1.15-1.25, 1.2-1.3, 1.25-1.35, or >1.35 g/ml. In some embodiments, the targeted lipid particle composition comprises less than 0.01%, 0.05%, 0.1%, 0.5%, 1%, 1.5%, 2%, 2.5%, 3%, 4%, 5%, or 10% source cells by protein mass, or less than 0.01%, 0.05%, 0.1%, 0.5%, 1%, 1.5%, 2%, 2.5%, 3%, 4%, 5%, or 10% of cells having a functional nucleus.

In embodiments, the targeted lipid particle has a size, or the population of targeted lipid particles have an average size, that is less than about 0.01%, 0.05%, 0.1%, 0.5%, 1%, 2%, 3%, 4%, 5%, 10%, 20%, 30%, 40%, 50%, 60%, 70%, 80%, 90%, of that of the source cell.

In some embodiments the targeted lipid particle comprises an extracellular vesicle, e.g., a cell-derived vesicle comprising a membrane that encloses an internal space and has a smaller diameter than the cell from which it is derived. In embodiments the extracellular vesicle has a diameter from 20 nm to 1000 nm. In embodiments the targeted lipid particle comprises an apoptotic body, a fragment of a cell, a vesicle derived from a cell by direct or indirect manipulation, a vesiculated organelle, and a vesicle produced by a living cell (e.g., by direct plasma membrane budding or fusion of the late endosome with the plasma membrane). In embodiments the extracellular vesicle is derived from a living or dead organism, explanted tissues or organs, or cultured cells.

In embodiments, the targeted lipid particle comprises a nanovesicle, e.g., a cell-derived small (e.g., between 20-250 nm in diameter, or 30-150 nm in diameter) vesicle comprising a membrane that encloses an internal space, and which is generated from said cell by direct or indirect manipulation. The production of nanovesicles can, in some instances, result in the destruction of the source cell. The nanovesicle may comprise a lipid or fatty acid and polypeptide.

In embodiments, the targeted lipid particle comprises an exosome. In embodiments, the exosome is a cell-derived small (e.g., between 20-300 nm in diameter, or 40-200 nm in diameter) vesicle comprising a membrane that encloses an internal space, and which is generated from said cell by direct plasma membrane budding or by fusion of the late endosome with the plasma membrane. In embodiments, production of exosomes does not result in the destruction of the source cell. In embodiments, the exosome comprises lipid or fatty acid and polypeptide.

In some embodiments, the targeted lipid particle is derived from a source cell with a genetic modification which results in increased expression of an immunomodulatory agent. In some embodiments, the immunosuppressive agent is on an exterior surface of the cell. In some embodiments, the immunosuppressive agent is incorporated into the exterior surface of the targeted lipid particle. In some embodiments, the targeted lipid particle comprises an immunomodulatory agent attached to the surface of the solid particle by a covalent or non-covalent bond.

### Generation of Cell-Derived Particles

In some embodiments, targeted lipid particles are generated by inducing budding of an exosome, microvesicle, membrane vesicle, extracellular membrane vesicle, plasma membrane vesicle, giant plasma membrane vesicle, apoptotic body, mitoparticle, pyrenocyte, lysosome, or other membrane enclosed vesicle.

In some embodiments, targeted lipid particles are generated by inducing cell enucleation. Enucleation is performed using assays such as genetic, chemical (e.g., using Actinomycin D, see Bayona-Bafaluy et al., “A chemical enucleation method for the transfer of mitochondrial DNA to ρ° cells” Nucleic Acids Res. 2003 Aug. 15; 31(16): e98), or mechanical methods (e.g., squeezing or aspiration, see Lee et al., “A comparative study on the efficiency of two enucleation methods in pig somatic cell nuclear transfer: effects of the squeezing and the aspiration methods.” Anim Biotechnol. 2008; 19(2):71-9), or combinations thereof.

In some embodiments, the targeted lipid particles are generated by inducing cell fragmentation. In some embodiments, cell fragmentation is performed using the following methods, including, but not limited to: chemical methods, mechanical methods (e.g., centrifugation (e.g., ultracentrifugation, or density centrifugation), freeze-thaw, or sonication), or combinations thereof.

In some embodiments, the targeted lipid particle is a microvesicle. In some embodiments the microvesicle has a diameter of about 100 nm to about 2000 nm. In some embodiments, a targeted lipid particle comprises a cell ghost. In some embodiments, a vesicle is a plasma membrane vesicle, e.g., a giant plasma membrane vesicle.

In some embodiments, a characteristic of a targeted lipid particle is described by comparison to a reference cell. In embodiments, the reference cell is the source cell. In embodiments, the reference cell is a HeLa, HEK293, HFF-1, MRC-5, WI-38, IMR 90, IMR 91, PER.C6, HT-1080, or BJ cell. In some embodiments, for example when the source cell used to make the targeted lipid particle is not available for testing after the targeted lipid particle is made, a characteristic of a population of targeted lipid particle is described by comparison to a population of reference cells, e.g., a population of source cells, or a population of HeLa, HEK293, HFF-1, MRC-5, WI-38, IMR 90, IMR 91, PER.C6, HT-1080, or BJ cells.

NiV-F protein has a sequence that has at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:116.

In some embodiments, the F protein is a mutant NiV-F protein that is a biologically active portion thereof that comprises a 22 amino acid truncation at or near the C-terminus of the wild-type NiV-F protein (SEQ ID NO:111). In some embodiments, the NiV-F protein comprises an amino acid sequence set forth in SEQ ID NO: 117. In some embodiments, the NiV-F protein has a sequence with at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:117. In some embodiments, the NiV-F protein comprises an amino acid sequence set forth in SEQ ID NO: 118. In some embodiments, the NiV-F protein has a sequence with at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:118. In some embodiments, the NiV-F protein comprises an amino acid sequence set forth in SEQ ID NO:119. In some embodiments, the NiV-F protein has a sequence with at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO: 119. In some embodiments, the variant F protein is a mutant Niv-F protein that has the sequence of amino acids set forth in SEQ ID NO: 133. In some embodiments, the NiV-F protein has a sequence with at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:133.

### GPRC5D Fusion Protein

Also provided herein are fusion proteins targeting GPRC5D. In some embodiments, the GPRC5D binders disclosed herein are fused to an envelope glycoprotein G, H, and/or an F protein of the Paramyxoviridae family. In some embodiments the fusogen contains a Nipah virus protein F, a measles virus F protein, a tupaia paramyxovirus F protein, a paramyxovirus F protein, a Hendra virus F protein, a Henipavirus F protein, a Morbilivirus F protein, a respirovirus F protein, a Sendai virus F protein, a rubulavirus F protein, or an avulavirus F protein. In some embodiments, the lipid particle contains a henipavirus envelope attachment glycoprotein G (G protein) or a biologically active portion thereof and/or a henipavirus envelope fusion glycoprotein F (F protein) or a biologically active portion thereof.

In some embodiments, the fusogen is glycoprotein GP64 of baculovirus, or glycoprotein GP64 variant E45K/T259A.

In some embodiments, the fusogen is a hemagglutinin-neuraminidase (HN) and/or fusion (F) protein (F/HN) from a respiratory paramyxovirus. In some embodiments, the respiratory paramyxovirus is a Sendai virus. The HN and F glycoproteins of Sendai viruses function to attach to sialic acids via the HN protein, and to mediate cell fusion for entry into cells via the F protein. In some embodiments, the fusogen is a F and/or HN protein from the murine parainfluenza virus type 1 (see e.g., U.S. Pat. No. 10,704,061).

In some embodiments, the lipid particle (e.g., vector) is pseudotyped with viral glycoproteins as described herein such as a NiV-F and/or NiV-G protein.

In some embodiments, the vector further comprises a vector-surface targeting moiety which specifically binds to a target ligand. In some embodiments, the vector-surface targeting moiety is a polypeptide. In some embodiments, a nucleic acid encoding the Paramyxovirus envelope protein (e.g., G protein) is modified with a targeting moiety to specifically bind to a target molecule on a target cells. In some embodiments, the targeting moiety is any targeting protein, including but not necessarily limited to antibodies and antigen binding fragments thereof.

It has been reported that the henipavirus F proteins from various species exhibit compatibility with G proteins from other species to trigger fusion (Brandel-Tretheway et al. Journal of Virology. 2019. 93(13):e00577-19). In some aspects of the provided lipid particles (e.g., lentiviral vectors), the F protein is heterologous to the G protein, i.e., the F and G proteins or biologically active portions thereof are from different henipavirus species. For example, in some embodiments the G protein is from Hendra virus and the F protein is a NiV-F as described. In other aspects, the F and/or G protein are chimeric F and/or G protein containing regions of F and/or G proteins from different species of Henipavirus. In some embodiments, replacing a portion of the F protein with amino acids from a heterologous sequence of Henipavirus results in fusion to the G protein with the heterologous sequence. (Brandel-Tretheway et al. 2019). In some embodiments, the chimeric F and/or G protein contains an extracellular domain from one henipavirus species and a transmembrane and/or cytoplasmic domain from a different henipavirus species. For example, in some embodiments the F protein contains an extracellular domain of Hendra virus and a transmembrane/cytoplasmic domain of Nipah virus.

In some embodiments, the fusion protein contains a henipavirus envelope attachment glycoprotein G (G protein) or a biologically active portion thereof and a single domain antibody (sdAb) variable domain or a single chain variable fragment (scFv) that binds GPRC5D as disclosed herein. In some embodiments, the sdAb variable domain or scFv is linked directly or indirectly to the G protein. In some embodiments, the sdAb variable domain or scFv is linked to the C-terminus (C-terminal amino acid) of the G protein or the biologically active portion thereof. In some embodiments, the linkage is via a peptide linker, such as a flexible peptide linker. Table 22 provides a list of non-limiting examples of G proteins.

In some embodiments the G protein is a Henipavirus G protein or a biologically active portion thereof. In some embodiments, the Henipavirus G protein is a Hendra (HeV) virus G protein, a Nipah (NiV) virus G-protein (NiV-G), a Cedar (CedPV) virus G-protein, a Mojiang virus G-protein, a bat Paramyxovirus G-protein, or a biologically active portion thereof. Non-limiting examples of G proteins include those corresponding to SEQ ID NOs: 129, 138, 139, 140, and 141.

In some embodiments, the attachment G proteins are type II transmembrane glycoproteins containing an N-terminal cytoplasmic tail (e.g., corresponding to amino acids 1-49 of SEQ ID NO: 120), a transmembrane domain (e.g., corresponding to amino acids 50-70 of SEQ ID NO: 120), and an extracellular domain containing an extracellular stalk (e.g., corresponding to amino acids 71-187 of SEQ ID NO: 120), and a globular head (corresponding to amino acids 188-602 of SEQ ID NO: 120). In such embodiments, the N-terminal cytoplasmic domain is within the inner lumen of the lipid bilayer and the C-terminal portion is the extracellular domain that is exposed on the outside of the lipid bilayer. Regions of the stalk in the C-terminal region (e.g., corresponding to amino acids 159-167 of NiV-G) have been shown to be involved in interactions with F protein and triggering of F protein fusion (Liu et al. 2015 J of Virology 89:1838). In wild-type G protein, the globular head mediates receptor binding to henipavirus entry receptors ephrin B2 and ephrin B3, but is dispensable for membrane fusion (Brandel-Tretheway et al. Journal of Virology. 2019. 93(13)e00577-19). In some embodiments herein, tropism of the G protein is altered by linkage of the G protein or biologically active fragment thereof (e.g., cytoplasmic truncation) to a sdAb variable domain. Binding of the G protein to a binding partner can trigger fusion mediated by a compatible F protein or a biologically active portion thereof. G protein sequences disclosed herein are predominantly disclosed as expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

G glycoproteins are highly conserved among henipavirus species. For example, the G proteins of NiV and HeV viruses share 79% amino acid identity. Studies have shown a high degree of compatibility among G proteins with F proteins of different species as demonstrated by heterotypic fusion activation (Brandel-Tretheway et al. Journal of Virology. 2019). As described further below, in some embodiments, a targeted lipid particle contains heterologous G and F proteins from different species.

In some embodiments, the G protein has a sequence set forth in any of SEQ ID NOs: 120, 129, 138, 139, 140, 141, 148, 156, or 158-160, or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOs: 120, 129, 138, 139, 140, 141, 148, 156, or 158-160. In some embodiments, the G protein or functionally active variant or biologically active portion is a protein that retains fusogenic activity in conjunction with a Henipavirus F protein, such as an F protein (e.g., NiV-F or HeV-F). Fusogenic activity includes the activity of the G protein in conjunction with a Henipavirus F protein to promote or facilitate fusion of two membrane lumens, such as the lumen of the targeted lipid particle having embedded in its lipid bilayer a henipavirus F and G protein, and a cytoplasm of a target cell, e.g., a cell that contains a surface receptor or molecule that is recognized or bound by the antibody or antigen binding fragment thereof on the targeted lipid particle. In some embodiments, the F protein and G protein are from the same Henipavirus species (e.g., NiV-G and NiV-F). In some embodiments, the F protein and G protein are from different Henipavirus species (e.g., NiV-G and HeV-F).

In some embodiments, the G protein has the sequence of amino acids set forth in SEQ ID NOs: 120, 129, 138, 139, 140, 141, 148, 156, or 158-160, or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to any one of SEQ ID NOs: 120, 129, 138, 139, 140, 141, 148, 156, or 158-160 and retains fusogenic activity in conjunction with a Henipavirus F protein (e.g., NiV-F or HeV-F). In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to any one of SEQ ID NOs: 120, 129, 138, 139, 140, 141, 148, 156, or 158-160 and retains fusogenic activity in conjunction with a Henipavirus F protein (e.g., NiV-F or HeV-F).

Reference to retaining fusogenic activity includes activity (in conjunction with a Henipavirus F protein) that is at or about 10% to at or about 150% or more of the level or degree of binding of the corresponding wild-type G protein, such as set forth in any one of SEQ ID NOs: 120, 129, 138, 139, 140, 141, 148, 156, or 158-160, such as at least or at least about 10% of the level or degree of fusogenic activity of the corresponding wild-type G protein, such as at least or at least about 15% of the level or degree of fusogenic activity of the corresponding wild-type G protein, such as at least or at least about 20% of the level or degree of fusogenic activity of the corresponding wild-type G protein, such as at least or at least about 25% of the level or degree of fusogenic activity of the corresponding wild-type G protein, such as at least or at least about 30% of the level or degree of fusogenic activity of the corresponding wild-type G protein, such as at least or at least about 35% of the level or degree of fusogenic activity of the corresponding wild-type G protein, such as at least or at least about 40% of the level or degree of fusogenic activity of the corresponding wild-type G protein, such as at least or at least about 45% of the level or degree of fusogenic activity of the corresponding wild-type G protein, such as at least or at least about 50% of the level or degree of fusogenic activity of the corresponding wild-type G protein, such as at least or at least about 55% of the level or degree of fusogenic activity of the corresponding wild-type G protein, such as at least or at least about 60% of the level or degree of fusogenic activity of the corresponding wild-type G protein, such as at least or at least about 65% of the level or degree of fusogenic activity of the corresponding wild-type G protein, such as at least or at least about 70% of the level or degree of fusogenic activity of the corresponding wild-type G protein, such as at least or at least about 75% of the level or degree of fusogenic activity of the corresponding wild-type G protein, such as at least or at least about 80% of the level or degree of fusogenic activity of the corresponding wild-type G protein, such as at least or at least about 85% of the level or degree of fusogenic activity of the corresponding wild-type G protein, such as at least or at least about 90% of the level or degree of fusogenic activity of the corresponding wild-type G protein, such as at least or at least about 95% of the level or degree of fusogenic activity of the corresponding wild-type G protein, such as at least or at least about 100% of the level or degree of fusogenic activity of the corresponding wild-type G protein, or such as at least or at least about 120% of the level or degree of fusogenic activity of the corresponding wild-type G protein.

In some embodiments, the G protein is a mutant G protein that is a functionally active variant or biologically active portion containing one or more amino acid mutations, such as one or more amino acid insertions, deletions, substitutions, or truncations. In some embodiments, the mutations described herein relate to amino acid insertions, deletions, substitutions, or truncations of amino acids compared to a reference G protein sequence. In some embodiments, the reference G protein sequence is the wild-type sequence of a G protein or a biologically active portion thereof. In some embodiments, the functionally active variant or the biologically active portion thereof is a mutant of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein, or biologically active portions thereof. In some embodiments, the wild-type G protein has the sequence set forth in any one of SEQ ID NOs: 120, 129, 138, 139, 140, 141, 148, 156, or 158-160.

In some embodiments, the G protein is a mutant G protein that is a biologically active portion that is an N-terminally and/or C-terminally truncated fragment of a wild-type_Hendra (HeV) virus G protein, a wild-type Nipah (NiV) virus G-protein (NiV-G), a wild-type Cedar (CedPV) virus G-protein, a wild-type Mojiang virus G-protein, or a wild-type bat Paramyxovirus G-protein. In some embodiments, the truncation is an N-terminal truncation of all or a portion of the cytoplasmic domain. In some embodiments, the mutant G protein is a biologically active portion that is truncated and lacks up to 49 contiguous amino acid residues at or near the N-terminus of the wild-type G protein, such as a wild-type G protein set forth in any one of SEQ ID NOs: 120, 129, 138, 139, 140, 141, 148, 156, or 158-160. In some embodiments, the mutant G protein is truncated and lacks up to 49 contiguous amino acids, such as up to 49, 48, 47, 46, 45, 44, 43, 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acid(s) at the N-terminus of the wild-type G protein.

In some embodiments, the G protein is a wild-type Nipah virus G (NiV-G) protein or a Hendra virus G protein, or is a functionally active variant or biologically active portion thereof. In some embodiments, the G protein is a NiV-G protein that has the sequence set forth in SEQ ID NO:120, SEQ ID NO:138, or SEQ ID NO:148, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:120, SEQ ID NO:138, or SEQ ID NO:148.

In some embodiments, the G protein is a mutant NiV-G protein that is a biologically active portion of a wild-type NiV-G. In some embodiments, the biologically active portion is an N-terminally truncated fragment. In some embodiments, the mutant NiV-G protein is truncated and lacks up to 5 contiguous amino acid residues at or near the N-terminus of the wild-type NiV-G protein (SEQ ID NO:120, SEQ ID NO:138, or SEQ ID NO:148), up to 6 contiguous amino acid residues at or near the N-terminus of the wild-type NiV-G protein (SEQ ID NO:120, SEQ ID NO:138, or SEQ ID NO:148), up to 7 contiguous amino acid residues at or near the N-terminus of the wild-type NiV-G protein (SEQ ID NO:120, SEQ ID NO:138, or SEQ ID NO:148), up to 8 contiguous amino acid residues at or near the N-terminus of the wild-type NiV-G protein (SEQ ID NO:120, SEQ ID NO:138, or SEQ ID NO:148), up to 9 contiguous amino acid residues at or near the N-terminus of the wild-type NiV-G protein (SEQ ID NO:120, SEQ ID NO:138, or SEQ ID NO:148), up to 10 contiguous amino acid residues at or near the N-terminus of the wild-type NiV-G protein (SEQ ID NO:120, SEQ ID NO:138, or SEQ ID NO:148), up to 11 contiguous amino acid residues at or near the N-terminus of the wild-type NiV-G protein (SEQ ID NO:120, SEQ ID NO:138, or SEQ ID NO:148), up to 12 contiguous amino acid residues at or near the N-terminus of the wild-type NiV-G protein (SEQ ID NO:120, SEQ ID NO:138, or SEQ ID NO:148), up to 13 contiguous amino acid residues at or near the N-terminus of the wild-type NiV-G protein (SEQ ID NO:120, SEQ ID NO:138, or SEQ ID NO:148), up to 14 contiguous amino acid residues at or near the N-terminus of the wild-type NiV-G protein (SEQ ID NO:120, SEQ ID NO:138, or SEQ ID NO:148), up to 15 contiguous amino acid residues at or near the N-terminus of the wild-type NiV-G protein (SEQ ID NO:120, SEQ ID NO:138, or SEQ ID NO:148), up to 16 contiguous amino acid residues at or near the N-terminus of the wild-type NiV-G protein (SEQ ID NO:120, SEQ ID NO:138, or SEQ ID NO:148), up to 17 contiguous amino acid residues at or near the N-terminus of the wild-type NiV-G protein (SEQ ID NO:120, SEQ ID NO:138, or SEQ ID NO:148), up to 18 contiguous amino acid residues at or near the N-terminus of the wild-type NiV-G protein (SEQ ID NO:120, SEQ ID NO:138, or SEQ ID NO:148), up to 19 contiguous amino acid residues at or near the N-terminus of the wild-type NiV-G protein (SEQ ID NO:120, SEQ ID NO:138, or SEQ ID NO:148), up to 20 contiguous amino acid residues at or near the N-terminus of the wild-type NiV-G protein (SEQ ID NO:120, SEQ ID NO:138, or SEQ ID NO:148), up to 21 contiguous amino acid residues at or near the N-terminus of the wild-type NiV-G protein (SEQ ID NO:120, SEQ ID NO:138, or SEQ ID NO:148), up to 22 contiguous amino acid residues at or near the N-terminus of the wild-type NiV-G protein (SEQ ID NO:120, SEQ ID NO:138, or SEQ ID NO:148), up to 23 contiguous amino acid residues at or near the N-terminus of the wild-type NiV-G protein (SEQ ID NO:120, SEQ ID NO:138, or SEQ ID NO:148), up to 24 contiguous amino acid residues at or near the N-terminus of the wild-type NiV-G protein (SEQ ID NO:120, SEQ ID NO:138, or SEQ ID NO:148), up to 25 contiguous amino acid residues at or near the N-terminus of the wild-type NiV-G protein (SEQ ID NO:120, SEQ ID NO:138, or SEQ ID NO:148), up to 26 contiguous amino acid residues at or near the N-terminus of the wild-type NiV-G protein (SEQ ID NO:120, SEQ ID NO:138, or SEQ ID NO:148), up to 27 contiguous amino acid residues at or near the N-terminus of the wild-type NiV-G protein (SEQ ID NO:120, SEQ ID NO:138, or SEQ ID NO:148), up to 28 contiguous amino acid residues at or near the N-terminus of the wild-type NiV-G protein (SEQ ID NO:120, SEQ ID NO:138, or SEQ ID NO:148), up to 29 contiguous amino acid residues at or near the N-terminus of the wild-type NiV-G protein (SEQ ID NO:120, SEQ ID NO:138, or SEQ ID NO:148), up to 30 contiguous amino acid residues at or near the N-terminus of the wild-type NiV-G protein (SEQ ID NO:120, SEQ ID NO:138, or SEQ ID NO:148), up to 31 contiguous amino acid residues at or near the N-terminus of the wild-type NiV-G protein (SEQ ID NO:120, SEQ ID NO:138, or SEQ ID NO:148), up to 32 contiguous amino acid residues at or near the N-terminus of the wild-type NiV-G protein (SEQ ID NO:120, SEQ ID NO:138, or SEQ ID NO:148), up to 33 contiguous amino acid residues at or near the N-terminus of the wild-type NiV-G protein (SEQ ID NO:120, SEQ ID NO:138, or SEQ ID NO:148), up to 34 contiguous amino acid residues at or near the N-terminus of the wild-type NiV-G protein (SEQ ID NO:120, SEQ ID NO:138, or SEQ ID NO:148), up to 35 contiguous amino acid residues at or near the N-terminus of the wild-type NiV-G protein (SEQ ID NO:120, SEQ ID NO:138, or SEQ ID NO:148), up to 36 contiguous amino acid residues at or near the N-terminus of the wild-type NiV-G protein (SEQ ID NO:120, SEQ ID NO:138, or SEQ ID NO:148), up to 37 contiguous amino acid residues at or near the N-terminus of the wild-type NiV-G protein (SEQ ID NO:120, SEQ ID NO:138, or SEQ ID NO:148), up to 38 contiguous amino acid residues at or near the N-terminus of the wild-type NiV-G protein (SEQ ID NO:120, SEQ ID NO:138, or SEQ ID NO:148), up to 39 contiguous amino acid residues at or near the N-terminus of the wild-type NiV-G protein (SEQ ID NO:120, SEQ ID NO:138, or SEQ ID NO:148), up to 40 contiguous amino acid residues at or near the N-terminus of the wild-type NiV-G protein (SEQ ID NO:120, SEQ ID NO:138, or SEQ ID NO:148), up to 41 contiguous amino acid residues at or near the N-terminus of the wild-type NiV-G protein (SEQ ID NO:120, SEQ ID NO:138, or SEQ ID NO:148), up to 42 contiguous amino acid residues at or near the N-terminus of the wild-type NiV-G protein (SEQ ID NO:120, SEQ ID NO:138, or SEQ ID NO:148), up to 43 contiguous amino acid residues at or near the N-terminus of the wild-type NiV-G protein (SEQ ID NO:120, SEQ ID NO:138, or SEQ ID NO:148), up to 44 contiguous amino acid residues at or near the N-terminus of the wild-type NiV-G protein (SEQ ID NO:134, SEQ ID NO: 152, or SEQ ID NO: 162), or up to 45 contiguous amino acid residues at or near the N-terminus of the wild-type NiV-G protein (SEQ ID NO:120, SEQ ID NO:138, or SEQ ID NO:148).

In some embodiments, the NiV-G protein is a biologically active portion that does not contain a cytoplasmic domain. In some embodiments, the NiV-G protein without the cytoplasmic domain is encoded by SEQ ID NO:142.

In some embodiments, the mutant NiV-G protein comprises a sequence set forth in any of SEQ ID NOs: 121-126, 149-154, 132, 142, or 157, or is a functional variant thereof that has an amino acid sequence having at least at or about 80%, at least at or about 81%, at least at or about 82%, at least at or about 83%, at least at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NOs: 121-126, 149-154, 132, 142, or 157.

In some embodiments, the mutant NiV-G protein has a 5 amino acid truncation at or near the N-terminus of the wild-type NiV-G protein (SEQ ID NO:120, SEQ ID NO:138, or SEQ ID NO:148), such as set forth in SEQ ID NO: 121 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:121, or as set forth in SEQ ID NO:149 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:149 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:149.

In some embodiments, the mutant NiV-G protein has a 10 amino acid truncation at or near the N-terminus of the wild-type NiV-G protein (SEQ ID NO:120, SEQ ID NO:138, or SEQ ID NO:148), such as set forth in SEQ ID NO:122 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:122, or such as set forth in SEQ ID NO:150 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:150.

In some embodiments, the mutant NiV-G protein has a 15 amino acid truncation at or near the N-terminus of the wild-type NiV-G protein (SEQ ID NO:120, SEQ ID NO:138, or SEQ ID NO:148), such as set forth in SEQ ID NO:123 or a functional variant thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:123, or such as set forth in SEQ ID NO:151 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:151.

In some embodiments, the mutant NiV-G protein has a 20 amino acid truncation at or near the N-terminus of the wild-type NiV-G protein (SEQ ID NO:120, SEQ ID NO:138, or SEQ ID NO:148) such as set forth in SEQ ID NO: 124, or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least or about 96%, at least at or about 97%, at least at or about 98%, or at least at or about 99% sequence identity to SEQ ID NO:124, or such as set forth in SEQ ID NO:152 or a functional variant thereof having at least at or about 80%, at least at or about 81%, at least at or about 82%, at least at or about 83%, at least or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:152.

In some embodiments, the mutant NiV-G protein has a 25 amino acid truncation at or near the N-terminus of the wild-type NiV-G protein (SEQ ID NO:120, SEQ ID NO:138, or SEQ ID NO:148), such as set forth in SEQ ID NO:125 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least or about 96%, at least at or about 97%, at least at or about 98%, or at least at or about 99% sequence identity to SEQ ID NO:125, or such as set forth in SEQ ID NO:153 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:153.

In some embodiments, the mutant NiV-G protein has a 30 amino acid truncation at or near the N-terminus of the wild-type NiV-G protein (SEQ ID NO:120, SEQ ID NO:138, or SEQ ID NO:148), such as set forth in SEQ ID NO:126 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:126, or such as set forth in SEQ ID NO:154 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:154.

In some embodiments, the mutant NiV-G protein has a 33 amino acid truncation at or near the N-terminus of the wild-type NiV-G protein (SEQ ID NO:120, SEQ ID NO:138, or SEQ ID NO:148) or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:132, or such as set forth in SEQ ID NO:155 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:155.

In some embodiments, the mutant NiV-G protein has a 34 amino acid truncation at or near the N-terminus of the wild-type NiV-G protein (SEQ ID NO:120, SEQ ID NO:138, or SEQ ID NO:148), such as set forth in SEQ ID NO:132 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:132, or such as set forth in SEQ ID NO:155 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:155.

In some embodiments, the NiV-G protein has a 34 amino acid truncation at or near the N-terminus of the wild-type NiV-G protein (SEQ ID NO:120, SEQ ID NO:138, or SEQ ID NO:148) and one or more amino acid substitutions corresponding to amino acid substitutions selected from E501A, W504A, Q530A, and E533A with reference to the numbering set forth in SEQ ID NO:138.

In some embodiments, the mutant NiV-G protein lacks the N-terminal cytoplasmic domain of the wild-type NiV-G protein (SEQ ID NO:120, SEQ ID NO:138, or SEQ ID NO:148), such as set forth in SEQ ID NO:156 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:156.

In some embodiments, the mutant G protein is a mutant HeV-G protein that has the sequence set forth in SEQ ID NO: 129 or 159, or is a functional variant or biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least or about 96%, at least at or about 97%, at least at or about 98%, or at least at or about 99% sequence identity to SEQ ID NO:129 or 159.

In some embodiments, the G protein is a mutant HeV-G protein that is a biologically active portion of a wild-type HeV-G. In some embodiments, the biologically active portion is an N-terminally truncated fragment. In some embodiments, the mutant HeV-G protein is truncated and lacks up to 5 contiguous amino acid residues at or near the N-terminus of the wild-type HeV-G protein (SEQ ID NO: 129 or 159), up to 6 contiguous amino acid residues at or near the N-terminus of the wild-type HeV-G protein (SEQ ID NO:129 or 159), up to 7 contiguous amino acid residues at or near the N-terminus of the wild-type HeV-G protein (SEQ ID NO:129 or 159), up to 8 contiguous amino acid residues at or near the N-terminus of the wild-type HeV-G protein (SEQ ID NO:129 or 159), up to 9 contiguous amino acid residues at or near the N-terminus of the wild-type HeV-G protein (SEQ ID NO:129 or 159), up to 10 contiguous amino acid residues at or near the N-terminus of the wild-type HeV-G protein (SEQ ID NO:129 or 159), up to 11 contiguous amino acid residues at or near the N-terminus of the wild-type HeV-G protein (SEQ ID NO:129 or 159), up to 12 contiguous amino acid residues at or near the N-terminus of the wild-type HeV-G protein (SEQ ID NO:129 or 159), up to 13 contiguous amino acid residues at or near the N-terminus of the wild-type HeV-G protein (SEQ ID NO:129 or 159), up to 14 contiguous amino acid residues at or near the N-terminus of the wild-type HeV-G protein (SEQ ID NO:129 or 159), up to 15 contiguous amino acid residues at or near the N-terminus of the wild-type HeV-G protein (SEQ ID NO:129 or 159), up to 16 contiguous amino acid residues at or near the N-terminus of the wild-type HeV-G protein (SEQ ID NO:129 or 159), up to 17 contiguous amino acid residues at or near the N-terminus of the wild-type HeV-G protein (SEQ ID NO:129 or 159), up to 18 contiguous amino acid residues at or near the N-terminus of the wild-type HeV-G protein (SEQ ID NO:129 or 159), up to 19 contiguous amino acid residues at or near the N-terminus of the wild-type HeV-G protein (SEQ ID NO:129 or 159), up to 20 contiguous amino acid residues at or near the N-terminus of the wild-type HeV-G protein (SEQ ID NO:129 or 159), up to 21 contiguous amino acid residues at or near the N-terminus of the wild-type HeV-G protein (SEQ ID NO:129 or 159), up to 22 contiguous amino acid residues at or near the N-terminus of the wild-type HeV-G protein (SEQ ID NO:129 or 159), up to 23 contiguous amino acid residues at or near the N-terminus of the wild-type HeV-G protein (SEQ ID NO:129 or 159), up to 24 contiguous amino acid residues at or near the N-terminus of the wild-type HeV-G protein (SEQ ID NO:129 or 159), up to 25 contiguous amino acid residues at or near the N-terminus of the wild-type HeV-G protein (SEQ ID NO:129 or 159), up to 26 contiguous amino acid residues at or near the N-terminus of the wild-type HeV-G protein (SEQ ID NO:129 or 159), up to 27 contiguous amino acid residues at or near the N-terminus of the wild-type HeV-G protein (SEQ ID NO:129 or 159), up to 28 contiguous amino acid residues at or near the N-terminus of the wild-type HeV-G protein (SEQ ID NO:129 or 159), up to 29 contiguous amino acid residues at or near the N-terminus of the wild-type HeV-G protein (SEQ ID NO:129 or 159), up to 30 contiguous amino acid residues at or near the N-terminus of the wild-type HeV-G protein (SEQ ID NO:129 or 159), up to 31 contiguous amino acid residues at or near the N-terminus of the wild-type HeV-G protein (SEQ ID NO:129 or 159), up to 32 contiguous amino acid residues at or near the N-terminus of the wild-type HeV-G protein (SEQ ID NO:129 or 159), up to 33 contiguous amino acid residues at or near the N-terminus of the wild-type HeV-G protein (SEQ ID NO:129 or 159), up to 34 contiguous amino acid residues at or near the N-terminus of the wild-type HeV-G protein (SEQ ID NO:129 or 159), up to 35 contiguous amino acid residues at or near the N-terminus of the wild-type HeV-G protein (SEQ ID NO:129 or 159), up to 36 contiguous amino acid residues at or near the N-terminus of the wild-type HeV-G protein (SEQ ID NO:129 or 159), up to 37 contiguous amino acid residues at or near the N-terminus of the wild-type HeV-G protein (SEQ ID NO:129 or 159), up to 38 contiguous amino acid residues at or near the N-terminus of the wild-type HeV-G protein (SEQ ID NO:129 or 159), up to 39 contiguous amino acid residues at or near the N-terminus of the wild-type HeV-G protein (SEQ ID NO:129 or 159), up to 40 contiguous amino acid residues at or near the N-terminus of the wild-type HeV-G protein (SEQ ID NO:129 or 159), up to 41 contiguous amino acid residues at or near the N-terminus of the wild-type HeV-G protein (SEQ ID NO:129 or 159), up to 42 contiguous amino acid residues at or near the N-terminus of the wild-type HeV-G protein (SEQ ID NO:129 or 159), up to 43 contiguous amino acid residues at or near the N-terminus of the wild-type HeV-G protein (SEQ ID NO:129 or 159), up to 44 contiguous amino acid residues at or near the N-terminus of the wild-type HeV-G protein (SEQ ID NO:129 or 159), or up to 45 contiguous amino acid residues at or near the N-terminus of the wild-type HeV-G protein (SEQ ID NO:129 or 159).

In some embodiments, the HeV-G protein is a biologically active portion that does not contain a cytoplasmic domain. In some embodiments, the mutant HeV-G protein lacks the N-terminal cytoplasmic domain of the wild-type HeV-G protein (SEQ ID NO:129 or 159), such as set forth in SEQ ID NO: 143 or a functional variant thereof having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO: 143.

In some embodiments, the G protein or the functionally active variant or biologically active portion thereof binds to Ephrin B2 or Ephrin B3. In some aspects, the G protein has the sequence of amino acids set forth in any one of SEQ ID NO:120, SEQ ID NO:129, SEQ ID NO:138, SEQ ID NO:139, SEQ ID NO:148, SEQ ID NO:140, or SEQ ID NO:141, or is a functionally active variant thereof or a biologically active portion thereof that is able to bind to Ephrin B2 or Ephrin B3. In some embodiments, the functionally active variant or biologically active portion has an amino acid sequence having at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, and retains binding to Ephrin B2 or B3.

Reference to retaining binding to Ephrin B2 or B3 includes binding that is at least or at least about 5% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 10% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 15% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO: 140, or SEQ ID NO:141, or a functionally active variant or biologically active portion thereof, 20% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 25% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion, 30% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 35% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 40% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 45% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 50% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO: 140, or SEQ ID NO:141, or a functionally active variant or biologically active portion thereof, 55% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 60% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 65% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, 70% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, such as at least or at least about 75% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, such as at least or at least about 80% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, such as at least or at least about 85% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, such as at least or at least about 90% of the level or degree of binding of the corresponding wild-type G protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof, or such as at least or at least about 95% of the level or degree of binding of the corresponding wild-type protein, such as set forth in SEQ ID NO:120, SEQ ID NO:129, SEQ ID NO:138, SEQ ID NO:139, SEQ ID NO:148, SEQ ID NO:140, or SEQ ID NO:141, or a functionally active variant or biologically active portion thereof.

In some embodiments, the G protein is NiV-G or a functionally active variant or biologically active portion thereof and binds to Ephrin B2 or Ephrin B3. In some aspects, the NiV-G has the sequence of amino acids set forth in SEQ ID NO:120, SEQ ID NO:138, or SEQ ID NO:148, or is a functionally active variant thereof or a biologically active portion thereof that is able to bind to Ephrin B2 or Ephrin B3. In some embodiments, the functionally active variant or biologically active portion has an amino acid sequence having at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:120, SEQ ID NO:138, or SEQ ID NO:148 and retains binding to Ephrin B2 or B3. Exemplary biologically active portions include N-terminally truncated variants lacking all or a portion of the cytoplasmic domain, e.g., 1 or more, such as 1 to 49 contiguous N-terminal amino acid residues, e.g., set forth in any one of SEQ ID NOS: 121-126, 142, and 149-154.

Reference to retaining binding to Ephrin B2 or B3 includes binding that is at least or at least about 5% of the level or degree of binding of the corresponding wild-type NiV-G, such as set forth in SEQ ID NO:120, SEQ ID NO:138, or SEQ ID NO:148, 10% of the level or degree of binding of the corresponding wild-type NiV-G, such as set forth in SEQ ID NO:120, SEQ ID NO:138, or SEQ ID NO:148, 15% of the level or degree of binding of the corresponding wild-type NiV-G, such as set forth in SEQ ID NO:120, SEQ ID NO:138, or SEQ ID NO:148, 20% of the level or degree of binding of the corresponding wild-type NiV-G, such as set forth in SEQ ID NO:120, SEQ ID NO:138, or SEQ ID NO:148, 25% of the level or degree of binding of the corresponding wild-type NiV-G, such as set forth in SEQ ID NO:120, SEQ ID NO:138, or SEQ ID NO:148, 30% of the level or degree of binding of the corresponding wild-type NiV-G, such as set forth in S SEQ ID NO:120, SEQ ID NO:138, or SEQ ID NO:148, 35% of the level or degree of binding of the corresponding wild-type NiV-G, such as set forth in SEQ ID NO:120, SEQ ID NO:138, or SEQ ID NO:148, 40% of the level or degree of binding of the corresponding wild-type NiV-G, such as set forth in SEQ ID NO:120, SEQ ID NO:138, or SEQ ID NO:148, 45% of the level or degree of binding of the corresponding wild-type NiV-G, such as set forth in SEQ ID NO:120, SEQ ID NO:138, or SEQ ID NO:148, 50% of the level or degree of binding of the corresponding wild-type NiV-G, such as set forth in SEQ ID NO:120, SEQ ID NO:138, or SEQ ID NO:148, 55% of the level or degree of binding of the corresponding wild-type NiV-G, such as set forth in SEQ ID NO:120, SEQ ID NO:138, or SEQ ID NO:148, 60% of the level or degree of binding of the corresponding wild-type NiV-G, such as set forth in SEQ ID NO:120, SEQ ID NO:138, or SEQ ID NO:148, 65% of the level or degree of binding of the corresponding wild-type NiV-G, such as set forth in SEQ ID NO:120, SEQ ID NO:138, or SEQ ID NO:148, 70% of the level or degree of binding of the corresponding wild-type NiV-G, such as set forth in SEQ ID NO:120, SEQ ID NO:138, or SEQ ID NO:148, such as at least or at least about 75% of the level or degree of binding of the corresponding wild-type NiV-G, such as set forth in SEQ ID NO:120, SEQ ID NO:138, or SEQ ID NO:148, such as at least or at least about 80% of the level or degree of binding of the corresponding wild-type NIV-G, such as set forth in SEQ ID NO:120, SEQ ID NO:138, or SEQ ID NO:148, such as at least or at least about 85% of the level or degree of binding of the corresponding wild-type NiV-G, such as set forth in SEQ ID NO:120, SEQ ID NO:138, or SEQ ID NO:148, such as at least or at least about 90% of the level or degree of binding of the corresponding wild-type NiV-G, such as set forth in SEQ ID NO:120, SEQ ID NO:138, or SEQ ID NO:148, or such as at least or at least about 95% of the level or degree of binding of the corresponding wild-type NiV-G, such as set forth in SEQ ID NO:120, SEQ ID NO:138, or SEQ ID NO: 148.

In some embodiments, the G protein is HeV-G or a functionally active variant or biologically active portion thereof and binds to Ephrin B2 or Ephrin B3. In some aspects, the HeV-G has the sequence of amino acids set forth in SEQ ID NO:129 or 159, or is a functionally active variant thereof or a biologically active portion thereof that is able to bind to Ephrin B2 or Ephrin B3. In some embodiments, the functionally active variant or biologically active portion has an amino acid sequence having at least at or about 80%, at least at or about 85%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO:129 or 159 and retains binding to Ephrin B2 or B3. Exemplary biologically active portions include N-terminally truncated variants lacking all or a portion of the cytoplasmic domain, e.g., 1 or more, such as 1 to 49 contiguous N-terminal amino acid residues, e.g., set forth in any one of SEQ ID NO:143.

Reference to retaining binding to Ephrin B2 or B3 includes binding that is at least or at least about 5% of the level or degree of binding of the corresponding wild-type HeV-G, such as set forth in SEQ ID NO: 129 or 159, 10% of the level or degree of binding of the corresponding wild-type HeV-G, such as set forth in SEQ ID NO:129 or 159, 15% of the level or degree of binding of the corresponding wild-type HeV-G, such as set forth in SEQ ID NO:129 or 159, 20% of the level or degree of binding of the corresponding wild-type HeV-G, such as set forth in SEQ ID NO:129 or 159, 25% of the level or degree of binding of the corresponding wild-type HeV-G, such as set forth in SEQ ID NO: 129 or 159, 30% of the level or degree of binding of the corresponding wild-type HeV-G, such as set forth in SEQ ID NO:129 or 159, 35% of the level or degree of binding of the corresponding wild-type HeV-G, such as set forth in SEQ ID NO:129 or 159, 40% of the level or degree of binding of the corresponding wild-type HeV-G, such as set forth in SEQ ID NO:129 or 159, 45% of the level or degree of binding of the corresponding wild-type HeV-G, such as set forth in SEQ ID NO:129 or 159, 50% of the level or degree of binding of the corresponding wild-type HeV-G, such as set forth in SEQ ID NO:129 or 159, 55% of the level or degree of binding of the corresponding wild-type HeV-G, such as set forth in SEQ ID NO:129 or 159, 60% of the level or degree of binding of the corresponding wild-type HeV-G, such as set forth in SEQ ID NO: 129 or 159, 65% of the level or degree of binding of the corresponding wild-type HeV-G, such as set forth in SEQ ID NO:129 or 159, 70% of the level or degree of binding of the corresponding wild-type HeV-G, such as set forth in SEQ ID NO:129 or 159, such as at least or at least about 75% of the level or degree of binding of the corresponding wild-type HeV-G, such as set forth in SEQ ID NO:129 or 159, such as at least or at least about 80% of the level or degree of binding of the corresponding wild-type NIV-G, such as set forth in SEQ ID NO:129 or 159, such as at least or at least about 85% of the level or degree of binding of the corresponding wild-type HeV-G, such as set forth in SEQ ID NO:129 or 159, such as at least or at least about 90% of the level or degree of binding of the corresponding wild-type HeV-G, such as set forth in SEQ ID NO: 129 or 159, or such as at least or at least about 95% of the level or degree of binding of the corresponding wild-type HeV-G, such as set forth in SEQ ID NO:129 or 159.

In some embodiments, the G protein or the biologically thereof is a mutant G protein that exhibits reduced binding for the native binding partner of a wild-type G protein. In some embodiments, the mutant G protein or the biologically active portion thereof is a mutant of wild-type Niv-G and exhibits reduced binding to one or both of the native binding partners Ephrin B2 or Ephrin B3. In some embodiments, the mutant G-protein or the biologically active portion, such as a mutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the mutations described herein can improve transduction efficiency. In some embodiments, the mutations described herein allow for specific targeting of other desired cell types that are not Ephrin B2 or Ephrin B3. In some embodiments, the mutations described herein result in at least the partial inability to bind at least one natural receptor, such as to reduce the binding to at least one of Ephrin B2 or Ephrin B3. In some embodiments, the mutations described herein interfere with natural receptor recognition.

In some embodiments, the mutant NiV-G protein or the biologically active portion thereof is truncated and lacks up to 5 contiguous amino acid residues at or near the N-terminus of the wild-type NiV-G protein (SEQ ID NO:138), 6 contiguous amino acid residues at or near the N-terminus of the wild-type NiV-G protein (SEQ ID NO:138), 7 contiguous amino acid residues at or near the N-terminus of the wild-type NiV-G protein (SEQ ID NO:138), 8 contiguous amino acid residues at or near the N-terminus of the wild-type NiV-G protein (SEQ ID NO:138), 9 contiguous amino acid residues at or near the N-terminus of the wild-type NiV-G protein (SEQ ID NO:138), 10 contiguous amino acid residues at or near the N-terminus of the wild-type NiV-G protein (SEQ ID NO:138), 11 contiguous amino acid residues at or near the N-terminus of the wild-type NiV-G protein (SEQ ID NO:138), 12 contiguous amino acid residues at or near the N-terminus of the wild-type NiV-G protein (SEQ ID NO:138), 13 contiguous amino acid residues at or near the N-terminus of the wild-type NiV-G protein (SEQ ID NO:138), 14 contiguous amino acid residues at or near the N-terminus of the wild-type NiV-G protein (SEQ ID NO:138), 15 contiguous amino acid residues at or near the N-terminus of the wild-type NiV-G protein (SEQ ID NO:138), 16 contiguous amino acid residues at or near the N-terminus of the wild-type NiV-G protein (SEQ ID NO: 138), 17 contiguous amino acid residues at or near the N-terminus of the wild-type NiV-G protein (SEQ ID NO:138), 18 contiguous amino acid residues at or near the N-terminus of the wild-type NiV-G protein (SEQ ID NO:138), 19 contiguous amino acid residues at or near the N-terminus of the wild-type NiV-G protein (SEQ ID NO:138), 20 contiguous amino acid residues at or near the N-terminus of the wild-type NiV-G protein (SEQ ID NO:138), 21 contiguous amino acid residues at or near the N-terminus of the wild-type NiV-G protein (SEQ ID NO:138), 22 contiguous amino acid residues at or near the N-terminus of the wild-type NiV-G protein (SEQ ID NO:138), 23 contiguous amino acid residues at or near the N-terminus of the wild-type NiV-G protein (SEQ ID NO:138), 24 contiguous amino acid residues at or near the N-terminus of the wild-type NiV-G protein (SEQ ID NO:138), 25 contiguous amino acid residues at or near the N-terminus of the wild-type NiV-G protein (SEQ ID NO:138), 26 contiguous amino acid residues at or near the N-terminus of the wild-type NiV-G protein (SEQ ID NO:138), 27 contiguous amino acid residues at or near the N-terminus of the wild-type NiV-G protein (SEQ ID NO: 138), 28 contiguous amino acid residues at or near the N-terminus of the wild-type NiV-G protein (SEQ ID NO:138), 29 contiguous amino acid residues at or near the N-terminus of the wild-type NiV-G protein (SEQ ID NO:138), 30 contiguous amino acid residues at or near the N-terminus of the wild-type NiV-G protein (SEQ ID NO:138), 31 contiguous amino acid residues at or near the N-terminus of the wild-type NiV-G protein (SEQ ID NO:138), 32 contiguous amino acid residues at or near the N-terminus of the wild-type NiV-G protein (SEQ ID NO:138), 33 contiguous amino acid residues at or near the N-terminus of the wild-type NiV-G protein (SEQ ID NO:138), 34 contiguous amino acid residues at or near the N-terminus of the wild-type NiV-G protein (SEQ ID NO:138), 35 contiguous amino acid residues at or near the N-terminus of the wild-type NiV-G protein (SEQ ID NO:138), 36 contiguous amino acid residues at or near the N-terminus of the wild-type NiV-G protein (SEQ ID NO:138), 37 contiguous amino acid residues at or near the N-terminus of the wild-type NiV-G protein (SEQ ID NO:138), 38 contiguous amino acid residues at or near the N-terminus of the wild-type NiV-G protein (SEQ ID NO:138), 39 contiguous amino acid residues at or near the N-terminus of the wild-type NiV-G protein (SEQ ID NO:138), or 40 contiguous amino acid residues at or near the N-terminus of the wild-type NiV-G protein (SEQ ID NO:138).

In some embodiments, the G protein contains one or more amino acid substitutions in a residue that is involved in the interaction with one or both of Ephrin B2 and Ephrin B3. In some embodiments, the amino acid substitutions correspond to mutations E501A, W504A, Q530A, and E533A with reference to numbering set forth in SEQ ID NO: 138.

In some embodiments, the G protein is a mutant G protein containing one or more amino acid substitutions selected from the group consisting of E501A, W504A, Q530A, and E533A with reference to numbering set forth in SEQ ID NO:138. In some embodiments, the G protein is a mutant G protein that contains one or more amino acid substitutions selected from the group consisting of E501A, W504A, Q530A, and E533A with reference to SEQ ID NO:138 or a biologically active portion thereof containing an N-terminal truncation. In some embodiments, the G protein is a mutant G protein that contains one or more amino acid substitutions selected from the group consisting of E501A, W504A, Q530A, and E533A in combination with any one of the N-terminal truncations disclosed above with reference to SEQ ID NO:138 or a biologically active portion thereof. In some embodiments, any of the mutant G proteins described above contains one, two, three, or all four amino acid selected from the group consisting of E501A, W504A, Q530A, and E533A with reference to numbering set forth in SEQ ID NO:138, in all pairwise and triple combinations thereof.

In some embodiments, the mutant NiV-G protein has the amino acid sequence set forth in SEQ ID NO: 127 or 155 or an amino acid sequence having at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% sequence identity to SEQ ID NO: 127 or 155. In some embodiments, the G protein has the sequence of amino acids set forth in SEQ ID NO: 127 or 155.

In some embodiments, the targeted envelope protein contains a G protein or a functionally active variant or biologically active portion thereof and an antibody or antigen binding fragment thereof, in which the targeted envelope protein exhibits increased binding for another molecule that is different from the native binding partner of a wild-type G protein. In some embodiments, the antibody or antigen binding fragment thereof is a scFv or sdAb. In some embodiments, the other molecule is a protein expressed on the surface of desired target cell. In some embodiments the other molecule that is different from the native binding partner of a wild-type G protein is GPRC5D. In some embodiments, the increased binding to the other molecule is increased by greater than at or about 25%, at or about 30%, at or about 40%, at or about 50%, at or about 60%, at or about 70%, at or about 80%, at or about 90%, or at or about 100%. In some embodiments, the binding confers re-targeted binding compared to the binding of a wild-type G protein in which a new or different binding activity is conferred.

In some embodiments, the C-terminus of the antibody or antigen binding fragment thereof is attached to the C-terminus of the G protein or biologically active portion thereof. In some embodiments, the N-terminus end of the antibody or antigen binding fragment thereof is exposed on the exterior surface of the lipid bilayer. In some embodiments, the N-terminus end of the antibody or antigen binding fragment thereof binds to a cell surface molecule of a target cell. In some embodiments, the antibody or antigen binding fragment thereof specifically binds to a cell surface molecule present on a target cell. In some embodiments, the cell surface molecule is a protein, glycan, lipid, or low molecular weight molecule. In some embodiments, the cell surface molecule is GPRC5D.

In some embodiments, the cell surface molecule of a target cell is an antigen or portion thereof. In some embodiments, the antibody or antigen binding fragment thereof is an antibody having a single monomeric domain antigen binding/recognition domain that is able to bind selectively to a specific antigen. In some embodiments, the single domain antibody binds an antigen present on a target cell.

Exemplary cells include immune effector cells, peripheral blood mononuclear cells (PBMC) such as lymphocytes (T cells, B cells, natural killer cells) and monocytes, granulocytes (neutrophils, basophils, eosinophils), macrophages, dendritic cells, cytotoxic T lymphocytes, polymorphonuclear cells (also known as PMN, PML, or PMNL), stem cells, embryonic stem cells, neural stem cells, mesenchymal stem cells (MSCs), hematopoietic stem cells (HSCs), human myogenic stem cells, muscle-derived stem cells (MuStem), embryonic stem cells (ES or ESCs), limbal epithelial stem cells, cardio-myogenic stem cells, cardiomyocytes, progenitor cells, allogenic cells, resident cardiac cells, induced pluripotent stem cells (iPS), adipose-derived or phenotypic modified stem or progenitor cells, CD133+ cells, aldehyde dehydrogenase-positive cells (ALDH+), umbilical cord blood (UCB) cells, peripheral blood stem cells (PBSCs), neurons, neural progenitor cells, pancreatic beta cells, glial cells, or hepatocytes.

In some embodiments, the target cell is a cell of a target tissue. In some embodiments, the target tissue is liver, lungs, heart, spleen, pancreas, gastrointestinal tract, kidney, testes, ovaries, brain, reproductive organs, central nervous system, peripheral nervous system, skeletal muscle, endothelium, inner ear, or eye.

In some embodiments, the target cell is a muscle cell (e.g., skeletal muscle cell), kidney cell, liver cell (e.g., hepatocyte), or a cardiac cell (e.g., cardiomyocyte). In some embodiments, the target cell is a cardiac cell, e.g., a cardiomyocyte (e.g., a quiescent cardiomyocyte), a hepatoblast (e.g., a bile duct hepatoblast), an epithelial cell, a T cell (e.g., a naive T cell), a macrophage (e.g., a tumor infiltrating macrophage), or a fibroblast (e.g., a cardiac fibroblast).

In some embodiments, the target cell is a tumor-infiltrating lymphocyte, a T cell, a neoplastic or tumor cell, a virus-infected cell, a stem cell, a central nervous system (CNS) cell, a hematopoietic stem cell (HSC), a liver cell or a fully differentiated cell. In some embodiments, the target cell is a CD3+ T cell, a CD4+ T cell, a CD8+ T cell, a hepatocyte, a hematopoietic stem cell, a CD34+ hematopoietic stem cell, a CD105+ hematopoietic stem cell, a CD 117+ hematopoietic stem cell, a CD105+ endothelial cell, a B cell, a CD20+B cell, a CD19+B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacytoid dendritic cell, a CD11c+ cell, a CD11b+ cell, a splenocyte, a B cell, a hepatocyte, an endothelial cell, or a non-cancerous cell. In some embodiments, the cell surface molecule is any one of CD8.

In some embodiments, the G protein or functionally active variant or biologically active portion thereof is linked directly to the sdAb variable domain (e.g., a VHH) or scFv. In some embodiments, the targeted envelope protein is a fusion protein that has the following structure: (N′-single domain antibody-C′)-(C′-G protein-N′). In some embodiments, the targeted envelope protein is a fusion protein that has the following structure: (N′-scFv-C′)-(C′-G protein-N′).

In some embodiments, the G protein or functionally active variant or biologically active portion thereof is linked indirectly via a linker to the sdAb variable domain or scFv. In some embodiments, the linker is a peptide linker. In some embodiments, the linker is a chemical linker.

In some embodiments, the linker is a peptide linker and the targeted envelope protein is a fusion protein containing the G protein or functionally active variant or biologically active portion thereof linked via a peptide linker to the sdAb variable domain or svFv. In some embodiments, the targeted envelope protein is a fusion protein that has the following structure: (N′-single domain antibody-C′)-Linker-(C′-G protein-N′). In some embodiments, the targeted envelope protein is a fusion protein that has the following structure: (N′-scFv-C′)-Linker-(C′-G protein-N′). In some embodiments, the peptide linker is up to 65 amino acids in length. In some embodiments, the peptide linker comprises from or from about 2 to 65 amino acids, 2 to 60 amino acids, 2 to 56 amino acids, 2 to 52 amino acids, 2 to 48 amino acids, 2 to 44 amino acids, 2 to 40 amino acids, 2 to 36 amino acids, 2 to 32 amino acids, 2 to 28 amino acids, 2 to 24 amino acids, 2 to 20 amino acids, 2 to 18 amino acids, 2 to 14 amino acids, 2 to 12 amino acids, 2 to 10 amino acids, 2 to 8 amino acids, 2 to 6 amino acids, 6 to 65 amino acids, 6 to 60 amino acids, 6 to 56 amino acids, 6 to 52 amino acids, 6 to 48 amino acids, 6 to 44 amino acids, 6 to 40 amino acids, 6 to 36 amino acids, 6 to 32 amino acids, 6 to 28 amino acids, 6 to 24 amino acids, 6 to 20 amino acids, 6 to 18 amino acids, 6 to 14 amino acids, 6 to 12 amino acids, 6 to 10 amino acids, 6 to 8 amino acids, 8 to 65 amino acids, 8 to 60 amino acids, 8 to 56 amino acids, 8 to 52 amino acids, 8 to 48 amino acids, 8 to 44 amino acids, 8 to 40 amino acids, 8 to 36 amino acids, 8 to 32 amino acids, 8 to 28 amino acids, 8 to 24 amino acids, 8 to 20 amino acids, 8 to 18 amino acids, 8 to 14 amino acids, 8 to 12 amino acids, 8 to 10 amino acids, 10 to 65 amino acids, 10 to 60 amino acids, 10 to 56 amino acids, 10 to 52 amino acids, 10 to 48 amino acids, 10 to 44 amino acids, 10 to 40 amino acids, 10 to 36 amino acids, 10 to 32 amino acids, 10 to 28 amino acids, 10 to 24 amino acids, 10 to 20 amino acids, 10 to 18 amino acids, 10 to 14 amino acids, 10 to 12 amino acids, 12 to 65 amino acids, 12 to 60 amino acids, 12 to 56 amino acids, 12 to 52 amino acids, 12 to 48 amino acids, 12 to 44 amino acids, 12 to 40 amino acids, 12 to 36 amino acids, 12 to 32 amino acids, 12 to 28 amino acids, 12 to 24 amino acids, 12 to 20 amino acids, 12 to 18 amino acids, 12 to 14 amino acids, 14 to 65 amino acids, 14 to 60 amino acids, 14 to 56 amino acids, 14 to 52 amino acids, 14 to 48 amino acids, 14 to 44 amino acids, 14 to 40 amino acids, 14 to 36 amino acids, 14 to 32 amino acids, 14 to 28 amino acids, 14 to 24 amino acids, 14 to 20 amino acids, 14 to 18 amino acids, 18 to 65 amino acids, 18 to 60 amino acids, 18 to 56 amino acids, 18 to 52 amino acids, 18 to 48 amino acids, 18 to 44 amino acids, 18 to 40 amino acids, 18 to 36 amino acids, 18 to 32 amino acids, 18 to 28 amino acids, 18 to 24 amino acids, 18 to 20 amino acids, 20 to 65 amino acids, 20 to 60 amino acids, 20 to 56 amino acids, 20 to 52 amino acids, 20 to 48 amino acids, 20 to 44 amino acids, 20 to 40 amino acids, 20 to 36 amino acids, 20 to 32 amino acids, 20 to 28 amino acids, 20 to 26 amino acids, 20 to 24 amino acids, 24 to 65 amino acids, 24 to 60 amino acids, 24 to 56 amino acids, 24 to 52 amino acids, 24 to 48 amino acids, 24 to 44 amino acids, 24 to 40 amino acids, 24 to 36 amino acids, 24 to 32 amino acids, 24 to 30 amino acids, 24 to 28 amino acids, 28 to 65 amino acids, 28 to 60 amino acids, 28 to 56 amino acids, 28 to 52 amino acids, 28 to 48 amino acids, 28 to 44 amino acids, 28 to 40 amino acids, 28 to 36 amino acids, 28 to 34 amino acids, 28 to 32 amino acids, 32 to 65 amino acids, 32 to 60 amino acids, 32 to 56 amino acids, 32 to 52 amino acids, 32 to 48 amino acids, 32 to 44 amino acids, 32 to 40 amino acids, 32 to 38 amino acids, 32 to 36 amino acids, 36 to 65 amino acids, 36 to 60 amino acids, 36 to 56 amino acids, 36 to 52 amino acids, 36 to 48 amino acids, 36 to 44 amino acids, 36 to 40 amino acids, 40 to 65 amino acids, 40 to 60 amino acids, 40 to 56 amino acids, 40 to 52 amino acids, 40 to 48 amino acids, 40 to 44 amino acids, 44 to 65 amino acids, 44 to 60 amino acids, 44 to 56 amino acids, 44 to 52 amino acids, 44 to 48 amino acids, 48 to 65 amino acids, 48 to 60 amino acids, 48 to 56 amino acids, 48 to 52 amino acids, 50 to 65 amino acids, 50 to 60 amino acids, 50 to 56 amino acids, 50 to 52 amino acids, 54 to 65 amino acids, 54 to 60 amino acids, 54 to 56 amino acids, 58 to 65 amino acids, 58 to 60 amino acids, or 60 to 65 amino acids. In some embodiments, the peptide linker is a polypeptide that is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 amino acids in length.

In some embodiments, the linker is a flexible peptide linker. In some such embodiments, the linker is 1-20 amino acids, such as 1-20 amino acids comprising glycine. In some embodiments, the linker is 1-20 amino acids, such as 1-20 amino acids comprising glycine and serine. In some embodiments, the linker is a flexible peptide linker containing amino acids Glycine and Serine, referred to as GS-linkers. In some embodiments, the peptide linker includes the sequences GS, GGS, GGGGS (SEQ ID NO:147), GGGGGS (SEQ ID NO:145) or combinations thereof. In some embodiments, the polypeptide linker has the sequence (GGS)n, wherein n is 1 to 10. In some embodiments, the polypeptide linker has the sequence (GGGGS)n, (SEQ ID NO:146) wherein n is 1 to 10. In some embodiments, the polypeptide linker has the sequence (GGGGGS)n (SEQ ID NO: 137), wherein n is 1 to 6.

Also provided herein are polynucleotides comprising a nucleic acid sequence encoding a targeted envelope protein. In some embodiments, the polynucleotides comprise a nucleic acid sequence encoding a G protein or biologically active portion thereof. In some embodiments, the polynucleotides further comprise a nucleic acid sequence encoding a single domain antibody (sdAb) variable domain or scFv or biologically active portion thereof. The polynucleotides may include a sequence of nucleotides encoding any of the targeted envelope proteins described above. In some embodiments, the polynucleotide is a synthetic nucleic acid. Also provided are expression vectors containing any of the provided polynucleotides.

In some embodiments, expression of natural or synthetic nucleic acids is achieved by operably linking a nucleic acid encoding the gene of interest to a promoter and incorporating the construct into an expression vector. In some embodiments, vectors are suitable for replication and integration in eukaryotes. In some embodiments, cloning vectors contain transcription and translation terminators, initiation sequences, and promoters useful for expression of the desired nucleic acid sequence. In some of any embodiments, a plasmid comprises a promoter suitable for expression in a cell.

In some embodiments, the polynucleotides contain at least one promoter that is operatively linked to control expression of the targeted envelope protein containing the G protein and the single domain antibody (sdAb) variable domain or scFv. For expression of the targeted envelope protein, at least one module in each promoter functions to position the start site for RNA synthesis. The best-known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

In some embodiments, additional promoter elements, e.g., enhancers, regulate the frequency of transcriptional initiation. In some embodiments, additional promoter elements are located in the region 30-110 bp upstream of the start site, although a number of promoters have been shown to contain functional elements downstream of the start site as well. In some embodiments, spacing between promoter elements frequently is flexible, so that promoter function is preserved when elements are inverted or moved relative to one another. In some embodiments, such as with the thymidine kinase (tk) promoter, the spacing between promoter elements is increased to 50 bp apart before activity begins to decline. In some embodiments, depending on the promoter, individual elements can function either cooperatively or independently to activate transcription.

In some embodiments, a promoter is one naturally associated with a gene or polynucleotide sequence, as is obtained by isolating the 5′ non-coding sequences located upstream of the coding segment and/or exon. In some embodiments, such a promoter is referred to as “endogenous.” In some embodiments, an enhancer is one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein.

In some embodiments, a suitable promoter is the immediate early cytomegalovirus (CMV) promoter sequence. In some embodiments, the promoter sequence is a strong constitutive promoter sequence capable of driving high levels of expression of any polynucleotide sequence operatively linked thereto. In some embodiments, a suitable promoter is Elongation Growth Factor-la (EF-l a). In some embodiments, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

In some embodiments, the promoter is an inducible promoter. In some embodiments, the inducible promoter provides a molecular switch capable of turning on expression of the polynucleotide sequence to which it is operatively linked when such expression is desired, or turning off the expression when expression is not desired. In some embodiments, inducible promoters comprise a metallothionine promoter, a glucocorticoid promoter, a progesterone promoter, and a tetracycline promoter.

In some embodiments, exogenously controlled inducible promoters are used to regulate expression of the G protein and single domain antibody (sdAb) variable domain or scFv. For example, radiation-inducible promoters, heat-inducible promoters, and/or drug-inducible promoters are used to selectively drive transgene expression in, for example, targeted regions. In such embodiments, the location, duration, and level of transgene expression are regulated by the administration of the exogenous source of induction.

In some embodiments, expression of the targeted envelope protein containing a G protein and single domain antibody (sdAb) variable domain or scFv is regulated using a drug-inducible promoter. For example, in some embodiments, the promoter, enhancer, or transactivator comprises a Lac operator sequence, a tetracycline operator sequence, a galactose operator sequence, a doxycycline operator sequence, a rapamycin operator sequence, a tamoxifen operator sequence, or a hormone-responsive operator sequence, or an analog thereof. In some instances, the inducible promoter comprises a tetracycline response element (TRE). In some embodiments, the inducible promoter comprises an estrogen response element (ERE), which can activate gene expression in the presence of tamoxifen. In some instances, a drug-inducible element, such as a TRE, is combined with a selected promoter to enhance transcription in the presence of drug, such as doxycycline. In some embodiments, the drug-inducible promoter is a small molecule-inducible promoter.

In some embodiments, any of the provided polynucleotides are modified to remove CpG motifs and/or to optimize codons for translation in a particular species, such as human, canine, feline, equine, ovine, bovine, etc. species. In some embodiments, the polynucleotides are optimized for human codon usage (i.e., human codon-optimized). In some embodiments, the polynucleotides are modified to remove CpG motifs. In other embodiments, the provided polynucleotides are modified to remove CpG motifs and are codon-optimized, such as human codon-optimized. Methods of codon optimization and CpG motif detection and modification are well-known. Typically, polynucleotide optimization enhances transgene expression, increases transgene stability and preserves the amino acid sequence of the encoded polypeptide.

In order to assess the expression of the targeted envelope protein, the expression vector to be introduced into a cell can also contain either a selectable marker gene or a reporter gene or both to facilitate identification and selection of expressing particles, e.g., viral particles. In other embodiments, the selectable marker is carried on a separate piece of DNA and used in a co-transfection procedure. In some embodiments, both selectable markers and reporter genes are flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000, FEBS Lett. 479:79-82). Suitable expression systems are well known and may be prepared using well known techniques or obtained commercially. Internal deletion constructs are generated using unique internal restriction sites or by partial digestion of non-unique restriction sites. Constructs may then be transfected into cells that display high levels of the desired polynucleotide and/or polypeptide expression. In general, the construct with the minimal 5′ flanking region showing the highest level of expression of reporter gene is identified as the promoter. In some embodiments, such promoter regions are linked to a reporter gene and used to evaluate agents for the ability to modulate promoter-driven transcription.

### Delivery of CAR by Targeted Vector

Provided herein are methods of administering a targeted lipid particle (e.g., vector) targeting a cell. Exemplary cells include immune effector cells, peripheral blood mononuclear cells (PBMC) such as lymphocytes (T cells, B cells, natural killer cells) and monocytes, granulocytes (neutrophils, basophils, eosinophils), macrophages, dendritic cells, cytotoxic T lymphocytes, polymorphonuclear cells (also known as PMN, PML, or PMNL), stem cells, embryonic stem cells, neural stem cells, mesenchymal stem cells (MSCs), hematopoietic stem cells (HSCs), human myogenic stem cells, muscle-derived stem cells (MuStem), embryonic stem cells (ES or ESCs), limbal epithelial stem cells, cardio-myogenic stem cells, cardiomyocytes, progenitor cells, allogenic cells, resident cardiac cells, induced pluripotent stem cells (iPS), adipose-derived or phenotypic modified stem or progenitor cells, CD133+ cells, aldehyde dehydrogenase-positive cells (ALDH+), umbilical cord blood (UCB) cells, peripheral blood stem cells (PBSCs), neurons, neural progenitor cells, pancreatic beta cells, glial cells, or hepatocytes. In some embodiments, the target cell is contacted with a targeted lipid particle.

In some embodiments, the cell is an immune cell. In some embodiments, the immune cell is a NK cell, a T cell, a macrophage, or a monocyte. In some embodiments, the immune cell is a T cell. In some embodiments, the T cell is a CD3+ T cell, a CD4+ T cell, a CDS+ T cell, a naive T cell, a regulatory T (Treg) cell, a non-regulatory T cell, a Th1 cell, a Th2 cell, a Th9 cell, a Th17 cell, a T-follicular helper (Tfh) cell, a cytotoxic T lymphocyte (CTL), an effector T (Teff) cell, a central memory T cell, an effector memory T cell, an effector memory T cell expressing CD45RA (TEMRA cell), a tissue-resident memory (Trm) cell, a virtual memory T cell, an innate memory T cell, a memory stem cell (Tse), or a γδ T cell. In some embodiments, the T cell is a cytotoxic T cell, a helper T cell, a memory T cell, a regulatory T cell, or a tumor infiltrating lymphocyte.

In some embodiments, the T cell is a human T cell. In some embodiments, the T cell is an autologous T cell. In other embodiments, the T cell is an allogeneic T cell. In some embodiments, the allogeneic T cell is a primary T cell. In some embodiments, the allogeneic T cell has been differentiated from an embryonic stem cell (ESC) or an induced pluripotent stem cell (iPSC).

In some embodiments, the method of administering a targeted lipid particle (e.g., vector) targeting a T cell comprise contacting a T cell with a targeted lipid particle comprising a targeting antibody or antigen binding fragment thereof and an exogenous agent to a subject as disclosed herein. In some embodiments, the exogenous agent is a polynucleotide encoding a CAR (e.g., CAR transgene). In some embodiments the method comprises a) obtaining whole blood from the subject; b) collecting the fraction of blood containing leukocyte components including T cells; c) contacting the leukocyte components including T cells with a composition comprising the lentiviral vector to create a transfection mixture; and d) reinfusing the contacted leukocyte components including T cells and/or the transfection mixture to the subject, thereby administering the lipid particle and the exogenous agent to the subject. In some embodiments, the T cells (e.g., CD4+ or CD8+ T cells) are not activated during the method. In some embodiments, step (c) of the method is carried out for no more than 24 hours, e.g., no more than 20, 16, 12, 8, 6, 5, 4, 3, 2, or 1 hour.

In some embodiments, the method according to the present disclosure is capable of delivering a targeted lipid particle to an ex vivo system. The method includes the use of a combination of various apheresis machine hardware components, a software control module, and a sensor module to measure citrate or other solute levels in-line to ensure the maximum accuracy and safety of treatment prescriptions, and the use of replacement fluids designed to fully exploit the design of the system according to the present methods. In some embodiments, components described for one system according to the present invention are implemented within other systems according to the present invention as well.

In some embodiments, the method for administration of the targeted lipid particle (e.g., a lentiviral vector) to the subject comprises the use of a blood processing set for obtaining whole blood from the subject, a separation chamber for collecting the fraction of blood containing leukocyte components including T cells, a contacting container for contacting the T cells with the composition comprising the lentiviral vector, and a further fluid circuit for reinfusion of T cells to the patient. In some embodiments, the method further comprises any of i) a washing component for concentrating T cells, and ii) a sensor and/or module for monitoring cell density and/or concentration. In some embodiments, the methods allow processing of blood directly from the patient, transduction with the lentiviral vector, and reinfusion directly to the patient without any steps of selection for T cells. Further, in some embodiments the methods are carried out without cryopreserving or freezing any cells before or between any one or more of the steps, such that there is no step of formulating cells with a cryoprotectant, e.g., DMSO. In some embodiments, the provided methods do not include a lymphodepletion regimen. In some embodiments, the method including steps (a)-(d) are carried out for a time of no more than 24 hours, such as between 2 hours and 12 hours, for example 3 hours to 6 hours.

In some embodiments, the method is performed in-line (or in situ). In some embodiments, the method is performed in a closed fluid circuit, or a functionally closed fluid circuit. In some embodiments, each of steps (a)-(d) are performed in-line in a closed fluid circuit in which all parts of the system are operably connected, such as via at least one tubing line. In some embodiments, the system is sterile. In some embodiments, the closed fluid circuit is sterile.

Also provided herein are systems for administration of a targeted lipid particle (e.g., lentiviral vector) comprising a C targeting antibody and an exogenous agent as herein disclosed to a subject.

In some embodiments, the targeted lipid particles (e.g., targeted viral vectors) provided herein, or pharmaceutical compositions thereof as described herein are administered to a subject, e.g., a mammal, e.g., a human. In such embodiments, the subject is at risk of, has a symptom of, or is diagnosed with or identified as having, a particular disease or condition. In one embodiment, the subject has cancer. In one embodiment, the subject has an infectious disease. In some embodiments, the targeted viral vector contains nucleic acid sequences encoding an exogenous agent for treating the disease or condition in the subject. In some embodiments, the exogenous agent comprises a polynucleotide encoding a CAR. For example, the exogenous agent is a polynucleotide encoding a CAR that targets or is specific for a protein of a neoplastic cells and the targeted lipid particle is administered to a subject for treating a tumor or cancer in the subject. In some examples, the exogenous agent is an inflammatory mediator or immune molecule, such as a cytokine, and targeted lipid particle is administered to a subject for treating any condition in which it is desired to modulate (e.g., increase) the immune response, such as a cancer or infectious disease. In some embodiments, the targeted viral vector is administered in an effective amount or dose to effect treatment of the disease, condition or disorder. Provided herein are uses of any of the provided targeted viral vector in such methods and treatments, and in the preparation of a medicament in order to carry out such therapeutic methods. In some embodiments, the methods are carried out by administering the targeted viral vector or compositions comprising the same, to the subject having, having had, or suspected of having the disease or condition or disorder. In some embodiments, the methods thereby treat the disease or condition or disorder in the subject. Also provided herein are uses of any of the compositions, such as pharmaceutical compositions provided herein, for the treatment of a disease, condition or disorder associated with a particular gene or protein targeted by or provided by the exogenous agent.

In some embodiments, the provided methods or uses involve administration of a pharmaceutical composition comprising oral, inhaled, transdermal or parenteral (including intravenous, intratumoral, intraperitoneal, intramuscular, intracavity, and subcutaneous) administration. In some embodiments, the targeted viral vector is administered alone or formulated as a pharmaceutical composition. In some embodiments, the targeted viral vector or compositions described herein are administered to a subject, e.g., a mammal, e.g., a human. In some of any embodiments, the subject is at risk of, has a symptom of, or is diagnosed with or identified as having, a particular disease or condition (e.g., a disease or condition described herein). In some embodiments, the disease is a disease or disorder. In some embodiments, the disease is a B cell malignancy.

In some embodiments, the targeted lipid particles is administered in the form of a unit-dose composition, such as a unit dose oral, parenteral, transdermal, or inhaled composition. In some embodiments, the compositions are prepared by admixture and are adapted for oral, inhaled, transdermal, or parenteral administration, and as such are in the form of tablets, capsules, oral liquid preparations, powders, granules, lozenges, reconstitutable powders, injectable, and infusable solutions or suspensions, or suppositories or aerosols.

In some embodiments, the regimen of administration may affect what constitutes an effective amount. In some embodiments, the therapeutic formulations are administered to the subject either prior to or after a diagnosis of disease. In some embodiments, several divided dosages, as well as staggered dosages are administered daily or sequentially, or the dose is continuously infused, or is a bolus injection. In some embodiments, the dosages of the therapeutic formulations are proportionally increased or decreased as indicated by the exigencies of the therapeutic or prophylactic situation.

In some embodiments, the administration of the compositions of the present disclosure to a subject, preferably a mammal, more preferably a human, is carried out using known procedures, at dosages and for periods of time effective to prevent or treat disease. In some embodiments, an effective amount of the targeted lipid particle of the disclosure necessary to achieve a therapeutic effect may vary according to factors such as the activity of the particular lipid particle employed; the time of administration; the rate of excretion; the duration of the treatment; other drugs, compounds or materials used in combination with the targeted lipid particle of the disclosure; the state of the disease or disorder, age, sex, weight, condition, general health and prior medical history of the subject being treated, and like factors well-known in the medical arts. In some embodiments, the dosage regimens are adjusted to provide the optimum therapeutic response. In some embodiments, several divided doses are administered daily, or the dose is proportionally reduced as indicated by the exigencies of the therapeutic situation. One of ordinary skill in the art would be able to study the relevant factors and make the determination regarding the effective amount of the therapeutic targeted lipid particle of the disclosure without undue experimentation.

In some embodiments, dosage levels of the targeted lipid particles in the pharmaceutical compositions of this disclosure are varied so as to obtain an amount that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. In some embodiments, the physician or veterinarian could start doses of the targeted lipid particles of the disclosure employed in the pharmaceutical composition at levels lower than that required in order to achieve the desired therapeutic effect and gradually increase the dosage until the desired effect is achieved.

In some embodiments, the term “container” includes any receptacle for holding the pharmaceutical composition. In some embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. It should be understood that the instructions for use of the pharmaceutical composition is contained on the packaging containing the pharmaceutical composition, and as such the instructions form an increased functional relationship to the packaged product. In some embodiments, instructions may contain information pertaining to the pharmaceutical composition's ability to perform its intended function, e.g., treating or preventing a disease in a subject, or delivering an imaging or diagnostic agent to a subject.

In some embodiments, routes of administration of any of the compositions disclosed herein include oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

In some of any embodiments, suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration, and the like.

In some embodiments, the targeted lipid particle composition comprising an exogenous agent or cargo, is used to deliver such exogenous agent or cargo to a cell tissue or subject. In some embodiments, delivery of a cargo by administration of a targeted lipid particle composition described herein may modify cellular protein expression levels. In some embodiments, the administered composition directs upregulation (via expression in the cell, delivery in the cell, or induction within the cell) of one or more cargo (e.g., a polypeptide or mRNA) that provide a functional activity which is substantially absent or reduced in the cell in which the polypeptide is delivered. In some embodiments, the missing functional activity is enzymatic, structural, or regulatory in nature. In some embodiments, the administered composition directs up-regulation of one or more polypeptides that increases (e.g., synergistically) a functional activity which is present but substantially deficient in the cell in which the polypeptide is upregulated. In some of any embodiments, the administered composition directs downregulation of (via expression in the cell, delivery in the cell, or induction within the cell) of one or more cargo (e.g., a polypeptide, siRNA, or miRNA) that repress a functional activity which is present or upregulated in the cell in which the polypeptide, siRNA, or miRNA is delivered. In some embodiments, the upregulated functional activity is enzymatic, structural, or regulatory in nature. In some embodiments, the administered composition directs down-regulation of one or more polypeptides that decreases (e.g., synergistically) a functional activity which is present or upregulated in the cell in which the polypeptide is downregulated. In some embodiments, the administered composition directs upregulation of certain functional activities and downregulation of other functional activities.

In some of any embodiments, the targeted lipid particle composition (e.g., one comprising mitochondria or DNA) mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the targeted viral vector composition comprises an exogenous protein), the effect lasts for less than 1, 2, 3, 4, 5, 6, or 7 days, 2, 3, or 4 weeks, or 1, 2, 3, 6, or 12 months.

In some of any embodiments, the targeted lipid particle composition described herein is delivered ex-vivo to a cell or tissue, e.g., a human cell or tissue. In embodiments, the composition improves function of a cell or tissue ex-vivo, e.g., improves cell viability, respiration, or other function (e.g., another function described herein).

In some embodiments, the composition is delivered to an ex vivo tissue that is in an injured state (e.g., from trauma, disease, hypoxia, ischemia or other damage).

In some embodiments, the composition is delivered to an ex-vivo transplant (e.g., a tissue explant or tissue for transplantation, e.g., a human vein, a musculoskeletal graft such as bone or tendon, cornea, skin, heart valves, nerves; or an isolated or cultured organ, e.g., an organ to be transplanted into a human, e.g., a human heart, liver, lung, kidney, pancreas, intestine, thymus, eye). In some embodiments, the composition is delivered to the tissue or organ before, during and/or after transplantation.

In some embodiments, the composition is delivered, administered, or contacted with a cell, e.g., a cell preparation. In some embodiments, the cell preparation is a cell therapy preparation (a cell preparation intended for administration to a human subject). In embodiments, the cell preparation comprises cells expressing a chimeric antigen receptor (CAR), e.g., expressing a recombinant CAR. The cells expressing the CAR is, e.g., T cells, Natural Killer (NK) cells, cytotoxic T lymphocytes (CTL), regulatory T cells. In embodiments, the cell preparation is a neural stem cell preparation. In embodiments, the cell preparation is a mesenchymal stem cell (MSC) preparation. In embodiments, the cell preparation is a hematopoietic stem cell (HSC) preparation. In embodiments, the cell preparation is an islet cell preparation.

In some embodiments, the viral vector comprising an anti-CD8 or anti-CD4 sdAb or scFv and an exogenous agent described herein is used to deliver a CAR. In some embodiments, the viral vector transduces a cell expressing CD4 or CD8 (e.g., a CD4+ T cell or a CD8+ T cell) and the transduced cell expresses and amplifies the CAR. The resulting CAR T cells then mediate targeted cell killing. Thus, the disclosure includes the use of viral vector comprising an anti-CD8 or anti-CD4 scFv or sdAb fusogen construct to elicit an immune response specific to the antigen binding moiety of the CAR. In some embodiments, the CAR is used to target a GPRC5D tumor antigen as herein disclosed. In some embodiments, the CAR is used to target a GPRC5D tumor antigen and another cell surface molecule selected from CD5, CD19, CD20, CD22, CD23, CD30, CD33, CD38, CD70, CD123, CD138, GPRC5D, LeY, NKG2D, WT1, GD2, HER2, EGFR, EGFRvIII, B7H3, PSMA, PSCA, CAIX, CD171, CEA, CSPG4, EPHA2, FAP, FRa, IL-13Ra, Mesothelin, MUC1, MUC16, ROR1, C-Met, CD133, Ep-CAM, GPC3, HPV16, IL13Ra2, MAGEA3, MAGEA4, MART1, NY-ESO, VEGFR2, α-Folate, CD24, CD44v7/8, EGP-2, EGP-40, erb-B2, erb-B, FBP, Fetal acetylcholine e receptor, G_{D2}, G_{D3}, HMW-MAA, IL-11Ra, KDR, Lewis Y, L1-cell adhesion molecule, MADE-A1, Oncofetal antigen (h5T4), TAG-72, CD19/22, Syndecan 1, or BCMA. In some embodiments, the CAR is engineered to comprise an intracellular signaling domain of the T cell antigen receptor complex zeta chain (e.g., CD3 zeta). In some embodiments, the intracellular domain is selected from a CD137 (4-1BB) signaling domain, a CD28 signaling domain, and a CD3zeta signaling domain.

Methods for introducing a CAR construct or producing a CAR-T cells are well known to those skilled in the art. Detailed descriptions are disclosed herein and are found, for example, in Vormittag et al., Curr Opin Biotechnol, 2018, 53, 162-181; and Eyquem et al., Nature, 2017, 543, 113-117.

### Cells Expressing CAR

In some aspects, the present technology provides cells expressing one or more chimeric antigen receptor (CAR) on the surface of the cell. These cells are referred to as “engineered cells.” In some embodiments, one or more CARs are delivered to a cell as herein disclosed, e.g., through a viral vector, and the cell expresses the CARs on its surface.

In some embodiments, the cell is an immune cell. In some embodiments, the immune cell is a NK cell, a T cell, a Macrophage, or a Monocyte. In some embodiments, the cell is a T cell. In some embodiments, the T cell is The method of claim 59 or 60, wherein the T cell is a CD3+ T cell, a CD4+ T cell, a CDS+ T cell, a naive T cell, a regulatory T (Treg) cell, a non-regulatory T cell, a Th1 cell, a Th2 cell, a Th9 cell, a Th17 cell, a T-follicular helper (Tfh) cell, a cytotoxic T lymphocyte (CTL), an effector T (Teff) cell, a central memory T cell, an effector memory T cell, an effector memory T cell expressing CD45RA (TEMRA cell), a tissue-resident memory (Trm) cell, a virtual memory T cell, an innate memory T cell, a memory stem cell (Tse), or a γδ T cell. In some embodiments, the T cell is a cytotoxic T cell. In some embodiments, the T cell is a helper T cell. In some embodiments, the T cell is a memory T cell. In some embodiments, the T cell is a regulatory T cell. In some embodiments, the T cell is a tumor infiltrating lymphocyte. In some embodiments, the T cell is a human T cell. In some embodiments, the T cell is an autologous T cell. In other embodiments, the T cell is an allogeneic T cell. In some embodiments, the allogeneic T cell is a primary T cell. In some embodiments, the allogeneic T cell has been differentiated from an embryonic stem cell (ESC) or an induced pluripotent stem cell (iPSC).

In some embodiments, two or more cells expressing CARs of the present disclosure are in a composition. In some embodiments, the composition comprises cells expressing the same CAR targeting GPRC5D. In other embodiments, the composition comprises cells expressing bispecific CARs targeting GPRC5D and one of CD5, CD19, CD20, CD22, CD23, CD30, CD33, CD38, CD70, CD123, CD138, GPRC5D, LeY, NKG2D, WT1, GD2, HER2, EGFR, EGFRvIII, B7H3, PSMA, PSCA, CAIX, CD171, CEA, CSPG4, EPHA2, FAP, FRa, IL-13Ra, Mesothelin, MUC1, MUC16, ROR1, C-Met, CD133, Ep-CAM, GPC3, HPV16, IL13Ra2, MAGEA3, MAGEA4, MART1, NY-ESO, VEGFR2, α-Folate, CD24, CD44v7/8, EGP-2, EGP-40, erb-B2, erb-B, FBP, Fetal acetylcholine e receptor, G_{D2}, G_{D3}, HMW-MAA, IL-11Ra, KDR, Lewis Y, L1-cell adhesion molecule, MADE-A1, Oncofetal antigen (h5T4), TAG-72, CD19/22, Syndecan 1, or BCMA. In some embodiments, the composition comprises cells expressing a CAR targeting GPRC5D and cells expressing a bispecific CAR targeting GPRC5D and one of CD5, CD19, CD20, CD22, CD23, CD30, CD33, CD38, CD70, CD123, CD138, GPRC5D, LeY, NKG2D, WT1, GD2, HER2, EGFR, EGFRvIII, B7H3, PSMA, PSCA, CAIX, CD171, CEA, CSPG4, EPHA2, FAP, FRa, IL-13Ra, Mesothelin, MUC1, MUC16, ROR1, C-Met, CD133, Ep-CAM, GPC3, HPV16, IL13Ra2, MAGEA3, MAGEA4, MART1, NY-ESO, VEGFR2, α-Folate, CD24, CD44v7/8, EGP-2, EGP-40, erb-B2, erb-B, FBP, Fetal acetylcholine e receptor, GD2, GD3, HMW-MAA, IL-11Ra, KDR, Lewis Y, L1-cell adhesion molecule, MADE-A1, Oncofetal antigen (h5T4), TAG-72, CD19/22, Syndecan 1, or BCMA. In other embodiments, the cells of the composition express the same CARs, e.g., CARs targeting the same cell surface molecule. In other embodiments, the cells of the composition express different CARs, e.g., CARs targeting different cell surface molecules.

In some embodiments, the cells used in connection with the provided uses, articles of manufacture and compositions include cells employing single-targeting strategies, such as expression of one genetically engineered receptor herein disclosed, e.g., a CAR, on the cell. In some embodiments, the cells used in connection with the provided methods, uses, articles of manufacture and compositions include cells employing multi-targeting strategies, such as expression of two or more genetically engineered receptors herein disclosed, e.g., CARs, on the cell, each recognizing the same of a different antigen and typically each including a different intracellular signaling component. Such multi-targeting strategies are described, for example, in WO 2014055668 (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5(215) (2013) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat).

For example, in some embodiments, the cells include a receptor expressing a first genetically engineered antigen receptor (e.g., CAR) which is capable of inducing an activating or stimulatory signal to the cell, generally upon specific binding to the antigen or cell surface molecule recognized by the first receptor, e.g., the first antigen. In some embodiments, the cell further includes a second genetically engineered antigen receptor (e.g., CAR), e.g., a chimeric costimulatory receptor, which is capable of inducing a costimulatory signal to the immune cell, generally upon specific binding to a second antigen or cell surface molecule recognized by the second receptor. In some embodiments, the first antigen and second antigen are the same. In some embodiments, the first antigen and second antigen are different. In some embodiments, the first antigen is GPRC5D and the second antigen is one of CD5, CD19, CD20, CD22, CD23, CD30, CD33, CD38, CD70, CD123, CD138, GPRC5D, LeY, NKG2D, WT1, GD2, HER2, EGFR, EGFRvIII, B7H3, PSMA, PSCA, CAIX, CD171, CEA, CSPG4, EPHA2, FAP, FRa, IL-13Ra, Mesothelin, MUC1, MUC16, ROR1, C-Met, CD133, Ep-CAM, GPC3, HPV16, IL13Ra2, MAGEA3, MAGEA4, MART1, NY-ESO, VEGFR2, α-Folate, CD24, CD44v7/8, EGP-2, EGP-40, erb-B2, erb-B, FBP, Fetal acetylcholine e receptor, G_{D2}, G_{D3}, HMW-MAA, IL-11Ra, KDR, Lewis Y, L1-cell adhesion molecule, MADE-A1, Oncofetal antigen (h5T4), TAG-72, CD19/22, Syndecan 1, or BCMA.

In some embodiments, the first and/or second genetically engineered antigen receptor (e.g., CAR) is capable of inducing an activating signal to the cell. In some embodiments, the receptor includes an intracellular signaling component containing ITAM or ITAM-like motifs. In some embodiments, the activation induced by the first receptor involves a signal transduction or change in protein expression in the cell resulting in initiation of an immune response, such as ITAM phosphorylation and/or initiation of IT AM-mediated signal transduction cascade, formation of an immunological synapse and/or clustering of molecules near the bound receptor (e.g., CD4 or CD8, etc.), activation of one or more transcription factors, such as NF-KB and/or AP-1, and/or induction of gene expression of factors such as cytokines, proliferation, and/or survival.

In some embodiments, the first and/or second receptor includes intracellular signaling domains or regions of costimulatory receptors such as CD28, CD137 (4-1BB), OX40, and/or ICOS. In some embodiments, the first and second receptor include an intracellular signaling domain of a costimulatory receptor that are different. In one embodiment, the first receptor contains a CD28 costimulatory signaling region and the second receptor contain a 4-IBB co-stimulatory signaling region or vice versa.

In some embodiments, the first and/or second receptor includes both an intracellular signaling domain containing ITAM or ITAM-like motifs and an intracellular signaling domain of a costimulatory receptor.

In some embodiments, the first receptor contains an intracellular signaling domain containing ITAM or IT AM-like motifs and the second receptor contains an intracellular signaling domain of a costimulatory receptor. The costimulatory signal in combination with the activating signal induced in the same cell is one that results in an immune response, such as a robust and sustained immune response, such as increased gene expression, secretion of cytokines and other factors, and T cell mediated effector functions such as cell killing.

In some embodiments, neither ligation of the first receptor alone nor ligation of the second receptor alone induces a robust immune response. In some aspects, if only one receptor is ligated, the cell becomes tolerized or unresponsive to antigen, or inhibited, and/or is not induced to proliferate or secrete factors or carry out effector functions. In some such embodiments, however, when the plurality of receptors are ligated, such as upon encounter of a cell expressing the first and second antigens, a desired response is achieved, such as full immune activation or stimulation, e.g., as indicated by secretion of one or more cytokine, proliferation, persistence, and/or carrying out an immune effector function such as cytotoxic killing of a cell that expresses the first and second antigens.

In some embodiments, the two receptors induce, respectively, an activating and an inhibitory signal to the cell, such that binding by one of the receptors to its antigen activates the cell or induces a response, but binding by the second inhibitory receptor to its antigen induces a signal that suppresses or dampens that response. Examples are combinations of activating CARs and inhibitory CARs or iCARs. Such a strategy is used, for example, in which the activating CAR binds an antigen expressed in a disease or condition but which is also expressed on normal cells, and the inhibitory receptor binds to a separate antigen which is expressed on the normal cells but not cells of the disease or condition.

In some embodiments, the multi-targeting strategy is employed in a case where an antigen associated with a particular disease or condition is expressed on a non-diseased cell and/or is expressed on the engineered cell itself, either transiently (e.g., upon stimulation in association with genetic engineering) or permanently. In such embodiments, by requiring ligation of two separate and individually specific antigen receptors, specificity, selectivity, and/or efficacy is improved.

In some embodiments, the plurality of antigens, e.g., the first and second antigens, are expressed on the cell, tissue, or disease or condition being targeted, such as on the cancer cell. In some aspects, the cell, tissue, disease or condition is multiple myeloma or a multiple myeloma cell. In some embodiments, one or more of the plurality of antigens generally also is expressed on a cell which it is not desired to target with the cell therapy, such as a normal or non-diseased cell or tissue, and/or the engineered cells themselves. In such embodiments, by requiring ligation of multiple receptors to achieve a response of the cell, specificity and/or efficacy is achieved.

### Hypoimmune CAR-T Cell

In some embodiments, the present disclosure is directed to pluripotent stem cells (e.g., pluripotent stem cells and induced pluripotent stem cells (iPSCs)), differentiated cells derived from such pluripotent stem cells (such as, but not limited to, T cells and NK cells), and primary cells (such as, but not limited to, primary T cells and primary NK cells) that express a CAR. In some embodiments, the pluripotent stem cells, differentiated cells derived therefrom, such as T cells and NK cells, and primary cells such as primary T cells and primary NK cells, are engineered for reduced expression or lack of expression of MHC class I and/or MHC class II human leukocyte antigens, and in some instances, for reduced expression or lack of expression of a T-cell receptor (TCR) complex. In some embodiments, the hypoimmune (HIP) T cells and primary T cells overexpress CD47 and a chimeric antigen receptor (CAR) in addition to reduced expression or lack of expression of MHC class I and/or MHC class II human leukocyte antigens, and have reduced expression or lack expression of a T-cell receptor (TCR) complex. In some embodiments, the CAR comprises an antigen binding domain that binds to GPRC5D. In some embodiments, the CAR comprises an antigen binding domain that specifically binds to GPRC5D and a second antigen binding domain that specifically binds to CD5, CD19, CD20, CD22, CD23, CD30, CD33, CD38, CD70, CD123, CD138, LeY, NKG2D, WT1, GD2, HER2, EGFR, EGFRvIII, B7H3, PSMA, PSCA, CAIX, CD171, CEA, CSPG4, EPHA2, FAP, FRa, IL-13Ra, Mesothelin, MUC1, MUC16, ROR1, C-Met, CD133, Ep-CAM, GPC3, HPV16, IL13Ra2, MAGEA3, MAGEA4, MART1, NY-ESO, VEGFR2, α-Folate, CD24, CD44v7/8, EGP-2, EGP-40, erb-B2, erb-B, FBP, Fetal acetylcholine e receptor, GD2, GD3, HMW-MAA, IL-11Ra, KDR, Lewis Y, L1-cell adhesion molecule, MADE-A1, Oncofetal antigen (h5T4), TAG-72, CD19/22, Syndecan 1, or BCMA. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

In some embodiments, engineered and/or hypoimmune (HIP) T cells and primary T cells overexpress CD47 and one or more chimeric antigen receptor (CAR), and include a genomic modification of the B2M gene. In some embodiments, engineered and/or hypoimmune (HIP) T cells and primary T cells overexpress CD47 and include a genomic modification of the CIITA gene. In some embodiments, engineered and/or hypoimmune (HIP) T cells and primary T cells overexpress CD47 and one or more CAR, and include a genomic modification of the TRAC gene. In some embodiments, engineered and/or hypoimmune (HIP) T cells and primary T cells overexpress CD47 and one or more CAR, and include a genomic modification of the TRB gene. In some embodiments, engineered and/or hypoimmune (HIP) T cells and primary T cells overexpress CD47 and one or more CAR, and include one or more genomic modifications selected from the group consisting of the B2M, CIITA, TRAC, and TRB genes. In some embodiments, engineered and/or hypoimmune (HIP) T cells and primary T cells overexpress CD47 and one or more CAR, and include genomic modifications of the B2M, CIITA, TRAC, and TRB genes. In some embodiments, the cells are B2M^{-/-}, CIITA^{-/-}, TRAC^{-/-}, CD47tg cells that also express CARs. In some embodiments, engineered and/or hypoimmune (HIP) T cells are produced by differentiating induced pluripotent stem cells such as engineered and/or hypoimmunogenic induced pluripotent stem cells. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

In some embodiments, the engineered and/or hypoimmune (HIP) T cells and primary T cells are B2M^{-/-}, CIITA^{-/-}, TRB^{-/-}, CD47tg cells that also express CARs. In some embodiments, the cells are B2M^{-/-}, CIITA^{-/-}, TRAC^{-/-}, TRB^{-/-}, CD47tg cells that also express CARs. In some embodiments, the cells are B2M^{indel/indel}, CIITA^{indel/indel}, TRAC^{indel/indel}, CD47tg cells that also express CARs. In some embodiments, the cells are B2M^{indel/indel}, CIITA^{indel/indel}, TRB^{indel/indel}, CD47tg cells that also express CARs. In some embodiments, the cells are B2M^{indel/indel}, CIITA^{indel/indel}, TRAC^{indel/indel}, TRB^{indel/indel}, CD47tg cells that also express CARs. In some embodiments, the engineered or modified cells described are pluripotent stem cells, induced pluripotent stem cells, NK cells differentiated from such pluripotent stem cells and induced pluripotent stem cells, T cells differentiated from such pluripotent stem cells and induced pluripotent stem cells, or primary T cells. Non-limiting examples of primary T cells include CD3+ T cells, CD4+ T cells, CDS+ T cells, naive T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tern) cells, effector memory T (Tern) cells, effector memory T cells express CD45RA (TEMRA cells), tissue-resident memory (Trm) cells, virtual memory T cells, innate memory T cells, memory stem cell (Tse), γδ T cells, and any other subtype of T cells. In some embodiments, the primary T cells are selected from a group that includes cytotoxic T-cells, helper T-cells, memory T-cells, regulatory T-cells, tumor infiltrating lymphocytes, and combinations thereof. Non-limiting examples of NK cells and primary NK cells include immature NK cells and mature NK cells. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

In some embodiments, the primary T cells are from a pool of primary T cells from one or more donor subjects that are different than the recipient subject (e.g., the patient administered the cells). In some embodiments, the primary T cells are obtained from 1, 2, 3, 4, 5, 6, 7, 8, 9, 10, 20, 50, 100 or more donor subjects and pooled together. In some embodiments, the primary T cells are obtained from 1 or more, 2 or more, 3 or more, 4 or more, 5 or more, 6 or more, 7 or more, 8 or more, 9 or more, 10, or more 20 or more, 50 or more, or 100 or more donor subjects and pooled together. In some embodiments, the primary T cells are harvested from one or a plurality of individuals, and in some instances, the primary T cells or the pool of primary T cells are cultured in vitro. In some embodiments, the primary T cells or the pool of primary T cells are engineered to exogenously express CD47 and cultured in vitro.

In some embodiments, the primary T cells or the pool of primary T cells are engineered to express a chimeric antigen receptor (CAR) as herein disclosed. In some embodiments, the CAR is any known to those skilled in the art.

In some embodiments, the primary T cells or the pool of primary T cells are engineered to exhibit reduced expression of an endogenous T cell receptor compared to unmodified primary T cells. In some embodiments, the primary T cells or the pool of primary T cells are engineered to exhibit reduced expression of CTLA-4, PD-1, or both CTLA-4 and PD-1, as compared to unmodified primary T cells. Methods of genetically modifying a cell including a T cell are described in detail, for example, in WO2020/018620 and WO2016/183041, the disclosures of which are herein incorporated by reference in their entireties, including the tables, appendices, sequence listing and figures.

In some embodiments, the cells derived from primary T cells comprise reduced expression of an endogenous T cell receptor, for example by disruption of an endogenous T cell receptor gene (e.g., T cell receptor alpha constant region (TRAC) or T cell receptor beta constant region (TRB)). In some embodiments, an exogenous nucleic acid encoding a polypeptide as disclosed herein (e.g., a chimeric antigen receptor, CD47, or another tolerogenic factor disclosed herein) is inserted at the disrupted T cell receptor gene. In some embodiments, an exogenous nucleic acid encoding a polypeptide is inserted at a TRAC or a TRB gene locus.

In some embodiments, the cells derived from primary T cells comprise reduced expression of cytotoxic T-lymphocyte-associated protein 4 (CTLA4) and/or programmed cell death (PD1). Methods of reducing or eliminating expression of CTLA4, PD1 and both CTLA4 and PD1 are any recognized by those skilled in the art, such as but not limited to, genetic modification technologies that utilize rare-cutting endonucleases and RNA silencing or RNA interference technologies. Non-limiting examples of a rare-cutting endonuclease include any Cas protein, T ALEN, zinc finger nuclease, meganuclease, and/or homing endonuclease. In some embodiments, an exogenous nucleic acid encoding a polypeptide as disclosed herein (e.g., a chimeric antigen receptor, CD47, or another tolerogenic factor disclosed herein) is inserted at a CTLA4 and/or PD1 gene locus. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell. In some embodiments, the exogenous polynucleotide is inserted into at least one allele of the cell using transfection or transduction, for example, with a vector as disclosed herein. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

In some embodiments, a CD47 transgene is inserted into a pre-selected locus of the cell. In some embodiments, a CD47 transgene is inserted into a random locus of the cell. In some embodiments, a transgene encoding a CAR as disclosed herein is inserted into a pre-selected locus of the cell. In some embodiments, a transgene encoding a CAR is inserted into a random locus of the cell. In some embodiments, a CD47 transgene and a transgene encoding a CAR are inserted into a pre-selected locus of the cell. In some embodiments, a transgene encoding a CAR is inserted into a random or pre-selected locus of the cell, including a safe harbor locus, via viral vector transduction/integration. In some embodiments, a CD47 transgene and a transgene encoding a CAR are inserted into a random or pre-selected locus of the cell, including a safe harbor locus, via viral vector transduction/integration. In some embodiments, the vector is a self-inactivating lentiviral vector pseudotyped with a vesicular stomatitis VSVG envelope. In some embodiments, the transgene encoding a CAR is inserted into at least one allele of the cell using viral transduction. In some embodiments, the exogenous polynucleotide is inserted into at least one allele of the cell using a lentivirus based viral vector. In some embodiments, the random and/or pre-selected locus is a safe harbor or target locus. Non-limiting examples of a safe harbor locus include, but are not limited to, a CCR5 gene locus, a PPP1R12C (also known as AAVS1) gene locus, and a CLYBL gene locus, a Rosa gene locus (e.g., ROSA26 gene locus). Non-limiting examples of a target locus include, but are not limited to, a CXCR4 gene locus, an albumin gene locus, a SHS231 gene locus, an F3 gene locus (also known as CD142), a MICA gene locus, a MICB gene locus, a LRP1 gene locus (also known as a CD91 gene locus), a HMGB1 gene locus, an ABO gene locus, ad RHD gene locus, a FUT1 locus, and a KDM5D gene locus. In some embodiments, the CD47 transgene is inserted in Introns 1 or 2 for PPP1R12C (i.e., AAVS1) or CCR5. In some embodiments, the CD47 transgene is inserted in Exons 1 or 2 or 3 for CCR5. In some embodiments, the CD47 transgene is inserted in intron 2 for CLYBL. In some embodiments, the CD47 transgene is inserted in a 500 bp window in Ch-4:58,976,613 (i.e., SHS231). In some embodiments, the CD47 trans gene is inserted in any suitable region of the aforementioned safe harbor or target loci that allows for expression of the exogenous polynucleotide, including, for example, an intron, an exon or a coding sequence region in a safe harbor or target locus. In some embodiments, the pre-selected locus is selected from the group consisting of the B2M locus, the CIITA locus, the TRAC locus, and the TRB locus. In some embodiments, the preselected locus is the B2Mlocus. In some embodiments, the pre-selected locus is the CIITA locus. In some embodiments, the pre-selected locus is the TRAC locus. In some embodiments, the pre-selected locus is the TRB locus. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

In some embodiments, a CD47 transgene and a transgene encoding a CAR are inserted into the same locus. In some embodiments, a CD47 transgene and a transgene encoding a CAR are inserted into different loci. In many instances, a CD47 transgene is inserted into a safe harbor or target locus. In many instances, a transgene encoding a CAR is inserted into a safe harbor or target locus. In some instances, a CD47 transgene is inserted into a B2M locus. In some instances, a trans gene encoding a CAR is inserted into a B2M locus. In some instances, a CD47 transgene is inserted into a CIITA locus. In some instances, a transgene encoding a CAR is inserted into a CIITA locus. In some instances, a CD47 transgene is inserted into a TRAC locus. In some instances, a transgene encoding a CAR is inserted into a TRAC locus. In many other instances, a CD47 transgene is inserted into a TRB locus. In many other instances, a trans gene encoding a CAR is inserted into a TRB locus. In some embodiments, a CD47 transgene and a transgene encoding a CAR are inserted into a safe harbor or target locus (e.g., a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus.

In some embodiments, a CD47 transgene and a transgene encoding a CAR are inserted into a safe harbor or target locus. In some embodiments, a CD47 transgene and a trans gene encoding a CAR are controlled by a single promoter and are inserted into a safe harbor or target locus. In some embodiments, a CD47 transgene and a transgene encoding a CAR are controlled by their own promoters and are inserted into a safe harbor or target locus. In some embodiments, a CD47 transgene and a transgene encoding a CAR are inserted into a TRAC locus. In some embodiments, a CD47 transgene and a transgene encoding a CAR are controlled by a single promoter and are inserted into a TRAC locus. In some embodiments, a CD4 7 transgene and a trans gene encoding a CAR are controlled by their own promoters and are inserted into a TRAC locus. In some embodiments, a CD47 transgene and a transgene encoding a CAR are inserted into a TRB locus. In some embodiments, a CD47 transgene and a transgene encoding a CAR are controlled by a single promoter and are inserted into a TRB locus. In some embodiments, a CD47 transgene and a transgene encoding a CAR are controlled by their own promoters and are inserted into a TRB locus. In other embodiments, a CD47 transgene and a transgene encoding a CAR are inserted into a B2Mlocus. In other embodiments, a CD47 transgene and a transgene encoding a CAR are controlled by a single promoter and are inserted into a B2M locus. In other embodiments, a CD47 transgene and a transgene encoding a CAR are controlled by their own promoters and are inserted into a B2M locus. In various embodiments, a CD47 transgene and a transgene encoding a CAR are inserted into a CIITA locus. In various embodiments, a CD47 transgene and a transgene encoding a CAR are controlled by a single promoter and are inserted into a CIITA locus. In various embodiments, a CD47 transgene and a transgene encoding a CAR are controlled by their own promoters and are inserted into a CIITA locus. In some instances, the promoter controlling expression of any transgene described is a constitutive promoter. In other instances, the promoter for any transgene described is an inducible promoter. In some embodiments, the promoter is an EF1 a promoter. In some embodiments, the promoter is CAG promoter. In some embodiments, a CD47 transgene and a transgene encoding a CAR are both controlled by a constitutive promoter. In some embodiments, a CD47 transgene and a transgene encoding a CAR are both controlled by an inducible promoter. In some embodiments, a CD47 transgene is controlled by a constitutive promoter and a transgene encoding a CAR is controlled by an inducible promoter. In some embodiments, a CD47 transgene is controlled by an inducible promoter and a transgene encoding a CAR is controlled by a constitutive promoter. In various embodiments, a CD47 transgene is controlled by an EF1α promoter and a transgene encoding a CAR is controlled by an EF1α promoter. In some embodiments, a CD47 transgene is controlled by a CAG promoter and a transgene encoding a CAR is controlled by a CAG promoter. In some embodiments, a CD47 transgene is controlled by a CAG promoter and a transgene encoding a CAR is controlled by an EF1α promoter. In some embodiments, a CD47 transgene is controlled by an EF1α promoter and a transgene encoding a CAR is controlled by a CAG promoter. In some embodiments, expression of both a CD47 transgene and a transgene encoding a CAR is controlled by a single EF1α promoter. In some embodiments, expression of both a CD47 transgene and a transgene encoding a CAR is controlled by a single CAG promoter.

In some embodiments, the present disclosure disclosed herein is directed to pluripotent stem cells, (e.g., pluripotent stem cells and induced pluripotent stem cells (iPSCs)), differentiated cells derived from such pluripotent stem cells (e.g., hypoimmune (HIP) T cells), and primary T cells that overexpress CD47 (such as exogenously express CD47 proteins), have reduced expression or lack expression of MHC class I and/or MHC class II human leukocyte antigens, and have reduced expression or lack expression of a T-cell receptor (TCR) complex. In some embodiments, the hypoimmune (HIP) T cells and primary T cells overexpress CD47 (such as exogenously express CD47 proteins), have reduced expression or lack expression of MHC class I and/or MHC class II human leukocyte antigens, and have reduced expression or lack expression of a T-cell receptor (TCR) complex.

In some embodiments, pluripotent stem cells, (e.g., pluripotent stem cells and induced pluripotent stem cells (iPSCs)), differentiated cells derived from such pluripotent stem cells (e.g., hypoimmune (HIP) T cells), and primary T cells overexpress CD47 and include a genomic modification of the B2M gene. In some embodiments, pluripotent stem cells, differentiated cell derived from such pluripotent stem cells and primary T cells overexpress CD47 and include a genomic modification of the CIITA gene. In some embodiments, pluripotent stem cells, T cells differentiated from such pluripotent stem cells and primary T cells overexpress CD47 and include a genomic modification of the TRAC gene. In some embodiments, pluripotent stem cells, T cells differentiated from such pluripotent stem cells and primary T cells overexpress CD47 and include a genomic modification of the TRB gene. In some embodiments, pluripotent stem cells, T cells differentiated from such pluripotent stem cells and primary T cells overexpress CD47 and include one or more genomic modifications selected from the group consisting of the B2M, CIITA, TRAC and TRB genes. In some embodiments, pluripotent stem cells, T cells differentiated from such pluripotent stem cells and primary T cells overexpress CD47 and include genomic modifications of the B2M, CIITA and TRAC genes. In some embodiments, pluripotent stem cells, T cells differentiated from such pluripotent stem cells and primary T cells overexpress CD47 and include genomic modifications of the B2M, CIITA and TRB genes. In some embodiments, pluripotent stem cells, T cells differentiated from such pluripotent stem cells and primary T cells overexpress CD47 and include genomic modifications of the B2M, CIITA, TRAC and TRB genes. In some embodiments, the pluripotent stem cells, differentiated cell derived from such pluripotent stem cells and primary T cells are B2M^{-/-}, CIITA^{-/-}, TRAC^{-/-}, CD47tg cells. In some embodiments, the cells are B2M^{-/-}, CIITA^{-/-}, TRB^{-/-}, CD47tg cells. In some embodiments, the cells are B2M^{-/-}, CIITA^{-/-}, TRAC^{-/-}, TRB^{-/-}, CD47tg cells. In some embodiments, the cells are B2M^{indel/indel}, CIITA^{indel/indel}, TRAC^{indel/indel} CD47tg cells. In some embodiments, the cells are B2M^{indel/indel}, CIITA^{indel/indel}, TRB^{indel/indel}, CD47tg cells. In some embodiments, the cells are B2M^{indel/indel}, CIITA^{indel/indel}, TRAC^{indel/indel}, TRB^{indel/indel}, CD47tg cells. In some embodiments, the engineered or modified cells described are pluripotent stem cells, T cells differentiated from such pluripotent stem cells or primary T cells. Non-limiting examples of primary T cells include CD3+ T cells, CD4+ T cells, CD8+ T cells, naive T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tcm) cells, effector memory T (Tem) cells, effector memory T cells express CD45RA (TEMRA cells), tissue-resident memory (Trm) cells, virtual memory T cells, innate memory T cells, memory stem cell (Tsc), gd T cells, and any other subtype of T cells. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

In some embodiments, a CD47 transgene is inserted into a pre-selected locus of the cell. In some embodiments, the pre-selected locus is a safe harbor or target locus. Non-limiting examples of a safe harbor or target locus includes a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus. In some embodiments, the pre-selected locus is the TRAC locus. In some embodiments, a CD47 transgene is inserted into a safe harbor or target locus (e.g., a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus. In some embodiments, a CD47 transgene is inserted into the B2M locus. In some embodiments, a CD47 transgene is inserted into the B2M locus. In some embodiments, a CD47 transgene is inserted into the TRAC locus. In some embodiments, a CD47 transgene is inserted into the TRB locus. In some embodiments, the CD47 transgene is inserted into a pre-selected locus of the cell, including a safe harbor locus, via viral vector transduction/integration. In some embodiments, the vector is a self-inactivating lentiviral vector pseudotyped with a vesicular stomatitis VSV-G envelope. In some embodiments, the CD47 transgene is inserted into at least one allele of the cell using viral transduction. In some embodiments, the exogenous polynucleotide is inserted into at least one allele of the cell using a lentivirus based viral vector.

In some instances, expression of a CD47 transgene is controlled by a constitutive promoter. In other instances, expression of a CD47 transgene is controlled by an inducible promoter. In some embodiments, the promoter is an EF1alpha (EF1α) promoter. In some embodiments, the promoter a CAG promoter.

In some embodiments, the present disclosure disclosed herein is directed to pluripotent stem cells, (e.g., pluripotent stem cells and induced pluripotent stem cells (iPSCs)), T cells derived from such pluripotent stem cells (e.g., hypoimmune (HIP) T cells), and primary T cells that have reduced expression or lack expression of MHC class I and/or MHC class II human leukocyte antigens and have reduced expression or lack expression of a T-cell receptor (TCR) complex. In some embodiments, the cells have reduced or lack expression of MHC class I antigens, MHC class II antigens, and TCR complexes.

In some embodiments, pluripotent stem cells (e.g., iPSCs), differentiated cells derived from such (e.g., T cells differentiated from such), and primary T cells include a genomic modification of the B2M gene. In some embodiments, pluripotent stem cells (e.g., iPSCs), differentiated cells derived from such (e.g., T cells differentiated from such), and primary T cells include a genomic modification of the CIITA gene. In some embodiments, pluripotent stem cells (e.g., iPSCs), T cells differentiated from such, and primary T cells include a genomic modification of the TRAC gene. In some embodiments, pluripotent stem cells (e.g., iPSCs), T cells differentiated from such, and primary T cells include a genomic modification of the TRB gene. In some embodiments, pluripotent stem cells (e.g., iPSCs), T cells differentiated from such, and primary T cells include one or more genomic modifications selected from the group consisting of the B2M, CIITA and TRAC genes. In some embodiments, pluripotent stem cells (e.g., iPSCs), T cells differentiated from such, and primary T cells include one or more genomic modifications selected from the group consisting of the B2M, CIITA and TRB genes. In some embodiments, pluripotent stem cells (e.g., iPSCs), T cells differentiated from such, and primary T cells include one or more genomic modifications selected from the group consisting of the B2M, CIITA, TRAC and TRB genes. In some embodiments, the cells including iPSCs, T cells differentiated from such, and primary T cells are B2M^{-/-}, CIITA^{-/-}, TRAC^{-/-} cells. In some embodiments, the cells including iPSCs, T cells differentiated from such, and primary T cells are B2M^{-/-}, CIITA^{-/-}, TRB^{-/-} cells. In some embodiments, the cells including iPSCs, T cells differentiated from such, and primary T cells are B2M^{indel/indel}, CIITA^{indel/indel}, TRAC^{indel/indel} cells. In some embodiments, the cells including iPSCs, T cells differentiated from such, and primary T cells are B2M^{indel/indel}, CIITA^{indel/indel}, TRB^{indel/indel} cells. In some embodiments, the cells including iPSCs, T cells differentiated from such, and primary T cells are B2M^{indel/indel}, CIITA^{indel/indel}, TRAC^{indel/indel}, TRB^{indel/indel} cells. In some embodiments, the modified cells described are pluripotent stem cells, induced pluripotent stem cells, T cells differentiated from such pluripotent stem cells and induced pluripotent stem cells, or primary T cells. Non-limiting examples of primary T cells include CD3+ T cells, CD4+ T cells, CD8+ T cells, naive T cells, regulatory T (Treg) cells, non-regulatory T cells, Th1 cells, Th2 cells, Th9 cells, Th17 cells, T-follicular helper (Tfh) cells, cytotoxic T lymphocytes (CTL), effector T (Teff) cells, central memory T (Tem) cells, effector memory T (Tem) cells, effector memory T cells express CD45RA (TEMRA cells), tissue-resident memory (Trm) cells, virtual memory T cells, innate memory T cells, memory stem cell (Tsc), gd T cells, and any other subtype of T cells. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

Cells of the present disclosure exhibit reduced or lack expression of MHC class I antigens, MHC class II antigens, and/or TCR complexes. In some embodiments, reduction of MHC I and/or MHC II expression is accomplished, for example, by one or more of the following: (1) targeting the polymorphic HLA alleles (HLA-A, HLA-B, HLA-C) and MHC-II genes directly; (2) removal of B2M, which will prevent surface trafficking of all MHC-I molecules; (3) removal of CIITA, which will prevent surface trafficking of all MHC-II molecules; and/or (4) deletion of components of the MHC enhanceosomes, such as LRC5, RFX5, RFXANK, RFXAP, IRF1, NF-Y (including NFY-A, NFY-B, NFY-C), and CIITA that are critical for HLA expression.

In some embodiments, HLA expression is interfered with by targeting individual HLAs (e.g., knocking out, knocking down, or reducing expression of HLA-A, HLA-B, HLA-C, HLA-DP, HLA-DQ, and/or HLA-DR), targeting transcriptional regulators of HLA expression (e.g., knocking out, knocking down, or reducing expression of NLRC5, CIITA, RFX5, RFXAP, RFXANK, NFY-A, NFY-B, NFY-C and/or IRF-1), blocking surface trafficking of MHC class I molecules (e.g., knocking out, knocking down, or reducing expression of B2M and/or TAP1), and/or targeting with HLA-Razor (see, e.g., WO2016183041).

In some embodiments, the cells disclosed herein including, but not limited to, pluripotent stem cells, induced pluripotent stem cells, differentiated cells derived from such stem cells, and primary T cells do not express one or more human leukocyte antigens (e.g., HLA-A, HLA-B, HLA-C, HLA-DP, HLA-DQ, and/or HLA-DR) corresponding to MHC-I and/or MHC-II and are thus characterized as being hypoimmunogenic. For example, in some embodiments, the pluripotent stem cells and induced pluripotent stem cells disclosed have been modified such that the stem cell or a differentiated stem cell prepared therefrom do not express or exhibit reduced expression of one or more of the following MHC-I molecules: HLA-A, HLA-B and HLA-C. In some embodiments, one or more of HLA-A, HLA-B and HLA-C is “knocked-out” of a cell. A cell that has a knocked-out HLA-A gene, HLA-B gene, and/or HLA-C gene may exhibit reduced or eliminated expression of each knocked-out gene.

In some embodiments, guide RNAs, shRNAs, siRNAs, or miRNAs that allow simultaneous deletion of all MHC class I alleles by targeting a conserved region in the HLA genes are identified as HLA Razors. In some embodiments, the gRNAs are part of a CRISPR system. In alternative embodiments, the gRNAs are part of a TALEN system. In some embodiments, an HLA Razor targeting an identified conserved region in HLAs is described in WO2016183041. In some embodiments, multiple HLA Razors targeting identified conserved regions are utilized. It is generally understood that any guide, siRNA, shRNA, or miRNA molecule that targets a conserved region in HLAs can act as an HLA Razor.

Methods provided are useful for inactivation or ablation of MHC class I expression and/or MHC class II expression in cells such as but not limited to pluripotent stem cells, differentiated cells, and primary T cells. In some embodiments, genome editing technologies utilizing rare-cutting endonucleases (e.g., the CRISPR/Cas, TALEN, zinc finger nuclease, meganuclease, and homing endonuclease systems) are also used to reduce or eliminate expression of genes involved in an immune response (e.g., by deleting genomic DNA of genes involved in an immune response or by insertions of genomic DNA into such genes, such that gene expression is impacted) in cells. In some embodiments, genome editing technologies or other gene modulation technologies are used to insert tolerance-inducing factors in human cells, rendering them and the differentiated cells prepared therefrom hypoimmunogenic cells. As such, the hypoimmunogenic cells have reduced or eliminated expression of MHC I and MHC II expression. In some embodiments, the cells are nonimmunogenic (e.g., do not induce an innate and/or an adaptive immune response) in a recipient subject.

In some embodiments, the cell includes a modification to increase expression of CD47 and one or more factors selected from the group consisting of DUX4, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, IL-39, FasL, CCL21, CCL22, Mfge8, CD16, CD52, H2-M3, CD16 Fc receptor, IL15-RF, H2-M3(HLA-G), B2M-HLA-E, A20/TNFAIP3, CR1, HLA-F, MANF, and/or Serpinb9.

In some embodiments, the cell comprises a genomic modification of one or more target polynucleotide sequences that regulate the expression of either MHC class I molecules, MHC class II molecules, or MHC class I and MHC class II molecules. In some embodiments, a genetic editing system is used to modify one or more target polynucleotide sequences. In some embodiments, the targeted polynucleotide sequence is one or more selected from the group including B2M, CIITA, and NLRC5. In some embodiments, the cell comprises a genetic editing modification to the B2M gene. In some embodiments, the cell comprises a genetic editing modification to the CIITA gene. In some embodiments, the cell comprises a genetic editing modification to the NLRC5 gene. In some embodiments, the cell comprises genetic editing modifications to the B2M and CIITA genes. In some embodiments, the cell comprises genetic editing modifications to the B2M and NLRC5 genes. In some embodiments, the cell comprises genetic editing modifications to the CIITA and NLRC5 genes. In numerous embodiments, the cell comprises genetic editing modifications to the B2M, CIITA and NLRC5 genes. In some embodiments, the genome of the cell has been altered to reduce or delete critical components of HLA expression. In some embodiments, the cells are modified or engineered as compared to a wild-type or control cell, including an unaltered or unmodified wild-type cell or control cell. In some embodiments, the wild-type cell or the control cell is a starting material. In some embodiments, the starting material is a primary cell collected from a donor. In some embodiments, the starting material is a primary blood cell collected from a donor, e.g., via a leukopak. In some embodiments, the starting material is otherwise modified or engineered to have altered expression of one or more genes to generate the engineered cell.

In some embodiments, the present disclosure provides a cell (e.g., stem cell, induced pluripotent stem cell, differentiated cell such as a primary NK cell, CAR-NK cell, primary T cell or CAR-T cell) or population thereof comprising a genome in which a gene has been edited to delete a contiguous stretch of genomic DNA, thereby reducing or eliminating surface expression of MHC class I molecules in the cell or population thereof. In some embodiments, the present disclosure provides a cell (e.g., stem cell, induced pluripotent stem cell, differentiated cell such as a primary NK cell, CAR-NK cell, primary T cell or CAR-T cell) or population thereof comprising a genome in which a gene has been edited to delete a contiguous stretch of genomic DNA, thereby reducing or eliminating surface expression of MHC class II molecules in the cell or population thereof. In numerous embodiments, the present disclosure provides a cell (e.g., stem cell, induced pluripotent stem cell, differentiated cell, hematopoietic stem cell, primary T cell or CAR-T cell) or population thereof comprising a genome in which one or more genes has been edited to delete a contiguous stretch of genomic DNA, thereby reducing or eliminating surface expression of MHC class I and II molecules in the cell or population thereof.

In some embodiments, the expression of MHC I molecules and/or MHC II molecules is modulated by targeting and deleting a contiguous stretch of genomic DNA, thereby reducing or eliminating expression of a target gene selected from the group consisting of B2M, CIITA, and NLRC5. In some embodiments, described herein are genetically edited cells (e.g., modified human cells) comprising exogenous CD47 proteins and inactivated or modified CIITA gene sequences, and in some instances, additional gene modifications that inactivate or modify B2M gene sequences. In some embodiments, described herein are genetically edited cells comprising exogenous CD47 proteins and inactivated or modified CIITA gene sequences, and in some instances, additional gene modifications that inactivate or modify NLRC5 gene sequences. In some embodiments, described herein are genetically edited cells comprising exogenous CD47 proteins and inactivated or modified B2M gene sequences, and in some instances, additional gene modifications that inactivate or modify NLRC5 gene sequences. In some embodiments, described herein are genetically edited cells comprising exogenous CD47 proteins and inactivated or modified B2M gene sequences, and in some instances, additional gene modifications that inactivate or modify CIITA gene sequences and NLRC5 gene sequences.

Provided herein are cells exhibiting a modification of one or more targeted polynucleotide sequences that regulates the expression of any one of the following: (a) MHC I antigens, (b) MHC II antigens, (c) TCR complexes, (d) both MHC I and II antigens, and (e) MHC I and II antigens and TCR complexes. In some embodiments, the modification includes increasing expression of CD47. In some embodiments, the cells include an exogenous or recombinant CD47 polypeptide. In some embodiments, the modification includes expression of a chimeric antigen receptor. In some embodiments, the cells comprise an exogenous or recombinant chimeric antigen receptor polypeptide.

In some embodiments, the cell includes a genomic modification of one or more targeted polynucleotide sequences that regulates the expression of MHC I antigens, MHC II antigens and/or TCR complexes. In some embodiments, a genetic editing system is used to modify one or more targeted polynucleotide sequences. In some embodiments, the polynucleotide sequence targets one or more genes selected from the group consisting of B2M, CIITA, TRAC, and TRB. In some embodiments, the genome of a T cell (e.g., a T cell differentiated from hypoimmunogenic iPSCs and a primary T cell) has been altered to reduce or delete critical components of HLA and TCR expression, e.g., HLA-A antigen, HLA-B antigen, HLA-C antigen, HLA-DP antigen, HLA-DQ antigen, HLA-DR antigens, TCR-alpha and TCR-beta.

In some embodiments, the present disclosure provides a cell or population thereof comprising a genome in which a gene has been edited to delete a contiguous stretch of genomic DNA, thereby reducing or eliminating surface expression of MHC class I molecules in the cell or population thereof. In some embodiments, the present disclosure provides a cell or population thereof comprising a genome in which a gene has been edited to delete a contiguous stretch of genomic DNA, thereby reducing or eliminating surface expression of MHC class II molecules in the cell or population thereof. In some embodiments, the present disclosure provides a cell or population thereof comprising a genome in which a gene has been edited to delete a contiguous stretch of genomic DNA, thereby reducing or eliminating surface expression of TCR molecules in the cell or population thereof. In numerous embodiments, the present disclosure provides a cell or population thereof comprising a genome in which one or more genes has been edited to delete a contiguous stretch of genomic DNA, thereby reducing or eliminating surface expression of MHC class I and II molecules and TCR complex molecules in the cell or population thereof.

In some embodiments, the cells and methods described herein include genomically editing human cells to cleave CIITA gene sequences as well as editing the genome of such cells to alter one or more additional target polynucleotide sequences such as, but not limited to, B2M TRAC, and TRB. In some embodiments, the cells and methods described herein include genomically editing human cells to cleave B2M gene sequences as well as editing the genome of such cells to alter one or more additional target polynucleotide sequences such as, but not limited to, CIITA, TRAC, and TRB. In some embodiments, the cells and methods described herein include genomically editing human cells to cleave TRAC gene sequences as well as editing the genome of such cells to alter one or more additional target polynucleotide sequences such as, but not limited to, B2M, CIITA, and TRB. In some embodiments, the cells and methods described herein include genomically editing human cells to cleave TRB gene sequences as well as editing the genome of such cells to alter one or more additional target polynucleotide sequences such as, but not limited to, B2M, CIITA, and TRAC.

Provided herein are hypoimmunogenic stem cells comprising reduced expression of HLA-A, HLA-B, HLA-C, HLA-DP, HLA-DQ, HLA-DR, B2M, CIITA, TCR-alpha, and TCR-beta relative to a wild-type stem cell, the hypoimmunogenic stem cell further comprising a set of exogenous polynucleotides comprising a first exogenous polynucleotide encoding CD47 and a second exogenous polynucleotide encoding a chimeric antigen receptor (CAR) as disclosed herein, wherein the first and/or second exogenous polynucleotides are inserted into a specific locus of at least one allele of the cell. Also provided herein are hypoimmunogenic primary T cells including any subtype of primary T cells comprising reduced expression of HLA-A, HLA-B, HLA-C, HLA-DP, HLA-DQ, HLA-DR, B2M, CIITA, TCR-alpha, and TCR-beta relative to a wild-type primary T cell, the hypoimmunogenic stem cell further comprising a set of exogenous polynucleotides comprising a first exogenous polynucleotide encoding CD47 and a second exogenous polynucleotide encoding a chimeric antigen receptor (CAR) as disclosed herein, wherein the first and/or second exogenous polynucleotides are inserted into a specific locus of at least one allele of the cell. Further provided herein are hypoimmunogenic T cells differentiated from hypoimmunogenic induced pluripotent stem cells comprising reduced expression of HLA-A, HLA-B, HLA-C, HLA-DP, HLA-DQ, HLA-DR, B2M, CIITA, TCR-alpha, and TCR-beta relative to a wild-type primary T cell, the hypoimmunogenic stem cell further comprising a set of exogenous polynucleotides comprising a first exogenous polynucleotide encoding CD47 and a second exogenous polynucleotide encoding a chimeric antigen receptor (CAR) as disclosed herein, wherein the first and/or second exogenous polynucleotides are inserted into a specific locus of at least one allele of the cell.

In some embodiments, the population of engineered cells described evades NK cell mediated cytotoxicity upon administration to a recipient patient. In some embodiments, the population of engineered cells evades NK cell mediated cytotoxicity by one or more subpopulations of NK cells. In some embodiments, the population of engineered is protected from cell lysis by NK cells, including immature and/or mature NK cells upon administration to a recipient patient. In some embodiments, the population of engineered cells evades macrophage engulfment upon administration to a recipient patient. In some embodiments, the population of engineered cells does not induce an innate and/or an adaptive immune response to the cell upon administration to a recipient patient.

In some embodiments, the cells described herein comprise a safety switch. The term “safety switch” used herein refers to a system for controlling the expression of a gene or protein of interest that, when downregulated or upregulated, leads to clearance or death of the cell, e.g., through recognition by the host's immune system. A safety switch is designed to be triggered by an exogenous molecule in case of an adverse clinical event. A safety switch is engineered by regulating the expression on the DNA, RNA and protein levels. A safety switch includes a protein or molecule that allows for the control of cellular activity in response to an adverse event. In one embodiment, the safety switch is a “kill switch” that is expressed in an inactive state and is fatal to a cell expressing the safety switch upon activation of the switch by a selective, externally provided agent. In one embodiment, the safety switch gene is cis-acting in relation to the gene of interest in a construct. Activation of the safety switch causes the cell to kill solely itself or itself and neighboring cells through apoptosis or necrosis. In some embodiments, the cells described herein, e.g., stem cells, induced pluripotent stem cells, hematopoietic stem cells, primary cells, or differentiated cell, including, but not limited to, T cells, CAR-T cells, NK cells, and/or CAR-NK cells, comprise a safety switch.

In some embodiments, the safety switch comprises a therapeutic agent that inhibits or blocks the interaction of CD47 and SIRPα. In some aspects, the CD47-SIRPα blockade agent is an agent that neutralizes, blocks, antagonizes, or interferes with the cell surface expression of CD47, SIRPα, or both. In some embodiments, the CD47-SIRPα blockade agent inhibits or blocks the interaction of CD47, SIRPα or both. In some embodiments, a CD47-SIRPα blockade agent (e.g., a CD47-SIRPα blocking, inhibiting, reducing, antagonizing, neutralizing, or interfering agent) comprises an agent selected from a group that includes an antibody or fragment thereof that binds CD47, a bispecific antibody that binds CD47, an immunocytokine fusion protein that bind CD47, a CD47 containing fusion protein, an antibody or fragment thereof that binds SIRPα, a bispecific antibody that binds SIRPα, an immunocytokine fusion protein that bind SIRPα, an SIRPα containing fusion protein, and a combination thereof.

In some embodiments, the cells described herein comprise a “suicide gene” (or “suicide switch”). The suicide gene can cause the death of the hypoimmunogenic cells should they grow and divide in an undesired manner. The suicide gene ablation approach includes a suicide gene in a gene transfer vector encoding a protein that results in cell killing only when activated by a specific compound. A suicide gene can encode an enzyme that selectively converts a nontoxic compound into highly toxic metabolites. In some embodiments, the cells described herein, e.g., stem cells, induced pluripotent stem cells, hematopoietic stem cells, primary cells, or differentiated cell, including, but not limited to, T cells, CAR-T cells, NK cells, and/or CAR-NK cells, comprise a suicide gene.

In some embodiments, the population of engineered cells described elicits a reduced level of immune activation or no immune activation upon administration to a recipient subject. In some embodiments, the cells elicit a reduced level of systemic TH1 activation or no systemic TH1 activation in a recipient subject. In some embodiments, the cells elicit a reduced level of immune activation of peripheral blood mononuclear cells (PBMCs) or no immune activation of PBMCs in a recipient subject. In some embodiments, the cells elicit a reduced level of donor-specific IgG antibodies or no donor specific IgG antibodies against the cells upon administration to a recipient subject. In some embodiments, the cells elicit a reduced level of IgM and IgG antibody production or no IgM and IgG antibody production against the cells in a recipient subject. In some embodiments, the cells elicit a reduced level of cytotoxic T cell killing of the cells upon administration to a recipient subject.

### A. CIITA

In some embodiments, the technologies disclosed herein modulate (e.g., reduces or eliminates) the expression of MHC II genes by targeting and modulating (e.g., reducing or eliminating) Class II transactivator (CIITA) expression. In some embodiments, the modulation occurs using a CRISPR/Cas system. CIITA is a member of the LR or nucleotide binding domain (NBD) leucine-rich repeat (LRR) family of proteins and regulates the transcription of MHC II by associating with the MHC enhanceosome.

In some embodiments, the target polynucleotide sequence of the present disclosure is a variant of CIITA. In some embodiments, the target polynucleotide sequence is a homolog of CIITA. In some embodiments, the target polynucleotide sequence is an ortholog of CIITA.

In some embodiments, reduced or eliminated expression of CIITA reduces or eliminates expression of one or more of the following MHC class II are HLA-DP, HLA-DM, HLA-DOA, HLA-DOB, HLA-DQ, and HLA-DR.

In some embodiments, the cells described herein comprise gene modifications at the gene locus encoding the CIITA protein. In other words, the cells comprise a genetic modification at the CIITA locus. In some instances, the nucleotide sequence encoding the CIITA protein is set forth in RefSeq. No. NM_000246.4 and NCBI Genbank No. U18259. In some instances, the CIITA gene locus is described in NCBI Gene ID No. 4261. In some embodiments, the amino acid sequence of CIITA is depicted as NCBI GenBank No. AAA88861.1. Additional descriptions of the CIITA protein and gene locus can be found in Uniprot No. P33076, HGNC Ref No. 7067, and OMIM Ref. No. 600005.

In some embodiments, the hypoimmunogenic cells outlined herein comprise a genetic modification targeting the CIITA gene. In some embodiments, the genetic modification targeting the CIITA gene by the rare-cutting endonuclease comprises a Cas protein or a polynucleotide encoding a Cas protein, and at least one guide ribonucleic acid sequence for specifically targeting the CIITA gene. In some embodiments, the at least one guide ribonucleic acid sequence for specifically targeting the CIITA gene is selected from the group consisting of SEQ ID NOs:5184-36352 of Table 12 of WO2016183041, which is herein incorporated by reference. In some embodiments, the cell has a reduced ability to induce an innate and/or an adaptive immune response in a recipient subject. In some embodiments, an exogenous nucleic acid encoding a polypeptide as disclosed herein (e.g., a chimeric antigen receptor, CD47, or another tolerogenic factor disclosed herein) is inserted at the CIITA gene.

Assays to test whether the CIITA gene has been inactivated are known and described herein. In some embodiments, the resulting genetic modification of the CIITA gene by PCR and the reduction of HLA-II expression is assayed by FACS analysis. In some embodiments, CIITA protein expression is detected using a Western blot of cells lysates probed with antibodies to the CIITA protein. In some embodiments, reverse transcriptase polymerase chain reactions (RT-PCR) are used to confirm the presence of the inactivating genetic modification. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

### B. B2M

In some embodiments, the technologies disclosed herein modulate (e.g., reduce or eliminate) the expression of MHC-I genes by targeting and modulating (e.g., reducing or eliminating) expression of the accessory chain B2M. In some embodiments, the modulation occurs using a CRISPR/Cas system. By modulating (e.g., reducing or deleting) expression of B2M, surface trafficking of MHC-I molecules is blocked and the cell rendered hypoimmunogenic. In some embodiments, the cell has a reduced ability to induce an innate and/or an adaptive immune response in a recipient subject.

In some embodiments, the target polynucleotide sequence of the present disclosure is a variant of B2M. In some embodiments, the target polynucleotide sequence is a homolog of B2M. In some embodiments, the target polynucleotide sequence is an ortholog of B2M.

In some embodiments, decreased or eliminated expression of B2M reduces or eliminates expression of one or more of the following MHC I molecules: HLA-A, HLA-B, and HLA-C.

In some embodiments, the cells described herein comprise gene modifications at the gene locus encoding the B2M protein. In other words, the cells comprise a genetic modification at the B2M locus. In some instances, the nucleotide sequence encoding the B2M protein is set forth in RefSeq. No. NM_004048.4 and Genbank No. AB021288.1. In some instances, the B2M gene locus is described in NCBI Gene ID No. 567. In some embodiments, the amino acid sequence of B2M is depicted as NCBI GenBank No. BAA35182.1. Additional descriptions of the B2M protein and gene locus can be found in Uniprot No. P61769, HGNC Ref. No. 914, and OMIM Ref No. 109700.

In some embodiments, the hypoimmunogenic cells outlined herein comprise a genetic modification targeting the B2M gene. In some embodiments, the genetic modification targeting the B2M gene by the rare-cutting endonuclease comprises a Cas protein or a polynucleotide encoding a Cas protein, and at least one guide ribonucleic acid sequence for specifically targeting the B2M gene. In some embodiments, the at least one guide ribonucleic acid sequence for specifically targeting the B2M gene is selected from the group consisting of SEQ ID NOS:81240-85644 of Table 15 of WO2016183041, which is herein incorporated by reference. In some embodiments, an exogenous nucleic acid encoding a polypeptide as disclosed herein (e.g., a chimeric antigen receptor, CD47, or another tolerogenic factor disclosed herein) is inserted at the B2M gene. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

Assays to test whether the B2M gene has been inactivated are known and described herein. In some embodiments, the resulting genetic modification of the B2M gene by PCR and the reduction of HLA-I expression is assayed by FACS analysis. In some embodiments, B2M protein expression is detected using a Western blot of cells lysates probed with antibodies to the B2M protein. In some embodiments, reverse transcriptase polymerase chain reactions (RT-PCR) are used to confirm the presence of the inactivating genetic modification.

### C. NLRC5

In many embodiments, the technologies disclosed herein modulate (e.g., reduce or eliminate) the expression of MHC-I genes by targeting and modulating (e.g., reducing or eliminating) expression of the NLR family, CARD domain containing 5/NOD27/CLR16.1 (NLRC5). In some embodiments, the modulation occurs using a CRISPR/Cas system. NLRC5 is a critical regulator of MHC-I-mediated immune responses and, similar to CIITA, NLRC5 is highly inducible by IFN-7 and can translocate into the nucleus. NLRC5 activates the promoters of MHC-I genes and induces the transcription of MHC-I as well as related genes involved in MHC-I antigen presentation.

In some embodiments, the target polynucleotide sequence is a variant of NLRC5. In some embodiments, the target polynucleotide sequence is a homolog of NLRC5. In some embodiments, the target polynucleotide sequence is an ortholog of NLRC5.

In some embodiments, decreased or eliminated expression of NLRC5 reduces or eliminates expression of one or more of the following MHC I molecules—HLA-A, HLA-B, and HLA-C.

In some embodiments, the cells outlined herein comprise a genetic modification targeting the NLRC5 gene. In some embodiments, the genetic modification targeting the NLRC5 gene by the rare-cutting endonuclease comprises a Cas protein or a polynucleotide encoding a Cas protein, and at least one guide ribonucleic acid sequence for specifically targeting the NLRC5 gene. In some embodiments, the at least one guide ribonucleic acid sequence for specifically targeting the NLRC5 gene is selected from the group consisting of SEQ ID NOS:36353-81239 of Appendix 3 or Table 14 of WO2016183041, the disclosure is incorporated by reference in its entirety.

Assays to test whether the NLRC5 gene has been inactivated are known and described herein. In some embodiments, the resulting genetic modification of the NLRC5 gene by PCR and the reduction of HLA-I expression is assayed by FACS analysis. In some embodiments, NLRC5 protein expression is detected using a Western blot of cells lysates probed with antibodies to the NLRC5 protein. In some embodiments, reverse transcriptase polymerase chain reactions (RT-PCR) are used to confirm the presence of the inactivating genetic modification.

### D. TRAC

In many embodiments, the technologies disclosed herein modulate (e.g., reduce or eliminate) the expression of TCR genes including the TRAC gene by targeting and modulating (e.g., reducing or eliminating) expression of the constant region of the T cell receptor alpha chain. In some embodiments, the modulation occurs using a CRISPR/Cas system. By modulating (e.g., reducing or deleting) expression of TRAC, surface trafficking of TCR molecules is blocked. In some embodiments, the cell also has a reduced ability to induce an innate and/or an adaptive immune response in a recipient subject.

In some embodiments, the target polynucleotide sequence of the present disclosure is a variant of TRAC. In some embodiments, the target polynucleotide sequence is a homolog of TRAC. In some embodiments, the target polynucleotide sequence is an ortholog of TRAC.

In some embodiments, decreased or eliminated expression of TRAC reduces or eliminates TCR surface expression.

In some embodiments, the cells, such as, but not limited to, pluripotent stem cells, induced pluripotent stem cells, T cells differentiated from induced pluripotent stem cells, primary T cells, and cells derived from primary T cells comprise gene modifications at the gene locus encoding the TRAC protein. In other words, the cells comprise a genetic modification at the TRAC locus. In some instances, the nucleotide sequence encoding the TRAC protein is set forth in Genbank No. X02592.1. In some instances, the TRAC gene locus is described in RefSeq. No. NG_001332.3 and NCBI Gene ID No. 28755. In some embodiments, the amino acid sequence of TRAC is depicted as Uniprot No. P01848. Additional descriptions of the TRAC protein and gene locus can be found in Uniprot No. P01848, HGNC Ref. No. 12029, and OMIM Ref. No. 186880.

In some embodiments, the hypoimmunogenic cells outlined herein comprise a genetic modification targeting the TRAC gene. In some embodiments, the genetic modification targeting the TRAC gene by the rare-cutting endonuclease comprises a Cas protein or a polynucleotide encoding a Cas protein, and at least one guide ribonucleic acid sequence for specifically targeting the TRAC gene. In some embodiments, the at least one guide ribonucleic acid sequence for specifically targeting the TRAC gene is selected from the group consisting of SEQ ID NOS:532-609 and 9102-9797 of US20160348073, which is herein incorporated by reference.

Assays to test whether the TRAC gene has been inactivated are known and described herein. In some embodiments, the resulting genetic modification of the TRAC gene by PCR and the reduction of TCR expression is assayed by FACS analysis. In some embodiments, TRAC protein expression is detected using a Western blot of cells lysates probed with antibodies to the TRAC protein. In some embodiments, reverse transcriptase polymerase chain reactions (RT-PCR) are used to confirm the presence of the inactivating genetic modification.

### E. TRB

In many embodiments, the technologies disclosed herein modulate (e.g., reduce or eliminate) the expression of TCR genes including the gene encoding T cell antigen receptor, beta chain (e.g., the TRB, TRBC, or TCRB gene) by targeting and modulating (e.g., reducing or eliminating) expression of the constant region of the T cell receptor beta chain. In some embodiments, the modulation occurs using a CRISPR/Cas system. By modulating (e.g., reducing or deleting) expression of TRB, surface trafficking of TCR molecules is blocked. In some embodiments, the cell also has a reduced ability to induce an innate and/or an adaptive immune response in a recipient subject.

In some embodiments, the target polynucleotide sequence of the present disclosure is a variant of TRB. In some embodiments, the target polynucleotide sequence is a homolog of TRB. In some embodiments, the target polynucleotide sequence is an ortholog of TRB.

In some embodiments, decreased or eliminated expression of TRB reduces or eliminates TCR surface expression.

In some embodiments, the cells, such as, but not limited to, pluripotent stem cells, induced pluripotent stem cells, T cells differentiated from induced pluripotent stem cells, primary T cells, and cells derived from primary T cells comprise gene modifications at the gene locus encoding the TRB protein. In other words, the cells comprise a genetic modification at the TRB gene locus. In some instances, the nucleotide sequence encoding the TRB protein is set forth in UniProt No. P0DSE2. In some instances, the TRB gene locus is described in RefSeq. No. NG_001333.2 and NCBI Gene ID No. 6957. In some embodiments, the amino acid sequence of TRB is depicted as Uniprot No. P01848. Additional descriptions of the TRB protein and gene locus can be found in GenBank No. L36092.2, Uniprot No. PODSE2, and HGNC Ref. No. 12155.

In some embodiments, the hypoimmunogenic cells outlined herein comprise a genetic modification targeting the TRB gene. In some embodiments, the genetic modification targeting the TRB gene by the rare-cutting endonuclease comprises a Cas protein or a polynucleotide encoding a Cas protein, and at least one guide ribonucleic acid sequence for specifically targeting the TRB gene. In some embodiments, the at least one guide ribonucleic acid sequence for specifically targeting the TRB gene is selected from the group consisting of SEQ ID NOS:610-765 and 9798-10532 of US20160348073, which is herein incorporated by reference.

Assays to test whether the TRB gene has been inactivated are known and described herein. In some embodiments, the resulting genetic modification of the TRB gene by PCR and the reduction of TCR expression is assayed by FACS analysis. In some embodiments, TRB protein expression is detected using a Western blot of cells lysates probed with antibodies to the TRB protein. In some embodiments, reverse transcriptase polymerase chain reactions (RT-PCR) are used to confirm the presence of the inactivating genetic modification.

### F. CD142

In many embodiments, the technologies disclosed herein modulate (e.g., reduce or eliminate) the expression of CD142, which is also known as tissue factor, factor III, and F3. In some embodiments, the modulation occurs using a gene editing system (e.g., CRISPR/Cas).

In some embodiments, the target polynucleotide sequence is CD142 or a variant of CD142. In some embodiments, the target polynucleotide sequence is a homolog of CD142. In some embodiments, the target polynucleotide sequence is an ortholog of CD142.

In some embodiments, the cells outlined herein comprise a genetic modification targeting the CD142 gene. In some embodiments, the genetic modification targeting the CD142 gene by the rare-cutting endonuclease comprises a Cas protein or a polynucleotide encoding a Cas protein, and at least one guide ribonucleic acid (gRNA) sequence for specifically targeting the CD142 gene. Useful methods for identifying gRNA sequences to target CD142 are described below.

Assays to test whether the CD142 gene has been inactivated are known and described herein. In some embodiments, the resulting genetic modification of the CD142 gene by PCR and the reduction of CD142 expression is assayed by FACS analysis. In some embodiments, CD142 protein expression is detected using a Western blot of cells lysates probed with antibodies to the CD142 protein. In some embodiments, reverse transcriptase polymerase chain reactions (RT-PCR) are used to confirm the presence of the inactivating genetic modification.

Useful genomic, polynucleotide and polypeptide information about the human CD142 are provided in, for example, the GeneCard Identifier GC01M094530, HGNC No. 3541, NCBI Gene ID 2152, NCBI RefSeq Nos. NM_001178096.1, NM_001993.4, NP_001171567.1, and NP_001984.1, UniProt No. P13726, and the like.

### G. CTLA-4

In some embodiments, the target polynucleotide sequence is CTLA-4 or a variant of CTLA-4. In some embodiments, the target polynucleotide sequence is a homolog of CTLA-4. In some embodiments, the target polynucleotide sequence is an ortholog of CTLA-4.

In some embodiments, the cells outlined herein comprise a genetic modification targeting the CTLA-4 gene. In some embodiments, primary T cells comprise a genetic modification targeting the CTLA-4 gene. The genetic modification can reduce expression of CTLA-4 polynucleotides and CTLA-4 polypeptides in T cells includes primary T cells and CAR-T cells. In some embodiments, the genetic modification targeting the CTLA-4 gene by the rare-cutting endonuclease comprises a Cas protein or a polynucleotide encoding a Cas protein, and at least one guide ribonucleic acid (gRNA) sequence for specifically targeting the CTLA-4 gene. Useful methods for identifying gRNA sequences to target CTLA-4 are described below.

Assays to test whether the CTLA-4 gene has been inactivated are known and described herein. In some embodiments, the resulting genetic modification of the CTLA-4 gene by PCR and the reduction of CTLA-4 expression is assayed by FACS analysis. In some embodiments, CTLA-4 protein expression is detected using a Western blot of cells lysates probed with antibodies to the CTLA-4 protein. In some embodiments, reverse transcriptase polymerase chain reactions (RT-PCR) are used to confirm the presence of the inactivating genetic modification.

Useful genomic, polynucleotide and polypeptide information about the human CTLA-4 are provided in, for example, the GeneCard Identifier GC02P203867, HGNC No. 2505, NCBI Gene ID 1493, NCBI RefSeq Nos. NM_005214.4, NM_001037631.2, NP_001032720.1 and NP_005205.2, UniProt No. P16410, and the like.

### H. PD-1

In some embodiments, the target polynucleotide sequence is PD-1 or a variant of PD-1. In some embodiments, the target polynucleotide sequence is a homolog of PD-1. In some embodiments, the target polynucleotide sequence is an ortholog of PD-1.

In some embodiments, the cells outlined herein comprise a genetic modification targeting the gene encoding the programmed cell death protein 1 (PD-1) protein or the PDCD1 gene. In some embodiments, primary T cells comprise a genetic modification targeting the PDCD1 gene. The genetic modification can reduce expression of PD-1 polynucleotides and PD-1 polypeptides in T cells includes primary T cells and CAR-T cells. In some embodiments, the genetic modification targeting the PDCD1 gene by the rare-cutting endonuclease comprises a Cas protein or a polynucleotide encoding a Cas protein, and at least one guide ribonucleic acid (gRNA) sequence for specifically targeting the PDCD1 gene. Useful methods for identifying gRNA sequences to target PD-1 are described below.

Assays to test whether the PDCD1 gene has been inactivated are known and described herein. In some embodiments, the resulting genetic modification of the PDCD1 gene by PCR and the reduction of PD-1 expression is assayed by FACS analysis. In some embodiments, PD-1 protein expression is detected using a Western blot of cells lysates probed with antibodies to the PD-1 protein. In some embodiments, reverse transcriptase polymerase chain reactions (RT-PCR) are used to confirm the presence of the inactivating genetic modification.

Useful genomic, polynucleotide and polypeptide information about human PD-1 including the PDCD1 gene are provided in, for example, the GeneCard Identifier GC02M241849, HGNC No. 8760, NCBI Gene ID 5133, Uniprot No. Q15116, and NCBI RefSeq Nos. NM_005018.2 and NP_005009.2.

### I. CD47

In some embodiments, the present disclosure provides a cell or population thereof that has been modified to express the tolerogenic factor (e.g., immunomodulatory polypeptide) CD47. In some embodiments, the present disclosure provides a method for altering a cell genome to express CD47. In some embodiments, the stem cell expresses exogenous CD47. In some instances, the cell expresses an expression vector comprising a nucleotide sequence encoding a human CD47 polypeptide. In some embodiments, the cell is genetically modified to comprise an integrated exogenous polynucleotide encoding CD47 using homology-directed repair. In some instances, the cell expresses a nucleotide sequence encoding a human CD47 polypeptide such that the nucleotide sequence is inserted into at least one allele of a safe harbor or target locus. In some instances, the cell expresses a nucleotide sequence encoding a human CD47 polypeptide wherein the nucleotide sequence is inserted into at least one allele of an AAVS1 locus. In some instances, the cell expresses a nucleotide sequence encoding a human CD47 polypeptide wherein the nucleotide sequence is inserted into at least one allele of a CCR5 locus. In some instances, the cell expresses a nucleotide sequence encoding a human CD47 polypeptide wherein the nucleotide sequence is inserted into at least one allele of a safe harbor or target gene locus, such as, but not limited to, a CCR5 gene locus, a CXCR4 gene locus, a PPP1R12C gene locus, an albumin gene locus, a SHS231 gene locus, a CLYBL gene locus, a Rosa gene locus, an F3 (CD142) gene locus, a MICA gene locus, a MICB gene locus, a LRP1 (CD91) gene locus, a HMGB1 gene locus, an ABO gene locus, an RHD gene locus, a FUT1 locus, and a KDM5D gene locus. In some instances, the cell expresses a nucleotide sequence encoding a human CD47 polypeptide wherein the nucleotide sequence is inserted into at least one allele of a TRAC locus.

CD47 is a leukocyte surface antigen and has a role in cell adhesion and modulation of integrins. It is expressed on the surface of a cell and signals to circulating macrophages not to eat the cell.

In some embodiments, the cell outlined herein comprises a nucleotide sequence encoding a CD47 polypeptide has at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises a nucleotide sequence encoding a CD47 polypeptide having an amino acid sequence as set forth in NCBI Ref. Sequence Nos. NP_001768.1 and NP_942088.1. In some embodiments, the cell comprises a nucleotide sequence for CD47 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_001777.3 and NM_198793.2. In some embodiments, the cell comprises a nucleotide sequence for CD47 as set forth in NCBI Ref. Sequence Nos. NM_001777.3 and NM_198793.2. In some embodiments, the nucleotide sequence encoding a CD47 polynucleotide is a codon optimized sequence. In some embodiments, the nucleotide sequence encoding a CD47 polynucleotide is a human codon optimized sequence.

In some embodiments, the cell comprises a CD47 polypeptide having at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises a CD47 polypeptide having an amino acid sequence as set forth in NCBI Ref. Sequence Nos. NP_001768.1 and NP_942088.1.

Exemplary amino acid sequences of human CD47 with a signal sequence and without a signal sequence are provided in Table 23.

In some embodiments, the cell comprises a CD47 polypeptide having at least 95% sequence identity (e.g., 95%, 96%, 97%, 98%, 99%, or more) to the amino acid sequence of SEQ ID NO:167. In some embodiments, the cell comprises a CD47 polypeptide having the amino acid sequence of SEQ ID NO:167. In some embodiments, the cell comprises a CD47 polypeptide having at least 95% sequence identity (e.g., 95%, 96%, 97%, 98%, 99%, or more) to the amino acid sequence of SEQ ID NO:168. In some embodiments, the cell comprises a CD47 polypeptide having the amino acid sequence of SEQ ID NO:168.

In some embodiments, the cell comprises a nucleotide sequence encoding a CD47 polypeptide having at least 95% sequence identity (e.g., 95%, 96%, 97%, 98%, 99%, or more) to the amino acid sequence of SEQ ID NO:167. In some embodiments, the cell comprises a nucleotide sequence encoding a CD47 polypeptide having the amino acid sequence of SEQ ID NO:167. In some embodiments, the cell comprises a nucleotide sequence encoding a CD47 polypeptide having at least 95% sequence identity (e.g., 95%, 96%, 97%, 98%, 99%, or more) to the amino acid sequence of SEQ ID NO: 168. In some embodiments, the cell comprises a nucleotide sequence encoding a CD47 polypeptide having the amino acid sequence of SEQ ID NO:169-171. In some embodiments, the nucleotide sequence is codon optimized for expression in a particular cell.

In some embodiments, a suitable gene editing system (e.g., CRISPR/Cas system or any of the gene editing systems described herein) is used to facilitate the insertion of a polynucleotide encoding CD47, into a genomic locus of the hypoimmunogenic cell. In some embodiments, the polynucleotide encoding CD47 is inserted into a safe harbor or target locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding CD47 is inserted into a B2M gene locus, a CIITA gene locus, a TRAC gene locus, or a TRB gene locus. In some embodiments, the polynucleotide encoding CD47 is operably linked to a promoter.

In some embodiments, the polynucleotide encoding CD47 is inserted into at least one allele of the T cell using viral transduction. In some embodiments, the polynucleotide encoding CD47 is inserted into at least one allele of the T cell using a lentivirus based viral vector. In some embodiments, the lentivirus based viral vector is a pseudotyped, self-inactivating lentiviral vector that carries the polynucleotide encoding CD47. In some embodiments, the lentivirus based viral vector is a self-inactivating lentiviral vector pseudotyped with a vesicular stomatitis VSV-G envelope, and which carries the polynucleotide encoding CD47.

In some embodiments, CD47 protein expression is detected using a Western blot of cell lysates probed with antibodies against the CD47 protein. In some embodiments, reverse transcriptase polymerase chain reactions (RT-PCR) are used to confirm the presence of the exogenous CD47 mRNA.

### J. CD24

In some embodiments, the present disclosure provides a cell or population thereof that has been modified to express the tolerogenic factor (e.g., immunomodulatory polypeptide) CD24. In some embodiments, the present disclosure provides a method for altering a cell genome to express CD24. In some embodiments, the stem cell expresses exogenous CD24. In some instances, the cell expresses an expression vector comprising a nucleotide sequence encoding a human CD24 polypeptide. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

CD24 which is also referred to as a heat stable antigen or small-cell lung cancer cluster 4 antigen is a glycosylated glycosylphosphatidylinositol-anchored surface protein (Pirruccello et al., J Immunol, 1986, 136, 3779-3784; Chen et al., Glycobiology, 2017, 57, 800-806). It binds to Siglec-10 on innate immune cells. Recently it has been shown that CD24 via Siglec-10 acts as an innate immune checkpoint (Barkal et al., Nature, 2019, 572, 392-396).

In some embodiments, the cell outlined herein comprises a nucleotide sequence encoding a CD24 polypeptide has at least 95% sequence identity (e.g., 95%, 96%, 97%, 98%, 99%, or more) to an amino acid sequence set forth in NCBI Ref Nos. NP_001278666.1, NP_001278667.1, NP_001278668.1, and NP_037362.1. In some embodiments, the cell outlined herein comprises a nucleotide sequence encoding a CD24 polypeptide having an amino acid sequence set forth in NCBI Ref Nos. NP_001278666.1, NP_001278667.1, NP_001278668.1, and NP_037362.1.

In some embodiments, the cell comprises a nucleotide sequence having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref Nos. NM_00129737.1, NM_00129738.1, NM_001291739.1, and NM_013230.3. In some embodiments, the cell comprises a nucleotide sequence as set forth in NCBI Ref Nos. NM_00129737.1, NM_00129738.1, NM_001291739.1, and NM_013230.3.

In some embodiments, a suitable gene editing system (e.g., CRISPR/Cas system or any of the gene editing systems described herein) is used to facilitate the insertion of a polynucleotide encoding CD24, into a genomic locus of the hypoimmunogenic cell. In some embodiments, the polynucleotide encoding CD24 is inserted into a safe harbor or target locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding CD24 is inserted into a B2M gene locus, a CIITA gene locus, a TRAC gene locus, or a TRB gene locus. In some embodiments, the polynucleotide encoding CD24 is operably linked to a promoter.

In some embodiments, CD24 protein expression is detected using a Western blot of cells lysates probed with antibodies against the CD24 protein. In some embodiments, reverse transcriptase polymerase chain reactions (RT-PCR) are used to confirm the presence of the exogenous CD24 mRNA.

In some embodiments, a suitable gene editing system (e.g., CRISPR/Cas system or any of the gene editing systems described herein) is used to facilitate the insertion of a polynucleotide encoding CD24, into a genomic locus of the hypoimmunogenic cell. In some embodiments, the polynucleotide encoding CD24 is inserted into a safe harbor or target locus, such as but not limited to, an AAVS1, CCR5, CLYBL, ROSA26, SHS231, F3 (also known as CD142), MICA, MICB, LRP1 (also known as CD91), HMGB1, ABO, RHD, FUT1, or KDM5D gene locus. In some embodiments, the polynucleotide encoding CD24 is inserted into a B2M gene locus, a CIITA gene locus, a TRAC gene locus, or a TRB gene locus. In some embodiments, the polynucleotide encoding CD24 is operably linked to a promoter.

### K. DUX4

In some embodiments, the present disclosure provides a cell (e.g., stem cell, induced pluripotent stem cell, differentiated cell, hematopoietic stem cell, primary T cell or CAR-T cell) or population thereof comprising a genome modified to increase expression of a tolerogenic or immunosuppressive factor such as DUX4. In some embodiments, the present disclosure provides a method for altering a cell's genome to provide increased expression of DUX4, including through a exogenous polynucleotide. In some embodiments, the disclosure provides a cell or population thereof comprising exogenously expressed DUX4 proteins. In some embodiments, increased expression of DUX4 suppresses, reduces or eliminates expression of one or more of the following MHC I molecules—HLA-A, HLA-B, and HLA-C. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

DUX4 is a transcription factor that is active in embryonic tissues and induced pluripotent stem cells, and is silent in normal, healthy somatic tissues (Feng et al., 2015, ELife4; De Iaco et al., 2017, Nat Genet, 49, 941-945; Hendrickson et al., 2017, Nat Genet, 49, 925-934; Snider et al., 2010, PLoS Genet, e1001181; Whiddon et al., 2017, Nat Genet). DUX4 expression acts to block IFN-gamma mediated induction of major histocompatibility complex (MHC) class I gene expression (e.g., expression of B2M, HLA-A, HLA-B, and HLA-C). DUX4 expression has been implicated in suppressed antigen presentation by MHC class I (Chew et al., Developmental Cell, 2019, 50, 1-14). DUX4 functions as a transcription factor in the cleavage-stage gene expression (transcriptional) program. Its target genes include, but are not limited to, coding genes, noncoding genes, and repetitive elements.

There are at least two isoforms of DUX4, with the longest isoform comprising the DUX4 C-terminal transcription activation domain. The isoforms are produced by alternative splicing. See, e.g., Geng et al., 2012, Dev Cell, 22, 38-51; Snider et al., 2010, PLoS Genet, e1001181. Active isoforms for DUX4 comprise its N-terminal DNA-binding domains and its C-terminal activation domain. See, e.g., Choi et al., 2016, Nucleic Acid Res, 44, 5161-5173.

It has been shown that reducing the number of CpG motifs of DUX4 decreases silencing of a DUX4 transgene (Jagannathan et al., Human Molecular Genetics, 2016, 25(20):4419-4431). The nucleic acid sequence provided in Jagannathan et al., supra represents a codon altered sequence of DUX4 comprising one or more base substitutions to reduce the total number of CpG sites while preserving the DUX4 protein sequence. The nucleic acid sequence is commercially available from Addgene, Catalog No. 99281.

In many embodiments, at least one or more polynucleotides is utilized to facilitate the exogenous expression of DUX4 by a cell, e.g., a stem cell, induced pluripotent stem cell, differentiated cell, hematopoietic stem cell, primary T cell or CAR-T cell.

In some embodiments, a suitable gene editing system (e.g., CRISPR/Cas system or any of the gene editing systems described herein) is used to facilitate the insertion of a polynucleotide encoding DUX4, into a genomic locus of the hypoimmunogenic cell. In some embodiments, the polynucleotide encoding DUX4 is inserted into a safe harbor or target locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding DUX4 is inserted into a B2M gene locus, a CIITA gene locus, a TRAC gene locus, or a TRB gene locus. In some embodiments, the polynucleotide encoding DUX4 is operably linked to a promoter.

In some embodiments, the polynucleotide encoding DUX4 is inserted into at least one allele of the T cell using viral transduction. In some embodiments, the polynucleotide encoding DUX4 is inserted into at least one allele of the T cell using a lentivirus based viral vector. In some embodiments, the lentivirus based viral vector is a pseudotyped, self-inactivating lentiviral vector that carries the polynucleotide encoding DUX4. In some embodiments, the lentivirus based viral vector is a self-inactivating lentiviral vector pseudotyped with a vesicular stomatitis VSV-G envelope, and which carries the polynucleotide encoding DUX4.

In some embodiments, the polynucleotide sequence encoding DUX4 comprises a polynucleotide sequence comprising a codon altered nucleotide sequence of DUX4 comprising one or more base substitutions to reduce the total number of CpG sites while preserving the DUX4 protein sequence. In some embodiments, the polynucleotide sequence encoding DUX4 comprising one or more base substitutions to reduce the total number of CpG sites has at least 85% (e.g., 85%, 86%, 87%, 88%, 89%, 90%, 91%, 92%, 93%, 94%, 95%, 96%, 97%, 98%, 99% or 100%) sequence identity to SEQ ID NO:1 of PCT/US2020/44635, filed Jul. 31, 2020. In some embodiments, the polynucleotide sequence encoding DUX4 is SEQ ID NO:1 of PCT/US2020/44635.

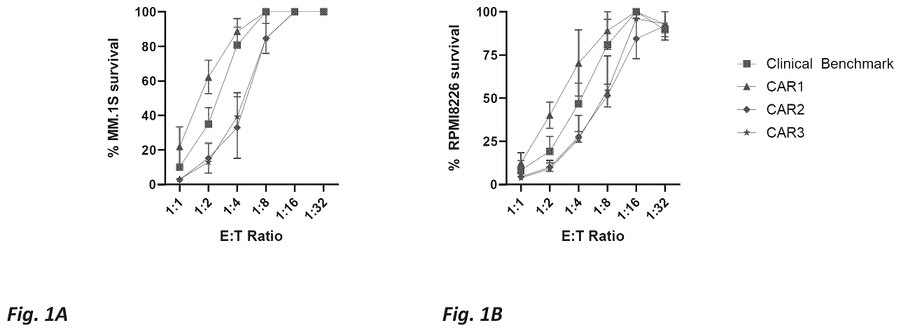

In some embodiments, the polynucleotide sequence encoding DUX4 is a nucleotide sequence encoding a polypeptide sequence having at least 95% (e.g., 95%, 96%, 97%, 98%, 99% or 100%) sequence identity to a sequence selected from a group including SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29, as provided in PCT/US2020/44635. In some embodiments, the polynucleotide sequence encoding DUX4 is a nucleotide sequence encoding a polypeptide sequence is selected from a group including SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29. Amino acid sequences set forth as SEQ ID NOS:2-29 are shown in FIG. 1A-1G of PCT/US2020/44635.

In some instances, the DUX4 polypeptide comprises an amino acid sequence having at least 95% sequence identity to the sequence set forth in GenBank Accession No. ACN62209.1 or an amino acid sequence set forth in GenBank Accession No. ACN62209.1. In some instances, the DUX4 polypeptide comprises an amino acid sequence having at least 95% sequence identity to the sequence set forth in NCBI RefSeq No. NP_001280727.1 or an amino acid sequence set forth in NCBI RefSeq No. NP_001280727.1. In some instances, the DUX4 polypeptide comprises an amino acid sequence having at least 95% sequence identity to the sequence set forth in GenBank Accession No. ACP30489.1 or an amino acid sequence set forth in GenBank Accession No. ACP30489.1. In some instances, the DUX4 polypeptide comprises an amino acid sequence having at least 95% sequence identity to the sequence set forth in UniProt No. POCJ85.1 or an amino acid sequence set forth in UniProt No. POCJ85.1. In some instances, the DUX4 polypeptide comprises an amino acid sequence having at least 95% sequence identity to the sequence set forth in GenBank Accession No. AUA60622.1 or an amino acid sequence set forth in GenBank Accession No. AUA60622.1. In some instances, the DUX4 polypeptide comprises an amino acid sequence having at least 95% sequence identity to the sequence set forth in GenBank Accession No. ADK24683.1 or an amino acid sequence set forth in GenBank Accession No. ADK24683.1. In some instances, the DUX4 polypeptide comprises an amino acid sequence having at least 95% sequence identity to the sequence set forth in GenBank Accession No. ACN62210.1 or an amino acid sequence set forth in GenBank Accession No. ACN62210.1. In some instances, the DUX4 polypeptide comprises an amino acid sequence having at least 95% sequence identity to the sequence set forth in GenBank Accession No. ADK24706.1 or an amino acid sequence set forth in GenBank Accession No. ADK24706.1. In some instances, the DUX4 polypeptide comprises an amino acid sequence having at least 95% sequence identity to the sequence set forth in GenBank Accession No. ADK24685.1 or an amino acid sequence set forth in GenBank Accession No. ADK24685.1. In some instances, the DUX4 polypeptide comprises an amino acid sequence having at least 95% sequence identity to the sequence set forth in GenBank Accession No. ACP30488.1 or an amino acid sequence set forth in GenBank Accession No. ACP30488.1. In some instances, the DUX4 polypeptide comprises an amino acid sequence having at least 95% sequence identity to the sequence set forth in GenBank Accession No. ADK24687.1 or an amino acid sequence set forth in GenBank Accession No. ADK24687.1. In some instances, the DUX4 polypeptide comprises an amino acid sequence having at least 95% sequence identity to the sequence set forth in GenBank Accession No. ACP30487.1 or an amino acid sequence set forth in GenBank Accession No. ACP30487.1. In some instances, the DUX4 polypeptide comprises an amino acid sequence having at least 95% sequence identity to the sequence set forth in GenBank Accession No. ADK24717.1 or an amino acid sequence set forth in GenBank Accession No. ADK24717.1. In some instances, the DUX4 polypeptide comprises an amino acid sequence having at least 95% sequence identity to the sequence set forth in GenBank Accession No. ADK24690.1 or an amino acid sequence set forth in GenBank Accession No. ADK24690.1. In some instances, the DUX4 polypeptide comprises an amino acid sequence having at least 95% sequence identity to the sequence set forth in GenBank Accession No. ADK24689.1 or an amino acid sequence set forth in GenBank Accession No. ADK24689.1. In some instances, the DUX4 polypeptide comprises an amino acid sequence having at least 95% sequence identity to the sequence set forth in GenBank Accession No. ADK24692.1 or an amino acid sequence set forth in GenBank Accession No. ADK24692.1. In some instances, the DUX4 polypeptide comprises an amino acid sequence having at least 95% sequence identity to the sequence set forth in GenBank Accession No. ADK24693.1 or an amino acid sequence of set forth in GenBank Accession No. ADK24693.1. In some instances, the DUX4 polypeptide comprises an amino acid sequence having at least 95% sequence identity to the sequence set forth in GenBank Accession No. ADK24712.1 or an amino acid sequence set forth in GenBank Accession No. ADK24712.1. In some instances, the DUX4 polypeptide comprises an amino acid sequence having at least 95% sequence identity to the sequence set forth in GenBank Accession No. ADK24691.1 or an amino acid sequence set forth in GenBank Accession No. ADK24691.1. In some instances, the DUX4 polypeptide comprises an amino acid sequence having at least 95% sequence identity to the sequence set forth in UniProt No. POCJ87.1 or an amino acid sequence of set forth in UniProt No. POCJ87.1. In some instances, the DUX4 polypeptide comprises an amino acid sequence having at least 95% sequence identity to the sequence set forth in GenBank Accession No. ADK24714.1 or an amino acid sequence set forth in GenBank Accession No. ADK24714.1. In some instances, the DUX4 polypeptide comprises an amino acid sequence having at least 95% sequence identity to the sequence set forth in GenBank Accession No. ADK24684.1 or an amino acid sequence of set forth in GenBank Accession No. ADK24684.1. In some instances, the DUX4 polypeptide comprises an amino acid sequence having at least 95% sequence identity to the sequence set forth in GenBank Accession No. ADK24695.1 or an amino acid sequence set forth in GenBank Accession No. ADK24695.1. In some instances, the DUX4 polypeptide comprises an amino acid sequence having at least 95% sequence identity to the sequence set forth in GenBank Accession No. ADK24699.1 or an amino acid sequence set forth in GenBank Accession No. ADK24699.1. In some instances, the DUX4 polypeptide comprises an amino acid sequence having at least 95% sequence identity to the sequence set forth in NCBI RefSeq No. NP_001768.1 or an amino acid sequence set forth in NCBI RefSeq No. NP_001768. In some instances, the DUX4 polypeptide comprises an amino acid sequence having at least 95% sequence identity to the sequence set forth in NCBI RefSeq No. NP_942088.1 or an amino acid sequence set forth in NCBI RefSeq No. NP_942088.1. In some instances, the DUX4 polypeptide comprises an amino acid sequence having at least 95% sequence identity to SEQ ID NO:28 provided in PCT/US2020/44635 or an amino acid sequence of SEQ ID NO:28 provided in PCT/US2020/44635. In some instances, the DUX4 polypeptide comprises an amino acid sequence having at least 95% sequence identity to SEQ ID NO:29 provided in PCT/US2020/44635 or an amino acid sequence of SEQ ID NO:29 provided in PCT/US2020/44635.

In other embodiments, expression of tolerogenic factors is facilitated using an expression vector. In some embodiments, the expression vector comprises a polynucleotide sequence encoding DUX4 is a codon altered sequence comprising one or more base substitutions to reduce the total number of CpG sites while preserving the DUX4 protein sequence. In some embodiments, the codon altered sequence of DUX4 comprises SEQ ID NO:1 of PCT/US2020/44635. In some embodiments, the codon altered sequence of DUX4 is SEQ ID NO:1 of PCT/US2020/44635. In other embodiments, the expression vector comprises a polynucleotide sequence encoding DUX4 comprising SEQ ID NO:1 of PCT/US2020/44635. In some embodiments, the expression vector comprises a polynucleotide sequence encoding a DUX4 polypeptide sequence having at least 95% sequence identity to a sequence selected from a group including SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29 of PCT/US2020/44635. In some embodiments, the expression vector comprises a polynucleotide sequence encoding a DUX4 polypeptide sequence selected from a group including SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, and SEQ ID NO:29 of PCT/US2020/44635.

An increase of DUX4 expression is assayed using known techniques, such as Western blots, ELISA assays, FACS assays, immunoassays, and the like.

### L. Additional Tolerogenic Factors

In some embodiments, one or more tolerogenic factors is inserted or reinserted into genome-edited cells to create immune-privileged universal donor cells, such as universal donor stem cells, universal donor T cells, or universal donor cells. In some embodiments, the hypoimmunogenic cells disclosed herein have been further modified to express one or more tolerogenic factors. Exemplary tolerogenic factors include, without limitation, one or more of CD47, DUX4, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, IL-39 FasL, CCL21, CCL22, Mfge8, CD16, CD52, H2-M3, CD16 Fc receptor, IL15-RF, H2-M3(HLA-G), B2M-HLA-E, A20/TNFAIP3, CR1, HLA-F, and MANF, and Serpinb9. In some embodiments, the tolerogenic factors are selected from the group consisting of CD200, HLA-G, HLA-E, HLA-C, HLA-E heavy chain, PD-L1, IDO1, CTLA4-Ig, IL-10, IL-35, FasL, Serpinb9, CCL21, CCL22, and Mfge8. In some embodiments, the tolerogenic factors are selected from the group consisting of DUX4, HLA-C, HLA-E, HLA-F, HLA-G, PD-L1, CTLA-4-Ig, C1-inhibitor, and IL-35. In some embodiments, the tolerogenic factors are selected from the group consisting of HLA-C, HLA-E, HLA-F, HLA-G, PD-L1, CTLA-4-Ig, C1-inhibitor, and IL-35. In some embodiments, the tolerogenic factors are selected from a group including CD47, DUX4, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, IL-39 FasL, CCL21, CCL22, Mfge8, CD16, CD52, H2-M3, CD16 Fc receptor, IL15-RF, H2-M3(HLA-G), B2M-HLA-E, A20/TNFAIP3, CR1, HLA-F, and MANF, and Serpinb9.

In some embodiments, the polynucleotide encoding the one or more tolerogenic factors is inserted into at least one allele of the T cell using viral transduction. In some embodiments, the polynucleotide encoding the one or more tolerogenic factors is inserted into at least one allele of the T cell using a lentivirus based viral vector. In some embodiments, the lentivirus based viral vector is a pseudotyped, self-inactivating lentiviral vector that carries the polynucleotide encoding the one or more tolerogenic factors. In some embodiments, the lentivirus based viral vector is a self-inactivating lentiviral vector pseudotyped with a vesicular stomatitis VSV-G envelope, and which carries the polynucleotide encoding the one or more tolerogenic factors.

Useful genomic, polynucleotide and polypeptide information about human CD27 (which is also known as CD27L receptor, Tumor Necrosis Factor Receptor Superfamily Member 7, TNFSF7, T Cell Activation Antigen S152, Tp55, and T14) are provided in, for example, the GeneCard Identifier GC12P008144, HGNC No. 11922, NCBI Gene ID 939, Uniprot No. P26842, and NCBI RefSeq Nos. NM_001242.4 and NP_001233.1.

Useful genomic, polynucleotide and polypeptide information about human CD46 are provided in, for example, the GeneCard Identifier GC01P207752, HGNC No. 6953, NCBI Gene ID 4179, Uniprot No. P15529, and NCBI RefSeq Nos. NM_002389.4, NM_153826.3, NM_172350.2, NM_172351.2, NM_172352.2 NP_758860.1, NM 172353.2, NM_172359.2, NM_172361.2, NP 002380.3, NP 722548.1, NP 758860.1, NP 758861.1, NP_758862.1, NP_758863.1, NP_758869.1, and NP_758871.1.

Useful genomic, polynucleotide and polypeptide information about human CD55 (also known as complement decay-accelerating factor) are provided in, for example, the GeneCard Identifier GC01P207321, HGNC No. 2665, NCBI Gene ID 1604, Uniprot No. P08174, and NCBI RefSeq Nos. NM_000574.4, NM_001114752.2, NM_001300903.1, NM_001300904.1, NP_000565.1, NP_001108224.1, NP_001287832.1, and NP_001287833.1.

Useful genomic, polynucleotide and polypeptide information about human CD59 are provided in, for example, the GeneCard Identifier GC11M033704, HGNC No. 1689, NCBI Gene ID 966, Uniprot No. P13987, and NCBI RefSeq Nos. NP_000602.1, NM_000611.5, NP_001120695.1, NM_001127223.1, NP 001120697.1, NM_001127225.1, NP_001120698.1, NM 001127226.1, NP 001120699.1, NM 001127227.1, NP_976074.1, NM_203329.2, NP_976075.1, NM 203330.2, NP 976076.1, and NM_203331.2.

Useful genomic, polynucleotide and polypeptide information about human CD200 are provided in, for example, the GeneCard Identifier GC03P112332, HGNC No. 7203, NCBI Gene ID 4345, Uniprot No. P41217, and NCBI RefSeq Nos. NP_001004196.2, NM_001004196.3, NP 001305757.1, NM 001318828.1, NP_005935.4, NM 005944.6, XP_005247539.1, and XM_005247482.2.

Useful genomic, polynucleotide and polypeptide information about human HLA-C are provided in, for example, the GeneCard Identifier GC06M031272, HGNC No. 4933, NCBI Gene ID 3107, Uniprot No. P10321, and NCBI RefSeq Nos. NP_002108.4 and NM_002117.5.

Useful genomic, polynucleotide and polypeptide information about human HLA-E are provided in, for example, the GeneCard Identifier GC06P047281, HGNC No. 4962, NCBI Gene ID 3133, Uniprot No. P13747, and NCBI RefSeq Nos. NP_005507.3 and NM_005516.5.

Useful genomic, polynucleotide and polypeptide information about human HLA-G are provided in, for example, the GeneCard Identifier GC06P047256, HGNC No. 4964, NCBI Gene ID 3135, Uniprot No. P17693, and NCBI RefSeq Nos. NP_002118.1 and NM_002127.5.

Useful genomic, polynucleotide and polypeptide information about human PD-L1 or CD274 are provided in, for example, the GeneCard Identifier GC09P005450, HGNC No. 17635, NCBI Gene ID 29126, Uniprot No. Q9NZQ7, and NCBI RefSeq Nos. NP_001254635.1, NM_001267706.1, NP_054862.1, and NM_014143.3.

Useful genomic, polynucleotide and polypeptide information about human IDO1 are provided in, for example, the GeneCard Identifier GC08P039891, HGNC No. 6059, NCBI Gene ID 3620, Uniprot No. P14902, and NCBI RefSeq Nos. NP_002155.1 and NM_002164.5.

Useful genomic, polynucleotide and polypeptide information about human IL-10 are provided in, for example, the GeneCard Identifier GC01M206767, HGNC No. 5962, NCBI Gene ID 3586, Uniprot No. P22301, and NCBI RefSeq Nos. NP_000563.1 and NM_000572.2.

Useful genomic, polynucleotide and polypeptide information about human Fas ligand (which is known as FasL, FASLG, CD178, TNFSF6, and the like) are provided in, for example, the GeneCard Identifier GC01P172628, HGNC No. 11936, NCBI Gene ID 356, Uniprot No.

P48023, and NCBI RefSeq Nos. NP_000630.1, NM_000639.2, NP_001289675.1, and NM_001302746.1.

Useful genomic, polynucleotide and polypeptide information about human CCL21 are provided in, for example, the GeneCard Identifier GC09M034709, HGNC No. 10620, NCBI Gene ID 6366, Uniprot No. 000585, and NCBI RefSeq Nos. NP_002980.1 and NM_002989.3.

Useful genomic, polynucleotide and polypeptide information about human CCL22 are provided in, for example, the GeneCard Identifier GC16P057359, HGNC No. 10621, NCBI Gene ID 6367, Uniprot No. 000626, and NCBI RefSeq Nos. NP_002981.2, NM_002990.4, XP_016879020.1, and XM_017023531.1.

Useful genomic, polynucleotide and polypeptide information about human Mfge8 are provided in, for example, the GeneCard Identifier GC15M088898, HGNC No. 7036, NCBI Gene ID 4240, Uniprot No. Q08431, and NCBI RefSeq Nos. NP_001108086.1, NM_001114614.2, NP_001297248.1, NM_001310319.1, NP 001297249.1, NM_001310320.1, NP_001297250.1, NM_001310321.1, NP_005919.2, and NM_005928.3.

Useful genomic, polynucleotide and polypeptide information about human Serpinf19 are provided in, for example, the GeneCard Identifier GC06M002887, HGNC No. 8955, NCBI Gene ID 5272, Uniprot No. P50453, and NCBI RefSeq Nos. NP_004146.1, NM_004155.5, XP_005249241.1, and XM_005249184.4.

Methods for modulating expression of genes and factors (proteins) include genome editing technologies, RNA or protein expression technologies, and the like. For all of these technologies, well known recombinant techniques are used, to generate recombinant nucleic acids as outlined herein.

In some embodiments, the cells (e.g., stem cell, induced pluripotent stem cell, differentiated cell, hematopoietic stem cell, primary T cell or CAR-T cell) possess genetic modifications that inactivate the B2M and CIITA genes and express a plurality of exogenous polypeptides selected from the group including CD47 and DUX4, CD47 and CD24, CD47 and CD27, CD47 and CD35, CD47 and CD46, CD47 and CD55, CD47 and CD59, CD47 and CD200, CD47 and HLA-C, CD47 and HLA-E, CD47 and HLA-E heavy chain, CD47 and HLA-G, CD47 and PD-L1, CD47 and IDO1, CD47 and CTLA4-Ig, CD47 and C1-Inhibitor, CD47 and IL-10, CD47 and IL-35, CD47 and IL-39, CD47 and FasL, CD47 and CCL21, CD47 and CCL22, CD47 and Mfge8, CD47 and CD16, CD47 and CD52, CD47 and CD16 Fc receptor, CD47 and IL15-RF, CD47 and H2-M3((HLA-G), CD47 and B2M-HLA-E, CD47 and A20/TNFAIP3, CD47 and CR1, CD47 and HLA-F, CD47 and MANF, and CD47 and Serpinb9, and any combination thereof. In some instances, such cells also possess a genetic modification that inactivates the CD142 gene.

In some instances, a gene editing system such as the CRISPR/Cas system is used to facilitate the insertion of tolerogenic factors, such as the tolerogenic factors into a safe harbor or target locus, such as the AAVS1 locus, to actively inhibit immune rejection. In some instances, the tolerogenic factors are inserted into a safe harbor or target locus using an expression vector. In some embodiments, the safe harbor or target locus is an AAVS1, CCR5, CLYBL, ROSA26, SHS231, F3 (also known as CD142), MICA, MICB, LRP1 (also known as CD91), HMGB1, ABO, RHD, FUT1, or KDM5D gene locus.

In some embodiments, expression of a target gene (e.g., DUX4, CD47, or another tolerogenic factor gene) is increased by expression of fusion protein or a protein complex containing (1) a site-specific binding domain specific for the endogenous target gene (e.g., DUX4, CD47, or another tolerogenic factor gene) and (2) a transcriptional activator.

In some embodiments, the regulatory factor is comprised of a site specific DNA-binding nucleic acid molecule, such as a guide RNA (gRNA). In some embodiments, the method is achieved by site specific DNA-binding targeted proteins, such as zinc finger proteins (ZFP) or fusion proteins containing ZFP, which are also known as zinc finger nucleases (ZFNs). In some embodiments, the method is achieved by a genome-modifying protein described herein, including for example, a CRISPR-associated transposase, prime editing, or Programmable Addition via Site-specific Targeting Elements (PASTE). In some embodiments, the method is achieved by a genome-modifying protein described herein, including for example, TnpB polypeptides.

In some embodiments, the regulatory factor comprises a site-specific binding domain, such as using a DNA binding protein or DNA-binding nucleic acid, which specifically binds to or hybridizes to the gene at a targeted region. In some embodiments, the provided polynucleotides or polypeptides are coupled to or complexed with a site-specific nuclease, such as a modified nuclease. For example, in some embodiments, the administration is effected using a fusion comprising a DNA-targeting protein of a modified nuclease, such as a meganuclease or an RNA-guided nuclease such as a clustered regularly interspersed short palindromic nucleic acid (CRISPR)-Cas system, such as CRISPR-Cas9 system. In some embodiments, the nuclease is modified to lack nuclease activity. In some embodiments, the modified nuclease is a catalytically dead dCas9.

In some embodiments, the site specific binding domain is derived from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. See also U.S. Pat. Nos. 5,420,032; 6,833,252; Belfort et al., (1997) Nucleic Acids Res. 25:3379-3388; Dujon et al., (1989) Gene 82:115-118; Perler et al, (1994) Nucleic Acids Res. 22, 1125-1127; Jasin (1996) Trends Genet. 12:224-228; Gimble et al., (1996) J. Mol. Biol. 263:163-180; Argast et al, (1998) J. Mol. Biol. 280:345-353 and the New England Biolabs catalogue. In some embodiments, the DNA-binding specificity of homing endonucleases and meganucleases are engineered to bind non-natural target sites. See, for example, Chevalier et al, (2002) Molec. Cell 10:895-905; Epinat et al, (2003) Nucleic Acids Res. 31:2952-2962; Ashworth et al, (2006) Nature 441:656-659; Paques et al, (2007) Current Gene Therapy 7:49-66; U.S. Patent Publication No. 2007/0117128.

In some embodiments, Zinc finger, TALE, and CRISPR system binding domains are “engineered” to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

In some embodiments, the site-specific binding domain comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion.

Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP is altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins (Gaj et al., Trends in Biotechnology, 2013, 31(7), 397-405). In some embodiments, commercially available zinc fingers are used or are custom designed.

In some embodiments, the site-specific binding domain comprises a naturally occurring or engineered (non-naturally occurring) transcription activator-like protein (TAL) DNA binding domain, such as in a transcription activator-like protein effector (TALE) protein, See, e.g., U.S. Patent Publication No. 20110301073, incorporated by reference in its entirety herein.

In some embodiments, the site-specific binding domain is derived from the CRISPR/Cas system. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system, or a “targeting sequence”), and/or other sequences and transcripts from a CRISPR locus.

In general, a guide sequence includes a targeting domain comprising a polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some examples, the targeting domain of the gRNA is complementary, e.g., at least 80, 85, 90, 95, 98 or 99% complementary, e.g., fully complementary, to the target sequence on the target nucleic acid.

In some embodiments, the target site is upstream of a transcription initiation site of the target gene. In some embodiments, the target site is adjacent to a transcription initiation site of the gene. In some embodiments, the target site is adjacent to an RNA polymerase pause site downstream of a transcription initiation site of the gene.

In some embodiments, the targeting domain is configured to target the promoter region of the target gene to promote transcription initiation, binding of one or more transcription enhancers or activators, and/or RNA polymerase. In some embodiments, one or more gRNA are used to target the promoter region of the gene. In some embodiments, one or more regions of the gene are targeted. In some aspects, the target sites are within 600 base pairs on either side of a transcription start site (TSS) of the gene.

It is within the level of a skilled artisan to design or identify a gRNA sequence that is or comprises a sequence targeting a gene, including the exon sequence and sequences of regulatory regions, including promoters and activators. A genome-wide gRNA database for CRISPR genome editing is publicly available, which contains exemplary single guide RNA (sgRNA) target sequences in constitutive exons of genes in the human genome or mouse genome (see e.g., genescript.com/gRNA-database.html; see also, Sanjana et al. (2014) Nat. Methods, 11:783-4; www.e-crisp.org/E-CRISP/; crispr.mit.edu/). In some embodiments, the gRNA sequence is or comprises a sequence with minimal off-target binding to a non-target gene.

In some embodiments, the regulatory factor further comprises a functional domain, e.g., a transcriptional activator.

In some embodiments, the transcriptional activator is or contains one or more regulatory elements, such as one or more transcriptional control elements of a target gene, whereby a site-specific domain as provided above is recognized to drive expression of such gene. In some embodiments, the transcriptional activator drives expression of the target gene. In some embodiments, the transcriptional activator, is or contains all or a portion of an heterologous transactivation domain. For example, in some embodiments, the transcriptional activator is selected from Herpes simplex-derived transactivation domain, Dnmt3a methyltransferase domain, p65, VP16, and VP64.

In some embodiments, the regulatory factor is a zinc finger transcription factor (ZF-TF). In some embodiments, the regulatory factor is VP64-p65-Rta (VPR).

In some embodiments, the regulatory factor further comprises a transcriptional regulatory domain. Common domains include, e.g., transcription factor domains (activators, repressors, co-activators, co-repressors), silencers, oncogenes (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g., kinases, acetylases and deacetylases); and DNA modifying enzymes (e.g., methyltransferases such as members of the DNMT family (e.g., DNMT1, DNMT3A, DNMT3B, DNMT3L, etc., topoisomerases, helicases, ligases, kinases, phosphatases, polymerases, endonucleases) and their associated factors and modifiers. See, e.g., U.S. Publication No. 2013/0253040, incorporated by reference in its entirety herein. Suitable domains for achieving activation include the HSV VP 16 activation domain (see, e.g., Hagmann et al, J. Virol. 71, 5952-5962 (1 97)) nuclear hormone receptors (see, e.g., Torchia et al., Curr. Opin. Cell. Biol. 10:373-383 (1998)); the p65 subunit of nuclear factor kappa B (Bitko & Bank, J. Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Beerli et al., (1998) Proc. Natl. Acad. Sci. USA 95:14623-33), and degron (Molinari et al., (1999) EMBO J. 18, 6439-6447). Additional exemplary activation domains include, Oct 1, Oct-2A, Spl, AP-2, and CTF1 (Seipel etal, EMBOJ. 11, 4961-4968 (1992) as well as p300, CBP, PCAF, SRC1 PvALF, AtHD2A and ERF-2. See, for example, Robyr et al, (2000) Mol. Endocrinol. 14:329-347; Collingwood et al, (1999) J. Mol. Endocrinol 23:255-275; Leo et al, (2000) Gene 245:1-11; Manteuffel-Cymborowska (1999) Acta Biochim. Pol. 46:77-89; McKenna et al, (1999) J. Steroid Biochem. Mol. Biol. 69:3-12; Malik et al, (2000) Trends Biochem. Sci. 25:277-283; and Lemon et al, (1999) Curr. Opin. Genet. Dev. 9:499-504. Additional exemplary activation domains include, but are not limited to, OsGAI, HALF-1, Cl, AP1, ARF-5, -6, -1, and -8, CPRF1, CPRF4, MYC-RP/GP, and TRAB1, See, for example, Ogawa et al, (2000) Gene 245:21-29; Okanami et al, (1996) Genes Cells 1:87-99; Goff et al, (1991) Genes Dev. 5:298-309; Cho et al, (1999) Plant Mol Biol 40:419-429; Ulmason et al, (1999) Proc. Natl. Acad. Sci. USA 96:5844-5849; Sprenger-Haussels et al, (2000) Plant J. 22:1-8; Gong et al, (1999) Plant Mol. Biol. 41:33-44; and Hobo et al., (1999) Proc. Natl. Acad. Sci. USA 96:15,348-15,353.

Exemplary repression domains that are used to make genetic repressors include, but are not limited to, KRAB A/B, KOX, TGF-beta-inducible early gene (TIEG), v-erbA, SID, MBD2, MBD3, members of the DNMT family (e.g., DNMT1, DNMT3A, DNMT3B, DNMT3L, etc.), Rb, and MeCP2. See, for example, Bird et al, (1999) Cell 99:451-454; Tyler et al, (1999) Cell 99:443-446; Knoepfler et al, (1999) Cell 99:447-450; and Robertson et al, (2000) Nature Genet. 25:338-342. Additional exemplary repression domains include, but are not limited to, ROM2 and AtHD2A. See, for example, Chem et al, (1996) Plant Cell 8:305-321; and Wu et al, (2000) Plant J. 22:19-27.

In some instances, the domain is involved in epigenetic regulation of a chromosome. In some embodiments, the domain is a histone acetyltransferase (HAT), e.g., type-A, nuclear localized such as MYST family members MOZ, Ybf2/Sas3, MOF, and Tip60, GNAT family members Gcn5 or pCAF, the p300 family members CBP, p300 or Rttl09 (Bemdsen and Denu (2008) Curr Opin Struct Biol 18(6):682-689). In other instances the domain is a histone deacetylase (HD AC) such as the class I (HDAC-1, 2, 3, and 8), class II (HDAC IIA (HDAC-4, 5, 7 and 9), HD AC IIB (HDAC 6 and 10)), class IV (HDAC-1 1), class III (also known as sirtuins (SIRTs); SIRT1-7) (see Mottamal et al., (2015) Molecules 20(3):3898-3941). Another domain that is used in some embodiments is a histone phosphorylase or kinase, where examples include MSK1, MSK2, ATR, ATM, DNA-PK, Bubl, VprBP, IKK-a, PKCpi, Dik/Zip, JAK2, PKC5, WSTF and CK2. In some embodiments, a methylation domain is used and is chosen from groups such as Ezh2, PRMT1/6, PRMT5/7, PRMT 2/6, CARM1, set7/9, MLL, ALL-1, Suv 39h, G9a, SETDB1, Ezh2, Set2, Dotl, PRMT 1/6, PRMT 5/7, PR-Set7 and Suv4-20h, Domains involved in sumoylation and biotinylation (Lys9, 13, 4, 18 and 12) may also be used in some embodiments (review see Kousarides (2007) Cell 128:693-705).

Fusion molecules are constructed by methods of cloning and biochemical conjugation that are well known to those of skill in the art. Fusion molecules comprise a DNA-binding domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, IL) Catalogue. Methods and compositions for making fusions between a minor groove binder and a polypeptide have been described. Mapp et al, (2000) Proc. Natl. Acad. Sci. USA 97:3930-3935. Likewise, CRISPR/Cas TFs and nucleases comprising a sgRNA nucleic acid component in association with a polypeptide component function domain are also known to those of skill in the art and detailed herein.

In some embodiments, the present disclosure provides a cell (e.g., a primary T cell and a hypoimmunogenic stem cell and derivative thereof) or population thereof comprising a genome in which the cell genome has been modified to express CD47. In some embodiments, the present disclosure provides a method for altering a cell genome to express CD47. In some embodiments, at least one ribonucleic acid or at least one pair of ribonucleic acids is utilized to facilitate the insertion of CD47 into a cell line. In some embodiments, the at least one ribonucleic acid or the at least one pair of ribonucleic acids is selected from the group consisting of SEQ ID NOS:200784-231885 of Table 29 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-C. In some embodiments, the present disclosure provides a method for altering a cell genome to express HLA-C. In some embodiments, at least one ribonucleic acid or at least one pair of ribonucleic acids is utilized to facilitate the insertion of HLA-C into a cell line. In some embodiments, the at least one ribonucleic acid or the at least one pair of ribonucleic acids is selected from the group consisting of SEQ ID NOS:3278-5183 of Table 10 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-E. In some embodiments, the present disclosure provides a method for altering a cell genome to express HLA-E. In some embodiments, at least one ribonucleic acid or at least one pair of ribonucleic acids is utilized to facilitate the insertion of HLA-E into a cell line. In some embodiments, the at least one ribonucleic acid or the at least one pair of ribonucleic acids is selected from the group consisting of SEQ ID NOS:189859-193183 of Table 19 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-F. In some embodiments, the present disclosure provides a method for altering a cell genome to express HLA-F. In some embodiments, at least one ribonucleic acid or at least one pair of ribonucleic acids is utilized to facilitate the insertion of HLA-F into a cell line. In some embodiments, the at least one ribonucleic acid or the at least one pair of ribonucleic acids is selected from the group consisting of SEQ ID NOS: 688808-399754 of Table 45 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express HLA-G. In some embodiments, the present disclosure provides a method for altering a cell genome to express HLA-G. In some embodiments, at least one ribonucleic acid or at least one pair of ribonucleic acids is utilized to facilitate the insertion of HLA-G into a stem cell line. In some embodiments, the at least one ribonucleic acid or the at least one pair of ribonucleic acids is selected from the group consisting of SEQ ID NOS:188372-189858 of Table 18 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express PD-L1. In some embodiments, the present disclosure provides a method for altering a cell genome to express PD-L1. In some embodiments, at least one ribonucleic acid or at least one pair of ribonucleic acids is utilized to facilitate the insertion of PD-L1 into a stem cell line. In some embodiments, the at least one ribonucleic acid or the at least one pair of ribonucleic acids is selected from the group consisting of SEQ ID NOS:193184-200783 of Table 21 of WO2016183041, which is herein incorporated by reference.

In some embodiments, the present disclosure provides a cell (e.g., a primary T cell and a hypoimmunogenic stem cell and derivative thereof) or population thereof comprising a genome in which the cell genome has been modified to express CTLA4-Ig. In some embodiments, the present disclosure provides a method for altering a cell genome to express CTLA4-Ig. In some embodiments, at least one ribonucleic acid or at least one pair of ribonucleic acids is utilized to facilitate the insertion of CTLA4-Ig into a stem cell line. In some embodiments, the at least one ribonucleic acid or the at least one pair of ribonucleic acids is selected from any one disclosed in WO2016183041, including the sequence listing.

In some embodiments, the present disclosure provides a cell (e.g., a primary T cell and a hypoimmunogenic stem cell and derivative thereof) or population thereof comprising a genome in which the cell genome has been modified to express CI-inhibitor. In some embodiments, the present disclosure provides a method for altering a cell genome to express CI-inhibitor. In some embodiments, at least one ribonucleic acid or at least one pair of ribonucleic acids is utilized to facilitate the insertion of CI-inhibitor into a stem cell line. In some embodiments, the at least one ribonucleic acid or the at least one pair of ribonucleic acids is selected from any one disclosed in WO2016183041, including the sequence listing.

In some embodiments, the present disclosure provides a cell (e.g., a primary T cell and a hypoimmunogenic stem cell and derivative thereof) or population thereof comprising a genome in which the cell genome has been modified to express IL-35. In some embodiments, the present disclosure provides a method for altering a cell genome to express IL-35. In some embodiments, at least one ribonucleic acid or at least one pair of ribonucleic acids is utilized to facilitate the insertion of IL-35 into a stem cell line. In some embodiments, the at least one ribonucleic acid or the at least one pair of ribonucleic acids is selected from any one disclosed in WO2016183041, including the sequence listing.

In some embodiments, the tolerogenic factors are expressed in a cell using an expression vector. In some embodiments, the tolerogenic factors are introduced to the cell using a viral expression vector that mediates integration of the tolerogenic factor sequence into the genome of the cell. For example, the expression vector for expressing CD47 in a cell comprises a polynucleotide sequence encoding CD47. In some embodiments, the expression vector is an inducible expression vector. In some embodiments, the expression vector is a viral vector, such as but not limited to, a lentiviral vector. In some embodiments, the tolerogenic factors are introduced into the cells using fusogen-mediated delivery or a transposase system selected from the group consisting of conditional or inducible transposases, conditional or inducible PiggyBac transposons, conditional or inducible Sleeping Beauty (SB11) transposons, conditional or inducible Mos1 transposons, and conditional or inducible Tol2 transposons.

In some embodiments, the present disclosure provides a cell (e.g., a primary T cell and a hypoimmunogenic stem cell and derivative thereof) or population thereof comprising a genome in which the cell genome has been modified to express any one of the polypeptides selected from the group consisting of HLA-A, HLA-B, HLA-C, RFX-ANK, CIITA, NFY-A, NLRC5, B2M, RFX5, RFX-AP, HLA-G, HLA-E, NFY-B, PD-L1, NFY-C, IRF1, TAP1, GITR, 4-1BB, CD28, B7-1, CD47, B7-2, OX40, CD27, HVEM, SLAM, CD226, ICOS, LAG3, TIGIT, TIM3, CD160, BTLA, CD244, LFA-1, ST2, HLA-F, CD30, B7-H3, VISTA, TLT, PD-L2, CD58, CD2, HELIOS, and IDOL. In some embodiments, the present disclosure provides a method for altering a cell genome to express any one of the polypeptides selected from the group consisting of HLA-A, HLA-B, HLA-C, RFX-ANK, CIITA, NFY-A, NLRC5, B2M, RFX5, RFX-AP, HLA-G, HLA-E, NFY-B, PD-L1, NFY-C, IRF1, TAP1, GITR, 4-1BB, CD28, B7-1, CD47, B7-2, OX40, CD27, HVEM, SLAM, CD226, ICOS, LAG3, TIGIT, TIM3, CD160, BTLA, CD244, LFA-1, ST2, HLA-F, CD30, B7-H3, VISTA, TLT, PD-L2, CD58, CD2, HELIOS, and IDOL. In some embodiments, at least one ribonucleic acid or at least one pair of ribonucleic acids is utilized to facilitate the insertion of the selected polypeptide into a stem cell line. In some embodiments, the at least one ribonucleic acid or the at least one pair of ribonucleic acids is selected from any one disclosed in Appendices 1-47 and the sequence listing of WO2016183041, the disclosure is incorporated herein by references.

In some embodiments, a suitable gene editing system (e.g., CRISPR/Cas system or any of the gene editing systems described herein) is used to facilitate the insertion of a polynucleotide encoding a tolerogenic factor, into a genomic locus of the hypoimmunogenic cell. In some embodiments, the polynucleotide encoding the tolerogenic factor is inserted into a safe harbor or target locus, such as but not limited to, an AAVS1, CCR5, CLYBL, ROSA26, SHS231, F3 (CD142), MICA, MICB, LRP1 (CD91), HMGB1, ABO, RHD, FUT1, or KDM5D gene locus. In some embodiments, the polynucleotide encoding the tolerogenic factor is inserted into a B2M gene locus, a CIITA gene locus, a TRAC gene locus, or a TRB gene locus. In some embodiments, the polynucleotide encoding the tolerogenic factor is operably linked to a promoter.

In some embodiments, the cells are engineered to expresses an increased amount of one or more of CD47, DUX4, CD24, CD27, CD35, CD46, CD55, CD59, CD200, HLA-C, HLA-E, HLA-E heavy chain, HLA-G, PD-L1, IDO1, CTLA4-Ig, C1-Inhibitor, IL-10, IL-35, IL-39, FasL, CCL21, CCL22, Mfge8, CD16, CD52, H2-M3, CD16 Fc receptor, IL15-RF, H2-M3(HLA-G), B2M-HLA-E, A20/TNFAIP3, CR1, HLA-F, MANF, and/or Serpinb9 relative to a cell of the same cell type that does not comprise the modifications.

### M. Characteristics of Hypoimmune Cells

In some embodiments, the population of hypoimmunogenic stem cells retains pluripotency as compared to a control stem cell (e.g., a wild-type stem cell or immunogenic stem cell). In some embodiments, the population of hypoimmunogenic stem cells retains differentiation potential as compared to a control stem cell (e.g., a wild-type stem cell or immunogenic stem cell).

In some embodiments, the administered population of hypoimmunogenic cells such as hypoimmunogenic CAR-T cells elicits a decreased or lower level of immune activation in the subject or patient. In some instances, the level of immune activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation produced by the administration of immunogenic cells. In some embodiments, the administered population of hypoimmunogenic cells fails to elicit immune activation in the subject or patient.

In some embodiments, the administered population of hypoimmunogenic cells such as hypoimmunogenic CAR-T cells elicits a decreased or lower level of T cell response in the subject or patient. In some instances, the level of T cell response elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of T cell response produced by the administration of immunogenic cells. In some embodiments, the administered population of hypoimmunogenic cells fails to elicit a T cell response to the cells in the subject or patient.

In some embodiments, the administered population of hypoimmunogenic cells such as hypoimmunogenic CAR-T cells elicits a decreased or lower level of NK cell response in the subject or patient. In some instances, the level of NK cell response elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of NK cell response produced by the administration of immunogenic cells. In some embodiments, the administered population of hypoimmunogenic cells fails to elicit an NK cell response to the cells in the subject or patient.

In some embodiments, the administered population of hypoimmunogenic cells such as hypoimmunogenic CAR-T cells elicits a decreased or lower level of macrophage engulfment in the subject or patient. In some instances, the level of NK cell response elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of macrophage engulfment produced by the administration of immunogenic cells. In some embodiments, the administered population of hypoimmunogenic cells fails to elicit macrophage engulfment of the cells in the subject or patient.

In some embodiments, the administered population of hypoimmunogenic cells such as hypoimmunogenic CAR-T cells elicits a decreased or lower level of systemic TH1 activation in the subject or patient. In some instances, the level of systemic TH1 activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of systemic TH1 activation produced by the administration of immunogenic cells. In some embodiments, the administered population of hypoimmunogenic cells fails to elicit systemic TH1 activation in the subject or patient.

In some embodiments, the administered population of hypoimmunogenic cells such as hypoimmunogenic CAR-T cells elicits a decreased or lower level of NK cell killing in the subject or patient. In some instances, the level of NK cell killing elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of NK cell killing produced by the administration of immunogenic cells. In some embodiments, the administered population of hypoimmunogenic cells fails to elicit NK cell killing in the subject or patient.

In some embodiments, the administered population of hypoimmunogenic cells such as hypoimmunogenic CAR-T cells elicits a decreased or lower level of immune activation of peripheral blood mononuclear cells (PBMCs) in the subject or patient. In some instances, the level of immune activation of PBMCs elicited by the cells is at least 5%, 10%, 15%, 20%, 25%, 30%, 35%0, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation of PBMCs produced by the administration of immunogenic cells. In some embodiments, the administered population of hypoimmunogenic cells fails to elicit immune activation of PBMCs in the subject or patient.

In some embodiments, the administered population of hypoimmunogenic cells such as hypoimmunogenic CAR-T cells elicits a decreased or lower level of donor-specific IgG antibodies in the subject or patient. In some instances, the level of donor-specific IgG antibodies elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of donor-specific IgG antibodies produced by the administration of immunogenic cells. In some embodiments, the administered population of hypoimmunogenic cells fails to elicit donor-specific IgG antibodies in the subject or patient.

In some embodiments, the administered population of hypoimmunogenic cells such as hypoimmunogenic CAR-T cells elicits a decreased or lower level of donor-specific IgM antibodies in the subject or patient. In some instances, the level of donor-specific IgM antibodies elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of donor-specific IgM antibodies produced by the administration of immunogenic cells. In some embodiments, the administered population of hypoimmunogenic cells fails to elicit donor-specific IgM antibodies in the subject or patient.

In some embodiments, the administered population of hypoimmunogenic cells such as hypoimmunogenic CAR-T cells elicits a decreased or lower level of IgM and IgG antibody production in the subject or patient. In some instances, the level of IgM and IgG antibody production elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of IgM and IgG antibody production produced by the administration of immunogenic cells. In some embodiments, the administered population of hypoimmunogenic cells fails to elicit IgM and IgG antibody production in the subject or patient.

In some embodiments, the administered population of hypoimmunogenic cells such as hypoimmunogenic CAR-T cells elicits a decreased or lower level of cytotoxic T cell killing in the subject or patient. In some instances, the level of cytotoxic T cell killing elicited by the cells is at least 5%, 10%, 15%, 20%, 25%, 30%, 35%, 40%, 45%, 50%, 55% 60%, 65%0, 70%, 75%, 80%, 85%, 90%, 91%, 92%, 93%, 94%, 95%, 96%, 97%, 98%, or 99% lower compared to the level of cytotoxic T cell killing produced by the administration of immunogenic cells. In some embodiments, the administered population of hypoimmunogenic cells fails to elicit cytotoxic T cell killing in the subject or patient.

In some embodiments, the administered population of hypoimmunogenic cells such as hypoimmunogenic CAR-T cells elicits a decreased or lower level of complement-dependent cytotoxicity (CDC) in the subject or patient. In some instances, the level of CDC elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of CDC produced by the administration of immunogenic cells. In some embodiments, the administered population of hypoimmunogenic cells fails to elicit CDC in the subject or patient.

N. Therapeutic Cells from Primary T Cells

Provided herein are hypoimmunogenic cells including, but not limited to, primary T cells that evade immune recognition. In some embodiments, the hypoimmunogenic cells are produced (e.g., generated, cultured, or derived) from T cells such as primary T cells. In some instances, primary T cells are obtained (e.g., harvested, extracted, removed, or taken) from a subject or an individual. In some embodiments, primary T cells are produced from a pool of T cells such that the T cells are from one or more subjects (e.g., one or more human including one or more healthy humans). In some embodiments, the pool of primary T cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of T cells do not include cells from the patient. In some embodiments, one or more of the donor subjects from which the pool of T cells is obtained are different from the patient.

In some embodiments, the hypoimmunogenic cells do not activate an innate and/or an adaptive immune response in the patient (e.g., recipient upon administration). Provided are methods of treating a disorder by administering a population of hypoimmunogenic cells to a subject (e.g., recipient) or patient in need thereof. In some embodiments, the hypoimmunogenic cells described herein comprise T cells engineered (e.g., are modified) to express a chimeric antigen receptor including but not limited to a chimeric antigen receptor described herein. In some instances, the T cells are populations or subpopulations of primary T cells from one or more individuals. In some embodiments, the T cells described herein such as the engineered or modified T cells comprise reduced expression of an endogenous T cell receptor.

In some embodiments, the present disclosure is directed to hypoimmunogenic primary T cells that overexpress CD47 and CARs as disclosed herein, and have reduced expression or lack expression of MHC class I and/or MHC class II human leukocyte antigens and have reduced expression or lack expression of TCR complex molecules. The cells outlined herein overexpress CD47 and CARs and evade immune recognition. In some embodiments, the primary T cells display reduced levels or activity of MHC class I antigens, MHC class II antigens, and/or TCR complex molecules. In some embodiments, primary T cells overexpress CD47 and CARs and harbor a genomic modification in the B2M gene. In some embodiments, T cells overexpress CD47 and CARs and harbor a genomic modification in the CIITA gene. In some embodiments, primary T cells overexpress CD47 and CARs and harbor a genomic modification in the TRAC gene. In some embodiments, primary T cells overexpress CD47 and CARs and harbor a genomic modification in the TRB gene. In some embodiments, T cells overexpress CD47 and CARs and harbor genomic modifications in one or more of the following genes: the B2M, CIITA, TRAC and TRB genes.

Exemplary T cells of the present disclosure are selected from the group consisting of cytotoxic T cells, helper T cells, memory T cells, central memory T cells, effector memory T cells, effector memory RA T cells, regulatory T cells, tissue infiltrating lymphocytes, and combinations thereof. In some embodiments, the T cells express CCR7, CD27, CD28, and CD45RA. In some embodiments, the central T cells express CCR7, CD27, CD28, and CD45RO. In other embodiments, the effector memory T cells express PD-1, CD27, CD28, and CD45RO. In other embodiments, the effector memory RA T cells express PD-1, CD57, and CD45RA.

In some embodiments, the T cell is a modified (e.g., an engineered) T cell. In some embodiments, the modified T cell comprise a modification causing the cell to express at least one chimeric antigen receptor as disclosed herein. Useful modifications to primary T cells are described in detail in US2016/0348073 and WO2020/018620, the disclosures of which are incorporated herein in their entireties.

In some embodiments, the hypoimmunogenic cells described herein comprise T cells that are engineered (e.g., are modified) to express a chimeric antigen receptor including but not limited to a chimeric antigen receptor described herein. In some instances, the T cells are populations or subpopulations of primary T cells from one or more individuals. In some embodiments, the T cells described herein such as the engineered or modified T cells include reduced expression of an endogenous T cell receptor. In some embodiments, the T cells described herein such as the engineered or modified T cells include reduced expression of cytotoxic T-lymphocyte-associated protein 4 (CTLA-4). In other embodiments, the T cells described herein such as the engineered or modified T cells include reduced expression of programmed cell death (PD-1). In some embodiments, the T cells described herein such as the engineered or modified T cells include reduced expression of CTLA-4 and PD-1. Methods of reducing or eliminating expression of CTLA-4, PD-1 and both CTLA-4 and PD-1 are any recognized by those skilled in the art, such as but not limited to, genetic modification technologies that utilize rare-cutting endonucleases and RNA silencing or RNA interference technologies. Non-limiting examples of a rare-cutting endonuclease include any Cas protein, TALEN, zinc finger nuclease, meganuclease, and homing endonuclease. In some embodiments, an exogenous nucleic acid encoding a polypeptide as disclosed herein (e.g., a chimeric antigen receptor, CD47, or another tolerogenic factor disclosed herein) is inserted at a CTLA-4 and/or PD-1 gene locus. In some embodiments, the exogenous polynucleotide is inserted into at least one allele of the cell using viral transduction, for example, with a vector. In some embodiments, the vector is a pseudotyped, self-inactivating lentiviral vector that carries the exogenous polynucleotide. In some embodiments, the vector is a self-inactivating lentiviral vector pseudotyped with a vesicular stomatitis VSV-G envelope, and which carries the exogenous polynucleotide. In some embodiments, the exogenous polynucleotide is inserted into at least one allele of the cell using viral transduction. In some embodiments, the exogenous polynucleotide is inserted into at least one allele of the cell using a lentivirus based viral vector.

In some embodiments, the T cells described herein such as the engineered or modified T cells include enhanced expression of PD-L1.

In some embodiments, the hypoimmunogenic T cell includes a polynucleotide encoding a CAR as herein disclosed, wherein the polynucleotide is inserted in a genomic locus. In some embodiments, the polynucleotide encoding the CAR is randomly integrated into the genome of the cell. In some embodiments, the polynucleotide encoding the CAR is randomly integrated into the genome of the cell via viral vector transduction. In some embodiments, the polynucleotide encoding the CAR is randomly integrated into the genome of the cell via lentiviral vector transduction. In some embodiments, the polynucleotide is inserted into a safe harbor or target locus, such as but not limited to, an AAVS1, CCR5, CLYBL, ROSA26, SHS231, F3 (also known as CD142), MICA, MICB, LRP1 (also known as CD91), HMGB1, ABO, RHD, FUT1, or KDM5D gene locus. In some embodiments, the polynucleotide is inserted in a B2M, CIITA, TRAC, TRB, PD-1 or CTLA-4 gene.

In some embodiments, the hypoimmunogenic T cell includes a polynucleotide encoding a CAR that is expressed in a cell using an expression vector. In some embodiments, the CAR is introduced to the cell using a viral expression vector that mediates integration of the CAR sequence into the genome of the cell. For example, the expression vector for expressing the CAR in a cell comprises a polynucleotide sequence encoding the CAR. In some embodiments, the expression vector is an inducible expression vector. In some embodiments, the expression vector is a viral vector, such as but not limited to, a lentiviral vector.

Hypoimmunogenic T cells provided herein are useful for the treatment of suitable cancers including, but not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

O. Therapeutic Cells Differentiated from Hypoimmune Pluripotent Stem Cells

Provided herein are hypoimmunogenic cells including, cells derived from pluripotent stem cells, that evade immune recognition. In some embodiments, the cells do not activate an innate and/or an adaptive immune response in the patient or subject (e.g., recipient upon administration). Provided are methods of treating a disorder comprising repeat dosing of a population of hypoimmunogenic cells to a recipient subject in need thereof.

In some embodiments, the pluripotent stem cell and any cell differentiated from such a pluripotent stem cell is modified to exhibit reduced expression of MHC class I human leukocyte antigens. In other embodiments, the pluripotent stem cell and any cell differentiated from such a pluripotent stem cell is modified to exhibit reduced expression of MHC class II human leukocyte antigens. In some embodiments, the pluripotent stem cell and any cell differentiated from such a pluripotent stem cell is modified to exhibit reduced expression of TCR complexes. In some embodiments, the pluripotent stem cell and any cell differentiated from such a pluripotent stem cell is modified to exhibit reduced expression of MHC class I and II human leukocyte antigens. In some embodiments, the pluripotent stem cell and any cell differentiated from such a pluripotent stem cell is modified to exhibit reduced expression of MHC class I and II human leukocyte antigens and TCR complexes.

In some embodiments, the pluripotent stem cell and any cell differentiated from such a pluripotent stem cell is modified to exhibit reduced expression of MHC class I and/or II human leukocyte antigens and exhibit increased CD47 expression. In some instances, the cell overexpresses CD47 by harboring one or more CD47 transgenes. In some embodiments, the pluripotent stem cell and any cell differentiated from such a pluripotent stem cell is modified to exhibit reduced expression of MHC class I and II human leukocyte antigens and exhibit increased CD47 expression. In some embodiments, the pluripotent stem cell and any cell differentiated from such a pluripotent stem cell is modified to exhibit reduced expression of MHC class I and II human leukocyte antigens and TCR complexes and exhibit increased CD47 expression.

In some embodiments, the pluripotent stem cell and any cell differentiated from such a pluripotent stem cell is modified to exhibit reduced expression of MHC class I and/or II human leukocyte antigens, to exhibit increased CD47 expression, and to exogenously express a chimeric antigen receptor as disclosed herein. In some instances, the cell overexpresses CD47 polypeptides by harboring one or more CD47 transgenes. In some instances, the cell overexpresses CAR polypeptides by harboring one or more CAR transgenes. In some embodiments, the pluripotent stem cell and any cell differentiated from such a pluripotent stem cell is modified to exhibit reduced expression of MHC class I and II human leukocyte antigens, exhibit increased CD47 expression, and to exogenously express a chimeric antigen receptor. In some embodiments, the pluripotent stem cell and any cell differentiated from such a pluripotent stem cell is modified to exhibit reduced expression of MHC class I and II human leukocyte antigens and TCR complexes, to exhibit increased CD47 expression, and to exogenously express a chimeric antigen receptor.

Such pluripotent stem cells are hypoimmunogenic stem cells. Such differentiated cells are hypoimmunogenic cells.

In some embodiments, any of the pluripotent stem cells described herein are differentiated into any cells of an organism and tissue. In some embodiments, the cells exhibit reduced expression of MHC class I and/or II human leukocyte antigens and reduced expression of TCR complexes. In some instances, expression of MHC class I and/or II human leukocyte antigens is reduced compared to unmodified or wild-type cell of the same cell type. In some instances, expression of TCR complexes is reduced compared to unmodified or wild-type cell of the same cell type. In some embodiments, the cells exhibit increased CD47 expression. In some instances, expression of CD47 is increased in cells encompassed by the present disclosure as compared to unmodified or wild-type cells of the same cell type. In some embodiments, the cells exhibit exogenous CAR expression. Methods for reducing levels of MHC class I and/or II human leukocyte antigens and TCR complexes and increasing the expression of CD47 and CARs are described herein.

In some embodiments, the cells used in the methods described herein evade immune recognition and responses when administered to a patient (e.g., recipient subject). The cells can evade killing by immune cells in vitro and in vivo. In some embodiments, the cells evade killing by macrophages and NK cells. In some embodiments, the cells are ignored by immune cells or a subject's immune system. In other words, the cells administered in accordance with the methods described herein are not detectable by immune cells of the immune system. In some embodiments, the cells are cloaked and therefore avoid immune rejection.

Methods of determining whether a pluripotent stem cell and any cell differentiated from such a pluripotent stem cell evades immune recognition include, but are not limited to, IFN-γ Elispot assays, microglia killing assays, cell engraftment animal models, cytokine release assays, ELISAs, killing assays using bioluminescence imaging or chromium release assay or a real-time, quantitative microelectronic biosensor system for cell analysis (xCELLigence® RTCA system, Agilent), mixed-lymphocyte reactions, immunofluorescence analysis, etc.

Therapeutic cells outlined herein are useful to treat a disorder such as, but not limited to, a cancer, a genetic disorder, a chronic infectious disease, an autoimmune disorder, a neurological disorder, and the like.

i. T Lymphocytes Differentiated from Hypoimmunogenic Pluripotent Cells

Provided herein, T lymphocytes (T cells, including primary T cells) are derived from the HIP cells described herein (e.g., hypoimmunogenic iPSCs). Methods for generating T cells, including CAR-T cells, from pluripotent stem cells (e.g., iPSCs) are described, for example, in Iriguchi et al., Nature Communications 12, 430 (2021); Themeli et al., Cell Stem Cell, 16(4):357-366 (2015); Themeli et al., Nature Biotechnology 31:928-933 (2013).

T lymphocyte derived hypoimmunogenic cells include, but are not limited to, primary T cells that evade immune recognition. In some embodiments, the hypoimmunogenic cells are produced (e.g., generated, cultured, or derived) from T cells such as primary T cells. In some instances, primary T cells are obtained (e.g., harvested, extracted, removed, or taken) from a subject or an individual. In some embodiments, primary T cells are produced from a pool of T cells such that the T cells are from one or more subjects (e.g., one or more human including one or more healthy humans). In some embodiments, the pool of primary T cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of T cells does not include cells from the patient. In some embodiments, one or more of the donor subjects from which the pool of T cells is obtained are different from the patient.

In some embodiments, the hypoimmunogenic cells do not activate an immune response in the patient (e.g., recipient upon administration). Provided are methods of treating a disorder by administering a population of hypoimmunogenic cells to a subject (e.g., recipient) or patient in need thereof. In some embodiments, the hypoimmunogenic cells described herein comprise T cells engineered (e.g., are modified) to express a chimeric antigen receptor including but not limited to a chimeric antigen receptor described herein. In some instances, the T cells are populations or subpopulations of primary T cells from one or more individuals. In some embodiments, the T cells described herein such as the engineered or modified T cells comprise reduced expression of an endogenous T cell receptor.

In some embodiments, the HIP-derived T cell includes a chimeric antigen receptor (CAR) as described herein. In some embodiments, any suitable CAR described herein is included in the hyHIP-derived T cell. In some embodiments, the hypoimmunogenic induced pluripotent stem cell-derived T cell includes a polynucleotide encoding a CAR, wherein the polynucleotide is inserted in a genomic locus. In some embodiments, the polynucleotide is inserted into a safe harbor or target locus. In some embodiments, the polynucleotide is inserted in a B2M, CIITA, TRAC, TRB, PD-1 or CTLA-4 gene. In some embodiments, any suitable sub-sub-method is used to insert the CAR into the genomic locus of the hypoimmunogenic cell including the gene editing methods described herein (e.g., a CRISPR/Cas system).

HIP-derived T cells provided herein are useful for the treatment of suitable cancers including, but not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

ii. NK Cells Derived from Hypoimmunogenic Pluripotent Cells

Provided herein, natural killer (NK) cells are derived from the HIP cells described herein (e.g., hypoimmunogenic iPSCs).

NK cells (also defined as ‘large granular lymphocytes’) represent a cell lineage differentiated from the common lymphoid progenitor (which also gives rise to B lymphocytes and T lymphocytes). Unlike T-cells, NK cells do not naturally comprise CD3 at the plasma membrane. Importantly, NK cells do not express a TCR and typically also lack other antigen-specific cell surface receptors (as well as TCRs and CD3, they also do not express immunoglobulin B-cell receptors, and instead typically express CD16 and CD56). NK cell cytotoxic activity does not require sensitization but is enhanced by activation with a variety of cytokines including IL-2. NK cells are generally thought to lack appropriate or complete signaling pathways necessary for antigen-receptor-mediated signaling, and thus are not thought to be capable of antigen receptor-dependent signaling, activation and expansion. NK cells are cytotoxic, and balance activating and inhibitory receptor signaling to modulate their cytotoxic activity. For instance, NK cells expressing CD16 may bind to the Fc domain of antibodies bound to an infected cell, resulting in NK cell activation. By contrast, activity is reduced against cells expressing high levels of MHC class I proteins. On contact with a target cell NK cells release proteins such as perforin, and enzymes such as proteases (granzymes). Perforin can form pores in the cell membrane of a target cell, inducing apoptosis or cell lysis.

There are a number of techniques that are used to generate NK cells, including CAR-NK-cells, from pluripotent stem cells (e.g., iPSC); see, for example, Zhu et al., Methods Mol Biol. 2019; 2048:107-119; Knorr et al., Stem Cells Transl Med. 2013 2(4):274-83. doi: 10.5966/sctm.2012-0084; Zeng et al., Stem Cell Reports. 2017 Dec. 12; 9(6):1796-1812; Ni et al., Methods Mol Biol. 2013; 1029:33-41; Bernareggi et al., Exp Hematol. 2019 71:13-23; Shankar et al., Stem Cell Res Ther. 2020;11(1):234, all of which are incorporated herein by reference in their entirety and specifically for the methodologies and reagents for differentiation. Differentiation is assayed as is known in the art, generally by evaluating the presence of NK cell associated and/or specific markers, including, but not limited to, CD56, KIRs, CD16, NKp44, NKp46, NKG2D, TRAIL, CD122, CD27, CD244, NK1.1, NKG2A/C, NCR1, Ly49, CD49b, CD11b, KLRG1, CD43, CD62L, and/or CD226.

In some embodiments, the hypoimmunogenic pluripotent cells are differentiated into hepatocytes to address loss of the hepatocyte functioning or cirrhosis of the liver. There are a number of techniques that are used to differentiate HIP cells into hepatocytes; see for example, Pettinato et al., doi: 10.1038/spre32888, Snykers et al., Methods Mol Biol., 2011 698:305-314, Si-Tayeb et al., Hepatology, 2010, 51:297-305 and Asgari et al., Stem Cell Rev., 2013, 9(4):493-504, all of which are incorporated herein by reference in their entirety and specifically for the methodologies and reagents for differentiation. Differentiation is assayed as is known in the art, generally by evaluating the presence of hepatocyte associated and/or specific markers, including, but not limited to, albumin, alpha fetoprotein, and fibrinogen. Differentiation can also be measured functionally, such as the metabolization of ammonia, LDL storage and uptake, ICG uptake and release, and glycogen storage.

In some embodiments, the NK cells do not activate an innate and/or an adaptive immune response in the patient (e.g., recipient upon administration). Provided are methods of treating a disorder by administering a population of NK cells to a subject (e.g., recipient) or patient in need thereof. In some embodiments, the NK cells described herein comprise NK cells engineered (e.g., are modified) to express a chimeric antigen receptor including but not limited to a chimeric antigen receptor described herein. In some embodiments, any suitable CAR is included in the NK cells, including the CARs described herein. In some embodiments, the NK cell includes a polynucleotide encoding a CAR, wherein the polynucleotide is inserted in a genomic locus. In some embodiments, the polynucleotide is inserted into a safe harbor or a target locus. In some embodiments, the polynucleotide is inserted in a B2M, CIITA, PD1 or CTLA4 gene. In some embodiments, any suitable method is used to insert the CAR into the genomic locus of the NK cell including the gene editing methods described herein (e.g., a CRISPR/Cas system).

### Methods of Inserting CAR Transgenes to Produce Cells Expressing CARs

In some aspects, the present technology provides methods for generating a population of cells expressing a CAR, such as immune evasive allogeneic T cells, for cell therapy. In some embodiments, the method comprises (a) inserting a first transgene encoding a tolerogenic factor into an endogenous TCR gene locus (e.g., the TRAC and/or TRBC loci including TRBC1 and/or TRBC2) of the T cells, and (b) selecting for T cells that have the transgene inserted by CD3 depletion and/or positive selection for the tolerogenic factor (e.g., selection for expression of the tolerogenic factor). The endogenous TCR gene locus is a genomic locus within any gene encoding a TCR or a component thereof, including, for example, the TRAC and/or TRBC (including TRBC1 and TRBC2) loci. Inserting a tolerogenic factor at the endogenous TCR gene locus may achieve the dual purposes of reducing or eliminating TCR expression and increasing expression of the tolerogenic factor in the T cells (especially allogenic T cells) in one manufacturing step, so that the resulting T cells are made immune evasive and not subject to immune rejection when transplanted into a recipient, thereby increasing both the efficiency of the manufacturing process and the effectiveness of cell-based therapies. In some embodiments, the methods further comprise modifying the expression of MHC class I and/or MHC class II molecules in the T cells. In some embodiments, methods further comprise inserting a second transgene encoding a CAR to a genomic locus of the T cells.

### F. Insertion of a First Polynucleotide Encoding a Tolerogenic Factor

i. Tolerogenic Factors

In some embodiments, the tolerogenic factor is selected from the group consisting of CD16, CD24, CD35, CD39, CD46, CD47, CD52, CD55, CD59, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, MANF, and any combinations, truncations, modifications, or fusions of the above.

In some embodiments, the tolerogenic factor is CD47. CD47 is a leukocyte surface antigen and has a role in cell adhesion and modulation of integrins. It is expressed on the surface of a cell (e.g., a T cell) and signals to circulating macrophages not to phagocytize the cell. Overexpression of CD47 thus can reduce the immunogenicity of the cell when grafted and improve immune protection in allogeneic recipients.

In some embodiments, the CD47 is human CD47, and in some of these embodiments, the human CD47 comprises or consists of an amino acid sequence set forth in SEQ ID NO: 167 or SEQ ID NO: 168 or is at least 80% identical (e.g., at least 80%, at least 85%, at least 90%, at least 95%, at least 96%, at least 97%, at least 98%, at least 99%, or 100% identical) to the amino acid sequence set forth in SEQ ID NO: 167 or SEQ ID NO: 168 as set forth in Table 23. In some embodiments, the transgene encoding CD47 comprises a nucleotide sequence corresponding to an mRNA sequence of human CD47. In some embodiments, the transgene encoding CD47 has a nucleotide sequence that is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 169 (coding sequence (CDS) of the nucleotide sequence set forth in NCBI Ref. No. NM_001777.4) or SEQ ID NO: 170 (CDS of the nucleotide sequence set forth in NCBI Ref. No. NM_198793.2).

In some embodiments, the polynucleotide (e.g., transgene) encoding CD47 is codon-optimized for expression in a mammalian cell, for example, a human cell. In some embodiments, the codon-optimized polynucleotide encoding CD47 has a nucleotide sequence that is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 171.

In some embodiments, a first transgene encoding a first tolerogenic factor at an insertion site at a TCR gene locus has a reverse sequence orientation (5′ to 3′) relative to the sequence of the TCR gene locus. In some embodiments, a first transgene encoding a first tolerogenic factor at an insertion site at a TCR gene locus comprises a promoter that has a reverse sequence orientation (5′ to 3′) relative to the sequence of the TCR gene locus. In some embodiments, the promoter that has a reverse sequence orientation (5′ to 3′) relative to the sequence of the TCR gene locus drives transcription of a first transgene encoding a first tolerogenic factor in a reverse sequence orientation relative to the TCR gene locus. In some embodiments, a first transgene encoding a first tolerogenic factor at an insertion site at a TCR gene locus comprises (in 5′ to 3′ order relative to the TCR gene locus) a poly-A tail sequence, a reverse orientation transgene sequence, and a reverse orientation promoter sequence. In some embodiments, a TCR gene locus comprises a first transgene encoding a first tolerogenic factor and a second transgene encoding a CAR as disclosed herein in a reverse sequence orientation (5′ to 3′) relative to the sequence of the TCR gene locus. In some embodiments, a TCR gene locus comprises a first transgene encoding a first tolerogenic factor in a reverse sequence orientation (5′ to 3′) relative to the sequence of the TCR gene locus and a second transgene encoding a CAR in the forward orientation (i.e., the same orientation) relative to the sequence of the TCR gene locus. In some embodiments, a TCR gene locus comprises a first transgene encoding a first tolerogenic factor and a second transgene encoding a second tolerogenic factor in a reverse sequence orientation (5′ to 3′) relative to the sequence of the TCR gene locus. In some embodiments, a TCR gene locus comprises a first transgene encoding a first tolerogenic factor in a reverse sequence orientation (5′ to 3′) relative to the sequence of the TCR gene locus and a second transgene encoding a second tolerogenic factor in the forward orientation (i.e., the same orientation) relative to the sequence of the TCR gene locus. In some embodiments, a TCR gene locus comprises a first transgene encoding a first tolerogenic factor, a second transgene encoding a second tolerogenic factor, and a third transgene encoding a CAR in a reverse sequence orientation (5′ to 3′) relative to the sequence of the TCR gene locus. In some embodiments, a TCR gene locus comprises a first transgene encoding a first tolerogenic factor and a second transgene encoding a second tolerogenic factor in a reverse sequence orientation (5′ to 3′) relative to the sequence of the TCR gene locus, and a third transgene encoding a CAR in the forward orientation (i.e., the same orientation) relative to the sequence of the TCR gene locus. In some embodiments, a TCR gene locus comprises a first transgene encoding a first tolerogenic factor in a reverse sequence orientation (5′ to 3′) relative to the sequence of the TCR gene locus, a second transgene encoding a second tolerogenic factor in the forward orientation (i.e., the same orientation) relative to the sequence of the TCR gene locus, and a third transgene encoding a CAR in the forward orientation (i.e., the same orientation) relative to the sequence of the TCR gene locus.

ii. Regulatory Elements

In some embodiments, a transgene comprises a gene and one or more regulatory elements. In some embodiments, expression of the tolerogenic factor is operably linked to an endogenous promoter at the TCR gene locus (e.g., TRAC, TRBC1, and/or TRBC2). In some of these embodiments, the first transgene encoding the tolerogenic factor to be inserted need not include an exogenous promoter however, in some embodiments, the transgene may include an exogenous insulator and/or an exogenous enhancer.

Alternatively, in other embodiments, the first transgene encoding a tolerogenic factor may additionally comprise an exogenous promoter to drive expression of the tolerogenic factor in the host cell. In some of these embodiments, the exogenous promoter is one that drives constitutive gene expression in mammalian cells. Those frequently used include, for example, elongation factor 1 alpha (EF 1α) promoter, cytomegalovirus (CMV) immediate-early promoter (Greenaway et al., Gene 18: 355-360 (1982)), simian vacuolating virus 40 (SV40) early promoter (Fiers et al., Nature 273:113-120 (1978)), spleen focus-forming virus (SFFV) promoter, phosphoglycerate kinase (PGK) promoter (Adra et al., Gene 60(1):65-74 (1987)), human beta actin promoter, polyubiquitin C gene (UBC) promoter, CAG promoter (Nitoshi et al., Gene 108:193-199 (1991)), MND (MPSV LTR, NCR deleted, and d/587 PBS; Challita et al., J. Virol 69(2):748-755 (1995)) promoter, SSFV promoter, and ICOS promoter. An example of a promoter that is capable of expressing a transgene in a mammalian cell (e.g., a T cell) is the EF1 a promoter. The native EF1 a promoter drives expression of the alpha subunit of the elongation factor-1 complex, which is responsible for the enzymatic delivery of aminoacyl tRNAs to the ribosome. The EFla promoter has been extensively used in mammalian expression plasmids and has been shown to be effective in driving CAR expression from transgenes cloned into a lentiviral vector. See, e.g., Milone et al., Mol. Ther. 17(8):1453-1464 (2009). For another example, an MND promoter is a synthetic promoter that contains the U3 region of a modified gammaretrovirus-derived MoMuLV LTR with myeloproliferative sarcoma virus enhancer, and this promoter has been shown to be highly and constitutively active in the hematopoietic system and to resist transcriptional silencing. See, e.g., Halene et al., Blood 94(10):3349-3357 (1999).

In some embodiments, the first transgene encoding a tolerogenic factor may comprise additional regulatory elements operatively linked to the tolerogenic factor sequence and/or promoter, including, for example, insulators, enhancers, polyadenylation (poly(A)) tails, and/or ubiquitous chromatin opening elements. As known to a skilled artisan, these regulatory elements may be needed to affect the expression and processing of coding sequences to which they are operatively linked. Regulatory elements used for transgene expression modulation may include appropriate transcription initiation, termination, promoter, and enhancer sequences; efficient RNA processing signals, such as splicing and polyadenylation signals; sequences that stabilize cytoplasmic mRNA; sequences that enhance translation efficiency; sequences that enhance protein stability; and possibly sequences that enhance protein secretion.

In some embodiments, the first transgene encoding a tolerogenic factor may additionally comprise an insulator to modulate the expression of the tolerogenic factor in the host cell. Insulators are DNA elements (usually about 50 nucleotides in length) that can shelter genes from inappropriate regulatory interactions. In some embodiments, insulators insulate genes located in one domain from promiscuous regulation by enhancers or silencers in neighboring domains. Insulators that disrupt communication between an enhancer and its promoter when positioned between the two are called enhancer-blockers, and insulators that are located between a silencer and a promoter and protect the promoter from silencing are called barriers. In some embodiments, insulators that are barriers prevent the advance of nearby condensed chromatin and protect gene expression from positive and negative chromatin effects. Thus, in the design of a transgene, insulators are usually placed upstream of the promoter. Non-limiting examples of insulators include 5′HS5, DMD/ICR, BEAD-1, apoB (−57 kb), apoB (+43 kb), DM1 site 1, DM1 site 2 (from human); BEAD-1, HS2-6, DMR/ICR, SINE (from mouse); SF1, scs/scs′, gypsy, Fab-7, Fab-8, faswab, eve (from fruit fly); HMR tRNAThr, Chal UAS, UASrpg, STAR (from yeast); Lys 5′A, HS4, or 3′HS (from chicken); sns, URI (from sea urchin); and RO (from frog). Other examples of insulators include Mcp, Neighbor of Homie (Nhomie) insulator and Homing insulator at eve (Homie), and Su(Hw)-dependent insulators. In some embodiments, the first transgene encoding a tolerogenic factor may comprise an insulator having a sequence that is at least 80% identical (e.g., at least 80%, at least 85%, at least 90%, at least 95%, at least 96%, at least 97%, at least 98%, at least 99%, or 100% identical) to any of the described insulators.

In some embodiments, the first transgene encoding a tolerogenic factor comprises one copy of an insulator. In some embodiments, the transgene comprises a multimerized insulator. In some embodiments, a transgene comprises two copies of an insulator. In some embodiments, a transgene comprises three copies of an insulator. In some embodiments, a transgene comprises four copies of an insulator. In some embodiments, a transgene comprises five or more copies of an insulator. Insulator effectiveness is influenced by its structure and by the nature of the enhancer, promoter, and genomic context. In some embodiments, the first transgene encoding a tolerogenic factor may comprise two or more heterologous insulators. In some embodiments, the two or more heterologous insulators interact with each other. In some embodiments, the first transgene encoding a tolerogenic factor comprises an insulator and a regulatory protein that binds to the insulator.

In some embodiments, the first transgene encoding a tolerogenic factor may additionally comprise an enhancer to increase expression of the tolerogenic factor in the host cell. Enhancer sequences are regulatory DNA sequences that, when bound by specific proteins called transcription factors, enhance the transcription of an associated gene. Enhancers are regions of DNA, typically 100 to 1000 bp in size, that contain transcription factor-binding sites that stimulate the initiation and elongation of transcription from promoters. In most housekeeping genes, enhancers are located in close proximity to promoters. Some genes feature complex regulatory regions that can consist of dozens of enhancers located at variable distances from the regulated promoter. During transcriptional activation, enhancers are usually located in close proximity to gene promoters. Some promoters described herein already have an enhancer incorporated; for example, the CAG promoter is constructed by combining the CMV early enhancer element, the chicken beta actin gene promoter, and the splice acceptor of the rabbit beta globin gene.

Enhancers may consist of combinations of short, degenerate sites, 6-12 bp in length, that are recognized by DNA-binding transcription factors, which determine enhancer activity. The combination of DNA-binding transcription factors on a given enhancer creates a platform that attracts co-activators and co-repressors that determine the enhancer activity in each specific group of cells. The ability of an enhancer to stimulate transcription depends on the combination of transcription factor sites that positively or negatively affect enhancer activity and the relative concentrations of enhancer-binding transcription factors within the nuclei of a given group of cells. Recently, super-enhancers have been identified, representing a special class of regulatory elements, characterized by large sizes, sometimes reaching tens of thousands of bp, with a high degree of transcription factor and co-activator enrichment. Super-enhancers are often located adjacent to genes known to be critical for cell differentiation. A more detailed study of super-enhancers has shown that they often consist of separate domains that can either function together to enhance the overall activity of each domain or play independent roles during the simultaneous activation of a large number of promoters.

During the activation of transcription, enhancers recruit several key complexes. The p300/CBP and M113/M114/COMPASS complexes have acetyltransferase and methyltransferase activities, respectively. The proteins M113 and M114 both contain a C-terminal SET (suppressor of variegation, enhancer of zeste, trithorax) domain, which is responsible for the monomethylation of lysine 4 of histone H3 (H3K4mel). The complexes formed by M113 and M114 have partially overlapping and insufficiently studied functions in the regulation of enhancer activity. M113 and M114 are also known to be involved in the recruitment of the p300/CBP co-activator, which is responsible for the acetylation of histone H3 at lysine 27 (H3K27ac). H3K27ac and H3K4mel histone marks are distinctive features of active enhancers and are used to identify enhancers in genomes.

In some embodiments, the first transgene encoding a tolerogenic factor may additionally comprise a poly(A) tail. A poly(A) tail is a long chain of adenine nucleotides that is added to an mRNA molecule during RNA processing to increase the stability of the molecule. Immediately after a gene in a eukaryotic cell is transcribed, the new RNA molecule undergoes several modifications known as RNA processing. These modifications alter both ends of the primary RNA transcript to produce a mature mRNA molecule. The processing of the 3′ end adds a poly-A tail to the RNA molecule. First, the 3′ end of the transcript is cleaved to free a 3′ hydroxyl. Then an enzyme called poly-A polymerase adds a chain of adenine nucleotides to the RNA. This process, called polyadenylation, adds a poly-A tail that is between 100 and 250 residues long. The poly-A tail makes the RNA molecule more stable and prevents its degradation. Additionally, the poly-A tail allows the mature messenger RNA molecule to be exported from the nucleus and translated into a protein by ribosomes in the cytoplasm.

In some embodiments, the first transgene encoding a tolerogenic factor may additionally comprise a ubiquitous chromatin opening element (UCOE). The integration of a transgene into a heterochromatic chromatin environment and the methylation of promoter DNA are major mechanisms that are antagonistic to gene expression, resulting in a variegated pattern of gene expression or silencing. Because stable and high level transgene expression are essential for the efficient and rapid production of clonal cell lines in biomanufacturing as well as for the lifelong expression of a transgene at a therapeutic level in gene therapy, genetic regulatory elements that can prevent gene silencing and maintain high levels of expression for long periods of time are crucial.

Genetic regulatory elements that confer a transcriptionally permissive state are broadly dichotomized into those that actively function through dominant chromatin remodeling mechanisms and those that function as border or boundary elements to restrict the spread of heterochromatin marks into regions of euchromatin. The latter include insulators, scaffold/matrix attachment regions (S/MARs), and stabilizing anti-repressor (STAR) elements, whilst the former comprise locus control regions (LCRs) and UCOEs. LCRs and UCOEs are defined by their ability to consistently confer site of integration-independent stable transgene expression that is proportional to transgene copy number, even when integrated into heterochromatin. LCRs are tissue-specific regulatory elements that consist of multiple subcomponents characterized by DNase I hypersensitivity and a high density of transcription factor binding sites. In contrast, UCOEs function ubiquitously and neither consist of multiple DNase I hypersensitive sites that are characteristic of LCRs, nor are they required to flank a transgene at both 5′ and 3′ ends in order to exert their function as in the case of insulators and S/MARs. Thus, structurally and functionally UCOEs represent a distinct class of genetic regulatory element. UCOEs have found widespread usage in protein therapeutic biomanufacturing applications as a means to manage costs and resources as well as to reliably expedite the generation of highly expressing recombinant cell clones. In some embodiments, UCOEs provide stable ubiquitous or tissue-specific expression in somatic tissues as well as in adult, embryonic, and induced pluripotent stem cells and their differentiated progeny.

iii. Site-Directed Genomic Insertion

In some embodiments, the first transgene encoding a tolerogenic factor and/or regulatory elements are delivered into a host cell for targeted genomic insertion in the form of a vector or targeted lipid particle. In some embodiments, the delivery vector is any type of vector suitable for introduction of nucleotide sequences into a cell, including, for example, plasmids, adenoviral vectors, adeno-associated viral (AAV) vectors, retroviral vectors, lentiviral vectors, phages, and HDR-based donor vectors. The different components are introduced into a cell together or separately, and are delivered in a single vector or multiple vectors. The vector is introduced into a cell by any known method in the field, including, for example, viral transformation, calcium phosphate transfection, lipid-mediated transfection, DEAE-dextran, electroporation, microinjection, nucleoporation, liposomes, nanoparticles, or other methods. Insertion of the first transgene encoding a tolerogenic factor and/or regulatory elements into an endogenous TCR gene locus is carried out using any of the site-directed insertion methods and/or systems described herein, including, for example, zinc finger nucleases (ZFNs), transcription activator-like effector nucleases (TALENs), meganucleases, transposases, and clustered regularly interspaced short palindromic repeat (CRISPR)/Cas systems. Insertion of the first transgene encoding a tolerogenic factor and/or regulatory elements into an endogenous TCR gene locus is carried out using a genome-modifying protein described herein, including for example, a CRISPR-associated transposase, prime editing, or Programmable Addition via Site-specific Targeting Elements (PASTE). Insertion of the first transgene encoding a tolerogenic factor and/or regulatory elements into an endogenous TCR gene locus is carried out using a genome-modifying protein described herein, including for example, TnpB polypeptides. In embodiments where a homology directed repair (HDR)-based approach as described is used, the transgene is usually flanked by homology arms (i.e., left homology arm (LHA) and right homology arm (RHA)) that are specific to the target site of insertion. The homology arms are specifically designed for the target genomic locus for the fragment to serve as a template for HDR. The length of each homology arm is generally dependent on the size of the insert being introduced, with larger insertions requiring longer homology arms.

G. TCR Depletion, CD3 Depletion, and/or Positive Selection for the Tolerogenic Factor

In some embodiments, the methods described herein for generating a population of T cells, such as immune evasive allogeneic T cells, comprise selecting for cells containing the first transgene encoding a tolerogenic factor integrated into an endogenous TCR gene locus of the T cells, wherein integration of the first transgene into the TCR gene locus reduces or eliminates expression of a functional TCR complex at a surface of the T cells, which in turn prevents CD3 from locating to the cell surface. In some embodiments, the selecting comprises CD3 depletion. In some embodiments, the selecting comprises positive selection for the tolerogenic factor (e.g., selection for expression of the tolerogenic factor). In some embodiments, CD3 depletion comprises selecting for T cells that have reduced or eliminated expression of endogenous TCR on a cell surface and therefore have reduced or eliminated CD3 associated with a functional TCR complex on the cell surface. In some embodiments, T cells with reduced or eliminated CD3 expression on the cell surface have reduced or eliminated binding to CD3-binding antibodies and/or other CD3-binding proteins. In some embodiments, T cells with reduced or eliminated CD3 expression on the cell surface do not bind to a column and/or a sorting surface with attached CD3-binding antibodies and/or other CD3-binding proteins. In some embodiments, the population of T cells which fails to bind to the CD3-binding antibodies flows through the column and is collected. This population of T cells may also be referred to as enriched for CD3-negative T cells or enriched for T cells having reduced surface expression of CD3. In some embodiments, the selecting comprises TCR depletion. In some embodiments, TCR depletion comprises selecting for T cells that have reduced or eliminated expression of endogenous TCR on a cell surface and therefore have reduced or eliminated TCR complex on the cell surface. In some embodiments, T cells with reduced or eliminated TCR expression on the cell surface have reduced or eliminated binding to TCR-binding antibodies and/or other TCR-binding proteins. In some embodiments, T cells with reduced or eliminated TCR expression on the cell surface do not bind to a column and/or a sorting surface with attached TCR-binding antibodies and/or other TCR-binding proteins. In some embodiments, the population of T cells which fails to bind to the TCR-binding antibodies flows through the column and is collected. This population of T cells may also be referred to as enriched for TCR-negative T cells or enriched for T cells having reduced surface expression of TCR. In some embodiments, positive selection for the tolerogenic factor (e.g., CD47) comprises selecting for T cells that express the tolerogenic factor on the cell surface, for example, at a higher level than endogenous expression levels of the tolerogenic factor. In some embodiments, positive selection for the tolerogenic factor comprises selecting for T cells that express the tolerogenic factor on the cell surface, for example, at a higher level than endogenous expression levels of the tolerogenic factor if the cell expresses any endogenous tolerogenic factor. In these embodiments, antibodies and/or proteins that bind the tolerogenic factor are selected based on a desired affinity and/or avidity for the tolerogenic factor. For example, antibodies and/or proteins having higher affinities and/or avidities for the tolerogenic factor are selected over lower affinities and/or avidities for use with cells which express endogenous levels of the tolerogenic factor. In some embodiments, T cells expressing the tolerogenic factor on the cell surface bind to antibodies and/or proteins that bind to the tolerogenic factor. In some embodiments, T cells expressing the tolerogenic factor on the cell surface bind to a column and/or a sorting surface with attached antibodies and/or other proteins binding the tolerogenic factor.

In some embodiments, the methods described herein for generating a population of T cells, such as immune evasive allogeneic T cells, comprises selecting for cells containing the first transgene encoding a tolerogenic factor integrated into an endogenous TCR gene locus of the T cells, wherein integration of the first transgene into the endogenous TCR gene locus reduces or eliminates expression of a functional TCR complex at a surface of the T cells. In some embodiments, the selecting comprises CD3 depletion, wherein the T cells with reduced or eliminated expression of CD3 on the cell surface are sorted by affinity binding, flow cytometry, and/or immunomagnetic selection using CD3-binding antibodies and/or other CD3-binding proteins. In some embodiments, the selecting comprises TCR depletion, wherein the T cells with reduced or eliminated expression of TCR on the cell surface are sorted by affinity binding, flow cytometry, and/or immunomagnetic selection using TCR-binding antibodies and/or other TCR-binding proteins. In some embodiments, the methods described herein for generating T cells, such as immune evasive allogeneic T cells, comprises selecting for cells containing the first transgene encoding a tolerogenic factor using positive selection for the tolerogenic factor. In some embodiments, the positive selection for the tolerogenic factor comprises selecting for T cells that express the tolerogenic factor on the cell surface by affinity binding, flow cytometry, and/or immunomagnetic selection using antibodies and/or other proteins that bind the tolerogenic factor. In some embodiments, the tolerogenic factor is CD47.

Several methods of sorting living cells based on whether and/or how much they express or do not express a specific protein on their cell surface are known to those of skill in the art. For example, fluorescence activated cell sorting (FACS) of live cells separates a population of cells into sub-populations based on fluorescent labeling using a flow cytometer. Cells stained using fluorophore-conjugated antibodies to an antigen or marker of interest, such as CD3, TCR, or CD47, are separated from one another depending on which fluorophore they have been stained with. For example, a cell expressing one cell marker is detected using an FITC-conjugated antibody that recognizes the marker, and another cell type expressing a different marker could be detected using a PE-conjugated antibody specific for that marker.

Another example of a cell sorting method is magnetic-activated cell sorting (MACS). MACS is a method for separation of various cell populations depending on their surface antigens, such as CD3, TCR, or CD47. The method uses superparamagnetic nanoparticles and columns. The superparamagnetic nanoparticles are of the order of 100 nm. They are used to tag the targeted cells in order to capture them inside the column. The column is placed between permanent magnets so that when the magnetic particle-cell complex passes through it, the tagged cells are captured. The column consists of steel wool which increases the magnetic field gradient to maximize separation efficiency when the column is placed between the permanent magnets. The MACS method allows cells to be separated by using magnetic nanoparticles coated with antibodies against a particular surface antigen, such as CD3, TCR, and/or CD47. This causes the cells expressing this antigen to attach to the magnetic nanoparticles. After incubating the beads and cells, the solution is transferred to a column in a strong magnetic field. In this step, the cells attached to the nanoparticles (expressing the antigen) stay on the column, while other cells (not expressing the antigen) flow through. With this method, the cells are separated positively or negatively with respect to the particular antigen(s). With positive selection, the cells expressing the antigen(s) of interest, which are attached to the magnetic column, are washed out to a separate vessel, after removing the column from the magnetic field. In some embodiments, positive selection methods are used to distinguish cells expressing endogenous tolerogenic factors from cells expressing tolerogenic factors encoded by transgenes. For example, endogenous expression levels of tolerogenic factors are generally lower than expression levels of tolerogenic factors encoded by transgenes. In these instances, a positive selection method could include contacting the cells with beads conjugated to a first antibody against the tolerogenic factor having a first avidity and/or a first affinity which may bind preferentially to cells expressing both exogenous transgene encoded tolerogenic factors as well as endogenous tolerogenic factor molecules. Any cells expressing mostly the endogenous tolerogenic factor would flow through the column. With negative selection, the antibody used is against surface antigen(s) which are known to be present on cells that are not of interest. After administration of the cells/magnetic nanoparticles solution onto the column the cells expressing these antigens bind to the column and the fraction that goes through is collected, as it contains almost no cells with these undesired antigens.

Another example of a cell sorting method is the Streptamer technology, which allows reversible isolation and staining of antigen-specific T cells. In principle, the T cells are separated by establishing a specific interaction between the T cell of interest and a molecule that is conjugated to a marker, which enables the isolation. The reversibility of this interaction and the fact that it is performed at low temperatures is the reason for the successful isolation and characterization of functional T cells. Because T cells remain phenotypically and functionally indistinguishable from untreated cells, this method offers new strategies in clinical and basic T cell research. The Streptamer staining principle combines the classic method of T cell isolation by MHC-multimers with the Strep-tag/Strep-Tactin technology. The Strep-tag is a short peptide sequence that displays moderate binding affinity for the biotin-binding site of a mutated streptavidin molecule, called Strep-Tactin. For the Streptamer technology, the Strep-Tactin molecules are multimerized, thus creating a platform for binding to strep-tagged proteins. Further, the Strep-Tactin backbone has a fluorescent label to allow flow cytometry analysis. Incubation of MHC-Strep-tag fusion proteins with the Strep-Tactin backbone results in the formation of an MHC-multimer, which is capable for antigen-specific staining of T cells.

Other examples of cell separation using methodological standards that ensure high purity are rapid and label-free separation procedures based on surface marker density. Exemplary procedures involve the use of an anti-surface marker antibody-immobilized cell-rolling column, that can separate cells depending on the surface marker density of the cell surfaces. In some embodiments, various conditions for the cell-rolling column are optimized including adjustment of the column tilt angle and medium flow rate.

In some embodiments, the T cells generated by methods according to various embodiments of the present technology have at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the T cells in the population having the first transgene encoding a tolerogenic factor (e.g., CD47) inserted into an endogenous TCR gene locus. In some embodiments, have at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the generated T cells have reduced expression of CD3 and/or increased expression of a tolerogenic factor (e.g., CD47) encoded by a transgene. In some embodiments, have at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% of the generated T cells have reduced expression of TCR and/or increased expression of a tolerogenic factor (e.g., CD47) encoded by a transgene. In any of these embodiments, the remainder T cells in the population do not possess the described selection characteristic(s).

### H. Insertion of a Second Transgene Encoding a CAR

In some embodiments, the methods described herein for generating a population of cells, such as immune evasive allogeneic T cells, may further comprise inserting a second transgene encoding one or more CARs to a genomic locus of the T cells, in order to generate CAR-T cells for use in cell-based therapies against various target antigens and/or cell surface molecules. This step of inserting a second transgene encoding one or more CARs may occur before, with, or after the step of inserting a first transgene encoding a tolerogenic factor. In some embodiments, the CAR is a GPRC5D CAR, and in these embodiments, the second transgene comprises a nucleotide sequence encoding a GPRC5D CAR as disclosed herein.

i. Multiple CARs

In some embodiments, the second transgene comprises two or more nucleotide sequences, each encoding a CAR targeting a specific target antigen as herein disclosed. In these embodiments, the second transgene encodes two or more different CARs specific to different target cell surface molecules or antigens (e.g., a GPRC5D CAR and a CD22 CAR). The two or more CARs may each comprise an extracellular binding domain specific to a specific target cell surface molecule, and may comprise the same, or one or more different, non-antigen binding domains. For example, the two or more CARs may comprise different signal peptides, hinge domains, transmembrane domains, costimulatory domains, and/or intracellular signaling domains, in order to minimize the risk of recombination due to sequence similarities. Or, alternatively, the two or more CARs may comprise the same non-antigen binding domains. In the embodiments where the same non-antigen binding domain(s) and/or backbone are used, it is optional to introduce codon divergence at the nucleotide sequence level to minimize the risk of recombination. As one non-limiting example, the second transgene may comprise a nucleotide sequence encoding a GPRC5D CAR and a nucleotide sequence encoding a CD22 CAR. The GPRC5D CAR may comprise one transmembrane domain (e.g., CD28 transmembrane domain) while the CD22 CAR comprises a different transmembrane domain (e.g., CD8a transmembrane domain), or vice versa. As another non-limiting example, the GPRC5D CAR may comprise one costimulatory domain (e.g., 4-1BB costimulatory domain) while the CD22 CAR comprises a different costimulatory domain (e.g., CD28 costimulatory domain), or vice versa. Or, alternatively, the CD22 CAR and the GPRC5D CARs may comprise the same non-antigen binding domains but have codon divergence introduced at the nucleotide sequence level to minimize the risk of recombination. In any of these embodiments, the two or more nucleotide sequences of the second transgene are connected by one or more cleavage sites as described (e.g., a 2A site and/or a furin site), in the form of polycistronic constructs as described herein.

ii. Regulatory Elements

In some embodiments, the second transgene encoding a CAR may comprise additional regulatory elements operatively linked to the CAR encoding sequence as described, including, for example, promoters, insulators, enhancers, polyadenylation (poly(A)) tails, and/or ubiquitous chromatin opening elements.

### I. Genomic Insertion

In some embodiments, the second transgene encoding a CAR is delivered into a host cell in the form of a vector for insertion into the host genome. In some embodiments, the insertion is random (i.e., insertion into a random genomic locus of the host cell) or targeted (i.e., insertion into a specific genomic locus of the host cell), using any of the random or site-directed insertion methods described herein.

In some embodiments, the first transgene encoding a tolerogenic factor and the second transgene encoding a CAR are introduced into a host for genomic insertion separately. In some embodiments, the first transgene encoding a tolerogenic factor and the second transgene encoding a CAR are introduced into a host for genomic insertion at the same time, via a single vector or multiple vectors. In embodiments where the first and the second transgene are delivered into a host cell together in a single vector, the first and the second transgene are designed as a polycistronic construct as described below.

### J. Polycistronic Constructs

In some embodiments, the first transgene encoding a tolerogenic factor and the second transgene encoding a CAR, and/or the multiple CAR encoding sequences of the second transgene, are in the form of polycistronic constructs. Polycistronic constructs have two or more expression cassettes for co-expression of two or more proteins of interest in a host cell. In some embodiments, the polycistronic construct comprises two expression cassettes, i.e., is bicistronic. In some embodiments, the polycistronic construct comprises three expression cassettes, i.e., is tricistronic. In some embodiments, the polycistronic construct comprises four expression cassettes, i.e., is quadcistronic. In some embodiments, the polycistronic construct comprises more than four expression cassettes. In any of these embodiments, each of the expression cassettes comprises a nucleotide sequence encoding a protein of interest (e.g., a tolerogenic factor, a suicide switch, a regulatory factor, an antibody or antigen binding fragment thereof, or a CAR). In some embodiments, the two or more genes being expressed are under the control of a single promoter and are separated from one another by one or more cleavage sites to achieve co-expression of the proteins of interest from one transcript. In other embodiments, the two or more genes are under the control of separate promoters.

In some embodiments, the two or more expression cassettes of the polycistronic construct are separated by one or more cleavage sites. As the name suggests, a polycistronic construct allows simultaneous expression of two or more separate proteins from one mRNA transcript in a host cell. Cleavage sites are used in the design of a polycistronic construct to achieve such co-expression of multiple genes.

In some embodiments, the one or more cleavage sites comprise one or more self-cleaving sites. In some embodiments, the self-cleaving site comprises a 2A site. 2A peptides are a class of 18-22 amino acid-long peptides first discovered in picornaviruses and can induce ribosomal skipping during translation of a protein, thus producing equal amounts of multiple genes from the same mRNA transcript. 2A peptides function to “cleave” an mRNA transcript by making the ribosome skip the synthesis of a peptide bond at the C-terminus, between the glycine (G) and proline (P) residues, leading to separation between the end of the 2A sequence and the next peptide downstream. There are four 2A peptides commonly employed in molecular biology, T2A, P2A, E2A, and F2A, the sequences of which are summarized in Table 24. A glycine-serine-glycine (GSG) linker is optionally added to the N-terminal of a 2A peptide to increase cleavage efficiency. The use of “( )” around a sequence in the present disclosure means that the enclosed sequence is optional.

In some embodiments, the one or more cleavage sites additionally comprise one or more protease sites. The one or more protease sites can either precede or follow the self-cleavage sites (e.g., 2A sites) in the 5′ to 3′ order. The protease site is cleaved by a protease after translation of the full transcript or after translation of each expression cassette such that the first expression product is released prior to translation of the next expression cassette. In these embodiments, having a protease site in addition to the 2A site, especially preceding the 2A site in the 5′ to 3′ order, may reduce the number of extra amino acid residues attached to the expressed proteins of interest. In some embodiments, the protease site comprises a furin site, also known as a Paired basic Amino acid Cleaving Enzyme (PACE) site. There are at least three furin cleavage sequences, FC1, FC2, and FC3, the amino acid sequences of which are summarized in Table 25. In some embodiments, one or more optional glycine-serine-glycine (GSG) sequences are included for cleavage efficiency.

In some embodiments, the one or more cleavage sites comprise one or more self-cleaving sites, one or more protease sites, and/or any combination thereof. For example, the cleavage site includes a 2A site alone. For another example, the cleavage site includes a FC2 or FC3 site, followed by a 2A site. In these embodiments, the one or more self-cleaving sites are the same or different. In some embodiments, the one or more protease sites are the same or different.

In some embodiments, the polycistronic construct are in the form of a vector. In some embodiments, any type of vector suitable for introduction of nucleotide sequences into a host cell is used, including, for example, plasmids, adenoviral vectors, adenoviral-associated vectors, retroviral vectors, lentiviral vectors, phages, and homology-directed repair (HDR)-based donor vectors.

### Gene Editing Systems for Insertion of Polynucleotide Encoding CAR

In some aspects, the first polynucleotide encoding a tolerogenic factor and/or the second polynucleotide encoding a CAR, or the polycistronic construct as herein disclosed are integrated into the genome of a host cell (e.g., a T cell) using methods and compositions described herein.

### A. Random Insertion

In some embodiments, the first polynucleotide encoding a tolerogenic factor and/or the second polynucleotide encoding a CAR are inserted into a random genomic locus of a host cell. As known to a person skilled in the art, viral vectors, including, for example, retroviral vectors, lentiviral vectors, adenoviral vectors, and adeno-associated viral vectors, are commonly used to deliver genetic material into host cells and randomly insert the foreign or exogenous gene into the host cell genome to facilitate stable expression and replication of the gene.

### B. Site-Directed Insertion (Knock-In)

In some embodiments, the first polynucleotide encoding a tolerogenic factor and/or the second polynucleotide encoding a CAR are inserted into a specific genomic locus of the host cell. A number of gene editing methods are used to insert a polynucleotide (e.g., transgene) into a specific genomic locus of choice. Gene editing is a type of genetic engineering in which a nucleotide sequence is inserted, deleted, modified, or replaced in the genome of a living organism. In some embodiments, the gene editing technologies are systems involving nucleases, integrases, transposases, and/or recombinases. In some embodiments, the gene editing technology mediates single-strand breaks (SSB). In some embodiments, the gene editing technology mediates double-strand breaks (DSB), including in connection with non-homologous end-joining (NHEJ) or homology-directed repair (HDR). In some embodiments, the gene editing technologies are DNA-based editing or prime-editing. In some embodiments, the gene editing technology is Programmable Addition via Site-specific Targeting Elements (PASTE). In some embodiments, the gene editing technology is TnpB polypeptides. Many gene editing techniques generally utilize the innate mechanism for cells to repair double-strand breaks (DSBs) in DNA.

Eukaryotic cells repair DSBs by two primary repair pathways: non-homologous end-joining (NHEJ) and homology-directed repair (HDR). HDR typically occurs during late S phase or G2 phase, when a sister chromatid is available to serve as a repair template. NHEJ is more common and can occur during any phase of the cell cycle, but it is more error prone. In gene editing, NHEJ is generally used to produce insertion/deletion mutations (indels), which can produce targeted loss of function in a target gene by shifting the open reading frame (ORF) and producing alterations in the coding region or an associated regulatory region. HDR, on the other hand, is a preferred pathway for producing targeted knock-ins, knockouts, or insertions of specific mutations in the presence of a repair template with homologous sequences. Several methods are known to a skilled artisan to improve HDR efficiency, including, for example, chemical modulation (e.g., treating cells with inhibitors of key enzymes in the NHEJ pathway); timed delivery of the gene editing system at S and G2 phases of the cell cycle; cell cycle arrest at S and G2 phases; and introduction of repair templates with homology sequences. The methods provided herein may utilize HDR-mediated repair, NHEJ-mediated repair, or a combination thereof.

In some embodiments, the methods provided herein for HDR-mediated insertion utilize a site-directed nuclease, including, for example, zinc finger nucleases (ZFNs), transcription activator-like effector nucleases (TALENs), meganucleases, transposases, and clustered regularly interspaced short palindromic repeat (CRISPR)/Cas systems.

i. ZFNs

ZFNs are fusion proteins comprising an array of site-specific DNA binding domains adapted from zinc finger-containing transcription factors attached to the endonuclease domain of the bacterial FokI restriction enzyme. A ZFN may have one or more (e.g., 1, 2, 3, 4, 5, 6, 7, 8, 9, 10, or more) of the DNA binding domains or zinc finger domains. See, e.g., Carroll et al., Genetics Society of America (2011) 188:773-782; Kim et al., Proc. Natl. Acad. Sci. USA (1996) 93:1156-1160. Each zinc finger domain is a small protein structural motif stabilized by one or more zinc ions and usually recognizes a 3- to 4-bp DNA sequence. Tandem domains can thus potentially bind to an extended nucleotide sequence that is unique within a cell's genome.

Various zinc fingers of known specificity are combined to produce multi-finger polypeptides which recognize about 6, 9, 12, 15, or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers are engineered to bind a predetermined nucleic acid sequence. Criteria to engineer a zinc finger to bind to a predetermined nucleic acid sequence are known in the art. See, e.g., Sera et al., Biochemistry (2002) 41:7074-7081; Liu et al., Bioinformatics (2008) 24:1850-1857.

ZFNs containing FokI nuclease domains or other dimeric nuclease domains function as a dimer. Thus, a pair of ZFNs are required to target non-palindromic DNA sites. The two individual ZFNs must bind opposite strands of the DNA with their nucleases properly spaced apart. See Bitinaite et al., Proc. Natl. Acad. Sci. USA (1998) 95:10570-10575. To cleave a specific site in the genome, a pair of ZFNs are designed to recognize two sequences flanking the site, one on the forward strand and the other on the reverse strand. Upon binding of the ZFNs on either side of the site, the nuclease domains dimerize and cleave the DNA at the site, generating a DSB with 5′ overhangs. HDR can then be utilized to introduce a specific mutation, with the help of a repair template containing the desired mutation flanked by homology arms. The repair template is usually an exogenous double-stranded DNA vector introduced to the cell. See Miller et al., Nat. Biotechnol. (2011) 29:143-148; Hockemeyer et al., Nat. Biotechnol. (2011) 29:731-734.

ii. TALENs

TALENs are another example of an artificial nuclease which are used to edit a target gene. TALENs are derived from DNA binding domains termed TALE repeats, which usually comprise tandem arrays with 10 to 30 repeats that bind and recognize extended DNA sequences. Each repeat is 33 to 35 amino acids in length, with two adjacent amino acids (termed the repeat-variable di-residue, or RVD) conferring specificity for one of the four DNA base pairs. Thus, there is a one-to-one correspondence between the repeats and the base pairs in the target DNA sequences.

TALENs are produced artificially by fusing one or more TALE DNA binding domains (e.g., 1, 2, 3, 4, 5, 6, 7, 8, 9, 10 or more) to a nuclease domain, for example, a FokI endonuclease domain. See Zhang, Nature Biotech. (2011) 29:149-153. Several mutations to FokI have been made for its use in TALENs; these, for example, improve cleavage specificity or activity. See Cermak et al., Nucl. Acids Res. (2011) 39:e82; Miller et al., Nature Biotech. (2011) 29:143-148; Hockemeyer et al., Nature Biotech. (2011) 29:731-734; Wood et al., Science (2011) 333:307; Doyon et al., Nature Methods (2010) 8:74-79; Szczepek et al., Nature Biotech (2007) 25:786-793; Guo et al., J. Mol. Biol. (2010) 200:96. The FokI domain functions as a dimer, requiring two constructs with unique DNA binding domains for sites in the target genome with proper orientation and spacing. Both the number of amino acid residues between the TALE DNA binding domain and the FokI nuclease domain and the number of bases between the two individual TALEN binding sites appear to be important parameters for achieving high levels of activity. Miller et al., Nature Biotech. (2011) 29:143-148.

By combining engineered TALE repeats with a nuclease domain, a site-specific nuclease is produced specific to any desired DNA sequence. Similar to ZFNs, TALENs are introduced into a cell to generate DSBs at a desired target site in the genome, and so are used to knock out genes or knock in mutations in similar, HDR-mediated pathways. See Boch, Nature Biotech. (2011) 29:135-136; Boch et al., Science (2009) 326:1509-1512; Moscou et al., Science (2009) 326:3501.

iii. Meganucleases

Meganucleases are enzymes in the endonuclease family which are characterized by their capacity to recognize and cut large DNA sequences (from 14 to 40 base pairs). Meganucleases are grouped into families based on their structural motifs which affect nuclease activity and/or DNA recognition. The most widespread and best known meganucleases are the proteins in the LAGLIDADG family, which owe their name to a conserved amino acid sequence. See Chevalier et al., Nucleic Acids Res. (2001) 29(18): 3757-3774. On the other hand, the GIY-YIG family members have a GIY-YIG module, which is 70-100 residues long and includes four or five conserved sequence motifs with four invariant residues, two of which are required for activity. See Van Roey et al., Nature Struct. Biol. (2002) 9:806-811. The His-Cys family meganucleases are characterized by a highly conserved series of histidines and cysteines over a region encompassing several hundred amino acid residues. See Chevalier et al., Nucleic Acids Res. (2001) 29(18):3757-3774. Members of the NHN family are defined by motifs containing two pairs of conserved histidines surrounded by asparagine residues. See Chevalier et al., Nucleic Acids Res. (2001) 29(18):3757-3774.

Because the chance of identifying a natural meganuclease for a particular target DNA sequence is low due to the high specificity requirement, various methods including mutagenesis and high throughput screening methods have been used to create meganuclease variants that recognize unique sequences. Strategies for engineering a meganuclease with altered DNA-binding specificity, e.g., to bind to a predetermined nucleic acid sequence are known in the art. See, e.g., Chevalier et al., Mol. Cell. (2002) 10:895-905; Epinat et al., Nucleic Acids Res (2003) 31:2952-2962; Silva et al., J Mol. Biol. (2006) 361:744-754; Seligman et al., Nucleic Acids Res (2002) 30:3870-3879; Sussman et al., J Mol Biol (2004) 342:31-41; Doyon et al., J Am Chem Soc (2006) 128:2477-2484; Chen et al., Protein Eng Des Sel (2009) 22:249-256; Arnould et al., J Mol Biol. (2006) 355:443-458; Smith et al., Nucleic Acids Res. (2006) 363(2):283-294.

Like ZFNs and TALENs, Meganucleases can create DSBs in the genomic DNA, which can create a frame-shift mutation if improperly repaired, e.g., via NHEJ, leading to a decrease in the expression of a target gene in a cell. Alternatively, foreign DNA is introduced into the cell along with the meganuclease. Depending on the sequences of the foreign DNA and chromosomal sequence, this process is used to modify the target gene. See Silva et al., Current Gene Therapy (2011) 11:11-27.

iv. Transposases

Transposases are enzymes that bind to the end of a transposon and catalyze its movement to another part of the genome by a cut and paste mechanism or a replicative transposition mechanism. By linking transposases to other systems such as the CRISPR/Cas system, new gene editing tools are developed to enable site specific insertions or manipulations of the genomic DNA. There are two known DNA integration methods using transposons which use a catalytically inactive Cas effector protein and Tn7-like transposons. The transposase-dependent DNA integration does not provoke DSBs in the genome, which may guarantee safer and more specific DNA integration.

v. CRISPR/Cas

The CRISPR system was originally discovered in prokaryotic organisms (e.g., bacteria and archaea) as a system involved in defense against invading phages and plasmids that provides a form of acquired immunity. Now it has been adapted and used as a popular gene editing tool in research and clinical applications.

CRISPR/Cas systems generally comprise at least two components: one or more guide RNAs (gRNAs) and a Cas protein. The Cas protein is a nuclease that introduces a DSB into the target site. CRISPR-Cas systems fall into two major classes: class 1 systems use a complex of multiple Cas proteins to degrade nucleic acids; class 2 systems use a single large Cas protein for the same purpose. Class 1 is divided into types I, III, and IV; class 2 is divided into types II, V, and VI. Different Cas proteins adapted for gene editing applications include, but are not limited to, Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. See, e.g., Jinek et al., Science (2012) 337 (6096):816-821; Dang et al., Genome Biology (2015) 16:280; Ran et al., Nature (2015) 520:186-191; Zetsche et al., Cell (2015) 163:759-771; Strecker et al., Nature Comm. (2019) 10:212; Yan et al., Science (2019) 363:88-91. The most widely used Cas9 is a type II Cas protein and is described herein as illustrative. In some embodiments, these Cas proteins are originated from different source species. For example, in some embodiments, Cas9 is derived from S. pyogenes or S. aureus.

In the original microbial genome, the type II CRISPR system incorporates sequences from invading DNA between CRISPR repeat sequences encoded as arrays within the host genome. Transcripts from the CRISPR repeat arrays are processed into CRISPR RNAs (crRNAs) each harboring a variable sequence transcribed from the invading DNA, known as the “protospacer” sequence, as well as part of the CRISPR repeat. Each crRNA hybridizes with a second transactivating CRISPR RNA (tracrRNA), and these two RNAs form a complex with the Cas9 nuclease. The protospacer-encoded portion of the crRNA directs the Cas9 complex to cleave complementary target DNA sequences, provided that they are adjacent to short sequences known as “protospacer adjacent motifs” (PAMs).

While the foregoing description has focused on Cas9 nuclease, it should be appreciated that other RNA-guided nucleases exist which utilize gRNAs that differ in some ways from those described to this point. For instance, Cpf1 (CRISPR from Prevotella and Franciscella 1; also known as Cas12a) is an RNA-guided nuclease that only requires a crRNA and does not need a tracrRNA to function.

Since its discovery, the CRISPR system has been adapted for inducing sequence specific DSBs and targeted genome editing in a wide range of cells and organisms spanning from bacteria to eukaryotic cells including human cells. In its use in gene editing applications, artificially designed, synthetic gRNAs have replaced the original crRNA:tracrRNA complexes, including in some embodiments via a single gRNA. For example, in some embodiments, the gRNAs are single guide RNAs (sgRNAs) composed of a crRNA, a tetraloop, and a tracrRNA. The crRNA usually comprises a complementary region (also called a spacer, usually about 20 nucleotides in length) that is user-designed to recognize a target DNA of interest. The tracrRNA sequence comprises a scaffold region for Cas nuclease binding. The crRNA sequence and the tracrRNA sequence are linked by the tetraloop and each have a short repeat sequence for hybridization with each other, thus generating a chimeric sgRNA. One can change the genomic target of the Cas nuclease by simply changing the spacer or complementary region sequence present in the gRNA. The complementary region will direct the Cas nuclease to the target DNA site through standard RNA-DNA complementary base pairing rules.

In order for the Cas nuclease to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene. For example, the most commonly used Cas9 nuclease derived from S. pyogenes recognizes a PAM sequence of 5′-NGG-3′ or, at less efficient rates, 5′-NAG-3′, where “N” is any nucleotide. Other Cas nuclease variants with alternative PAMs have also been characterized and successfully used for genome editing, which are summarized in Table 26.

In some embodiments, Cas nucleases may comprise one or more mutations to alter their activity, specificity, recognition, and/or other characteristics. For example, the Cas nuclease may have one or more mutations that alter its fidelity to mitigate off-target effects (e.g., eSpCas9, SpCas9-HF1, HypaSpCas9, HeFSpCas9, and evoSpCas9 high-fidelity variants of SpCas9). For another example, the Cas nuclease may have one or more mutations that alter its PAM specificity.

In some embodiments, CRISPR systems of the present disclosure comprise TnpB polypeptides. In some embodiments, TnpB polypeptides may comprise a Ruv-C-like domain. In some embodiments, the RuvC domain is a split RuvC domain comprising RuvC-I, RuvC-II, and RuvC-III subdomains. In some embodiments, a TnpB may further comprise one or more of a HTH domain, a bridge helix domain and a zinc finger domain. TnpB polypeptides do not comprise an HNH domain. In one exemplary embodiment, a TnpB protein comprises, starting at the N-terminus: a HTH domain, a RuvC-I subdomain, a bridge helix domain, a RuvC-II sub-domain, a zinger finger domain, and a RuvC-III sub-domain. In some embodiments, a RuvC-III sub-domain forms the C-terminus of a TnpB polypeptide. In some embodiments, a TnpB polypeptide is from Epsilonproteobacteria bacterium, Actinoplanes lobatus strain DSM 43150, Actinomadura celluolosilytica strain DSM 45823, Actinomadura namibiensis strain DSM 44197, Alicyclobacillus macrosprangiidus strain DSM 17980, Lipingzhangella halophila strain DSM 102030, or Ktedonobacter recemifer. In some embodiments, a TnpB polypeptide is from Ktedonobacter racemifer, or comprises a conserved RNA region with similarity to the 5′ ITR of K. racemifer TnpB loci. In some embodiments, a TnpB may comprise a Fanzor protein, a TnpB homolog found in eukaryotic genomes. In some embodiments, a CRISPR system comprising a TnpB polypeptide binds a target adjacent motif (TAM) sequence 5′ of a target polynucleotide. In some embodiments, a TAM is a transposon-associated motif. In some embodiments, a TAM sequence comprises TCA. In some embodiments, a TAM sequence comprises TTCAN. In some embodiments, a TAM sequence comprises TTGAT. In some embodiments, a TAM sequence comprises ATAAA.

In some embodiments, the first and/or the second transgene may function as a DNA repair template to be integrated into the target site through HDR in associated with a gene editing system (e.g., the CRISPR/Cas system) as described. Generally, the transgene to be inserted would comprise at least the expression cassette encoding the protein of interest (e.g., the tolerogenic factor or CAR) and would optionally also include one or more regulatory elements (e.g., promoters, insulators, enhancers). In some of these embodiments, the transgene to be inserted would be flanked by homologous sequence immediately upstream and downstream of the target, i.e., left homology arm (LHA) and right homology arm (RHA), specifically designed for the target genomic locus to serve as template for HDR. The length of each homology arm is generally dependent on the size of the insert being introduced, with larger insertions requiring longer homology arms.

In some embodiments, target-primed reverse transcription (TPRT) or prime editing is used to engineer exogenous genes, such as exogenous transgenes encoding a tolerogenic factor (e.g., CD47) into specific loci. In some embodiments, prime editing mediates targeted insertions, deletions, all 12 possible base-to-base conversions, and combinations thereof in human cells without requiring DSBs or donor DNA templates.

Prime editing is a genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some embodiments, prime editing is thought of as a “search-and-replace” genome editing technology since the prime editors search and locate the desired target site to be edited, and encode a replacement strand containing a desired edit which is installed in place of the corresponding target site endogenous DNA strand at the same time. For example, in some embodiments, prime editing is adapted for conducting precision CRISPR/Cas-based genome editing in order to bypass double stranded breaks. In some embodiments, a homologous protein is or encodes for a Cas protein-reverse transcriptase fusions or related systems to target a specific DNA sequence with a guide RNA, generate a single strand nick at the target site, and use the nicked DNA as a primer for reverse transcription of an engineered reverse transcriptase template that is integrated with the guide RNA. In some embodiments, a prime editor protein is paired with two prime editing guide RNAs (pegRNAs) that template the synthesis of complementary DNA flaps on opposing strands of genomic DNA, resulting in the replacement of endogenous DNA sequence between the PE-induced nick sites with pegRNA-encoded sequences.

In some embodiments, a gene editing technology is associated with a prime editor that is a reverse transcriptase, or any DNA polymerase known in the art. Thus, in one aspect, a prime editor may comprise Cas9 (or an equivalent napDNAbp) which is programmed to target a DNA sequence by associating it with a specialized guide RNA (i.e., PEgRNA) containing a spacer sequence that anneals to a complementary protospacer in the target DNA. Such methods include any disclosed in Anzalone et al., (doi.org/10.1038/s41586-019-1711-4), or in PCT publication Nos. WO2020191248, WO2021226558, or WO2022067130, which are hereby incorporated in their entirety.

In some embodiments, the base editing technology is used to introduce single-nucleotide variants (SNVs) into DNA or RNA in living cells. Base editing is a CRISPR-Cas9-based genome editing technology that allows the introduction of point mutations in RNAs or DNAs without generating DSBs. Base editors (BEs) are typically fusions of a Cas (“CRISPR-associated”) domain and a nucleobase modification domain (e.g., a natural or evolved deaminase, such as a cytidine deaminase that include APOBEC1 (“apolipoprotein B mRNA editing enzyme, catalytic polypeptide 1”), CDA (“cytidine deaminase”), and AID (“activation-induced cytidine deaminase”)) domains. In some embodiments, base editors may also include proteins or domains that alter cellular DNA repair processes to increase the efficiency and/or stability of the resulting single-nucleotide change. Two major classes of base editors have been developed: cytidine base editors (CBEs) (e.g., BE4) that allow C:G to T:A conversions and adenine base editors (ABEs) (e.g., ABE7.10) that allow A:T to G:C conversions. Base editors are composed by a catalytically dead Cas9 (dCas9) or a nickase Cas9 (nCas9) fused to a deaminase and guided by a sgRNA to the locus of interest. The d/nCas9 recognizes a specific PAM sequence and the DNA unwinds thanks to the complementarity between the sgRNA and the DNA sequence usually located upstream of the PAM (also called protospacer). Then, the opposite DNA strand is accessible to the deaminase that converts the bases located in a specific DNA stretch of the protospacer. Compared to HDR-based strategies, base editing is a promising tool to precisely correct genetic mutations as it avoids gene disruption by NHEJ associated with failed HDR-mediated gene correction. Rat deaminase APOBEC1 (rAPOBEC1) fused to deactivated Cas9 (dCas9) has been used to successfully convert cytidines to thymidines upstream of the PAM of the sgRNA. In some embodiments, this first BE system was optimized by changing the dCas9 to a “nickase” Cas9 D10A, which nicks the strand opposite the deaminated cytidine. Without being bound by theory, this is expected to initiate long-patch base excision repair (BER), where the deaminated strand is preferentially used to template the repair to produce a U:A base pair, which is then converted to T:A during DNA replication.

In some embodiments, a base editor is a nucleobase editor containing a first DNA binding protein domain that is catalytically inactive, a domain having base editing activity, and a second DNA binding protein domain having nickase activity, where the DNA binding protein domains are expressed on a single fusion protein or are expressed separately (e.g., on separate expression vectors). In some embodiments, a base editor is a fusion protein comprising a domain having base editing activity (e.g., cytidine deaminase or adenosine deaminase), and two nucleic acid programmable DNA binding protein domains (napDNAbp), a first comprising nickase activity and a second napDNAbp that is catalytically inactive, wherein at least the two napDNAbp are joined by a linker. In some embodiments, a base editor is a fusion protein that comprises a DNA domain of a CRISPR-Cas (e.g., Cas9) having nickase activity (nCas; nCas9), a catalytically inactive domain of a CRISPR-Cas protein (e.g., Cas9) having nucleic acid programmable DNA binding activity (dCas; e.g., dCas9), and a deaminase domain, wherein the dCas is joined to the nCas by a linker, and the dCas is immediately adjacent to the deaminase domain. In some embodiments, a base editor is an adenine-to-thymine or “ATBE” (or thymine-to-adenine or “TABE”) transversion base editor. Exemplary base editor and base editor systems include any as described in patent publication Nos. US20220127622, US20210079366, US20200248169, US20210093667, US20210071163, WO2020181202, WO2021158921, WO2019126709, WO2020181178, WO2020181195, WO2020214842, WO2020181193, which are hereby incorporated in their entirety.

In some embodiments, a gene editing technology is Programmable Addition via Site-specific Targeting Elements (PASTE). In some aspects, PASTE is platform in which genomic insertion is directed via a CRISPR-Cas9 nickase fused to both a reverse transcriptase and serine integrase. As described in Ioannidi et al. (doi.org/10.1101/2021.11.01.466786), PASTE does not generate double stranded breaks, but allows for integration of sequences as large as ˜36 kb. In some embodiments, a serine integrase is any known in the art. In some embodiments, a serine integrase has sufficient orthogonality such that PASTE is used for multiplexed gene integration, simultaneously integrating at least two different genes at at least two genomic loci. In some embodiments, PASTE has editing efficiencies comparable to or better than those of homology directed repair or non-homologous end joining based integration, with activity in non-dividing cells and fewer detectable off-target events.

### C. Genomic Loci for Insertion of the First Polynucleotide

In some embodiments, the genomic locus for site-directed insertion of the first polynucleotide (e.g., transgene) encoding a tolerogenic factor is an endogenous TCR gene locus. In some embodiments, the endogenous TCR gene locus is selected from the group consisting of a TRAC locus, a TRBC1 locus, and a TRBC2 locus. The specific site for insertion within a gene locus is located within any suitable region of the gene, including but not limited to a gene coding region (also known as a coding sequence or “CDS”), an exon, an intron, a sequence spanning a portion of an exon and a portion of an adjacent intron, or a regulatory region (e.g., promoter, enhancer). In some embodiments, the insertion occurs in one allele of the specific genomic locus. In some embodiments, the insertion occurs in both alleles of the specific genomic locus. In either of these embodiments, the orientation of the polynucleotide inserted into the target genomic locus is either the same or the reverse of the direction of the endogenous gene in that locus.

i. TRAC

TCRs recognize foreign antigens which have been processed as small peptides and bound to NMC molecules at the surface of antigen presenting cells (APC). Each TCR is a dimer consisting of one alpha and one beta chain (most common) or one delta and one gamma chain. The genes encoding the TCR alpha chain are clustered on chromosome 14. The TCR alpha chain is formed when one of at least 70 variable (V) genes, which encode the N-terminal antigen recognition domain, rearranges to 1 of 61 joining (J) gene segments to create a functional variable region that is transcribed and spliced to a constant region gene segment encoding the C-terminal portion of the molecule. The beta chain, on the other hand, is generated by recombination of the V, D (diversity), and J segment genes.

The TRAC gene encodes the TCR alpha chain constant region. The human TRAC gene resides on chromosome 14 at 22,547,506-22,552,156, forward strand. The TRAC genomic sequence is set forth in Ensembl ID ENSG00000277734.

ii. TRBC1 and TRBC2

The TRBC gene encodes the TCR beta chain constant region. TRBC1 and TRBC2 are analogs of the same gene, and T cells mutually exclusively express either TRBC1 and TRBC2. The human TRBC1 gene resides on chromosome 7 at 142,791,694-142,793,368, forward strand, and its genomic sequence is set forth in Ensembl ID ENSG00000211751. The human TRBC2 gene resides on chromosome 7 at 142,801,041-142,802,748, forward strand, and its genomic sequence is set forth in Ensembl ID ENSG00000211772.

### D. Genomic Loci for Insertion of the Second Polynucleotide

In some embodiments, the genomic locus for insertion of the second polynucleotide encoding a CAR as disclosed herein is a random locus (by random insertion) or a specific locus (by site-directed insertion). If a specific locus is desired, it is the same as or a different locus from that of the first transgene. In some embodiments, the genomic locus for insertion of the second transgene encoding a CAR is a specific locus selected from the group consisting of a TRAC locus, a TRBC1 locus, a TRBC2 locus, a B2M locus, a CIITA locus, and a safe harbor locus. Non-limiting examples of safe harbor loci include, but are not limited to, an AAVS1 (also known as PPP1R12C), ABO, CCR5, CLYBL, CXCR4, F3 (also known as CD142), FUT1, HMGB1, KDM5D, LRP1 (also known as CD91), MICA, MICB, RHD, ROSA26, and SHS231 gene locus. In some embodiments, the genomic locus for insertion of the second transgene encoding a CAR is a specific locus comprising a TRAC locus, a TRBC1 locus, a TRBC2 locus, a B2M locus, a CIITA locus, an AAVS1 (also known as PPP1R12C) locus, an ABO locus, a CCR5 locus, a CLYBL locus, aCXCR4 locus, an F3 (also known as CD142) locus, a FUT1 locus, an HMGB1 locus, a KDM5D locus, an LRP1 (also known as CD91) locus, a MICA locus, an MICB locus, an RHD locus, a ROSA26 locus, or an SHS231 locus. The second polynucleotide is inserted within any suitable region of any of the described locus, including but not limited to a gene coding region (also known as a coding sequence or “CDS”), an exon, an intron, a sequence spanning a portion of an exon and a portion of an adjacent intron, or a regulatory region (e.g., promoter, enhancer). In some embodiments, the insertion occurs in one allele of the genomic locus. In some embodiments, the insertion occurs in both alleles of the genomic locus. In either of these embodiments, the orientation of the polynucleotide inserted into the genomic locus is either the same or the reverse of the direction of the original gene in that locus. In some embodiments, the second polynucleotide is inserted with the first polynucleotide such as the first polynucleotide and the second polynucleotide are carried by a polycistronic vector.

E. Guide RNAs (gRNAs) for Site-Directed Insertion

In some embodiments, provided are gRNAs for use in site-directed insertion of a polynucleotide in according to various embodiments provided herein, especially in association with the CRISPR/Cas system. The gRNAs comprise a crRNA sequence, which in turn comprises a complementary region (also called a spacer) that recognizes and binds a complementary target DNA of interest. The length of the spacer or complementary region is generally between 15 and 30 nucleotides, usually about 20 nucleotides in length, although will vary based on the requirements of the specific CRISPR/Cas system. In some embodiments, the spacer or complementary region is fully complementary to the target DNA sequence. In other embodiments, the spacer is partially complementary to the target DNA sequence, for example at least 80%, 85%, 90%, 95%, 98%, or 99% complementary.

In some embodiments, the gRNAs provided herein further comprise a tracrRNA sequence, which comprises a scaffold region for binding to a nuclease. The length and/or sequence of the tracrRNA may vary depending on the specific nuclease being used for editing. In some embodiments, nuclease binding by the gRNA does not require a tracrRNA sequence. In those embodiments where the gRNA comprises a tracrRNA, the crRNA sequence may further comprise a repeat region for hybridization with complementary sequences of the tracrRNA.

In some embodiments, the gRNAs provided herein comprise two or more gRNA molecules, for example, a crRNA and a tracrRNA, as two separate molecules. In other embodiments, the gRNAs are single guide RNAs (sgRNAs), including sgRNAs comprising a crRNA and a tracrRNA on a single RNA molecule. In some of these embodiments, the crRNA and tracrRNA are linked by an intervening tetraloop.

In some embodiments, one gRNA is used in association with a site-directed nuclease for targeted editing of a gene locus of interest. In other embodiments, two or more gRNAs targeting the same gene locus of interest are used in association with a site-directed nuclease.

In some embodiments, exemplary gRNAs (e.g., sgRNAs) for use with various common Cas nucleases that require both a crRNA and tracrRNA, including Cas9 and Cas12b (C2c1), are provided in Table 27. See, e.g., Jinek et al., Science (2012) 337 (6096):816-821; Dang et al., Genome Biology (2015) 16:280; Ran et al., Nature (2015) 520:186-191; Strecker et al., Nature Comm. (2019) 10:212. For each exemplary gRNA, sequences for different portions of the gRNA, including the complementary region or spacer, crRNA repeat region, tetraloop, and tracrRNA, are shown. In some embodiments, the gRNA comprises all or a portion of the nucleotide sequences set forth in Table 27 and SEQ ID NOs: 179-182. In some embodiments, the gRNA comprises all or a portion of the nucleotide sequences set forth in SEQ ID NOs: 183-186. In some embodiments, the gRNA comprises all or a portion of the nucleotide sequences set forth in Table 27 and SEQ ID NOs: 187-190. In some embodiments, the gRNA comprises all or a portion of the nucleotide sequences set forth in Table 27 and SEQ ID NOs: 191-194.

In some embodiments, the gRNA comprises a crRNA repeat region comprising, consisting of, or consisting essentially of the nucleotide sequence set forth in SEQ ID NO: 180, SEQ ID NO: 184, SEQ ID NO: 188, or SEQ ID NO: 193. In some embodiments, the gRNA comprises a tetraloop comprising, consisting of, or consisting essentially of the nucleotide sequence set forth in Table 27 and SEQ ID NO: 181 (gaaa) or SEQ ID NO: 192 (aaaa). In some embodiments, the gRNA comprises a tracrRNA comprising, consisting of, or consisting essentially of the nucleotide sequence set forth in SEQ ID NO: 182, SEQ ID NO: 186, SEQ ID NO: 190, or SEQ ID NO: 191.

In some embodiments, the gRNA comprises a complementary region specific to a target gene locus of interest, for example, the TRAC locus, the TRBC1 locus, the TRBC2 locus, B2M locus, the CIITA locus, or a safe harbor locus selected from the group consisting of an AAVS1, ABO, CCR5, CLYBL, CXCR4, F3, FUT1, HMGB1, KDM5D, LRP1, MICA, MICB, RHD, ROSA26, and SHS231 gene locus. The complementary region may bind a sequence in any region of the target gene locus, including for example, a CDS, an exon, an intron, a sequence spanning a portion of an exon and a portion of an adjacent intron, or a regulatory region (e.g., promoter, enhancer). Where the target sequence is a CDS, exon, intron, or sequence spanning portions of an exon and intron, the CDS, exon, intron, or exon/intron boundary are defined according to any splice variant of the target gene. In some embodiments, the genomic locus targeted by the gRNA is located within 4000 bp, within 3500 bp, within 3000 bp, within 2500 bp, within 2000 bp, within 1500 bp, within 1000 bp, or within 500 bp of any of the loci or regions thereof as described. Further provided herein are compositions comprising one or more gRNAs provided herein and a Cas protein or a nucleotide sequence encoding a Cas protein. In some of these embodiments, the one or more gRNAs and a nucleotide sequence encoding a Cas protein are comprised within a vector, for example, a viral vector.

In some embodiments, provided are methods of identifying new loci and/or gRNA sequences for use in the site-directed genomic insertion approaches as described. For example, for CRISPR/Cas systems, when an existing gRNA for a particular locus (e.g., within an endogenous TCR gene locus) is known, an “inch worming” approach is used to identify additional loci for targeted insertion of transgenes by scanning the flanking regions on either side of the locus for PAM sequences, which usually occurs about every 100 base pairs (bp) across the genome. The PAM sequence will depend on the particular Cas nuclease used because different nucleases usually have different corresponding PAM sequences. The flanking regions on either side of the locus are between about 500 to 4000 bp long, for example, about 500 bp, about 1000 bp, about 1500 bp, about 2000 bp, about 2500 bp, about 3000 bp, about 3500 bp, or about 4000 bp long. When a PAM sequence is identified within the search range, a new guide is designed according to the sequence of that locus for use in site-directed insertion of transgenes. Although the CRISPR/Cas system is described as illustrative, in some embodiments, any gene editing approach as described is used in this method of identifying new loci, including those using ZFNs, TALENs, meganucleases, and transposases.

In some embodiments, the activity, stability, and/or other characteristics of gRNAs are altered through the incorporation of chemical and/or sequential modifications. As one example, transiently expressed or delivered nucleic acids are prone to degradation by, e.g., cellular nucleases. Accordingly, the gRNAs described herein can contain one or more modified nucleosides or nucleotides which introduce stability toward nucleases. While not being bound by a particular theory, it is believed that some modified gRNAs described herein can exhibit a reduced innate immune response when introduced into a population of cells, particularly the cells of the present technology. As used herein, the term “innate immune response” includes a cellular response to exogenous nucleic acids, including single stranded nucleic acids, generally of viral or bacterial origin, which involves the induction of cytokine expression and release, particularly the interferons, and cell death. Other common chemical modifications of gRNAs to improve stabilities, increase nuclease resistance, and/or reduce immune response include 2′-O-methyl modification, 2′-fluoro modification, 2′-O-methyl phosphorothioate linkage modification, and 2′-O-methyl 3′ thioPACE modification.

One common 3′ end modification is the addition of a poly(A) tract comprising one or more (and typically 5-200) adenine (A) residues. In some embodiments, the poly(A) tract is contained in the nucleic acid sequence encoding the gRNA or is added to the gRNA during chemical synthesis, or following in vitro transcription using a polyadenosine polymerase (e.g., E. coli poly(A) polymerase). In vivo, poly(A) tracts is added to sequences transcribed from DNA vectors through the use of polyadenylation signals. Examples of such signals are provided in Maeder. Other suitable gRNA modifications include, without limitations, those described in U.S. Patent Application No. US 2017/0073674 A1 and International Publication No. WO 2017/165862 A1, the entire contents of each of which are incorporated by reference herein.

In some embodiments, a tool for designing a gRNA as disclosed herein comprises: Benchling, Broad Institute GPP, CasOFFinder, CHOPCHOP, CRISPick, CRISPOR, Deskgen, E-CRISP, Geneious, Guides, Horizon Discovery, IDT, Off-Spotter, Synthego, or TrueDesign (ThermoFisher). One of ordinary skill in the art would understand that a tool that predicts both activity and specificity (e.g., to limit off-target modification) would be useful for designing a gRNA in some instances as disclosed herein.

F. Delivery of Gene Editing Systems into a Host Cell

In some embodiments, provided are compositions comprising one or more components of a gene editing system described herein, including one or more gRNAs, a site-directed nuclease (e.g., a Cas nuclease) or a nucleotide sequence encoding a site-directed nuclease protein, and a transgene for targeted insertion. In some embodiments, the compositions are formulated for delivery into a cell.

In some embodiments, components of a gene editing system provided herein, including one or more gRNAs, a site-directed nuclease (e.g., a Cas nuclease) or a nucleotide sequence encoding a site-directed nuclease protein, and a transgene (e.g., the first transgene encoding a tolerogenic factor and/or the second transgene encoding a CAR) for targeted insertion, are delivered into a cell in the form of a delivery vector. The delivery vector is any type of vector suitable for introduction of nucleotide sequences into a cell, including, for example, plasmids, adenoviral vectors, adeno-associated viral (AAV) vectors, retroviral vectors, lentiviral vectors, phages, and HDR-based donor vectors. The different components are introduced into a cell together or separately, and, in some embodiments, are delivered in a single vector or multiple vectors.

In some embodiments, the delivery vector is introduced into a cell by any known method in the field, including, for example, viral transformation, calcium phosphate transfection, lipid-mediated transfection, DEAE-dextran, electroporation, microinjection, nucleoporation, liposomes, nanoparticles, or other methods.

In some embodiments, the present technology provides compositions comprising a delivery vector according to various embodiments disclosed herein. In some embodiments, the compositions may further comprise one or more pharmaceutically acceptable carriers, excipients, preservatives, or a combination thereof. A “pharmaceutically acceptable carrier or excipient” refers to a pharmaceutically acceptable material, composition, or vehicle that is involved in carrying or transporting a compound of interest from one tissue, organ, or portion of the body to another tissue, organ, or portion of the body. For example, the carrier or excipient is a liquid or solid filler, diluent, excipient, solvent, or encapsulating material, or some combination thereof. Each component of the carrier or excipient must be “pharmaceutically acceptable,” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits. Suitable excipients include water, saline, dextrose, glycerol, or the like and combinations thereof. In some embodiments, compositions comprising cells as disclosed herein further comprise a suitable infusion media.

In some embodiments, provided are cells or compositions thereof comprising one or more components of a gene editing system described herein, including one or more gRNAs, a site-directed nuclease (e.g., a Cas nuclease) or a nucleotide sequence encoding a site-directed nuclease protein, and a transgene for targeted insertion.

### Methods of Treatment

In some aspects, the present technology provides methods for treating and/or preventing a disease in a subject in need thereof using T cells, such as immune evasive allogeneic T cells, derived from or generated by methods according to various embodiments disclosed herein. The method entails administering to the subject a therapeutically effective amount of the T cell, or a pharmaceutical composition containing the same.

In some embodiments, the T cell is an autologous cell, i.e., obtained from the subject who will receive the T cell after modification. In some embodiments, the T cell is an allogeneic T cell, i.e., obtained from someone other than the subject who will receive the T cell after modification. In either of these embodiments, the T cells is primary T cells obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, the T cells is derived from ESCs or iPSCs.

In some embodiments, the T cell is a naive T cell, a helper T cell (CD4+), a cytotoxic T cell (CD8+), a regulatory T cell (Treg), a central memory T cell (TCM), an effector memory T cell (TEM), a stem cell memory T cell (TSCM), or any combination thereof. In some embodiments, the T cell expresses a tolerogenic factor (e.g., CD47, HLA-E, HLA-G, PD-L1, CTLA-4) and/or a CAR (e.g., GPRC5D CAR). In these embodiments, the T cell recognizes and initiates an immune response to a cell expressing the antigen the CAR is designed to target (e.g., GPRC5D), and the T cell possesses hypoimmunity in an allogeneic recipient due to expression of the tolerogenic factor.

In some embodiments, the disease is cancer, for example, one associated with GPRC5D expression, i.e., the cancer cell expresses GPRC5D. In these embodiments, the method comprises contacting the cancer cell with a T cell generated by methods of the present technology and expressing the corresponding CAR, such that the CAR is activated in response to the antigen expressed on the cancer cell and subsequently initiates killing of the cancer cell.

In some embodiments, the cancer is a hematologic malignancy. Non-limiting examples of hematologic malignancies include myeloid neoplasm, myelodysplastic syndromes (MDS), myeloproliferative/myelodysplastic syndromes, acute lymphoblastic leukemia (ALL), chronic lymphocytic leukemia (CLL), acute myeloid leukemia (AML), chronic myelogenous leukemia (CML), blast crisis chronic myelogenous leukemia (bcCML), B-cell acute lymphoid leukemia (B-ALL), T-cell acute lymphoid leukemia (T-ALL), multiple myeloma (MM), T-cell lymphoma, and B-cell lymphoma.

In some embodiments, a cancer is solid malignancy. Non-limiting examples of solid malignancies comprise: breast cancer, ovarian cancer, colon cancer, prostate cancer, epithelial cancer, renal-cell carcinoma, pancreatic adenocarcinoma, cervical carcinoma, colorectal cancer, glioblastoma, rhabdomyosarcoma, neuroblastoma, melanoma, Ewing sarcoma, osteosarcoma, mesothelioma and adenocarcinoma.

In some embodiments, the disease is an autoimmune disease, including, for example, lupus, systemic lupus erythematosus, rheumatoid arthritis, psoriasis, psoriatic arthritis, multiple sclerosis, Crohn's disease, ulcerative colitis, Addison's disease, Graves' disease, Sjogren's syndrome, Hashimoto's thyroiditis, and celiac disease.

In some embodiments, the disease is diabetes mellitus, including, for example, Type I diabetes, Type II diabetes, prediabetes, and gestational diabetes.

In some embodiments, the disease is a neurological disease, including, for example, catalepsy, epilepsy, encephalitis, meningitis, migraine, Huntington's, Alzheimer's, Parkinson's, Pelizaeus-Merzbacher disease, and multiple sclerosis.

Provided herein are compositions suitable for use in a subject, including therapeutic compositions and cell therapy compositions. Provided herein are pharmaceutical compositions comprising a population of engineered cells as described herein and a pharmaceutically acceptable additive, carrier, diluent or excipient. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); salts such as sodium chloride; and/or non-ionic surfactants such as polysorbates (TWEEN™), poloxamers (PLURONICS™) or polyethylene glycol (PEG). In some embodiments, the pharmaceutical composition includes a pharmaceutically acceptable buffer (e.g., neutral buffer saline or phosphate buffered saline). In some embodiments, the pharmaceutically acceptable additive, carrier, diluent or excipient comprises one or more of Plasma-Lyte A®, dextrose, dextran, sodium chloride, human serum albumin (HSA), dimethylsulfoxide (DMSO), or a combination thereof. In some embodiments, the composition further comprises a pharmaceutically acceptable buffer. In some embodiments, the pharmaceutically acceptable buffer is neutral buffer saline or phosphate buffered saline.

In some embodiments, the T cell, or a pharmaceutical composition containing the same, according to the present technology is administered in a manner appropriate to the disease, condition, or disorder to be treated as determined by persons skilled in the medical art. In any of the above embodiments, the T cell, or a pharmaceutical composition containing the same, is administered intravenously, intraperitoneally, intratumorally, into the bone marrow, into a lymph node, or into the cerebrospinal fluid, so as to encounter the target antigen or cells. An appropriate dose, suitable duration, and frequency of administration of the compositions will be determined by such factors as a condition of the patient; size, type, and severity of the disease, condition, or disorder; the undesired type or level or activity of the tagged cells, the particular form of the active ingredient; and the method of administration.

In some embodiments, the amount of the T cells in a pharmaceutical composition is typically greater than 10² cells, for example, about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×1×10⁹, 5×10⁹, 1×10¹⁰, 5×10¹⁰ cells, or more.

In some embodiments, the methods comprise administering to the subject the T cell, or a pharmaceutical composition containing the same, once a day, twice a day, three times a day, or four times a day for a period of about 3 days, about 5 days, about 7 days, about 10 days, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 1 year, about 1.25 years, about 1.5 years, about 1.75 years, about 2 years, about 2.25 years, about 2.5 years, about 2.75 years, about 3 years, about 3.25 years, about 3.5 years, about 3.75 years, about 4 years, about 4.25 years, about 4.5 years, about 4.75 years, about 5 years, or more than about 5 years. In some embodiments, the host cells or the pharmaceutical composition containing the same is administered every day, every other day, every third day, weekly, biweekly (i.e., every other week), every third week, monthly, every other month, or every third month.

In some embodiments, the T cell, or a pharmaceutical composition containing the same, is administered over a pre-determined time period. Alternatively, the T cell, or a pharmaceutical composition containing the same, is administered until a particular therapeutic benchmark is reached. In some embodiments, the methods provided herein include a step of evaluating one or more therapeutic benchmarks in a biological sample, such as, but not limited to, the level of a cancer biomarker, to determine whether to continue administration of the host cell, or the pharmaceutical composition containing the same.

In some embodiments, the method further entails administering one or more other cancer therapies such as surgery, immunotherapy, radiotherapy, and/or chemotherapy to the subject, sequentially or simultaneously.

In some embodiments, the methods further comprise administering the subject a pharmaceutically effective amount of one or more additional therapeutic agents to obtain improved or synergistic therapeutic effects. In some embodiments, the one or more additional therapeutic agents are selected from the group consisting of an immunotherapy agent, a chemotherapy agent, and a biologic agent. In some embodiments, the subject was administered the one or more additional therapeutic agents before administration of the T cell, or a pharmaceutical composition containing the same. In some embodiments, the subject is co-administered the one or more additional therapeutic agents and the T cell, or a pharmaceutical composition containing the same. In some embodiments, the subject was administered the one or more additional therapeutic agents after administration of the T cell, or a pharmaceutical composition containing the same.

As one of ordinary skill in the art would understand, the one or more additional therapeutic agents and the T cell, or a pharmaceutical composition containing the same, is administered to a subject in need thereof one or more times at the same or different doses, depending on the diagnosis and prognosis of the subject. One skilled in the art would be able to combine one or more of these therapies in different orders to achieve the desired therapeutic results. In some embodiments, the combinational therapy achieves improved or synergistic effects in comparison to any of the treatments administered alone.

### EXAMPLES

The present disclosure may be further described by the following non-limiting examples, in which standard techniques known to the skilled artisan and techniques analogous to those described in these examples may be used where appropriate. It is understood that the skilled artisan will envision additional embodiments consistent with the disclosure provided herein.

### Example 1: In Vitro Production of GPRC5D CAR-T Cells

This example describes methods to generate and characterize CAR expression in T cells. The sequences for CAR1, CAR2, and CAR3 are as described in Table 28.

Cryopreserved CD8+ and CD4+ cells were thawed in a 37° C. water bath and transferred to a 50 mL conical tube containing CTS Optimizer media with 100 IU/mL IL-2, and pelleted at 500×g for 5 minutes. Cells were resuspended at a concentration of 4×10⁶ cells/mL (for MM.1S experiment) or 2×10⁶ cells/mL (for RPMI8226 experiment) in Complete CTS Optimizer with 100 IU/mL IL-2 and allowed to recover at 37° C. for 5 hours. Cells were then stimulated with CD3/CD28 CTS Dynabeads at a 1:1 bead to cell ratio at a final concentration of 1×10⁶ cells/mL in Complete CTS Optimizer with 100 IU/mL IL-2 and incubated at 37° C. overnight. The next day, cells were counted and CD4/CD8 cells were combined at a 1:1 ratio. A total of 1×10⁶ cells (for MM.1S experiment) or 2×10⁶ cells/mL (for RPMI8226 experiment) were added per well, and cells were transduced with vesicular stomatitis virus (VSV-G) pseudotyped GPRC5D lentiviral vectors (LVV) at a concentration of 10 SupT1 IU/well. Cells were then centrifuged at 1000×g for 90 minutes (for MM.1S experiment) or 60 minutes (for RPMI8226 experiment) at 32° C. After centrifugation, cells were incubated at 37° C. and 5% CO₂ overnight. In circumstances where the functional titer was too low to transduce at a concentration of 10 SupT1 IU/well, wells were instead dosed at a higher volume. After transduction, the beads were removed, and cells were washed and expanded up to a G-REX plate to allow for analysis by flow cytometry. Cellular expression of the GPRC5D CAR constructs in the MM.1S experiment was measured by flow cytometric analysis using FITC-labeled human anti-G4S (1:100), PE-labeled human anti-Whitlow (1:100), and APC-labeled human anti-CD34 (1:20). Cellular expression of the GPRC5D CAR constructs in the RPMI8226 experiment was measured by flow cytometric analysis using Viakrome 808 (1:2000) and APC-labeled human anti-CD34 (1:20). All experiments were normalized to live, CAR+ cells. Following confirmation of CAR expression, remaining cells were frozen at −80° C. Unless noted otherwise, the CAR constructs comprised a CD8 hinge and TM-41BBz backbone.

### Example 2: In Vitro Characterization of GPRC5D CAR-T Cells

This example describes methods used to characterize the cytotoxic effects of GPRC5D CAR-T cells and results of said characterization.

CAR T cells were prepared as described above.

### Luciferase

Luciferase-based in vitro characterization of GPRC5D CAR T cell cytotoxic effects was performed as follows: Target cells (MM.1S-ffluc tumor cells or RPMI8226-ffluc tumor cells) were counted, resuspended at a concentration of 0.2×10⁶ cells/mL, and 100 μL of cells were plated in a 96-well plate. MM.1S and RPMI-8226 cells were obtained from American Type Culture Collection (ATCC) and maintained in RPMI-1640+10% FBS. For functional killing assays performed in vitro and in vivo, target cell lines were transduced with mWasabi:ffluc or iRFP713 and flow sorted for purity. Effector cells (T cells from 2-3 donors transduced with GPRC5D CAR constructs as disclosed in Table 28, control clinical benchmark GPRC5D CAR construct, or mock) were plated in wells with target cells at effector cell:target cell ratios (E:T ratios) of 1:1, 1:2, 1:4, 1:8, 1:16, and 1:32, normalized to CAR transduction efficiency. After a 24-hour incubation, cells were pelleted and cell culture media was collected for cytokine release analysis. Pelleted cells were resuspended in 50 μL of Complete RPMI media and transferred to a black walled plate. 100 μL of Bright-Glo luciferase was added to each well, except for a control row, which received 100 μL Complete RPMI+10% FBS. Luminescence in target cells was measured on a SpectraMax plate reader within 10 minutes of addition of the Bright-Glo reagent. Data was plotted and analyzed by two-way ANOVA. Asterisks (*) represent statistical significance relative to the bb2121 control benchmark Symbols placed above data points represent less efficacy than bb2121.

The impact of the GPRC5D CAR T cells on cytokine production was also assessed as part of the luciferase assay. Supernatants were assayed for levels of IFNγ, GM-CSF, and IL-2 through a Meso Scale Discovery immunoassay based on the manufacturer's instructions.

FIGS. 1A and 1B show the results of the luciferase-based cytotoxic characterization of the GPRC5D CAR T cells in MM.1S and RPMI8226 cells, respectively. In MM.1S cells, CAR2 and CAR3 exhibited similar cytotoxic effects that were more cytotoxic than the clinical benchmark control at all ratios below an effector cell:target cell ratio of 1:16 (FIG. 1A). At a ratio of 1:1, CAR2 and CAR3 exhibited robust cytotoxic effects and less than 5% of MM.1S cells survived. CAR2 and CAR3 also exhibited similar cytotoxic effects at a ratio of 1:2 (approx. 15% survival), 1:4 (approx. 35% survival), and 1:8 ratios (approx. 85% survival). CAR1 was slightly less cytotoxic than the clinical benchmark, but still exhibited cytotoxic effects up to the 1:4 effector cell:target cell ratio (approx. 20% survival at 1:1 ratio, approx. 60% survival at 1:2 ratio, approx. 88% survival at 1:4 ratio). A similar trend was observed when the luciferase assay employed RPMI8226 cells, where CAR2 and CAR3 exhibited robust cytotoxic effects that were greater than the clinical benchmark at all tested effector cell:target cell ratios up until 1:32 (FIG. 1B). CAR2 and CAR3 exhibited similar cytotoxic effects at the 1:1 ratio (approx. 3% survival), 1:2 ratio (approx. 10% survival), 1:4 ratio (approx. 25% survival), and the 1:8 ratio (approx. 50% survival). CAR1 also exhibited cytotoxic effects, but similar to the results from the MM.1S assay, CAR1 exhibited cytotoxic effects, albeit at lower levels relative to the clinical benchmark (approx. 12% survival at 1:1 ratio, approx. 40% survival at 1:2 ratio, approx. 70% survival at 1:4 ratio, approx. 90% survival at 1:8 ratio).

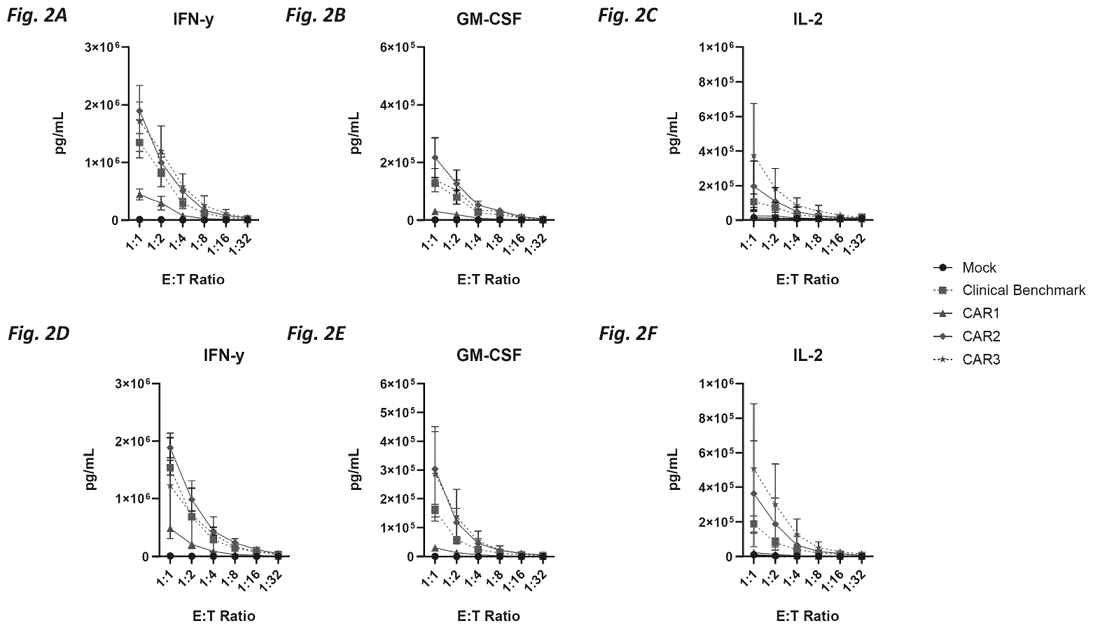

FIGS. 2A-2F show the impact of the GPRC5D CAR T cells on cytokine induction after a 24-hour incubation period with MM.1S cells (FIGS. 2A-2C) and RPMI8226 cells (FIGS. 2D-2F). In MM.1S cells, CAR2 and CAR3 robustly induced production of IFNγ, GM-CSF, and IL-2 at the 1:1 effector cell:target cell ratio (FIGS. 2A-2C). There was a dose-dependent decline as the effector cell:target cell ratio increased, but CAR2 and CAR3 still induced production of IFNγ, GM-CSF, and to a lesser extent, IL-2, at levels greater than or equivalent to the clinical benchmark at the 1:2 and 1:4 ratios. CAR1 induced modest production of IFNγ at the 1:1 and 1:2 ratios, but these levels were below the clinical benchmark. A similar trend was observed in the RPMI8226 cell line, where CAR2 and CAR3 induced robust production of IFNγ, GM-CSF, and IL-2 at both the 1:1 and 1:2 effector cell:target cell ratios that was greater than or approximately equivalent to the clinical benchmark (FIGS. 2D-2F). At the 1:4 ratio, CAR2 and CAR3 still induced production of IFNγ and GM-CSF at levels equivalent to or greater than the clinical benchmark. CAR1 induced modest production of IFNγ at the 1:1 and 1:2 effector cell:target cell ratios, but similar to the induction in MM.1S cells, these levels were below the clinical benchmark. For all tested CARs, and in both cell lines, a dose-dependent decrease was generally observed relating to the CAR-mediated cytokine production of IFNγ, GM-CSF, and IL-2.

### Live Cell Imaging

In vitro characterization of GPRC5D CAR-T cytotoxic effects by live cell imaging in the MM.1S cell experiment was performed as follows: CAR T cells were thawed and placed in CTS Optimizer media containing 100 IU/mL IL-2 overnight. Target cells (MM.1S cells) were harvested and resuspended at a concentration of 0.2×10⁶ cells/mL (for suspension targets) and 0.1×10⁶ cells/mL (for adherent targets). 100 μL of cells were pipetted into each well of a flat-bottom plate. Effector cells (T cells were collected from 1-2 donors transduced with GPRC5D CAR constructs as disclosed in Table 28, control GPRC5D CAR construct, or mock) were plated in wells with target cells at an effector cell:target cell ratio (E:T ratio) of 1:8. Cells were imaged every 4 hours for 6 days on a Sartorius IncuCyte instrument. Supernatant was collected after 24 hours of co-culturing to assess cytokine release. Tumor cells alone were added to assess alloreactivity.

In vitro characterization of GPRC5D CAR-T cytotoxic effects by live cell imaging in the RPMI8226 cell experiment was performed as follows: CAR T cells were thawed and placed in CTS Optimizer media containing 100 IU/mL IL-2 overnight. Target cells (RPMI8226 cells) were harvested and resuspended at a concentration of 0.2×10⁶ cells/mL in RPMI with 10% FBS. 100 μL of cells were pipetted into each well of a flat-bottom plate. Effector cells (T cells were collected from 1-2 donors transduced with GPRC5D CAR constructs as disclosed in Table 28, control GPRC5D CAR construct, or mock) were plated in wells with target cells at an effector cell:target cell ratio (E:T ratio) of 1:8. Cells were imaged every 4 hours for 6 days on a Sartorius IncuCyte instrument. Supernatant was collected after 24 hours of co-culturing to assess cytokine release. Tumor cells alone were added to assess alloreactivity.

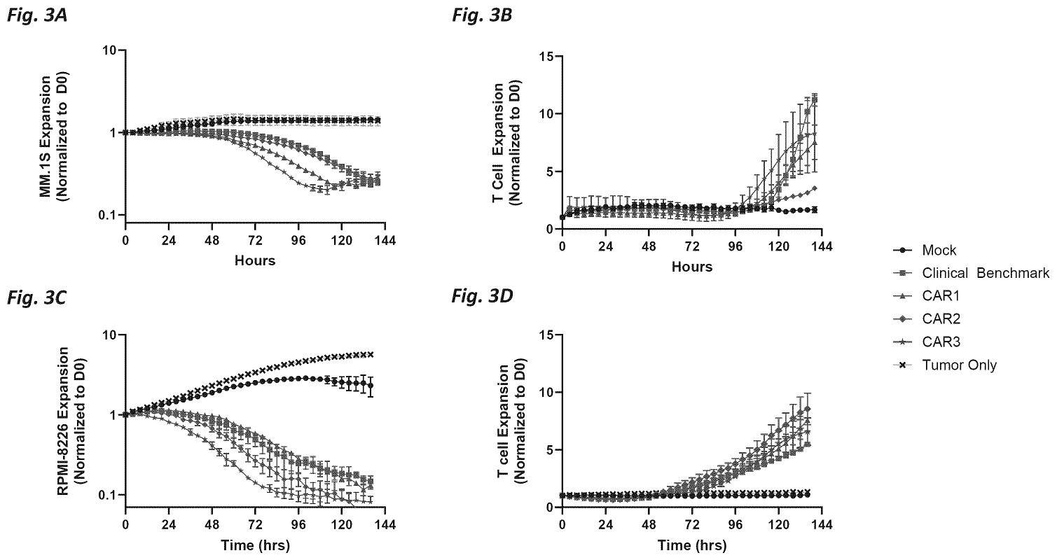

FIGS. 3A and 3B show the expansion of MM.1S cells and co-incubated CAR T cells, respectively, after culturing MM.1S target cells with T cells transduced with GPRC5D CARs over a 6-day period. CAR1, CAR2, and CAR3 all performed similar to the clinical GPRC5D CAR benchmark construct, where no measurable MM.1S cell expansion was recorded at any time point over the 6-day period (FIG. 3A), and there was an appreciable decrease in MM.1S cell expansion for all tested CARs, with CAR1 and CAR3 appearing slightly more efficacious at 96 and 120 hours. However, all tested CARs exhibited the same level of control of MM.1S expansion at the conclusion of the study. Robust CAR T cell expansion was also measured starting after 96 hours for the CAR1 and CAR3 T cells, and this expansion was comparable to the clinical benchmark control (FIG. 3B). CAR2 T cells also expanded after 96 hours, although this was at a level that was lower than CAR1, CAR3, and the clinical benchmark control.

FIGS. 3C and 3D show the expansion of RPMI8226 cells and co-incubated CAR T cells, after culturing RPMI8826 target cells with T cells transduced with GPRC5D CARs over a 6-day period. CAR1, CAR2, and CAR3 all performed similar to the clinical benchmark CAR, and prevented RPMI8226 cell expansion over the 6-day period, resulting in a decrease in RPMI8226 cell expansion at the conclusion of the study (FIG. 3C). CAR3 appeared to be the most efficacious and demonstrated the earliest control of RPMI8226 cell expansion of all the tested CARs. Further, robust CAR T cell expansion was measured for all tested CARs, with expansion for CAR1, CAR2, and CAR3 all at levels similar to the clinical benchmark control (FIG. 3D).

### CAR Optimization

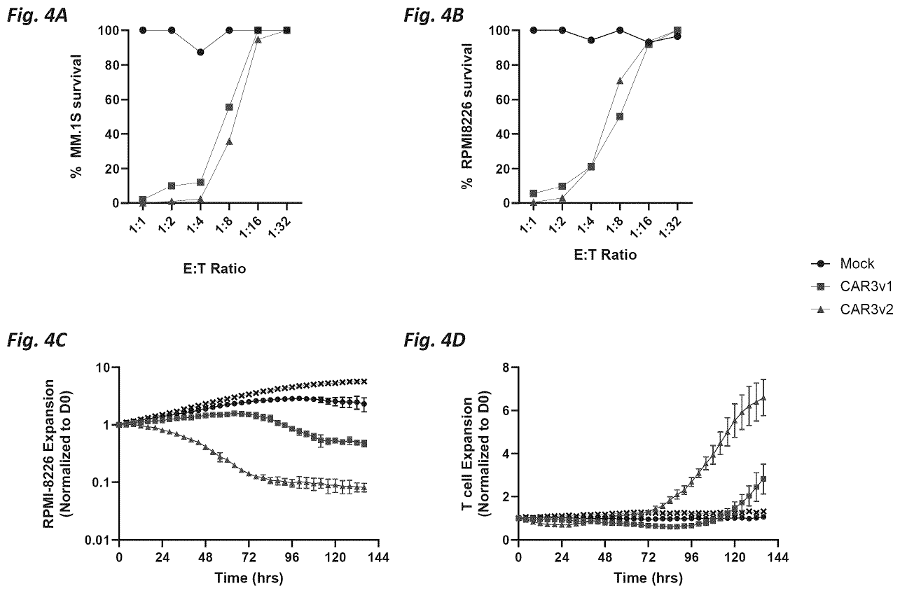

Specific GPRC5D CAR constructs were prepared as described above, and optimized using the in vitro luciferase cytotoxicity assay and live-cell imaging assay described above. FIGS. 4A and 4B show the impact of two different versions of CAR3 on MM.1S (FIG. 4A) and RPMI8226 (FIG. 4B) cell survival. Notably, both GPRC5D CAR constructs exhibited similar cytotoxic effects in controlling tumor cell growth, with CAR3v1 (GPRC5D CAR with a CD8 hinge/TM-41BBz; represented by squares in FIGS. 4A-4B) showing slightly greater cytotoxicity than CAR3v2 (GPRC5D CAR with a IgG4-CH₂—CH₃ hinge/CD28TM-41BBz; represented by triangles in FIGS. 4A-4B) in the MM.1S cell line. Both constructs exhibited significant cytotoxicity at 1:1, 1:2, and 1:4 effector cell:target cell ratios in MM.1S cells (mean of approx. 2% survival, approx. 5% survival, and approx. 7% survival, respectively, for the two constructs) and RPMI8226 cells (mean of approx. 3% survival, approx. 6% survival, and approx. 20% survival, respectively, for the two constructs). Cytotoxic effects were also observed at an effector cell:target cell ratio of 1:8 for both cell lines, although this was at lesser effect relative to the 1:1, 1:2, and 1:4 ratios.

FIG. 4C and FIG. 4D show the impact of the optimized GPRC5D CAR constructs on RPMI8226 expansion and on T cell expansion after co-incubation over a 6-day period, respectively. A slight increase in RPMI8226 cell expansion was noted over the first 3 days for CAR3v2 (GPRC5D CAR with a IgG4-CH₂—CH₃ hinge/CD28TM-41BBz; represented by triangles in FIGS. 4C-4D), before the construct controlled RPMI8226 cell expansion (FIG. 4C). Notably, CAR3v1 (GPRC5D CAR with a CD8 hinge/TM-41BBz; represented by squares in FIGS. 4C-4D) efficiently controlled RPMI8226 cell expansion over the course of the entire study and markedly decreased RPMI8226 cell expansion relative to the mock treated cells, and cells co-incubated with CAR3 (GPRC5D CAR with a CD8 hinge/TM-41BBz). There was also a robust expansion of T cells in the CAR3v1 (GPRC5D CAR with a CD8 hinge/TM-41BBz; represented by squares in FIGS. 4C-4D) co-incubated cell group, with rapid expansion starting at the 72-hour time point (FIG. 4D). T cell expansion in the CAR3v2 (GPRC5D CAR with a IgG4-CH₂—CH₃ hinge/CD28TM-41BBz; represented by triangles in FIGS. 4C-4D) group was also observed, although the T cell expansion was slightly delayed and at a lower level relative to CAR3v1 (GPRC5D CAR with a CD8 hinge/TM-41BBz; represented by squares in FIGS. 4C-4D).

This example describes methods used to characterize the efficacy of GPRC5D CAR-T cell constructs in a B-cell tumor animal model. Table 29 provides an overview of the different experimental groups for the in vivo study.

In vivo characterization of GPRC5D CAR efficacy was performed as follows: CAR T cells were produced as described above. Seven days prior to injection with CAR-T cells, 6-12 week old NOD.Cg-Prkd^{scid}Il2rg^{tm1Wj1}/SzJ (NSG) mice (The Jackson Laboratory) were intravenously injected with MM.1S:Wasabi-ffLuc cells (1×10⁷ cells/mouse) according to the experimental groups outlined in Table 29. Live imaging was performed one day later. Seven days after injection of the MM.1 S tumor cells, mice were intravenously injected with CAR-T cells (5×10⁶ or 1×10⁶ cells/mouse) or mock according to the experimental groups outlined in Table 29. Animals were imaged on day −6, day 4, day 7, day 11, day 14, day 18, day 21, day 25, day 28, day 33, day 36, day 39, and day 42. In vivo live imaging was performed at the indicated timepoints using an IVIS in vivo imaging instrument (Perkin-Elmer). In vivo live imaging measured bioluminescence via intraperitoneal injection of D-luciferin substrate (Perkin-Elmer). All images were analyzed using Living Image software (Perkin-Elmer). Survival of CAR-T treated mice was measured and reported using Kaplan-Meier curves. All in vivo animal studies were conducted in compliance with Institutional Animal Care and Use Committee (IACUC) approved protocols.

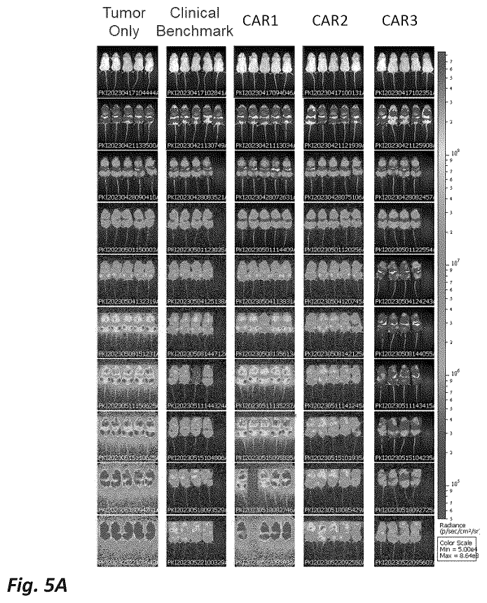

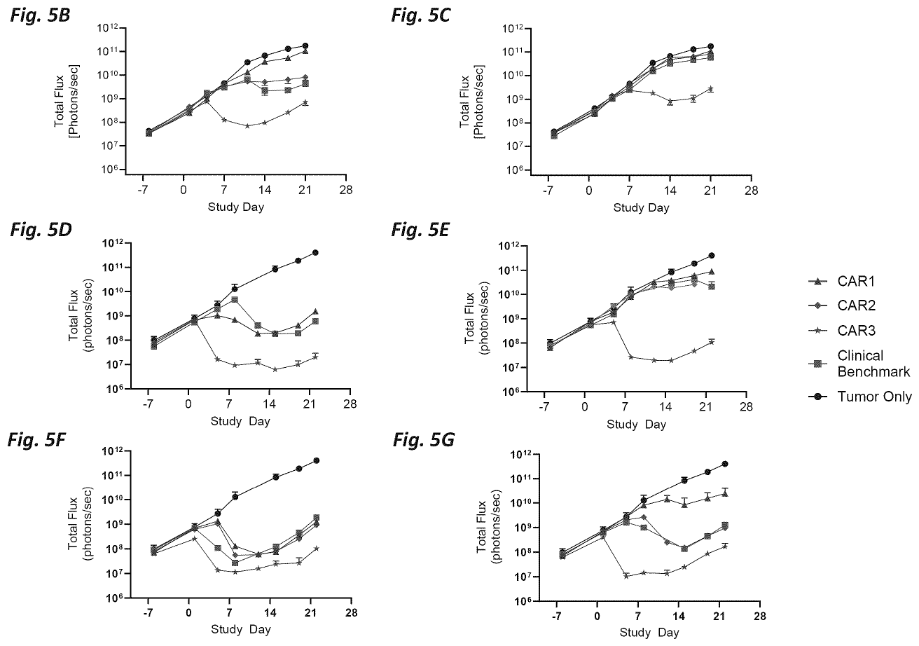

FIG. 5A shows representative images of animals in the different treatment groups and the flux associated with the different treatments. CAR2 and CAR3 exhibit a notable decrease in tumor cell flux relative to the tumor only control group. Animals in the CAR3 treatment group additionally exhibited decreased flux relative to the benchmark control group.

FIG. 5B-5G depicts normalized flux that illustrates tumor growth over the course of the study after mice were injected with GPRC5D CAR-T cells. FIGS. 5B-5C, 5D-5E, and 5F-5G depict results from CAR T cells generated from three different donors. Further, FIGS. 5B, 5D, and 5F depict results from animals receiving a CAR T cell dose of 5×10⁶ cells, and FIGS. 5C, 5E, and 5G depict results from animals receiving a CAR T cell dose of 1×10⁶ cells. Generally, animals receiving the higher dose of CAR cells (5×10⁶ cells) exhibited greater control of tumor growth. Notably, for all donor cells, and at both tested doses, CAR3 exhibited robust control of tumor growth relative to all other CARs tested, with tumor growth control starting around days 5-8. CAR1 and CAR2 also exhibited tumor growth control at the higher dose (5×10⁶ cells), which was generally similar to the tumor growth control observed in animals receiving the clinical benchmark. At the lower dose (1×10⁶ cells), CAR3 exhibited tumor growth control that was different than the mock control group. FIG. 5G also shows modest tumor growth control for CAR2 that was comparable to the benchmark control, and very modest tumor growth control for CAR1 starting at day 12.

### Example 3: In Vitro Production of GPRC5D CAR-T Cells

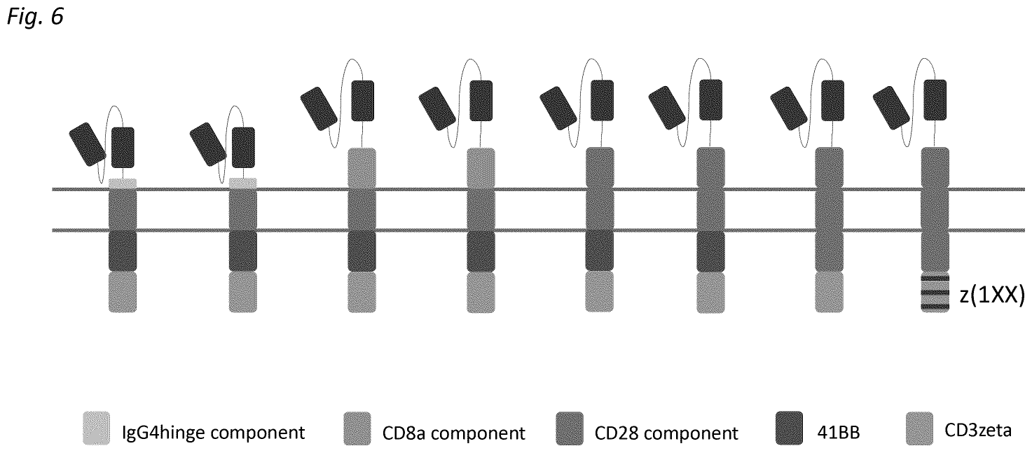

Cryopreserved CD8+ and CD4+ cells collected from 2 donors (apheresis donor 1 and apheresis donor 2) were thawed in a 37° C. water bath and transferred to a 50 mL conical tube containing CTS Optimizer media with 100 IU/mL IL-2, and pelleted at 500×g for 5 minutes. Cells from apheresis donor 1 and apheresis donor 2 were treated separately and the separation was maintained throughout in order to control for potential variability between donors. Cells were combined at a 1:1 ratio of CD8+ and CD4+ cells and were resuspended at a concentration of 1×10⁶ in Complete CTS Optimizer with 100 IU/mL IL-2. Cells were then stimulated with CD3/CD28 CTS Dynabeads at a 1:1 bead to cell ratio at a final concentration of 1×10⁶ cells/mL in Complete CTS Optimizer with 100 IU/mL IL-2 and incubated at 37° C. overnight. The next day, cells were counted and CD4/CD8 cells were combined at a 1:1 ratio. A total of 1×10⁶ cells were added per well, and cells were transduced with vesicular stomatitis virus (VSV-G) pseudotyped GPRC5D lentiviral vectors (LVV) at a concentration of 10 SupT1 IU/well. The cells were transduced with GPRC5D CAR constructs having the GPRC5D binding domain of GPRC5D Binder 3 (SEQ ID NO:27) and hinge, transmembrane, and signaling domains as disclosed in Table 30 and depicted in FIG. 6. Cells were then centrifuged at 1000×g for 60 minutes at 32° C. After centrifugation, cells were incubated at 37° C. and 5% CO₂ overnight. After transduction, the beads were removed, and cells were washed and expanded up in T Flasks to allow for analysis by flow cytometry. Cellular expression of the GPRC5D CAR constructs experiment was measured by flow cytometric analysis using PE-labeled human anti-G4S (1:100) and Viakrome 808 (1:2000). All experiments were normalized to live, CAR+ cells. Following confirmation of CAR expression, remaining cells were frozen at −80° C.

### Example 4: In Vitro Characterization of GPRC5D CAR-T Cells

This example describes methods used to characterize the cytotoxic effects of GPRC5D CAR-T cells and results of said characterization. The luciferase Cytotoxicity assay was intended to test the targeted cytotoxicity of GPRCSD-specific CAR T cells against GPRC5D antigen positive cells, MM.1S tagRFP:ffluc and NCI-H929 tagRFP:ffluc, via the loss of luciferase activity in targeted cell populations in a 18-24-hour period

CAR T cells were prepared as described above.

### Luciferase

Luciferase-based in vitro characterization of GPRC5D CAR T cell cytotoxic effects was performed as follows: Target cells (MM.1S-ffluc tumor cells or NCI-H929-ffluc tumor cells) were counted, resuspended at a concentration of 0.2×10⁶ cells/mL, and 100 μL of cells were plated in a 96-well plate. MM.1S and NCI-H929 cells were obtained from American Type Culture Collection (ATCC) and maintained in RPMI-1640+10% FBS. For functional killing assays performed in vitro, target cell lines were transduced with mWasabi:ffluc or iRFP713 and flow sorted for purity. Effector cells (T cells) from 2 donors expressing GPRC5D CAR constructs were plated in wells with target cells at effector cell:target cell ratios (E:T ratios) of 2:1 1:1, 1:2, 1:4, 1:8, 1:16, 1:32, and 1:64, normalized to CAR transduction efficiency. After a 24-hour incubation, cells were pelleted and were resuspended in 50 μL of Complete RPMI media and transferred to a black walled plate. 100 μL of Bright-Glo luciferase was added to each well, except for a control row, which received 100 μL Complete RPMI+10% FBS. Luminescence in target cells was measured on a SpectraMax plate reader within 10 minutes of addition of the Bright-Glo reagent.

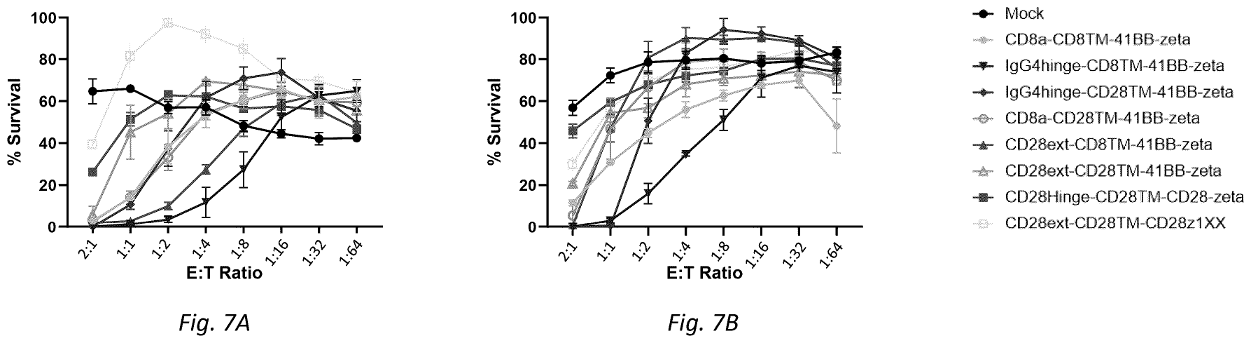

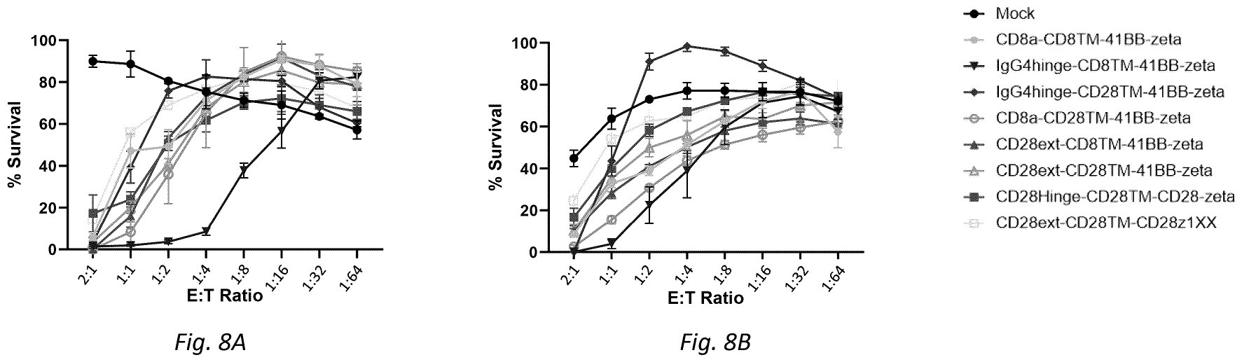

FIGS. 7 and 8 show higher E:T ratios (e.g., 2:1, 1:1, 1:2, 1:4) result in lower luciferase activity in targeted cell populations compared to lower ratios (e.g., 1:16, 1:32, 1:64). As the E:T ratio gets smaller there is an increase in the percent (%) survival of the GPRC5D antigen cells against the CAR positive cells. The data demonstrate that the various GPRC5D CAR constructs decrease the % survival of antigen positive target cells.

### Characterization of GPRC5D CAR T Cells Serial Tumor Rechallenge Assay

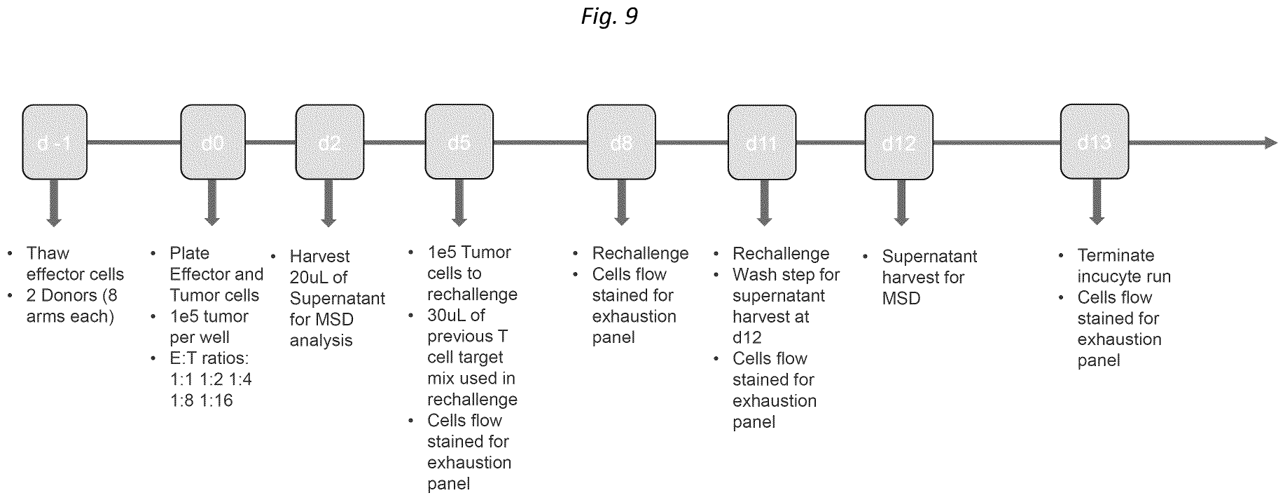

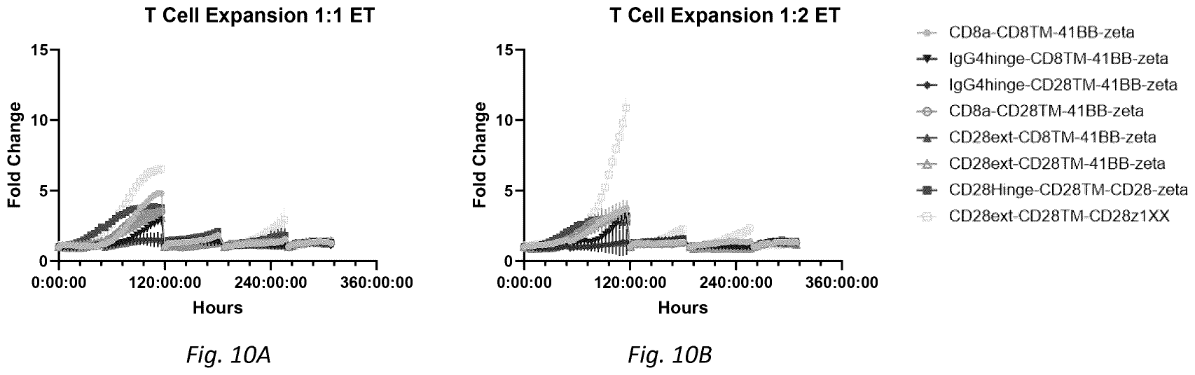

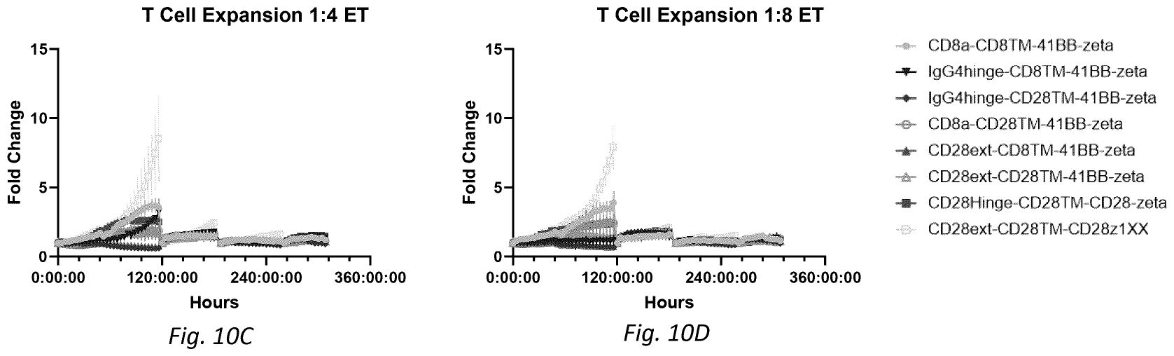

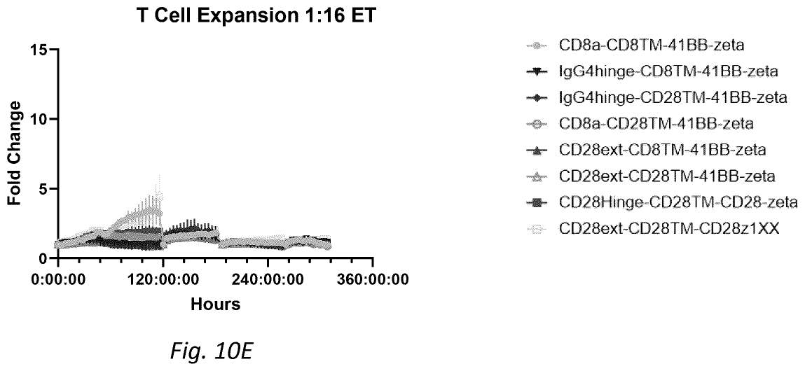

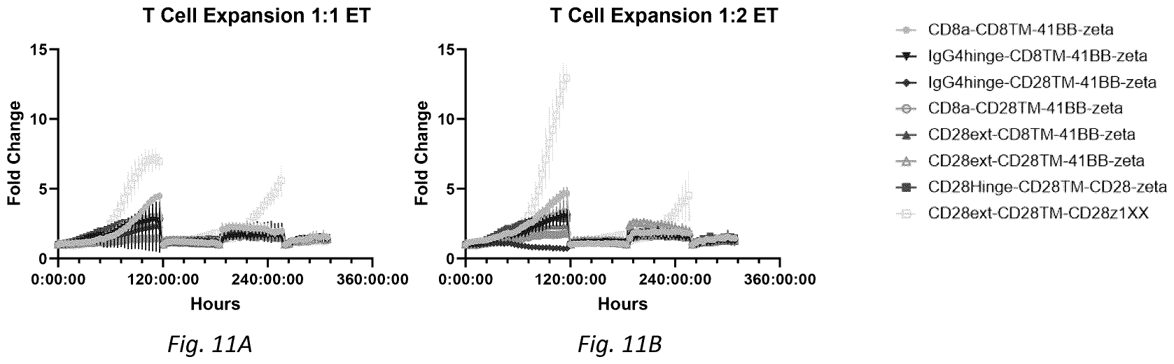

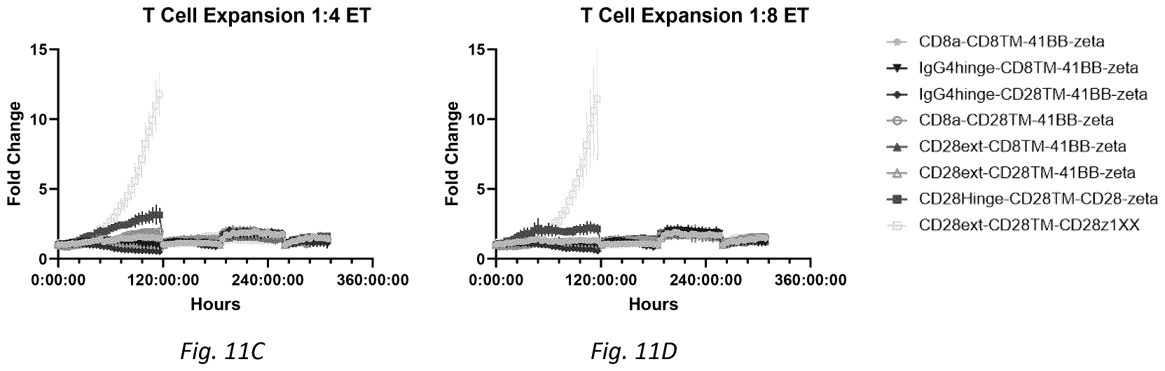

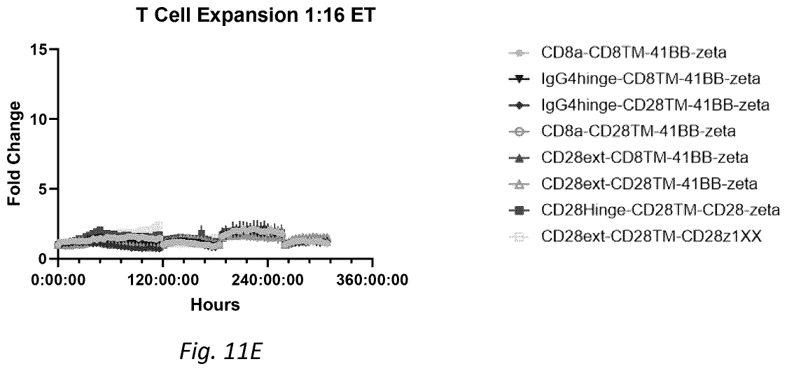

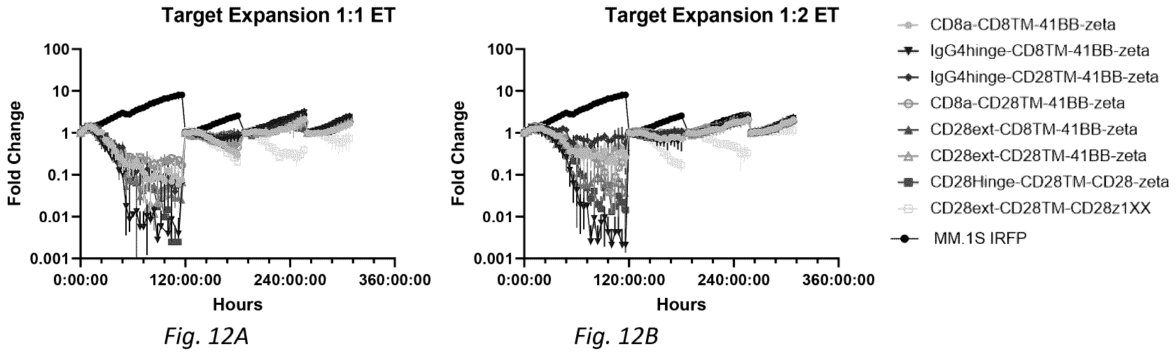

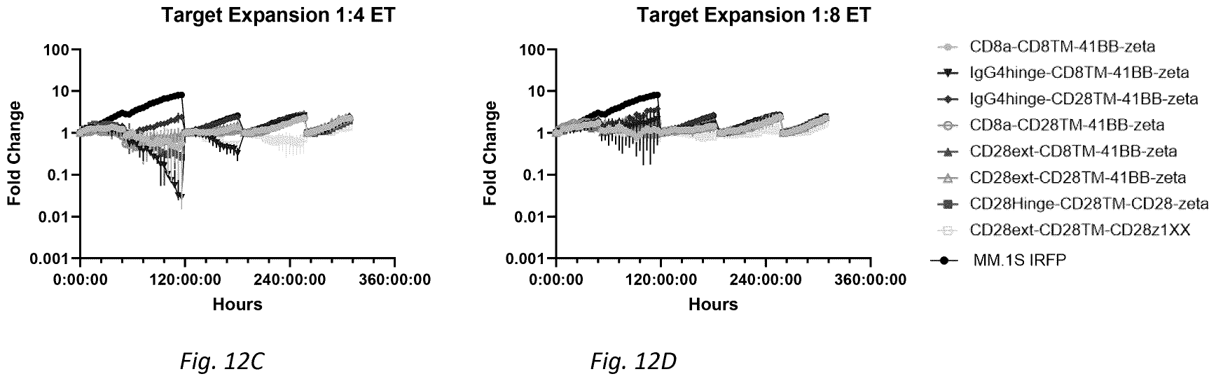

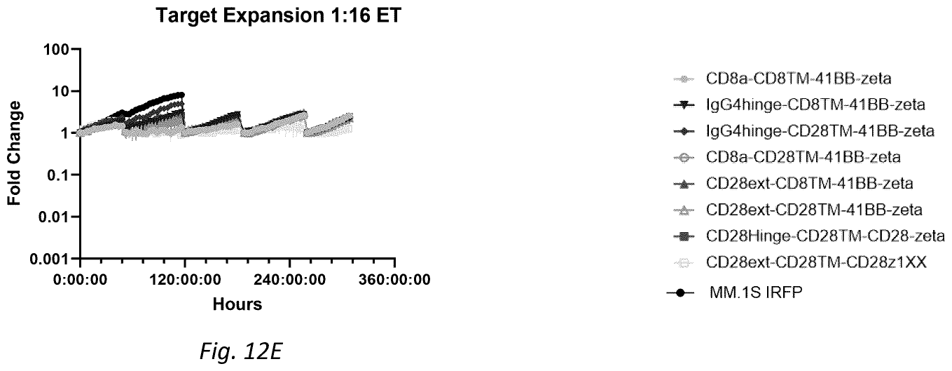

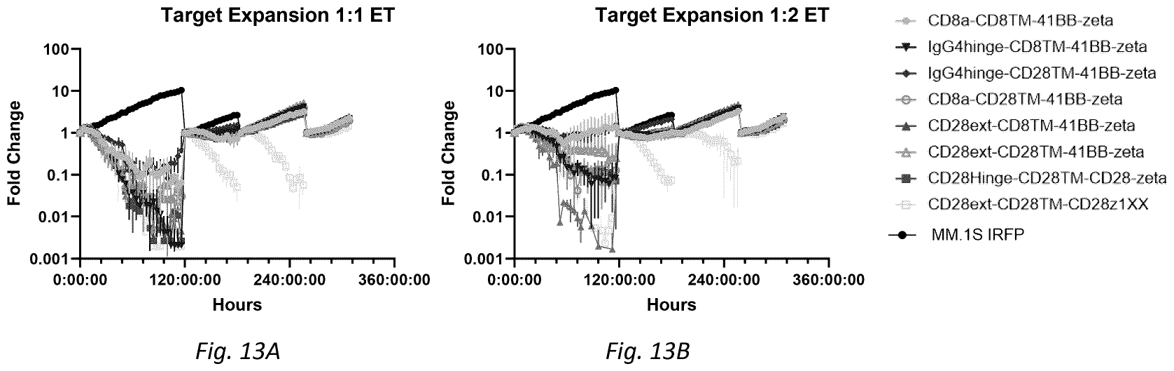

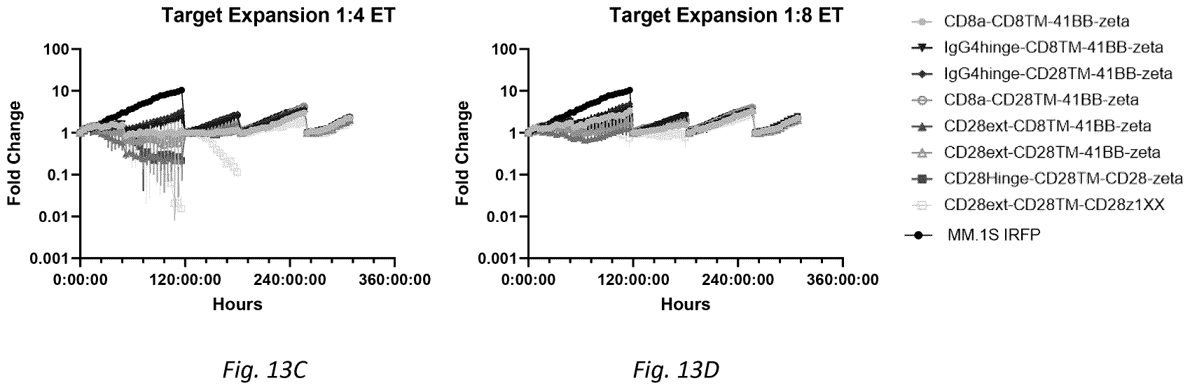

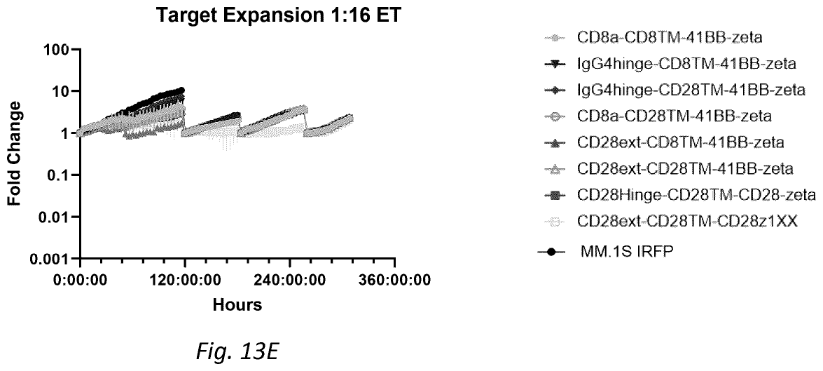

In vitro characterization of GPRC5D CAR-T cytotoxic effects by live cell imaging in the MM.1S cell experiment was performed as depicted in FIG. 9 and as follows: CAR T cells were thawed and placed in CTS Optimizer media containing 100 IU/mL IL-2 overnight. Target cells (MM.1S cells) were harvested and resuspended at a concentration of 0.1×10⁶ cells/mL in RPMI with 10% FBS. 100 μL of cells were pipetted into each well of a flat-bottom plate. T cells were generated as described above, i.e., transduced with CARs having the GPRC5D binding domain of GPRC5D Binder 3 (SEQ ID NO:27) and hinge, transmembrane, and signaling domains as disclosed in Table 30 and depicted in FIG. 6. The T cells were plated in wells with target cells at an effector cell:target cell ratio (E:T ratio) of 1:1, 1:2, 1:4, 1:8, 1:16. Cells were imaged every 4 hours for 13 days on a Sartorius IncuCyte instrument. Supernatant was collected after 48 hours of co-culturing to assess cytokine release. On Day 5, Day 8, and Day 11, each GPRC5D CAR at all E:T ratios were rechallenged with 1×10⁴ MM.1S Cells by adding 30 μL of cell suspension from the original flat bottom Incucyte plate to a new plate with 1×10⁴ MM.1S cells per well. FIGS. 10-11 show CAR T cell expansion at each stage and at each E:T ratio. FIGS. 12-13 show target cell expansion (MM.1S cells) at each stage and at each E:T ratio. The combined data show the potential of T cells expressing the various CAR construction to expand in response to target cells and control target cell expansion in vitro.

MSD Analysis of Supernatant from Incucyte Assay:

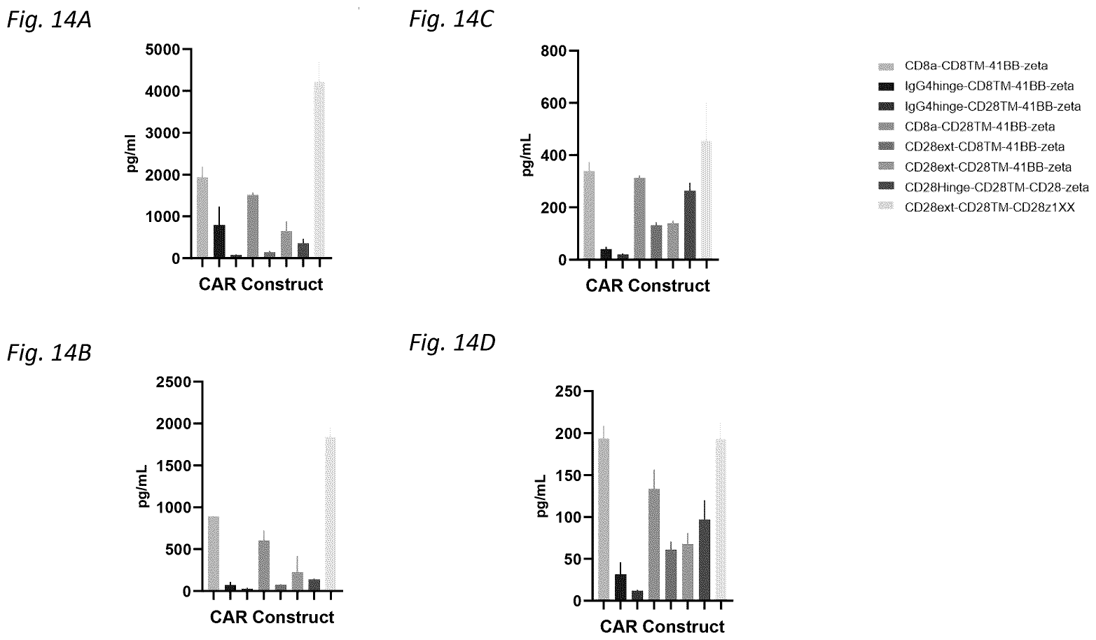

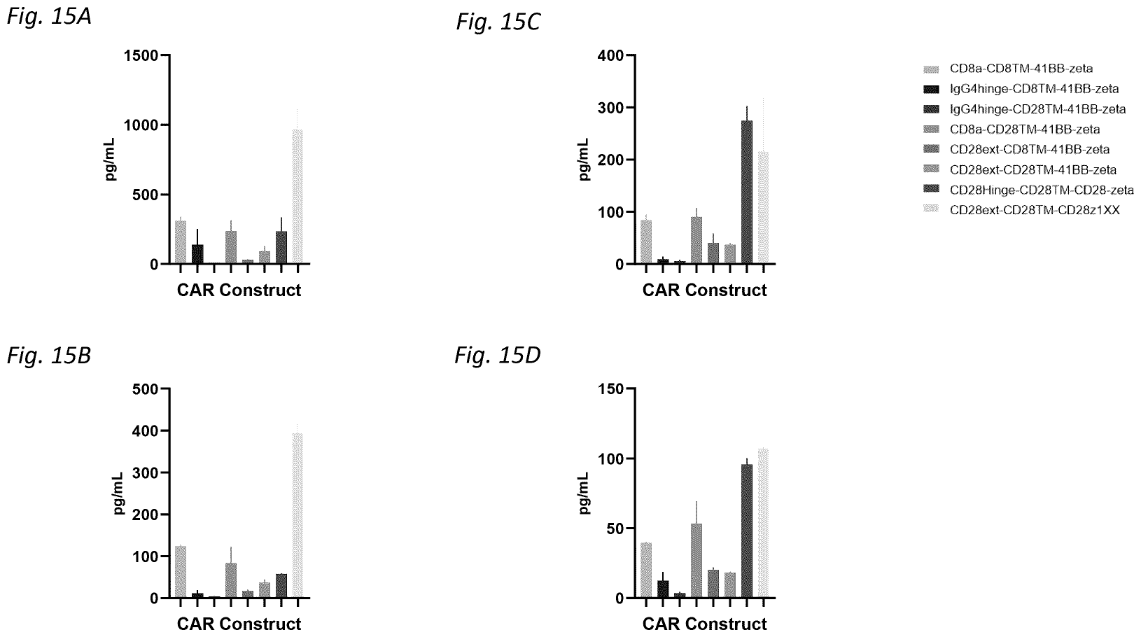

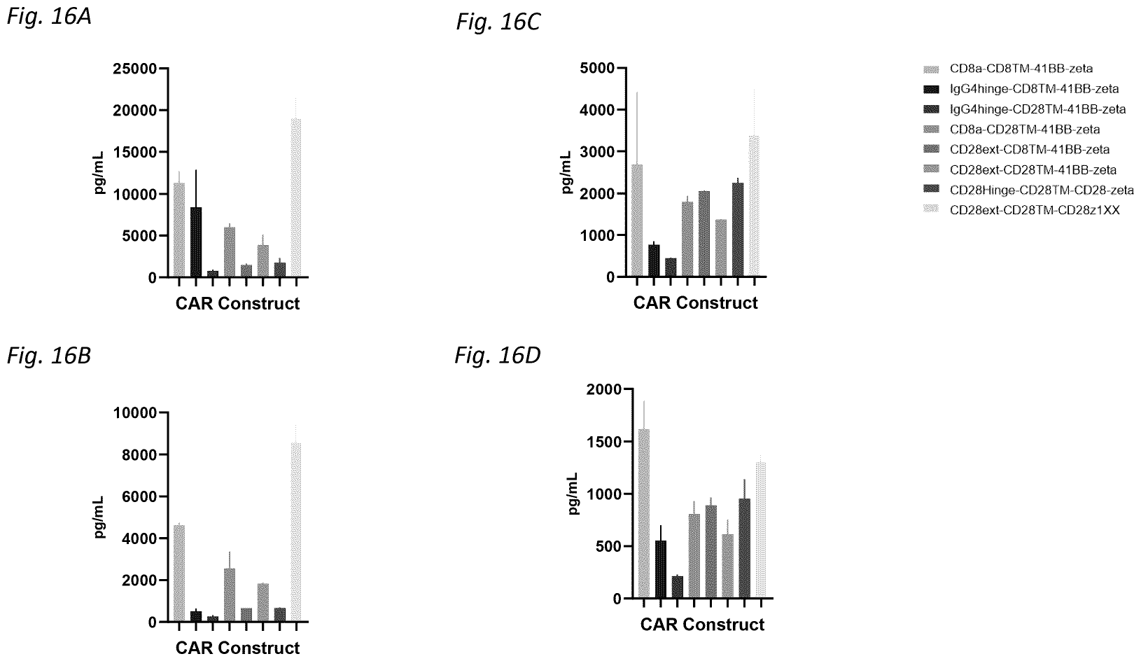

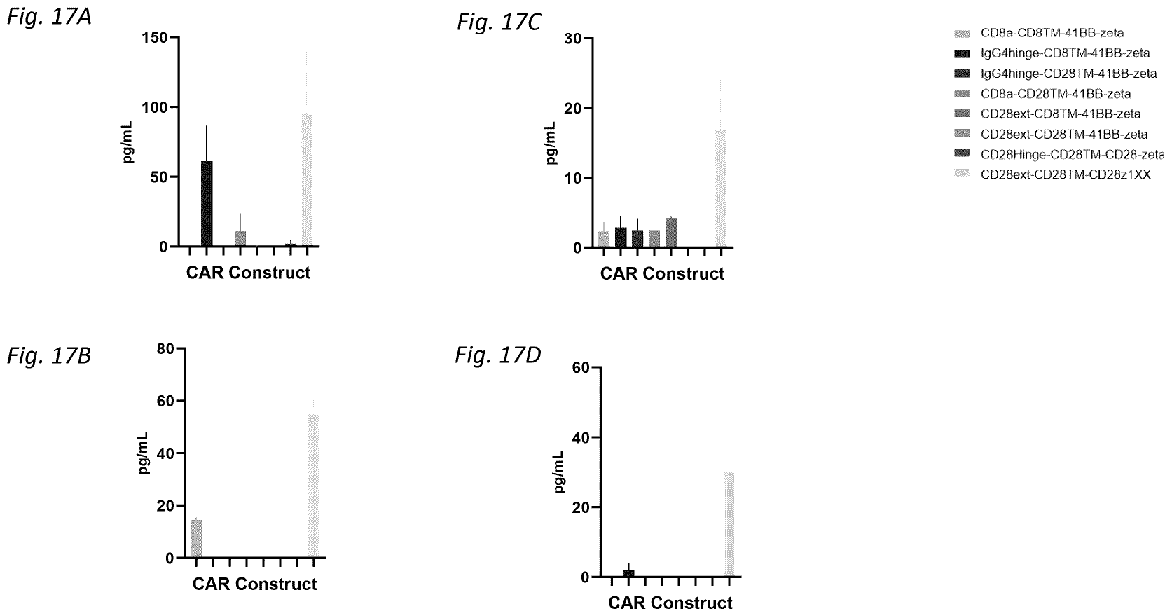

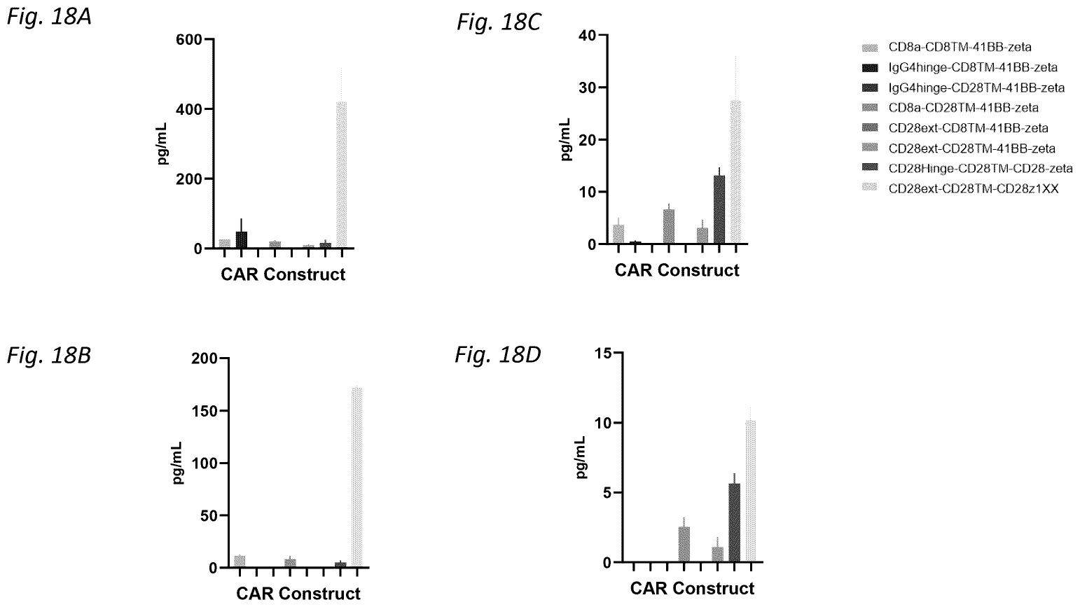

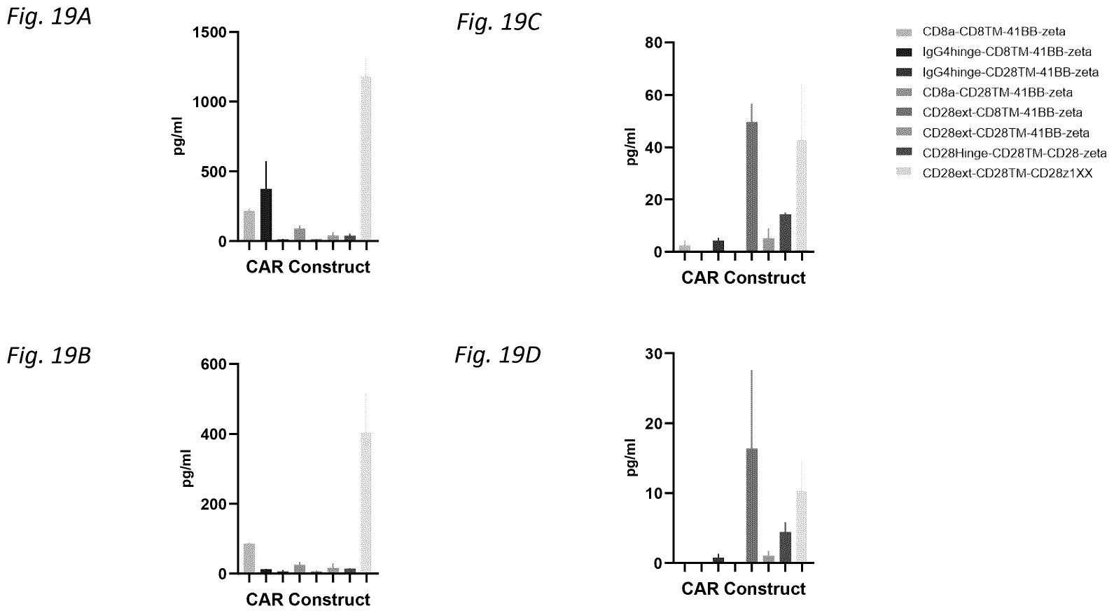

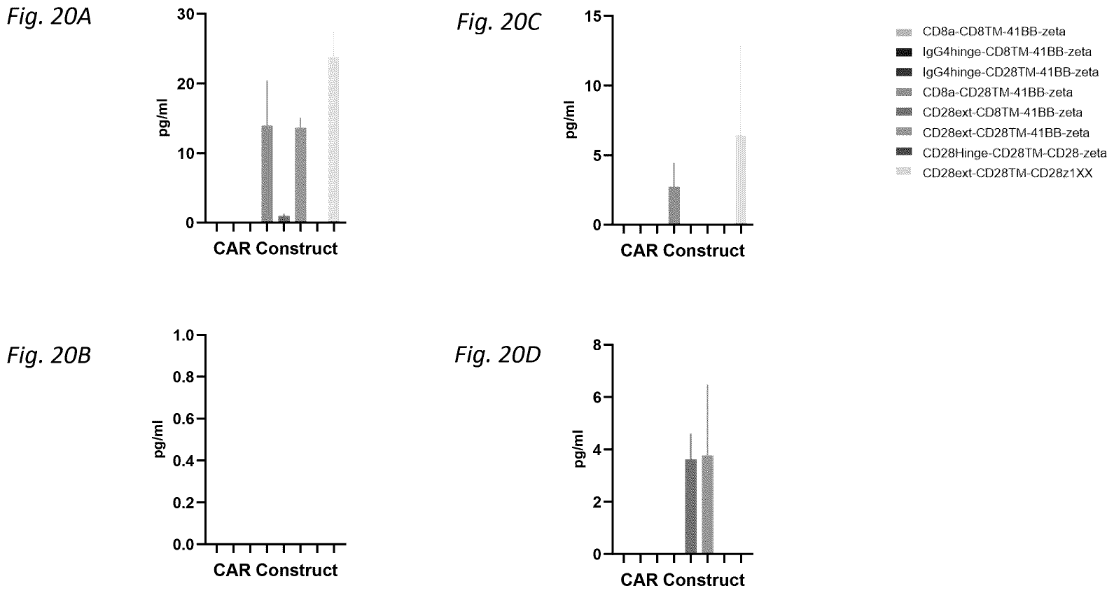

FIGS. 14-20 show the impact of the GPRC5D CAR T cells on cytokine production in the Incucyte assay described above. Supernatants were assayed for levels of GM-CSF (FIGS. 14A-14D), Granzyme B (FIGS. 15A-15D), IFNγ (FIGS. 16A-16D), IL-2 (FIGS. 17A-17D), TNF-α (FIGS. 18A-18D), IL-5 (FIGS. 19A-19D), and IL-17a (FIGS. 20A-20D) through a Meso Scale Discovery immunoassay based on the manufacturer's instructions.

### Tables

TABLE 1
Boundaries of CDRs according to various numbering schemes

| CDR | Kabat | Chothia | AbM | Contact |
|---|---|---|---|---|
| CDR-H1 | H31--H35B | H26-- | H26--H35B | H30--H35B |
| (Kabat | | H32 . . . 34 | | |
| Numbering¹) | | | | |
| CDR-H1 | H31--H35 | H26--H32 | H26--H35 | H30--H35 |
| (Chothia | | | | |
| Numbering²) | | | | |
| CDR-H2 | H50--H65 | H52--H56 | H50--H58 | H47--H58 |
| CDR-H3 | H95--H102 | H95--H102 | H95--H102 | H93--H101 |

¹Kabat et al. (1991), “Sequences of Proteins of Immunological Interest,” 5^{th} Ed. Public Health Service, National Institutes of Health, Bethesda, MD
²Al-Lazikani et al., (1997) JMB 273, 927-948

TABLE 2
Amino Acid Exemplary Substitutions

| Original Residue | Exemplary Substitution |
|---|---|
| Ala (A) | Val; Leu; Ile |
| Arg (R) | Lys; Gln; Asn |
| Asn (N) | Gln; His; Asp; Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

TABLE 3
HCDRS in Kabat Numbering Scheme

| | H-CDR1 | | H-CDR2 | | H-CDR3 | |
|---|---|---|---|---|---|---|
| | | SEQ | | SEQ | | SEQ |
| GPRC5D | Se- | ID | Se- | ID | Se- | ID |
| Binder | quence | NO: | quence | NO: | quence | NO: |
| 1 | GYTFTSYY | 1 | INPNSGGT | 4 | VRSKGRAAR | 7 |
| | | | | | NYYYMDV | |
| 2 | GFTFSSYA | 2 | ISGSGGST | 5 | ARDLYGYRY | 8 |
| | | | | | YYYGMDV | |
| 3 | GDSVSSNS | 3 | TYYRSKWYN | 6 | ARAYSPSRL | 9 |
| | AA | | | | RWSRAAAFD | |
| | | | | | I | |

TABLE 4
LCDRS in Kabat Numbering Scheme

| | L-CDR1 | | L-CDR2 | | L-CDR3 | |
|---|---|---|---|---|---|---|
| | | SEQ | | SEQ | | SEQ |
| GPRC5D | Se- | ID | Se- | ID | Se- | ID |
| Binder | quence | NO: | quence | NO: | quence | NO: |
| 1 | SVRTYY | 10 | GKN | | NSRDSSANPV | 16 |
| 2 | SLRRYF | 11 | GKN | | NSRDRSGTVV | 17 |
| 3 | SLRSYY | 12 | GKN | | SSRDSSGNHL | 18 |
| | | | | | VV | |

TABLE 5
VH Sequences

| GPRC5D | | SEQ ID |
|---|---|---|
| Binder | VH Sequence | NO: |
| 1 | QVQLVQSGAEVKKPGASVKVSCKASGYTF | 19 |
| | TSYYMHWVRQAPGQGLEWMGWINPNSGGT | |
| | NYAQKFQGRVTITADKSTSTAYMELNSLR | |
| | AEDTAVYYCVRSKGRAARNYYYMDVWGKG | |
| | TTVTVSS | |
| 2 | EVQLVESGGGVVQPGRSLRLSCAASGFTF | 20 |
| | SSYAMSWVRQAPGKGLEWVSAISGSGGST | |
| | YYAGSVKGRFTISRDNSKNTLYLQMNSLR | |
| | AEDTAVYYCARDLYGYRYYYYGMDVWGQG | |
| | TMVTVSS | |
| 3 | QVQLQQSGPGQVKPSQTLSLTCAISGDSV | 21 |
| | SSNSAAWNWIRQSPSRGLEWLGRTYYRSK | |
| | WYNDYAVSVKSRITINPDTSKNQFSLQLN | |
| | SVTPEDTAVYYCARAYSPSRLRWSRAAAF | |
| | DIWGQGTTVTVSS | |

TABLE 6
VL Sequences

| GPRC5D | | SEQ ID |
|---|---|---|
| Binder | VH Sequence | NO: |
| 1 | SSELTQDPAASVALGQTVRITCQGD | 22 |
| | SVRTYYAGWYQQKPGQAPVLVIYGK | |
| | NHRPSGIPDRFSGSTSGNTASLTIT | |
| | GVQAEDEADYYCNSRDSSANPVFGG | |
| | GTKVTVL | |
| 2 | SSELTQDPAASVALGQTVKITCQGD | 23 |
| | SLRRYFASWYQQKPGQAPTLVTYGK | |
| | NRRPSGVPDRLSGSSSGDTASLTIT | |
| | GAQAEDEGDYYCNSRDRSGTVVFGG | |
| | GTKLTVL | |
| 3 | SSELTQDPAVSVALGQTVRITCQGD | 24 |
| | SLRSYYANWYQQKPGQAPILVNYGK | |
| | NNRPSGIPDRFSGSSSGKTASLTIT | |
| | GAQAEDEADYYCSSRDSSGNHLVVF | |
| | GGGTQLTVL | |

TABLE 7
Full GPRC5D Binder scFv and sdAb Sequences

| GPRC5D | | SEQ ID |
|---|---|---|
| Binder | scFv Sequence | NO: |
| 1 | SSELTQDPAASVALGQTVRITCQGD | 25 |
| | SVRTYYAGWYQQKPGQAPVLVIYGK | |
| | NHRPSGIPDRFSGSTSGNTASLTIT | |
| | GVQAEDEADYYCNSRDSSANPVFGG | |
| | GTKVTVLGGGGSGGGGSGGGGSQVQ | |
| | LVQSGAEVKKPGASVKVSCKASGYT | |
| | FTSYYMHWVRQAPGQGLEWMGWINP | |
| | NSGGTNYAQKFQGRVTITADKSTST | |
| | AYMELNSLRAEDTAVYYCVRSKGRA | |
| | ARNYYYMDVWGKGTTVTVSS | |
| 2 | SSELTQDPAASVALGQTVKITCQGD | 26 |
| | SLRRYFASWYQQKPGQAPTLVTYGK | |
| | NRRPSGVPDRLSGSSSGDTASLTIT | |
| | GAQAEDEGDYYCNSRDRSGTVVFGG | |
| | GTKLTVLGGGGSGGGGSGGGGSEVQ | |
| | LVESGGGVVQPGRSLRLSCAASGFT | |
| | FSSYAMSWVRQAPGKGLEWVSAISG | |
| | SGGSTYYAGSVKGRFTISRDNSKNT | |
| | LYLQMNSLRAEDTAVYYCARDLYGY | |
| | RYYYYGMDVWGQGTMVTVSS | |
| 3 | SSELTQDPAVSVALGQTVRITCQGD | 27 |
| | SLRSYYANWYQQKPGQAPILVNYGK | |
| | NNRPSGIPDRFSGSSSGKTASLTIT | |
| | GAQAEDEADYYCSSRDSSGNHLVVF | |
| | GGGTQLTVLGGGGSGGGGSGGGGSQ | |
| | VQLQQSGPGQVKPSQTLSLTCAISG | |
| | DSVSSNSAAWNWIRQSPSRGLEWLG | |
| | RTYYRSKWYNDYAVSVKSRITINPD | |
| | TSKNQFSLQLNSVTPEDTAVYYCAR | |
| | AYSPSRLRWSRAAAFDIWGQGTTVT | |
| | VSS | |

TABLE 8
Exemplary sequences of signal peptides

| SEQ ID NO: | Sequence | Description |
|---|---|---|
| 28 | MALPVTALLLP | CD8α signal peptide |
| | LALLLHAARP | |
| 29 | METDTLLLWVL | IgK signal peptide |
| | LLWVPGSTG | |
| 30 | MLLLVTSLLLC | GMCSFR-α (CSF2RA) |
| | ELPHPAFLLIP | signal peptide |
| 31 | MEFGLSWLFLV | Immunoglobulin |
| | | heavy chain signal |
| | AILKGVQCSR | peptide |

TABLE 9
Exemplary sequences of linkers

| SEQ ID NO: | Sequence | Description |
|---|---|---|
| 32 | GGGGSGGGGSGGGGS | (G4S)3 linker |
| 33 | GSTSGSGKPGSGEGSTKG | Whitlow linker |

TABLE 10
sequences of hinge domains

| SEQ | | |
|---|---|---|
| ID | | |
| NO: | Sequence | Description |
| 34 | TTTPAPRPPTPAPTIASQPLSLRPE | CD8α hinge domain |
| | ACRPAAGGAVHTRGLDFACD | |
| 35 | IEVMYPPPYLDNEKSNGTIIHVKGK | CD28 hinge domain |
| | HLCPSPLFPGPSKP | |
| 36 | AAAIEVMYPPPYLDNEKSNGTIIHV | CD28 hinge domain |
| | KGKHLCPSPLFPGPSKP | |
| 37 | ESKYGPPCPPCP | IgG4 hinge domain |
| 38 | ESKYGPPCPSCP | IgG4 hinge domain |
| 39 | ESKYGPPCPPCPAPEFLGGPSVFLF | IgG4 hinge-CH2-CH3 |
| | PPKPKDTLMISRTPEVTCVVVDVSQ | domain |
| | EDPEVQFNWYVDGVEVHNAKTKPRE | |
| | EQFNSTYRVVSVLTVLHQDWLNGKE | |
| | YKCKVSNKGLPSSIEKTISKAKGQP | |
| | REPQVYTLPPSQEEMTKNQVSLTCL | |
| | VKGFYPSDIAVEWESNGQPENNYKT | |
| | TPPVLDSDGSFFLYSRLTVDKSRWQ | |
| | EGNVFSCSVMHEALHNHYTQKSLSL | |
| | SLGK | |

TABLE 11
Exemplary sequences of transmembrane domains

| SEQ | | |
|---|---|---|
| ID | | |
| NO: | Sequence | Description |
| 40 | IYIWAPLAGTCG | CD8α transmembrane domain |
| | VLLLSLVITLYC | |
| 41 | FWVLVVVGGVLA | CD28 transmembrane domain |
| | CYSLLVTVAFII | |
| | FWV | |
| 42 | MFWVLVVVGGVL | CD28 transmembrane domain |
| | ACYSLLVTVAFI | |
| | IFWV | |

TABLE 12
Exemplary sequences of intracellular costimulatory and/or signaling domains

| SEQ | | |
|---|---|---|
| ID | | |
| NO: | Sequence | Description |
| 43 | KRGRKKLLYIFKQPFMRPVQTTQEEDGCSCRFPEEEEGGCEL | 4-1BB costimulatory |
| | | domain |
| 44 | RSKRSRLLHSDYMNMTPRRPGPTRKHYQPYAPPRDFAAYRS | CD28 costimulatory |
| | | domain |
| 45 | RSKRSRGGHSDYMNMTPRRPGPTRKHYQPYAPPRDFAAYRS | CD28 costimulatory |
| | | domain (LL>GG |
| | | mutant) |
| 46 | RVKFSRSADAPAYQQGQNQLYNELNLGRREEYDVLDKRRGRDPEMGGKPRRKN | CD3ζ signaling |
| | PQEGLYNELQKDKMAEAYSEIGMKGERRRGKGHDGLYQGLSTATKDTYDALHM | domain |
| | QALPPR | |
| 47 | RVKFSRSADAPAYKQGQNQLYNELNLGRREEYDVLDKRRGRDPEMGGKPRRKNP | CD3ζ signaling |
| | QEGLYNELQKDKMAEAYSEIGMKGERRRGKGHDGLYQGLSTATKDTYDALHMQ | domain (with Q to K |
| | ALPPR | mutation at position |
| | | 14) |

TABLE 13
Exemplary sequences of CAR components

| | | SEQ |
|---|---|---|
| | | ID |
| Component | Sequence | NO: |
| Extracellular | | |
| binding domain | | |
| GPRC5D Binder 1 | SSELTQDPAASVALGQTVRITCQGDSVRTYYAGWYQQKPGQAPVLVIYGKNHRPSGIPDRF | 25 |
| | SGSTSGNTASLTITGVQAEDEADYYCNSRDSSANPVFGGGTKVTVLGGGGSGGGGSGGGG | |
| | SQVQLVQSGAEVKKPGASVKVSCKASGYTFTSYYMHWVRQAPGQGLEWMGWINPNSG | |
| | GTNYAQKFQGRVTITADKSTSTAYMELNSLRAEDTAVYYCVRSKGRAARNYYYMDVWGK | |
| | GTTVTVSS | |
| GPRC5D Binder 2 | SSELTQDPAASVALGQTVKITCQGDSLRRYFASWYQQKPGQAPTLVTYGKNRRPSGVPDRL | 26 |
| | SGSSSGDTASLTITGAQAEDEGDYYCNSRDRSGTVVFGGGTKLTVLGGGGSGGGGSGGGG | |
| | SEVQLVESGGGVVQPGRSLRLSCAASGFTFSSYAMSWVRQAPGKGLEWVSAISGSGGSTY | |
| | YAGSVKGRFTISRDNSKNTLYLQMNSLRAEDTAVYYCARDLYGYRYYYYGMDVWGQGTM | |
| | VTVSS | |
| GPRC5D Binder 3 | SSELTQDPAVSVALGQTVRITCQGDSLRSYYANWYQQKPGQAPILVNYGKNNRPSGIPDRF | 27 |
| | SGSSSGKTASLTITGAQAEDEADYYCSSRDSSGNHLVVFGGGTQLTVLGGGGSGGGGSGG | |
| | GGSQVQLQQSGPGQVKPSQTLSLTCAISGDSVSSNSAAWNWIRQSPSRGLEWLGRTYYRS | |
| | KWYNDYAVSVKSRITINPDTSKNQFSLQLNSVTPEDTAVYYCARAYSPSRLRWSRAAAFDI | |
| | WGQGTTVTVSS | |
| Spacer (e.g., | | |
| hinge) | | |
| IgG4 Hinge | ESKYGPPCPPCP | 37 |
| CD8 Hinge | TTTPAPRPPTPAPTIASQPLSLRPE | 48 |
| CD28 | IEVMYPPPYLDNEKSNGTIIHVKGKHLCPSPLFPGPSKP | 35 |
| Transmembrane | | |
| CD8 | ACRPAAGGAVHTRGLDFACDIYIWAPLAGTCGVLLLSLVITLYC | 49 |
| CD28 | FWVLVVVGGVLACYSLLVTVAFIIFWV | 41 |
| CD28 | MFWVLVVVGGVLACYSLLVTVAFIIFWV | 42 |
| Costimulatory | | |
| domain | | |
| CD28 | RSKRSRLLHSDYMNMTPRRPGPTRKHYQPYAPPRDFAAYRS | 44 |
| 4-1BB | KRGRKKLLYIFKQPFMRPVQTTQEEDGCSCRFPEEEEGGCEL | 43 |
| Primary | | |
| Signaling | | |
| Domain | | |
| CD3zeta | RVKFSRSADAPAYQQGQNQLYNELNLGRREEYDVLDKRRGRDPEMGGKPRRKNPQEGLY | 46 |
| | NELQKDKMAEAYSEIGMKGERRRGKGHDGLYQGLSTATKDTYDALHMQALPPR | |
| CD3zeta (Q > K) | RVKFSRSADAPAYKQGQNQLYNELNLGRREEYDVLDKRRGRDPEMGGKPRRKNPQEGLY | 47 |
| | NELQKDKMAEAYSEIGMKGERRRGKGHDGLYQGLSTATKDTYDALHMQALPPR | |

TABLE 14
HCDRS in Kabat Numbering Scheme

| | H-CDR1 | | H-CDR2 | | H-CDR3 | |
|---|---|---|---|---|---|---|
| CD4 | | SEQ ID | | SEQ ID | | SEQ ID |
| Binder | Sequence | NO: | Sequence | NO: | Sequence | NO: |
| 1 | SYWIE | 50 | EILPGSGSTSYNEKFKG | 54 | RGYGYDEGFDY | 58 |
| 2 | DYVIS | 51 | EIYPGSGSSYYNEKFKG | 55 | PGDLGFAY | 59 |
| 3 | THWMH | 52 | MINPSDGVTYYAQTFQG | 56 | EYYGEGFDY | 60 |
| 4 | GYWMY | 53 | AISPGGGSTYYPDSVKG | 57 | SLTATHTYEYDY | 61 |

TABLE 15
LCDRS in Kabat Numbering Scheme

| | L-CDR1 | | L-CDR2 | | L-CDR3 | |
|---|---|---|---|---|---|---|
| CD4 | | SEQ ID | | SEQ ID | | SEQ ID |
| Binder | Sequence | NO: | Sequence | NO: | Sequence | NO: |
| 1 | ASQDINSYLS | 62 | RANRLVD | 65 | LQYDEFPPT | 68 |
| 2 | ASQSVDYDGDSYMN | 63 | AASNLES | 66 | QQSNKDPFT | 69 |
| 3 | RASQGISNYLA | 64 | SASNLQS | 67 | QQSYSTPLT | 70 |

TABLE 16
VH Sequences

| CD4 | | SEQ ID |
|---|---|---|
| Binder | VH Sequence | NO: |
| 1 | QVQLQQSGAELMKPGASVKISCKATGYTFSSYWIEWVKQRPGHGLEWIGEILPGSGSTSYNEKFK | 71 |
| | GKATFTADTSSNTAYMQLSSLTSEDSAVYYCARRGYGYDEGFDYWGQGTTLTVSS | |
| 2 | QVQLQQSGPELVKPGASVKMSCKASGYTFTDYVISWVRQAPGQGLEWIGEIYPGSGSSYYNEKFK | 72 |
| | GRATLTADKSSNTAYMQLSSLRSEDSAVYFCARPGDLGFAYWGQGTLVTVSS | |
| 3 | QVQLVQSGAEVKKPGASVKVSCKASGYSLITHWMHWVRQAPGQGLEWMGMINPSDGVTYYA | 73 |
| | QTFQGRVTMTRDTSTSTVYMELSSLRSEDTAVYYCAREYYGEGFDYWGQGTLVTVSS | |
| 4 | EVQLVESGGGLVQSGGSLRLSCAASGFTFSGYWMYWVRQAPGKGLEWVSAISPGGGSTYYPDS | 74 |
| | VKGRFTISRDNAKNTLYLQMNSLEPEDTALYYCASSLTATHTYEYDYWGQGTQVTVSS | |

TABLE 17
VL Sequences

| CD4 | | SEQ ID |
|---|---|---|
| Binder | VH Sequence | NO: |
| 1 | DIKMTQSPSSMYASLGERVTITCKASQDINSYLSWFQQKPGKSPKTLIYRANRLVDGVPSRFSGSG | 75 |
| | SGQDYSLTISSLEYEDMGIYYCLQYDEFPPTFGAGTKLELKR | |
| 2 | DIVLTQSPSSLAVSLGQRATISCKASQSVDYDGDSYMNWYQQKPGQPPKLLIYAASNLESGIPARF | 76 |
| | SGSGSGTDFTLTIHPVEEEDAATYYCQQSNKDPFTFGGGTKLELKR | |
| 3 | DIQMTQSPSSLSASVGDRVTITCRASQGISNYLAWYQQKPGKAPKLLIYSASNLQSGVPSRFSGSG | 77 |
| | SGTDFTLTISSLQPEDFATYYCQQSYSTPLTFGGGTKVEIKR* | |

TABLE 18
HCDRS in Kabat Numbering Scheme

| | H-CDR1 | | H-CDR2 | | H-CDR3 | |
|---|---|---|---|---|---|---|
| CD8 | | SEQ ID | | SEQ ID | | SEQ ID |
| Binder | Sequence | NO: | Sequence | NO: | Sequence | NO: |
| 1 | SYAIS | 78 | IIDPSDGNTNYAQNFQG | 82 | ERAAAGYYYYMDV | 86 |
| 2 | DYYIQ | 79 | WINPNSGGTSYAQKFQG | 83 | EGDYYYGMDA | 87 |
| 3 | SYYMH | 80 | GFDPEDGETIYAQKFQG | 84 | DQGWGMDV | 88 |
| 4 | NHYMH | 81 | WMNPNSGNTGYAQKFQG | 85 | SESGSDLDY | 89 |

TABLE 19
LCDRS in Kabat Numbering Scheme

| | L-CDR1 | | L-CDR2 | | L-CDR3 | |
|---|---|---|---|---|---|---|
| CD8 | | SEQ ID | | SEQ ID | | SEQ ID |
| Binder | Sequence | NO: | Sequence | NO: | Sequence | NO: |
| 1 | RASQSISSYLN | 90 | AASSLQS | 94 | QQSYSTPLT | 98 |
| 2 | RSSQSLLHSNGYNYLD | 91 | LGSNRAS | 95 | MQGLQTPHT | 99 |
| 3 | RASQSISSYLN | 92 | AASSLQS | 96 | QQTYSTPYT | 100 |
| 4 | RASQTIGNYVN | 93 | GASNLHT | 97 | QQTYSAPLT | 101 |

TABLE 20
Sequences

| CD8 | | SEQ ID |
|---|---|---|
| Binder | VH Sequence | NO: |
| 1 | QVQLVQSGAEVKKPGASVKVSCKASGGTFSSYAISWVRQAPGQGLEWMGIIDPSDGNTNYAQN | 102 |
| | FQGRVTMTRDTSTSTVYMELSSLRSEDTAVYYCAKERAAAGYYYYMDVWGQGTTVTVSS | |
| 2 | QVQLVQSGAEVKKPGASVKVSCKASGYTFTDYYIQWVRQAPGQGLEWMGWINPNSGGTSYAQ | 103 |
| | KFQGRVTMTRDTSTSTVYMELSSLRSEDTAVYYCAKEGDYYYGMDAWGQGTMVTVSS | |
| 3 | QVQLVQSGAEVKKPGASVKVSCKASGYTFTSYYMHWVRQAPGQGLEWMGGFDPEDGETIYAQ | 104 |
| | KFQGRVTMTRDTSTSTVYMELSSLRSEDTAVYYCARDQGWGMDVWGQGTTVTVSS | |
| 4 | QVQLVQSGAEVKKPGASVKVSCKASGYTFTNHYMHWVRQAPGQGLEWMGWMNPNSGNTG | 105 |
| | YAQKFQGRVTMTRDTSTSTVYMELSSLRSEDTAVYYCASSESGSDLDYWGQGTLVTVSS | |

TABLE 21
VL Sequences

| CD8 | | SEQ ID |
|---|---|---|
| Binder | VH Sequence | NO: |
| 1 | DIQMTQSPSSLSASVGDRVTITCRASQSISSYLNWYQQKPGKAPKLLIYAASSLQSGVPSRFSGSGS | 106 |
| | GTDFTLTISSLQPEDFATYYCQQSYSTPLTFGGGTKVEIKR | |
| 2 | DIVMTQSPLSLPVTPGEPASISCRSSQSLLHSNGYNYLDWYLQKPGQSPQLLIYLGSNRASGVPDRF | 107 |
| | SGSGSGTDFTLKISRVEAEDVGVYYCMQGLQTPHTFGQGTKVEIKR | |
| 3 | DIQMTQSPSSLSASVGDRVTITCRASQSISSYLNWYQQKPGKAPKLLIYAASSLQSGVPSRFSGSGS | 108 |
| | GTDFTLTISSLQPEDFATYYCQQTYSTPYTFGQGTKLEIKR | |
| 4 | DIQMTQSPSSLSASVGDRVTITCRASQTIGNYVNWYQQKPGKAPKLLIYGASNLHTGVPSRFSGSG | 109 |
| | SGTDFTLTISSLQPEDFATYYCQQTYSAPLTFGGGTKVEIKR | |

TABLE 22
Exemplary G and F Protein Sequences

| SEQ ID | | |
|---|---|---|
| NO: | SEQUENCE | ANNOTATION |
| 110 | MVVILDKRCY CNLLILILMI SECSVGILHY EKLSKIGLVK GVTRKYKIKS | Nipah virus NiV-F with |
| | NPLTKDIVIK MIPNVSNMSQ CTGSVMENYK TRLNGILTPI KGALEIYKNN | signal sequence (aa 1-546) |
| | THDLVGDVRL AGVIMAGVAI GIATAAQITA GVALYEAMKN ADNINKLKSS | Uniprot Q9IH63 |
| | IESTNEAVVK LQETAEKTVY VLTALQDYIN TNLVPTIDKI SCKQTELSLD | |
| | LALSKYLSDL LFVFGPNLQD PVSNSMTIQA ISQAFGGNYE TLLRTLGYAT | |
| | EDFDDLLESD SITGQIIYVD LSSYYIIVRV YFPILTEIQQ AYIQELLPVS | |
| | FNNDNSEWIS IVPNFILVRN TLISNIEIGF CLITKRSVIC NQDYATPMTN | |
| | NMRECLTGST EKCPRELVVS SHVPRFALSN GVLFANCISV TCQCQTTGRA | |
| | ISQSGEQTLL MIDNTTCPTA VLGNVIISLG KYLGSVNYNS EGIAIGPPVF | |
| | TDKVDISSQI SSMNQSLQQS KDYIKEAQRL LDTVNPSLIS MLSMIILYVL | |
| | SIASLCIGLI TFISFIIVEK KRNTYSRLED RRVRPTSSGD LYYIGT | |
| 111 | ILHY EKLSKIGLVK GVTRKYKIKS NPLTKDIVIK MIPNVSNMSQ CTGSVMENYK | Nipah virus NiV-F F0 |
| | TRLNGILTPI KGALEIYKNN THDLVGDVRL AGVIMAGVAI GIATAAQITA | (aa 27-546) |
| | GVALYEAMKN ADNINKLKSS IESTNEAVVK LQETAEKTVY VLTALQDYIN | |
| | TNLVPTIDKI SCKQTELSLD LALSKYLSDL LFVFGPNLQD PVSNSMTIQA | |
| | ISQAFGGNYE TLLRTLGYAT EDFDDLLESD SITGQIIYVD LSSYYIIVRV | |
| | YFPILTEIQQ AYIQELLPVS FNNDNSEWIS IVPNFILVRN TLISNIEIGF | |
| | CLITKRSVIC NQDYATPMTN NMRECLTGST EKCPRELVVS SHVPRFALSN | |
| | GVLFANCISV TCQCQTTGRA ISQSGEQTLL MIDNTTCPTA VLGNVIISLG | |
| | KYLGSVNYNS EGIAIGPPVF TDKVDISSQI SSMNQSLQQS KDYIKEAQRL | |
| | LDTVNPSLIS MLSMIILYVL SIASLCIGLI TFISFIIVEK KRNTYSRLED | |
| | RRVRPTSSGD LYYIGT | |
| 112 | ILHYEKLSKIGLVKGVTRKYKIKSNPLTKDIVIKMIPNVSNMSQCTGSVMENYKTR | Nipah virus NiV-F F2 |
| | LNGILTPIKGALEIYKNNTHDLVGDVR | (aa 27-109) |
| 113 | LAGVIMAGVAIGIATAAQITAGVALYEAMKNADNINKLKSSIESTNEAVVKLQET | Nipah virus NiV F F1 |
| | AEKTVYVLTALQDYINTNLVPTIDKISCKQTELSLDLALSKYLSDLLFVFGPNLQDP | (aa 110-546) |
| | VSNSMTIQAISQAFGGNYETLLRTLGYATEDFDDLLESDSITGQIIYVDLSSYYIIVR | |
| | VYFPILTEIQQAYIQELLPVSFNNDNSEWISIVPNFILVRNTLISNIEIGFCLITKRSVI | |
| | CNQDYATPMTNNMRECLTGSTEKCPRELVVSSHVPRFALSNGVLFANCISVTC | |
| | QCQTTGRAISQSGEQTLLMIDNTTCPTAVLGNVIISLGKYLGSVNYNSEGIAIGPP | |
| | VFTDKVDISSQISSMNQSLQQSKDYIKEAQRLLDTVNPSLISMLSMIILYVLSIASL | |
| | CIGLITFISFIIVEKKRNTYSRLEDRRVRPTSSGDLYYIGT | |
| 114 | ILHY EKLSKIGLVK GVTRKYKIKS NPLTKDIVIK MIPNVSNMSQ CTGSVMENYK | Nipah virus NiV-F F0 |
| | TRLNGILTPI KGALEIYKNN THDLVGDVRL AGVIMAGVAI GIATAAQITA | truncation (aa 525-544) |
| | GVALYEAMKN ADNINKLKSS IESTNEAVVK LQETAEKTVY VLTALQDYIN | |
| | TNLVPTIDKI SCKQTELSLD LALSKYLSDL LFVFGPNLQD PVSNSMTIQA | |
| | ISQAFGGNYE TLLRTLGYAT EDFDDLLESD SITGQIIYVD LSSYYIIVRV | |
| | YFPILTEIQQ AYIQELLPVS FNNDNSEWIS IVPNFILVRN TLISNIEIGF | |
| | CLITKRSVIC NQDYATPMTN NMRECLTGST EKCPRELVVS SHVPRFALSN | |
| | GVLFANCISV TCQCQTTGRA ISQSGEQTLL MIDNTTCPTA VLGNVIISLG | |
| | KYLGSVNYNS EGIAIGPPVF TDKVDISSQI SSMNQSLQQS KDYIKEAQRL | |
| | LDTVNPSLIS MLSMIILYVL SIASLCIGLI TFISFIIVEK KRNTGT | |
| 115 | LAGVIMAGVAIGIATAAQITAGVALYEAMKNADNINKLKSSIESTNEAVVKLQET | Nipah virus NiV F F1 |
| | AEKTVYVLTALQDYINTNLVPTIDKISCKQTELSLDLALSKYLSDLLFVFGPNLQDP | (aa 110-546) truncation |
| | VSNSMTIQAISQAFGGNYETLLRTLGYATEDFDDLLESDSITGQIIYVDLSSYYIIVR | (aa 525-544) |
| | VYFPILTEIQQAYIQELLPVSFNNDNSEWISIVPNFILVRNTLISNIEIGFCLITKRSVI | |
| | CNQDYATPMTNNMRECLTGSTEKCPRELVVSSHVPRFALSNGVLFANCISVTC | |
| | QCQTTGRAISQSGEQTLLMIDNTTCPTAVLGNVIISLGKYLGSVNYNSEGIAIGPP | |
| | VFTDKVDISSQISSMNQSLQQSKDYIKEAQRLLDTVNPSLISMLSMIILYVLSIASL | |
| | CIGLITFISFIIVEKKRNTGT | |
| 116 | ILHY EKLSKIGLVK GVTRKYKIKS NPLTKDIVIK MIPNVSNMSQ CTGSVMENYK | Nipah virus NiV-F F0 |
| | TRLNGILTPI KGALEIYKNQ THDLVGDVRL AGVIMAGVAI GIATAAQITA | truncation (aa 525-544) |
| | GVALYEAMKN ADNINKLKSS IESTNEAVVK LQETAEKTVY VLTALQDYIN | AND mutation on N-linked |
| | TNLVPTIDKI SCKQTELSLD LALSKYLSDL LFVFGPNLQD PVSNSMTIQA | glycosylation site |
| | ISQAFGGNYE TLLRTLGYAT EDFDDLLESD SITGQIIYVD LSSYYIIVRV | |
| | YFPILTEIQQ AYIQELLPVS FNNDNSEWIS IVPNFILVRN TLISNIEIGF | |
| | CLITKRSVIC NQDYATPMTN NMRECLTGST EKCPRELVVS SHVPRFALSN | |
| | GVLFANCISV TCQCQTTGRA ISQSGEQTLL MIDNTTCPTA VLGNVIISLG | |
| | KYLGSVNYNS EGIAIGPPVF TDKVDISSQI SSMNQSLQQS KDYIKEAQRL | |
| | LDTVNPSLIS MLSMIILYVL SIASLCIGLI TFISFIIVEK KRNTGT | |
| 117 | MVVILDKRCY CNLLILILMI SECSVGILHY EKLSKIGLVK GVTRKYKIKS | Truncated NiV fusion |
| | NPLTKDIVIK MIPNVSNMSQ CTGSVMENYK TRLNGILTPI KGALEIYKNN | glycoprotein (FcDelta22) |
| | THDLVGDVRL AGVIMAGVAI GIATAAQITA GVALYEAMKN ADNINKLKSS | at cytoplasmic tail |
| | IESTNEAVVK LQETAEKTVY VLTALQDYIN TNLVPTIDKI SCKQTELSLD | (with signal sequence) |
| | LALSKYLSDL LFVFGPNLQD PVSNSMTIQA ISQAFGGNYE TLLRTLGYAT | |
| | EDFDDLLESD SITGQIIYVD LSSYYIIVRV YFPILTEIQQ AYIQELLPVS | |
| | FNNDNSEWIS IVPNFILVRN TLISNIEIGF CLITKRSVIC NQDYATPMTN | |
| | NMRECLTGST EKCPRELVVS SHVPRFALSN GVLFANCISV TCQCQTTGRA | |
| | ISQSGEQTLL MIDNTTCPTA VLGNVIISLG KYLGSVNYNS EGIAIGPPVF | |
| | TDKVDISSQI SSMNQSLQQS KDYIKEAQRL LDTVNPSLIS MLSMIILYVL | |
| | SIASLCIGLI TFISFIIVEK KRNT | |
| 118 | ILHY EKLSKIGLVK GVTRKYKIKS NPLTKDIVIK | Truncated NiV fusion |
| | MIPNVSNMSQ CTGSVMENYK TRLNGILTPI KGALEIYKNN THDLVGDVRL | glycoprotein (FcDelta22) |
| | AGVIMAGVAI GIATAAQITA GVALYEAMKN ADNINKLKSS IESTNEAVVK | F0 |
| | LQETAEKTVY VLTALQDYIN TNLVPTIDKI SCKQTELSLD LALSKYLSDL | |
| | LFVFGPNLQD PVSNSMTIQA ISQAFGGNYE TLLRTLGYAT EDFDDLLESD | |
| | SITGQIIYVD LSSYYIIVRV YFPILTEIQQ AYIQELLPVS FNNDNSEWIS | |
| | IVPNFILVRN TLISNIEIGF CLITKRSVIC NQDYATPMTN NMRECLTGST | |
| | EKCPRELVVS SHVPRFALSN GVLFANCISV TCQCQTTGRA ISQSGEQTLL | |
| | MIDNTTCPTA VLGNVIISLG KYLGSVNYNS EGIAIGPPVF TDKVDISSQI | |
| | SSMNQSLQQS KDYIKEAQRL LDTVNPSLIS MLSMIILYVL SIASLCIGLI | |
| | TFISFIIVEK KRNT | |
| 119 | LAGVIMAGVAI GIATAAQITA GVALYEAMKN ADNINKLKSS IESTNEAVVK | Truncated NiV fusion |
| | LQETAEKTVY VLTALQDYIN TNLVPTIDKI SCKQTELSLD LALSKYLSDL | glycoprotein (FcDelta22) |
| | LFVFGPNLQD PVSNSMTIQA ISQAFGGNYE TLLRTLGYAT EDFDDLLESD | F1 |
| | SITGQIIYVD LSSYYIIVRV YFPILTEIQQ AYIQELLPVS FNNDNSEWIS | |
| | IVPNFILVRN TLISNIEIGF CLITKRSVIC NQDYATPMTN NMRECLTGST | |
| | EKCPRELVVS SHVPRFALSN GVLFANCISV TCQCQTTGRA ISQSGEQTLL | |
| | MIDNTTCPTA VLGNVIISLG KYLGSVNYNS EGIAIGPPVF TDKVDISSQI | |
| | SSMNQSLQQS KDYIKEAQRL LDTVNPSLIS MLSMIILYVL SIASLCIGLI | |
| | TFISFIIVEK KRNT | |
| 120 | MGPAENKKVR FENTTSDKGK IPSKVIKSYY GTMDIKKINE GLLDSKILSA | NiVG protein attachment |
| | FNTVIALLGS IVIIVMNIMI IQNYTRSTDN QAVIKDALQG IQQQIKGLAD | glycoprotein (602 aa) |
| | KIGTEIGPKV SLIDTSSTIT IPANIGLLGS KISQSTASIN ENVNEKCKFT | |
| | LPPLKIHECN ISCPNPLPFR EYRPQTEGVS NLVGLPNNIC LQKTSNQILK | |
| | PKLISYTLPV VGQSGTCITD PLLAMDEGYF AYSHLERIGS CSRGVSKQRI | |
| | IGVGEVLDRG DEVPSLFMTN VWTPPNPNTV YHCSAVYNNE FYYVLCAVST | |
| | VGDPILNSTY WSGSLMMTRL AVKPKSNGGG YNQHQLALRS IEKGRYDKVM | |
| | PYGPSGIKQG DTLYFPAVGF LVRTEFKYND SNCPITKCQY SKPENCRLSM | |
| | GIRPNSHYIL RSGLLKYNLS DGENPKVVFI EISDQRLSIG SPSKIYDSLG | |
| | QPVFYQASFS WDTMIKFGDV LTVNPLVVNW RNNTVISRPG QSQCPRENTC | |
| | PEICWEGVYN DAFLIDRINW ISAGVFLDSN QTAENPVFTV FKDNEILYRA | |
| | QLASEDTNAQ KTITNCFLLK NKIWCISLVE IYDTGDNVIR PKLFAVKIPE QC | |
| 121 | MGKVR FENTTSDKGK IPSKVIKSYY GTMDIKKINE GLLDSKILSA FNTVIALLGS | NiVG protein attachment |
| | IVIIVMNIMI IQNYTRSTDN QAVIKDALQG IQQQIKGLAD KIGTEIGPKV | glycoprotein |
| | SLIDTSSTIT IPANIGLLGS KISQSTASIN ENVNEKCKFT LPPLKIHECN | Truncated A5 |
| | ISCPNPLPFR EYRPQTEGVS NLVGLPNNIC LQKTSNQILK PKLISYTLPV | |
| | VGQSGTCITD PLLAMDEGYF AYSHLERIGS CSRGVSKQRI IGVGEVLDRG | |
| | DEVPSLFMTN VWTPPNPNTV YHCSAVYNNE FYYVLCAVST VGDPILNSTY | |
| | WSGSLMMTRL AVKPKSNGGG YNQHQLALRS IEKGRYDKVM PYGPSGIKQG | |
| | DTLYFPAVGF LVRTEFKYND SNCPITKCQY SKPENCRLSM GIRPNSHYIL | |
| | RSGLLKYNLS DGENPKVVFI EISDQRLSIG SPSKIYDSLG QPVFYQASFS | |
| | WDTMIKFGDV LTVNPLVVNW RNNTVISRPG QSQCPRFNTC PEICWEGVYN | |
| | DAFLIDRINW ISAGVFLDSN QTAENPVFTV FKDNEILYRA QLASEDTNAQ | |
| | KTITNCFLLK NKIWCISLVE IYDTGDNVIR PKLFAVKIPE QC | |
| 122 | MGNTTSDKGK IPSKVIKSYY GTMDIKKINE GLLDSKILSA FNTVIALLGS | NiVG protein attachment |
| | IVIIVMNIMI IQNYTRSTDN QAVIKDALQG IQQQIKGLAD KIGTEIGPKV | glycoprotein |
| | SLIDTSSTIT IPANIGLLGS KISQSTASIN ENVNEKCKFT LPPLKIHECN | Truncated A10 |
| | ISCPNPLPFR EYRPQTEGVS NLVGLPNNIC LQKTSNQILK PKLISYTLPV | |
| | VGQSGTCITD PLLAMDEGYF AYSHLERIGS CSRGVSKQRI IGVGEVLDRG | |
| | DEVPSLFMTN VWTPPNPNTV YHCSAVYNNE FYYVLCAVST VGDPILNSTY | |
| | WSGSLMMTRL AVKPKSNGGG YNQHQLALRS IEKGRYDKVM PYGPSGIKQG | |
| | DTLYFPAVGF LVRTEFKYND SNCPITKCQY SKPENCRLSM GIRPNSHYIL | |
| | RSGLLKYNLS DGENPKVVFI EISDQRLSIG SPSKIYDSLG QPVFYQASFS | |
| | WDTMIKFGDV LTVNPLVVNW RNNTVISRPG QSQCPRFNTC PEICWEGVYN | |
| | DAFLIDRINW ISAGVFLDSN QTAENPVFTV FKDNEILYRA QLASEDTNAQ | |
| | KTITNCFLLK NKIWCISLVE IYDTGDNVIR PKLFAVKIPE QC | |
| 123 | MGKGK IPSKVIKSYY GTMDIKKINE GLLDSKILSA FNTVIALLGS IVIIVMNIMI | NiVG protein attachment |
| | IQNYTRSTDN QAVIKDALQG IQQQIKGLAD KIGTEIGPKV SLIDTSSTIT | glycoprotein |
| | IPANIGLLGS KISQSTASIN ENVNEKCKFT LPPLKIHECN ISCPNPLPFR | Truncated A15 |
| | EYRPQTEGVS NLVGLPNNIC LQKTSNQILK PKLISYTLPV VGQSGTCITD | |
| | PLLAMDEGYF AYSHLERIGS CSRGVSKQRI IGVGEVLDRG DEVPSLFMTN | |
| | VWTPPNPNTV YHCSAVYNNE FYYVLCAVST VGDPILNSTY WSGSLMMTRL | |
| | AVKPKSNGGG YNQHQLALRS IEKGRYDKVM PYGPSGIKQG DTLYFPAVGF | |
| | LVRTEFKYND SNCPITKCQY SKPENCRLSM GIRPNSHYIL RSGLLKYNLS | |
| | DGENPKVVFI EISDQRLSIG SPSKIYDSLG QPVFYQASFS WDTMIKFGDV | |
| | LTVNPLVVNW RNNTVISRPG QSQCPRFNTC PEICWEGVYN DAFLIDRINW | |
| | ISAGVFLDSN QTAENPVFTV FKDNEILYRA QLASEDTNAQ KTITNCFLLK | |
| | NKIWCISLVE IYDTGDNVIR PKLFAVKIPE QC | |
| 124 | MGSKVIKSYY GTMDIKKINE GLLDSKILSA FNTVIALLGS IVIIVMNIMI | NiVG protein attachment |
| | IQNYTRSTDN QAVIKDALQG IQQQIKGLAD KIGTEIGPKV SLIDTSSTIT | glycoprotein |
| | IPANIGLLGS KISQSTASIN ENVNEKCKFT LPPLKIHECN ISCPNPLPFR | Truncated A20 |
| | EYRPQTEGVS NLVGLPNNIC LQKTSNQILK PKLISYTLPV VGQSGTCITD | |
| | PLLAMDEGYF AYSHLERIGS CSRGVSKQRI IGVGEVLDRG DEVPSLFMTN | |
| | VWTPPNPNTV YHCSAVYNNE FYYVLCAVST VGDPILNSTY WSGSLMMTRL | |
| | AVKPKSNGGG YNQHQLALRS IEKGRYDKVM PYGPSGIKQG DTLYFPAVGF | |
| | LVRTEFKYND SNCPITKCQY SKPENCRLSM GIRPNSHYIL RSGLLKYNLS | |
| | DGENPKVVFI EISDQRLSIG SPSKIYDSLG QPVFYQASFS WDTMIKFGDV | |
| | LTVNPLVVNW RNNTVISRPG QSQCPRFNTC PEICWEGVYN DAFLIDRINW | |
| | ISAGVFLDSN QTAENPVFTV FKDNEILYRA QLASEDTNAQ KTITNCFLLK | |
| | NKIWCISLVE IYDTGDNVIR PKLFAVKIPE QC | |
| 125 | MGSYY GTMDIKKINE GLLDSKILSA FNTVIALLGS IVIIVMNIMI IQNYTRSTDN | NiVG protein attachment |
| | QAVIKDALQG IQQQIKGLAD KIGTEIGPKV SLIDTSSTIT IPANIGLLGS | glycoprotein |
| | KISQSTASIN ENVNEKCKFT LPPLKIHECN ISCPNPLPFR EYRPQTEGVS | Truncated A25 |
| | NLVGLPNNIC LQKTSNQILK PKLISYTLPV VGQSGTCITD PLLAMDEGYF | |
| | AYSHLERIGS CSRGVSKQRI IGVGEVLDRG DEVPSLFMTN VWTPPNPNTV | |
| | YHCSAVYNNE FYYVLCAVST VGDPILNSTY WSGSLMMTRL AVKPKSNGGG | |
| | YNQHQLALRS IEKGRYDKVM PYGPSGIKQG DTLYFPAVGF LVRTEFKYND | |
| | SNCPITKCQY SKPENCRLSM GIRPNSHYIL RSGLLKYNLS DGENPKVVFI | |
| | EISDQRLSIG SPSKIYDSLG QPVFYQASFS WDTMIKFGDV LTVNPLVVNW | |
| | RNNTVISRPG QSQCPRFNTC PEICWEGVYN DAFLIDRINW ISAGVFLDSN | |
| | QTAENPVFTV FKDNEILYRA QLASEDTNAQ KTITNCFLLK NKIWCISLVE | |
| | IYDTGDNVIR PKLFAVKIPE QC | |
| 126 | MGTMDIKKINE GLLDSKILSA FNTVIALLGS IVIIVMNIMI IQNYTRSTDN | NiVG protein attachment |
| | QAVIKDALQG IQQQIKGLAD KIGTEIGPKV SLIDTSSTIT IPANIGLLGS | glycoprotein |
| | KISQSTASIN ENVNEKCKFT LPPLKIHECN ISCPNPLPFR EYRPQTEGVS | Truncated A30 |
| | NLVGLPNNIC LQKTSNQILK PKLISYTLPV VGQSGTCITD PLLAMDEGYF | |
| | AYSHLERIGS CSRGVSKQRI IGVGEVLDRG DEVPSLFMTN VWTPPNPNTV | |
| | YHCSAVYNNE FYYVLCAVST VGDPILNSTY WSGSLMMTRL AVKPKSNGGG | |
| | YNQHQLALRS IEKGRYDKVM PYGPSGIKQG DTLYFPAVGF LVRTEFKYND | |
| | SNCPITKCQY SKPENCRLSM GIRPNSHYIL RSGLLKYNLS DGENPKVVFI | |
| | EISDQRLSIG SPSKIYDSLG QPVFYQASFS WDTMIKFGDV LTVNPLVVNW | |
| | RNNTVISRPG QSQCPRFNTC PEICWEGVYN DAFLIDRINW ISAGVFLDSN | |
| | QTAENPVFTV FKDNEILYRA QLASEDTNAQ KTITNCFLLK NKIWCISLVE | |
| | IYDTGDNVIR PKLFAVKIPE QC | |
| 127 | MKKINEGLLDSKILSA FNTVIALLGS IVIIVMNIMI IQNYTRSTDN | NiVG protein attachment |
| | QAVIKDALQG IQQQIKGLAD KIGTEIGPKV SLIDTSSTIT IPANIGLLGS | glycoprotein |
| | KISQSTASIN ENVNEKCKFT LPPLKIHECN ISCPNPLPFR EYRPQTEGVS | Truncated and mutated |
| | NLVGLPNNIC LQKTSNQILK PKLISYTLPV VGQSGTCITD PLLAMDEGYF | (E501A, W504A, Q530A, |
| | AYSHLERIGS CSRGVSKQRI IGVGEVLDRG DEVPSLFMTN VWTPPNPNTV | E533A) NiV G protein |
| | YHCSAVYNNE FYYVLCAVST VGDPILNSTY WSGSLMMTRL AVKPKSNGGG | (Gc Δ 34) |
| | YNQHQLALRS IEKGRYDKVM PYGPSGIKQG DTLYFPAVGF LVRTEFKYND | |
| | SNCPITKCQY SKPENCRLSM GIRPNSHYIL RSGLLKYNLS DGENPKVVFI | |
| | EISDQRLSIG SPSKIYDSLG QPVFYQASFS WDTMIKFGDV LTVNPLVVNW | |
| | RNNTVISRPG QSQCPRFNTC PAICAEGVYN DAFLIDRINW ISAGVFLDSN | |
| | ATAANPVFTV FKDNEILYRA QLASEDTNAQ KTITNCFLLK NKIWCISLVE | |
| | IYDTGDNVIR PKLFAVKIPE QCT | |
| 128 | MATQEVRLKC LLCGIIVLVL SLEGLGILHY EKLSKIGLVK GITRKYKIKS | Hendra virus F protein |
| | NPLTKDIVIK MIPNVSNVSK CTGTVMENYK SRLTGILSPI KGAIELYNNN | Uniprot O89342 (with |
| | THDLVGDVKL AGVVMAGIAI GIATAAQITA GVALYEAMKN ADNINKLKSS | signal sequence) |
| | IESTNEAVVK LQETAEKTVY VLTALQDYIN TNLVPTIDQI SCKQTELALD | |
| | LALSKYLSDL LFVFGPNLQD PVSNSMTIQA ISQAFGGNYE TLLRTLGYAT | |
| | EDFDDLLESD SIAGQIVYVD LSSYYIIVRV YFPILTEIQQ AYVQELLPVS | |
| | FNNDNSEWIS IVPNFVLIRN TLISNIEVKY CLITKKSVIC NQDYATPMTA | |
| | SVRECLTGST DKCPRELVVS SHVPRFALSG GVLFANCISV TCQCQTTGRA | |
| | ISQSGEQTLL MIDNTTCTTV VLGNIIISLG KYLGSINYNS ESIAVGPPVY | |
| | TDKVDISSQI SSMNQSLQQS KDYIKEAQKI LDTVNPSLIS MLSMIILYVL | |
| | SIAALCIGLI TFISFVIVEK KRGNYSRLDD RQVRPVSNGD LYYIGT | |
| 129 | MMADSKLVSL NNNLSGKIKD QGKVIKNYYG TMDIKKINDG LLDSKILGAF | Hendra virus G protein |
| | NTVIALLGSI IIIVMNIMII QNYTRTTDNQ ALIKESLQSV QQQIKALTDK | Uniprot O89343 |
| | IGTEIGPKVS LIDTSSTITI PANIGLLGSK ISQSTSSINE NVNDKCKFTL | |
| | PPLKIHECNI SCPNPLPFRE YRPISQGVSD LVGLPNQICL QKTTSTILKP | |
| | RLISYTLPIN TREGVCITDP LLAVDNGFFA YSHLEKIGSC TRGIAKQRII | |
| | GVGEVLDRGD KVPSMFMTNV WTPPNPSTIH HCSSTYHEDF YYTLCAVSHV | |
| | GDPILNSTSW TESLSLIRLA VRPKSDSGDY NQKYIAITKV ERGKYDKVMP | |
| | YGPSGIKQGD TLYFPAVGFL PRTEFQYNDS NCPIIHCKYS KAENCRLSMG | |
| | VNSKSHYILR SGLLKYNLSL GGDIILQFIE IADNRLTIGS PSKIYNSLGQ | |
| | PVFYQASYSW DTMIKLGDVD TVDPLRVQWR NNSVISRPGQ SQCPRFNVCP | |
| | EVCWEGTYND AFLIDRLNWV SAGVYLNSNQ TAENPVFAVF KDNEILYQVP | |
| | LAEDDTNAQK TITDCFLLEN VIWCISLVEI YDTGDSVIRP KLFAVKIPAQ CSES | |
| 130 | MVVILDKRCY CNLLILILMI SECSVGILHY EKLSKIGLVK GVTRKYKIKS | Nipah virus NiV-F F0 |
| | NPLTKDIVIK MIPNVSNMSQ CTGSVMENYK TRLNGILTPI KGALEIYKNN | truncation (aa 525-544) |
| | THDLVGDVRL AGVIMAGVAI GIATAAQITA GVALYEAMKN ADNINKLKSS | (with signal sequence) |
| | IESTNEAVVK LQETAEKTVY VLTALQDYIN TNLVPTIDKI SCKQTELSLD | |
| | LALSKYLSDL LFVFGPNLQD PVSNSMTIQA ISQAFGGNYE TLLRTLGYAT | |
| | EDFDDLLESD SITGQIIYVD LSSYYIIVRV YFPILTEIQQ AYIQELLPVS | |
| | FNNDNSEWIS IVPNFILVRN TLISNIEIGF CLITKRSVIC NQDYATPMTN | |
| | NMRECLTGST EKCPRELVVS SHVPRFALSN GVLFANCISV TCQCQTTGRA | |
| | ISQSGEQTLL MIDNTTCPTA VLGNVIISLG KYLGSVNYNS EGIAIGPPVF | |
| | TDKVDISSQI SSMNQSLQQS KDYIKEAQRL LDTVNPSLIS MLSMIILYVL | |
| | SIASLCIGLI TFISFIIVEK KRNTGT | |
| 131 | MVVILDKRCY CNLLILILMI SECSVGILHY EKLSKIGLVK GVTRKYKIKS | Nipah virus NiV-F F0 |
| | NPLTKDIVIK MIPNVSNMSQ CTGSVMENYK TRLNGILTPI KGALEIYKNQ | truncation (aa 525-544) |
| | THDLVGDVRL AGVIMAGVAI GIATAAQITA GVALYEAMKN ADNINKLKSS | AND mutation on N-linked |
| | IESTNEAVVK LQETAEKTVY VLTALQDYIN TNLVPTIDKI SCKQTELSLD | glycosylation site (with |
| | LALSKYLSDL LFVFGPNLQD PVSNSMTIQA ISQAFGGNYE TLLRTLGYAT | signal sequence) |
| | EDFDDLLESD SITGQIIYVD LSSYYIIVRV YFPILTEIQQ AYIQELLPVS | |
| | FNNDNSEWIS IVPNFILVRN TLISNIEIGF CLITKRSVIC NQDYATPMTN | |
| | NMRECLTGST EKCPRELVVS SHVPRFALSN GVLFANCISV TCQCQTTGRA | |
| | ISQSGEQTLL MIDNTTCPTA VLGNVIISLG KYLGSVNYNS EGIAIGPPVF | |
| | TDKVDISSQI SSMNQSLQQS KDYIKEAQRL LDTVNPSLIS MLSMIILYVL | |
| | SIASLCIGLI TFISFIIVEK KRNTGT | |
| 132 | MKKINEGLLDSKILSA FNTVIALLGS IVIIVMNIMI IQNYTRSTDN | NiVG protein attachment |
| | QAVIKDALQG IQQQIKGLAD KIGTEIGPKV SLIDTSSTIT IPANIGLLGS | glycoprotein |
| | KISQSTASIN ENVNEKCKFT LPPLKIHECN ISCPNPLPFR EYRPQTEGVS | Truncated (Gc Δ 34) |
| | NLVGLPNNIC LQKTSNQILK PKLISYTLPV VGQSGTCITD PLLAMDEGYF | |
| | AYSHLERIGS CSRGVSKQRI IGVGEVLDRG DEVPSLFMTN VWTPPNPNTV | |
| | YHCSAVYNNE FYYVLCAVST VGDPILNSTY WSGSLMMTRL AVKPKSNGGG | |
| | YNQHQLALRS IEKGRYDKVM PYGPSGIKQG DTLYFPAVGF LVRTEFKYND | |
| | SNCPITKCQY SKPENCRLSM GIRPNSHYIL RSGLLKYNLS DGENPKVVFI | |
| | EISDQRLSIG SPSKIYDSLG QPVFYQASFS WDTMIKFGDV LTVNPLVVNW | |
| | RNNTVISRPG QSQCPRFNTC PEICWEGVYN DAFLIDRINW ISAGVFLDSN | |
| | QTAENPVFTV FKDNEILYRA QLASEDTNAQ KTITNCFLLK NKIWCISLVE | |
| | IYDTGDNVIR PKLFAVKIPE QCT | |
| 133 | ILHY EKLSKIGLVK GVTRKYKIKS NPLTKDIVIK MIPNVSNMSQ CTGSVMENYK | Truncated mature NiV |
| | TRLNGILTPI KGALEIYKNN THDLVGDVRL AGVIMAGVAI GIATAAQITA | fusion glycoprotein |
| | GVALYEAMKN ADNINKLKSS IESTNEAVVK LQETAEKTVY VLTALQDYIN | (FcDelta22) at |
| | TNLVPTIDKI SCKQTELSLD LALSKYLSDL LFVFGPNLQD PVSNSMTIQA | cytoplasmic tail |
| | ISQAFGGNYE TLLRTLGYAT EDFDDLLESD SITGQIIYVD LSSYYIIVRV | |
| | YFPILTEIQQ AYIQELLPVS FNNDNSEWIS IVPNFILVRN TLISNIEIGF | |
| | CLITKRSVIC NQDYATPMTN NMRECLTGST EKCPRELVVS SHVPRFALSN | |
| | GVLFANCISV TCQCQTTGRA ISQSGEQTLL MIDNTTCPTA VLGNVIISLG | |
| | KYLGSVNYNS EGIAIGPPVF TDKVDISSQI SSMNQSLQQS KDYIKEAQRL | |
| | LDTVNPSLIS MLSMIILYVL SIASLCIGLI TFISFIIVEK KRNT | |
| 134 | MSNKRTTVLIIISYTLFYLNNAAIVGFDFDKLNKIGVVQGRVLNYKIKGDPMTKDL | gb: JQ001776:6129- |
| | VLKFIPNIVNITECVREPLSRYNETVRRLLLPIHNMLGLYLNNTNAKMTGLMIAGV | 8166\|Organism:Cedar |
| | IMGGIAIGIATAAQITAGFALYEAKKNTENIQKLTDSIMKTQDSIDKLTDSVGTSILI | virus\|Strain |
| | LNKLQTYINNQLVPNLELLSCRQNKIEFDLMLTKYLVDLMTVIGPNINNPVNKD | Name: CG1a\|Protein |
| | MTIQSLSLLFDGNYDIMMSELGYTPQDFLDLIESKSITGQIIYVDMENLYVVIRTYL | Name: fusion |
| | PTLIEVPDAQIYEFNKITMSSNGGEYLSTIPNFILIRGNYMSNIDVATCYMTKASVI | glycoprotein\|Gene |
| | CNQDYSLPMSQNLRSCYQGETEYCPVEAVIASHSPRFALTNGVIFANCINTICRC | Symbol: F (with signal |
| | QDNGKTITQNINQFVSMIDNSTCNDVMVDKFTIKVGKYMGRKDINNINIQIGP | sequence) |
| | QIIIDKVDLSNEINKMNQSLKDSIFYLREAKRILDSVNISLISPSVQLFLIIISVLSFIIL | |
| | LIIIVYLYCKSKHSYKYNKFIDDPDYYNDYKRERINGKASKSNNIYYVGD | |
| 135 | MALNKNMFSSLFLGYLLVYATTVQSSIHYDSLSKVGVIKGLTYNYKIKGSPSTKLM | gb: NC_025352:5950- |
| | VVKLIPNIDSVKNCTQKQYDEYKNLVRKALEPVKMAIDTMLNNVKSGNNKYRFA | 8712\|Organism: Mojiang |
| | GAIMAGVALGVATAATVTAGIALHRSNENAQAIANMKSAIQNTNEAVKQLQL | virus\|Strain |
| | ANKQTLAVIDTIRGEINNNIIPVINQLSCDTIGLSVGIRLTQYYSEIITAFGPALQNP | Name: Tongguan1\|Protein |
| | VNTRITIQAISSVFNGNFDELLKIMGYTSGDLYEILHSELIRGNIIDVDVDAGYIALE | Name: fusion |
| | IEFPNLTLVPNAVVQELMPISYNIDGDEWVTLVPRFVLTRTTLLSNIDTSRCTITDS | protein\|Gene Symbol: F |
| | SVICDNDYALPMSHELIGCLQGDTSKCAREKVVSSYVPKFALSDGLVYANCLNTI | (with signal sequence) |
| | CRCMDTDTPISQSLGATVSLLDNKRCSVYQVGDVLISVGSYLGDGEYNADNVEL | |
| | GPPIVIDKIDIGNQLAGINQTLQEAEDYIEKSEEFLKGVNPSIITLGSMVVLYIFMILI | |
| | AIVSVIALVLSIKLTVKGNVVRQQFTYTQHVPSMENINYVSH | |
| 136 | MKKKTDNPTISKRGHNHSRGIKSRALLRETDNYSNGLIVENLVRNCHHPSKNNL | gb: NC_025256:6865- |
| | NYTKTQKRDSTIPYRVEERKGHYPKIKHLIDKSYKHIKRGKRRNGHNGNIITIILLLIL | 8853\|Organism: Bat |
| | ILKTQMSEGAIHYETLSKIGLIKGITREYKVKGTPSSKDIVIKLIPNVTGLNKCTNIS | Paramyxovirus |
| | MENYKEQLDKILIPINNIIELYANSTKSAPGNARFAGVIIAGVALGVAAAAQITAGI | Eid_hel/GH- |
| | ALHEARQNAERINLLKDSISATNNAVAELQEATGGIVNVITGMQDYINTNLVPQI | M74a/GHA/2009\|Strain |
| | DKLQCSQIKTALDISLSQYYSEILTVFGPNLQNPVTTSMSIQAISQSFGGNIDLLLN | Name: BatPV/Eid_hel/GH- |
| | LLGYTANDLLDLLESKSITGQITYINLEHYFMVIRVYYPIMTTISNAYVQELIKISFNV | M74a/GHA/2009\|Protein |
| | DGSEWVSLVPSYILIRNSYLSNIDISECLITKNSVICRHDFAMPMSYTLKECLTGDT | Name: fusion |
| | EKCPREAVVTSYVPRFAISGGVIYANCLSTTCQCYQTGKVIAQDGSQTLMMIDN | protein\|Gene Symbol: F |
| | QTCSIVRIEEILISTGKYLGSQEYNTMHVSVGNPVFTDKLDITSQISNINQSIEQSKF | (with signal sequence) |
| | YLDKSKAILDKINLNLIGSVPISILFIIAILSLILSIITFVIVMIIVRRYNKYTPLINSDP | |
| | SSRRSTIQDVYIIPNPGEHSIRSAARSIDRDRD | |
| 137 | (GGGGGS)n wherein n is 1 to 6 | Peptide Linker |
| 138 | MPAENKKVRFENTTSDKGKIPSKVIKSYYGTMDIKKINEGLLDSKILSAFNTVIALL | gb: AF212302\|Organism: |
| | GSIVIIVMNIMIIQNYTRSTDNQAVIKDALQGIQQQIKGLADKIGTEIGPKVSLIDT | Nipah virus\|Strain |
| | SSTITIPANIGLLGSKISQSTASINENVNEKCKFTLPPLKIHECNISCPNPLPFREYRP | Name: UNKNOWN- |
| | QTEGVSNLVGLPNNICLQKTSNQILKPKLISYTLPVVGQSGTCITDPLLAMDEGYF | AF212302 Protein |
| | AYSHLERIGSCSRGVSKQRIIGVGEVLDRGDEVPSLFMTNVWTPPNPNTVYHCS | Name: attachment |
| | AVYNNEFYYVLCAVSTVGDPILNSTYWSGSLMMTRLAVKPKSNGGGYNQHQL | glycoprotein\|Gene |
| | ALRSIEKGRYDKVMPYGPSGIKQGDTLYFPAVGFLVRTEFKYNDSNCPITKCQYS | Symbol: G |
| | KPENCRLSMGIRPNSHYILRSGLLKYNLSDGENPKVVFIEISDQRLSIGSPSKIYDSL | (Uniprot Q9IH62) |
| | GQPVFYQASFSWDTMIKFGDVLTVNPLVVNWRNNTVISRPGQSQCPRFNTCP | |
| | EICWEGVYNDAFLIDRINWISAGVFLDSNQTAENPVFTVFKDNEILYRAQLASED | |
| | TNAQKTITNCFLLKNKIWCISLVEIYDTGDNVIRPKLFAVKIPEQCT | |
| 139 | MLSQLQKNYLDNSNQQGDKMNNPDKKLSVNFNPLELDKGQKDLNKSYYVKN | gb: JQ001776:8170- |
| | KNYNVSNLLNESLHDIKFCIYCIFSLLIIITIINIITISIVITRLKVHEENNGMESPNLQS | 10275\|Organism: Cedar |
| | IQDSLSSLTNMINTEITPRIGILVTATSVTLSSSINYVGTKTNQLVNELKDYITKSCGF | virus\|Strain |
| | KVPELKLHECNISCADPKISKSAMYSTNAYAELAGPPKIFCKSVSKDPDFRLKQIDY | Name: CG1a\|Protein |
| | VIPVQQDRSICMNNPLLDISDGFFTYIHYEGINSCKKSDSFKVLLSHGEIVDRGDY | Name: attachment |
| | RPSLYLLSSHYHPYSMQVINCVPVTCNQSSFVFCHISNNTKTLDNSDYSSDEYYIT | glycoprotein\|Gene |
| | YFNGIDRPKTKKIPINNMTADNRYIHFTFSGGGGVCLGEEFIIPVTTVINTDVFTH | Symbol: G |
| | DYCESFNCSVQTGKSLKEICSESLRSPTNSSRYNLNGIMIISQNNMTDFKIQLNGI | |
| | TYNKLSFGSPGRLSKTLGQVLYYQSSMSWDTYLKAGFVEKWKPFTPNWMNNT | |
| | VISRPNQGNCPRYHKCPEICYGGTYNDIAPLDLGKDMYVSVILDSDQLAENPEIT | |
| | VFNSTTILYKERVSKDELNTRSTTTSCFLFLDEPWCISVLETNRFNGKSIRPEIYSYKI | |
| | PKYC | |
| 140 | MPQKTVEFINMNSPLERGVSTLSDKKTLNQSKITKQGYFGLGSHSERNWKKQK | gb: NC_025256:9117- |
| | NQNDHYMTVSTMILEILVVLGIMFNLIVLTMVYYQNDNINQRMAELTSNITVLN | 11015\|Organism: Bat |
| | LNLNQLTNKIQREIIPRITLIDTATTITIPSAITYILATLTTRISELLPSINQKCEFKTPT | Paramyxovirus |
| | LVLNDCRINCTPPLNPSDGVKMSSLATNLVAHGPSPCRNFSSVPTIYYYRIPGLYN | Eid_hel/GH- |
| | RTALDERCILNPRLTISSTKFAYVHSEYDKNCTRGFKYYELMTFGEILEGPEKEPR | M74a/GHA/2009\|Strain |
| | MFSRSFYSPTNAVNYHSCTPIVTVNEGYFLCLECTSSDPLYKANLSNSTFHLVILR | Name: BatPV/Eid_hel/GH- |
| | HNKDEKIVSMPSFNLSTDQEYVQIIPAEGGGTAESGNLYFPCIGRLLHKRVTHPL | M74a/GHA/2009\|Protein |
| | CKKSNCSRTDDESCLKSYYNQGSPQHQVVNCLIRIRNAQRDNPTWDVITVDLT | Name: glycoprotein\|Gene |
| | NTYPGSRSRIFGSFSKPMLYQSSVSWHTLLQVAEITDLDKYQLDWLDTPYISRPG | Symbol: G |
| | GSECPFGNYCPTVCWEGTYNDVYSLTPNNDLFVTVYLKSEQVAENPYFAIFSRD | |
| | QILKEFPLDAWISSARTTTISCFMFNNEIWCIAALEITRLNDDIIRPIYYSFWLPTDC | |
| | RTPYPHTGKMTRVPLRSTYNY | |
| 141 | MATNRDNTITSAEVSQEDKVKKYYGVETAEKVADSISGNKVFILMNTLLILTGAII | gb: NC_025352:8716- |
| | TITLNITNLTAAKSQQNMLKIIQDDVNAKLEMFVNLDQLVKGEIKPKVSLINTAV | 11257\|Organism: Mojiang |
| | SVSIPGQISNLQTKFLQKYVYLEESITKQCTCNPLSGIFPTSGPTYPPTDKPDDDTT | virus\|Strain |
| | DDDKVDTTIKPIEYPKPDGCNRTGDHFTMEPGANFYTVPNLGPASSNSDECYTN | Name: Tongguan1\|Protein |
| | PSFSIGSSIYMFSQEIRKTDCTAGEILSIQIVLGRIVDKGQQGPQASPLLVWAVPN | Name: attachment |
| | PKIINSCAVAAGDEMGWVLCSVTLTAASGEPIPHMFDGFWLYKLEPDTEVVSYR | glycoprotein\|Gene |
| | ITGYAYLLDKQYDSVFIGKGGGIQKGNDLYFQMYGLSRNRQSFKALCEHGSCLG | Symbol: G |
| | TGGGGYQVLCDRAVMSFGSEESLITNAYLKVNDLASGKPVIIGQTFPPSDSYKGS | |
| | NGRMYTIGDKYGLYLAPSSWNRYLRFGITPDISVRSTTWLKSQDPIMKILSTCTN | |
| | TDRDMCPEICNTRGYQDIFPLSEDSEYYTYIGITPNNGGTKNFVAVRDSDGHIASI | |
| | DILQNYYSITSATISCFMYKDEIWCIAITEGKKQKDNPQRIYAHSYKIRQMCYNM | |
| | KSATVTVGNAKNITIRRY | |
| 142 | FNTVIALLGS IVIIVMNIMI IQNYTRSTDN QAVIKDALQG IQQQIKGLAD | NivG protein attachment |
| | KIGTEIGPKV SLIDTSSTIT IPANIGLLGS KISQSTASIN ENVNEKCKFT | glycoprotein |
| | LPPLKIHECN ISCPNPLPFR EYRPQTEGVS NLVGLPNNIC LQKTSNQILK | Without cytoplasmic tail |
| | PKLISYTLPV VGQSGTCITD PLLAMDEGYF AYSHLERIGS CSRGVSKQRI | Uniprot Q9IH62 |
| | IGVGEVLDRG DEVPSLFMTN VWTPPNPNTV YHCSAVYNNE FYYVLCAVST | |
| | VGDPILNSTY WSGSLMMTRL AVKPKSNGGG YNQHQLALRS IEKGRYDKVM | |
| | PYGPSGIKQG DTLYFPAVGF LVRTEFKYND SNCPITKCQY SKPENCRLSM | |
| | GIRPNSHYIL RSGLLKYNLS DGENPKVVFI EISDQRLSIG SPSKIYDSLG | |
| | QPVFYQASFS WDTMIKFGDV LTVNPLVVNW RNNTVISRPG QSQCPRENTC | |
| | PEICWEGVYN DAFLIDRINW ISAGVFLDSN QTAENPVFTV FKDNEILYRA | |
| | QLASEDTNAQ KTITNCFLLK NKIWCISLVE IYDTGDNVIR PKLFAVKIPE QC | |
| 143 | FNTVIALLGSI IIIVMNIMII QNYTRTTDNQ ALIKESLQSV QQQIKALTDK | Hendra virus G protein |
| | IGTEIGPKVS LIDTSSTITI PANIGLLGSK ISQSTSSINE NVNDKCKFTL | Uniprot O89343 |
| | PPLKIHECNI SCPNPLPFRE YRPISQGVSD LVGLPNQICL QKTTSTILKP | Without cytoplasmic tail |
| | RLISYTLPIN TREGVCITDP LLAVDNGFFA YSHLEKIGSC TRGIAKQRII | |
| | GVGEVLDRGD KVPSMFMTNV WTPPNPSTIH HCSSTYHEDF YYTLCAVSHV | |
| | GDPILNSTSW TESLSLIRLA VRPKSDSGDY NQKYIAITKV ERGKYDKVMP | |
| | YGPSGIKQGD TLYFPAVGFL PRTEFQYNDS NCPIIHCKYS KAENCRLSMG | |
| | VNSKSHYILR SGLLKYNLSL GGDIILQFIE IADNRLTIGS PSKIYNSLGQ | |
| | PVFYQASYSW DTMIKLGDVD TVDPLRVQWR NNSVISRPGQ SQCPRFNVCP | |
| | EVCWEGTYND AFLIDRLNWV SAGVYLNSNQ TAENPVFAVF KDNEILYQVP | |
| | LAEDDTNAQK TITDCFLLEN VIWCISLVEI YDTGDSVIRP KLFAVKIPAQ CSES | |
| 144 | MVVILDKRCY CNLLILILMI SECSVG | Signal sequence |
| 145 | GGGGGS | Peptide linker |
| 146 | (GGGGS)n wherein n is 1 to 10 | Peptide linker |
| 147 | GGGGS | Peptide linker |
| 148 | PAENKKVR FENTTSDKGK IPSKVIKSYY GTMDIKKINE GLLDSKILSA | NiVG protein attachment |
| | FNTVIALLGS IVIIVMNIMI IQNYTRSTDN QAVIKDALQG IQQQIKGLAD | glycoprotein (602 aa) |
| | KIGTEIGPKV SLIDTSSTIT IPANIGLLGS KISQSTASIN ENVNEKCKFT | Without N-terminal |
| | LPPLKIHECN ISCPNPLPFR EYRPQTEGVS NLVGLPNNIC LQKTSNQILK | methionine |
| | PKLISYTLPV VGQSGTCITD PLLAMDEGYF AYSHLERIGS CSRGVSKQRI | |
| | IGVGEVLDRG DEVPSLFMTN VWTPPNPNTV YHCSAVYNNE FYYVLCAVST | |
| | VGDPILNSTY WSGSLMMTRL AVKPKSNGGG YNQHQLALRS IEKGRYDKVM | |
| | PYGPSGIKQG DTLYFPAVGF LVRTEFKYND SNCPITKCQY SKPENCRLSM | |
| | GIRPNSHYIL RSGLLKYNLS DGENPKVVFI EISDQRLSIG SPSKIYDSLG | |
| | QPVFYQASFS WDTMIKFGDV LTVNPLVVNW RNNTVISRPG QSQCPRENTC | |
| | PEICWEGVYN DAFLIDRINW ISAGVFLDSN QTAENPVFTV FKDNEILYRA | |
| | QLASEDTNAQ KTITNCFLLK NKIWCISLVE IYDTGDNVIR PKLFAVKIPE QC | |
| 149 | KVR FENTTSDKGK IPSKVIKSYY GTMDIKKINE GLLDSKILSA FNTVIALLGS | NiVG protein attachment |
| | IVIIVMNIMI IQNYTRSTDN QAVIKDALQG IQQQIKGLAD KIGTEIGPKV | glycoprotein |
| | SLIDTSSTIT IPANIGLLGS KISQSTASIN ENVNEKCKFT LPPLKIHECN | Truncated 45 Without N- |
| | ISCPNPLPFR EYRPQTEGVS NLVGLPNNIC LQKTSNQILK PKLISYTLPV | terminal methionine |
| | VGQSGTCITD PLLAMDEGYF AYSHLERIGS CSRGVSKQRI IGVGEVLDRG | |
| | DEVPSLFMTN VWTPPNPNTV YHCSAVYNNE FYYVLCAVST VGDPILNSTY | |
| | WSGSLMMTRL AVKPKSNGGG YNQHQLALRS IEKGRYDKVM PYGPSGIKQG | |
| | DTLYFPAVGF LVRTEFKYND SNCPITKCQY SKPENCRLSM GIRPNSHYIL | |
| | RSGLLKYNLS DGENPKVVFI EISDQRLSIG SPSKIYDSLG QPVFYQASFS | |
| | WDTMIKFGDV LTVNPLVVNW RNNTVISRPG QSQCPRFNTC PEICWEGVYN | |
| | DAFLIDRINW ISAGVFLDSN QTAENPVFTV FKDNEILYRA QLASEDTNAQ | |
| | KTITNCFLLK NKIWCISLVE IYDTGDNVIR PKLFAVKIPE QC | |
| 150 | NTTSDKGK IPSKVIKSYY GTMDIKKINE GLLDSKILSA FNTVIALLGS | NiVG protein attachment |
| | IVIIVMNIMI IQNYTRSTDN QAVIKDALQG IQQQIKGLAD KIGTEIGPKV | glycoprotein |
| | SLIDTSSTIT IPANIGLLGS KISQSTASIN ENVNEKCKFT LPPLKIHECN | Truncated A10 Without N- |
| | ISCPNPLPFR EYRPQTEGVS NLVGLPNNIC LQKTSNQILK PKLISYTLPV | terminal methionine |
| | VGQSGTCITD PLLAMDEGYF AYSHLERIGS CSRGVSKQRI IGVGEVLDRG | |
| | DEVPSLFMTN VWTPPNPNTV YHCSAVYNNE FYYVLCAVST VGDPILNSTY | |
| | WSGSLMMTRL AVKPKSNGGG YNQHQLALRS IEKGRYDKVM PYGPSGIKQG | |
| | DTLYFPAVGF LVRTEFKYND SNCPITKCQY SKPENCRLSM GIRPNSHYIL | |
| | RSGLLKYNLS DGENPKVVFI EISDQRLSIG SPSKIYDSLG QPVFYQASFS | |
| | WDTMIKFGDV LTVNPLVVNW RNNTVISRPG QSQCPRENTC PEICWEGVYN | |
| | DAFLIDRINW ISAGVFLDSN QTAENPVFTV FKDNEILYRA QLASEDTNAQ | |
| | KTITNCFLLK NKIWCISLVE IYDTGDNVIR PKLFAVKIPE QC | |
| 151 | KGK IPSKVIKSYY GTMDIKKINE GLLDSKILSA FNTVIALLGS IVIIVMNIMI | NiVG protein attachment |
| | IQNYTRSTDN QAVIKDALQG IQQQIKGLAD KIGTEIGPKV SLIDTSSTIT | glycoprotein |
| | IPANIGLLGS KISQSTASIN ENVNEKCKFT LPPLKIHECN ISCPNPLPFR | Truncated A15 Without N- |
| | EYRPQTEGVS NLVGLPNNIC LQKTSNQILK PKLISYTLPV VGQSGTCITD | terminal methionine |
| | PLLAMDEGYF AYSHLERIGS CSRGVSKQRI IGVGEVLDRG DEVPSLFMTN | |
| | VWTPPNPNTV YHCSAVYNNE FYYVLCAVST VGDPILNSTY WSGSLMMTRL | |
| | AVKPKSNGGG YNQHQLALRS IEKGRYDKVM PYGPSGIKQG DTLYFPAVGF | |
| | LVRTEFKYND SNCPITKCQY SKPENCRLSM GIRPNSHYIL RSGLLKYNLS | |
| | DGENPKVVFI EISDQRLSIG SPSKIYDSLG QPVFYQASFS WDTMIKFGDV | |
| | LTVNPLVVNW RNNTVISRPG QSQCPRFNTC PEICWEGVYN DAFLIDRINW | |
| | ISAGVFLDSN QTAENPVFTV FKDNEILYRA QLASEDTNAQ KTITNCFLLK | |
| | NKIWCISLVE IYDTGDNVIR PKLFAVKIPE QC | |
| 152 | SKVIKSYY GTMDIKKINE GLLDSKILSA FNTVIALLGS IVIIVMNIMI | NiVG protein attachment |
| | IQNYTRSTDN QAVIKDALQG IQQQIKGLAD KIGTEIGPKV SLIDTSSTIT | glycoprotein |
| | IPANIGLLGS KISQSTASIN ENVNEKCKFT LPPLKIHECN ISCPNPLPFR | Truncated A20 Without N- |
| | EYRPQTEGVS NLVGLPNNIC LQKTSNQILK PKLISYTLPV VGQSGTCITD | terminal methionine |
| | PLLAMDEGYF AYSHLERIGS CSRGVSKQRI IGVGEVLDRG DEVPSLFMTN | |
| | VWTPPNPNTV YHCSAVYNNE FYYVLCAVST VGDPILNSTY WSGSLMMTRL | |
| | AVKPKSNGGG YNQHQLALRS IEKGRYDKVM PYGPSGIKQG DTLYFPAVGF | |
| | LVRTEFKYND SNCPITKCQY SKPENCRLSM GIRPNSHYIL RSGLLKYNLS | |
| | DGENPKVVFI EISDQRLSIG SPSKIYDSLG QPVFYQASFS WDTMIKFGDV | |
| | LTVNPLVVNW RNNTVISRPG QSQCPRFNTC PEICWEGVYN DAFLIDRINW | |
| | ISAGVFLDSN QTAENPVFTV FKDNEILYRA QLASEDTNAQ KTITNCFLLK | |
| | NKIWCISLVE IYDTGDNVIR PKLFAVKIPE QC | |
| 153 | SYY GTMDIKKINE GLLDSKILSA FNTVIALLGS IVIIVMNIMI IQNYTRSTDN | NiVG protein attachment |
| | QAVIKDALQG IQQQIKGLAD KIGTEIGPKV SLIDTSSTIT IPANIGLLGS | glycoprotein |
| | KISQSTASIN ENVNEKCKFT LPPLKIHECN ISCPNPLPFR EYRPQTEGVS | Truncated A25 Without N- |
| | NLVGLPNNIC LQKTSNQILK PKLISYTLPV VGQSGTCITD PLLAMDEGYF | terminal methionine |
| | AYSHLERIGS CSRGVSKQRI IGVGEVLDRG DEVPSLFMTN VWTPPNPNTV | |
| | YHCSAVYNNE FYYVLCAVST VGDPILNSTY WSGSLMMTRL AVKPKSNGGG | |
| | YNQHQLALRS IEKGRYDKVM PYGPSGIKQG DTLYFPAVGF LVRTEFKYND | |
| | SNCPITKCQY SKPENCRLSM GIRPNSHYIL RSGLLKYNLS DGENPKVVFI | |
| | EISDQRLSIG SPSKIYDSLG QPVFYQASFS WDTMIKFGDV LTVNPLVVNW | |
| | RNNTVISRPG QSQCPRFNTC PEICWEGVYN DAFLIDRINW ISAGVFLDSN | |
| | QTAENPVFTV FKDNEILYRA QLASEDTNAQ KTITNCFLLK NKIWCISLVE | |
| | IYDTGDNVIR PKLFAVKIPE QC | |
| 154 | TMDIKKINE GLLDSKILSA FNTVIALLGS IVIIVMNIMI IQNYTRSTDN | NiVG protein attachment |
| | QAVIKDALQG IQQQIKGLAD KIGTEIGPKV SLIDTSSTIT IPANIGLLGS | glycoprotein |
| | KISQSTASIN ENVNEKCKFT LPPLKIHECN ISCPNPLPER EYRPQTEGVS | Truncated A30 Without N- |
| | NLVGLPNNIC LQKTSNQILK PKLISYTLPV VGQSGTCITD PLLAMDEGYF | terminal methionine |
| | AYSHLERIGS CSRGVSKQRI IGVGEVLDRG DEVPSLFMTN VWTPPNPNTV | |
| | YHCSAVYNNE FYYVLCAVST VGDPILNSTY WSGSLMMTRL AVKPKSNGGG | |
| | YNQHQLALRS IEKGRYDKVM PYGPSGIKQG DTLYFPAVGF LVRTEFKYND | |
| | SNCPITKCQY SKPENCRLSM GIRPNSHYIL RSGLLKYNLS DGENPKVVFI | |
| | EISDQRLSIG SPSKIYDSLG QPVFYQASFS WDTMIKFGDV LTVNPLVVNW | |
| | RNNTVISRPG QSQCPRFNTC PEICWEGVYN DAFLIDRINW ISAGVFLDSN | |
| | QTAENPVFTV FKDNEILYRA QLASEDTNAQ KTITNCFLLK NKIWCISLVE | |
| | IYDTGDNVIR PKLFAVKIPE QC | |
| 155 | KKINEGLLDSKILSA FNTVIALLGS IVIIVMNIMI IQNYTRSTDN QAVIKDALQG | NiVG protein attachment |
| | IQQQIKGLAD KIGTEIGPKV SLIDTSSTIT IPANIGLLGS KISQSTASIN | glycoprotein |
| | ENVNEKCKFT LPPLKIHECN ISCPNPLPFR EYRPQTEGVS NLVGLPNNIC | Truncated and mutated |
| | LQKTSNQILK PKLISYTLPV VGQSGTCITD PLLAMDEGYF AYSHLERIGS | (E501 A, W504A, Q530A, |
| | CSRGVSKQRI IGVGEVLDRG DEVPSLFMTN VWTPPNPNTV YHCSAVYNNE | E533A) NiV G protein (Gc |
| | FYYVLCAVST VGDPILNSTY WSGSLMMTRL AVKPKSNGGG YNQHQLALRS | A 34) Without N-terminal |
| | IEKGRYDKVM PYGPSGIKQG DTLYFPAVGF LVRTEFKYND SNCPITKCQY | methionine |
| | SKPENCRLSM GIRPNSHYIL RSGLLKYNLS DGENPKVVFI EISDQRLSIG | |
| | SPSKIYDSLG QPVFYQASFS WDTMIKFGDV LTVNPLVVNW RNNTVISRPG | |
| | QSQCPRFNTC PAICAEGVYN DAFLIDRINW ISAGVFLDSN ATAANPVFTV | |
| | FKDNEILYRA QLASEDTNAQ KTITNCFLLK NKIWCISLVE IYDTGDNVIR | |
| | PKLFAVKIPE QCT | |
| 156 | MADSKLVSL NNNLSGKIKD QGKVIKNYYG TMDIKKINDG LLDSKILGAF | Hendra virus G protein |
| | NTVIALLGSI IIIVMNIMII QNYTRTTDNQ ALIKESLQSV QQQIKALTDK | Uniprot O89343 Without |
| | IGTEIGPKVS LIDTSSTITI PANIGLLGSK ISQSTSSINE NVNDKCKFTL | N-terminal methionine |
| | PPLKIHECNI SCPNPLPFRE YRPISQGVSD LVGLPNQICL QKTTSTILKP | |
| | RLISYTLPIN TREGVCITDP LLAVDNGFFA YSHLEKIGSC TRGIAKQRII | |
| | GVGEVLDRGD KVPSMFMTNV WTPPNPSTIH HCSSTYHEDF YYTLCAVSHV | |
| | GDPILNSTSW TESLSLIRLA VRPKSDSGDY NQKYIAITKV ERGKYDKVMP | |
| | YGPSGIKQGD TLYFPAVGFL PRTEFQYNDS NCPIIHCKYS KAENCRLSMG | |
| | VNSKSHYILR SGLLKYNLSL GGDIILQFIE IADNRLTIGS PSKIYNSLGQ | |
| | PVFYQASYSW DTMIKLGDVD TVDPLRVQWR NNSVISRPGQ SQCPRFNVCP | |
| | EVCWEGTYND AFLIDRLNWV SAGVYLNSNQ TAENPVFAVF KDNEILYQVP | |
| | LAEDDTNAQK TITDCFLLEN VIWCISLVEI YDTGDSVIRP KLFAVKIPAQ CSES | |
| 157 | KKINEGLLDSKILSA FNTVIALLGS IVIIVMNIMI IQNYTRSTDN QAVIKDALQG | NiVG protein attachment |
| | IQQQIKGLAD KIGTEIGPKV SLIDTSSTIT IPANIGLLGS KISQSTASIN | glycoprotein |
| | ENVNEKCKFT LPPLKIHECN ISCPNPLPFR EYRPQTEGVS NLVGLPNNIC | Truncated (Gc Δ 34) |
| | LQKTSNQILK PKLISYTLPV VGQSGTCITD PLLAMDEGYF AYSHLERIGS | Without N-terminal |
| | CSRGVSKQRI IGVGEVLDRG DEVPSLFMTN VWTPPNPNTV YHCSAVYNNE | methionine |
| | FYYVLCAVST VGDPILNSTY WSGSLMMTRL AVKPKSNGGG YNQHQLALRS | |
| | IEKGRYDKVM PYGPSGIKQG DTLYFPAVGF LVRTEFKYND SNCPITKCQY | |
| | SKPENCRLSM GIRPNSHYIL RSGLLKYNLS DGENPKVVFI EISDQRLSIG | |
| | SPSKIYDSLG QPVFYQASFS WDTMIKFGDV LTVNPLVVNW RNNTVISRPG | |
| | QSQCPRFNTC PEICWEGVYN DAFLIDRINW ISAGVFLDSN QTAENPVFTV | |
| | FKDNEILYRA QLASEDTNAQ KTITNCFLLK NKIWCISLVE IYDTGDNVIR | |
| | PKLFAVKIPE QCT | |
| 158 | LSQLQKNYLDNSNQQGDKMNNPDKKLSVNFNPLELDKGQKDLNKSYYVKNKN | gb: JQ001776:8170- |
| | YNVSNLLNESLHDIKFCIYCIFSLLIIITIINIITISIVITRLKVHEENNGMESPNLQSIQ | 10275\|Organism: Cedar |
| | DSLSSLTNMINTEITPRIGILVTATSVTLSSSINYVGTKTNQLVNELKDYITKSCGFKV | virus\|Strain |
| | PELKLHECNISCADPKISKSAMYSTNAYAELAGPPKIFCKSVSKDPDFRLKQIDYVI | Name: CG1a\|Protein |
| | PVQQDRSICMNNPLLDISDGFFTYIHYEGINSCKKSDSFKVLLSHGEIVDRGDYRP | Name: attachment |
| | SLYLLSSHYHPYSMQVINCVPVTCNQSSFVFCHISNNTKTLDNSDYSSDEYYITYF | glycoprotein\|Gene |
| | NGIDRPKTKKIPINNMTADNRYIHFTFSGGGGVCLGEEFIIPVTTVINTDVFTHDY | Symbol: G Without N- |
| | CESFNCSVQTGKSLKEICSESLRSPTNSSRYNLNGIMIISQNNMTDFKIQLNGITY | terminal methionine |
| | NKLSFGSPGRLSKTLGQVLYYQSSMSWDTYLKAGFVEKWKPFTPNWMNNTVI | |
| | SRPNQGNCPRYHKCPEICYGGTYNDIAPLDLGKDMYVSVILDSDQLAENPEITVF | |
| | NSTTILYKERVSKDELNTRSTTTSCFLFLDEPWCISVLETNRFNGKSIRPEIYSYKIPK | |
| | YC | |
| 159 | PQKTVEFINMNSPLERGVSTLSDKKTLNQSKITKQGYFGLGSHSERNWKKQKN | gb: NC_025256:9117- |
| | QNDHYMTVSTMILEILVVLGIMFNLIVLTMVYYQNDNINQRMAELTSNITVLNL | 11015\|Organism: Bat |
| | NLNQLTNKIQREIIPRITLIDTATTITIPSAITYILATLTTRISELLPSINQKCEFKTPTL | Paramyxovirus |
| | VLNDCRINCTPPLNPSDGVKMSSLATNLVAHGPSPCRNFSSVPTIYYYRIPGLYNRT | Eid_hel/GH- |
| | ALDERCILNPRLTISSTKFAYVHSEYDKNCTRGFKYYELMTFGEILEGPEKEPRMFS | M74a/GHA/2009\|Strain |
| | RSFYSPTNAVNYHSCTPIVTVNEGYFLCLECTSSDPLYKANLSNSTFHLVILRHNK | Name: BatPV/Eid_hel/GH- |
| | DEKIVSMPSFNLSTDQEYVQIIPAEGGGTAESGNLYFPCIGRLLHKRVTHPLCKKS | M74a/GHA/2009\|Protein |
| | NCSRTDDESCLKSYYNQGSPQHQVVNCLIRIRNAQRDNPTWDVITVDLTNTYP | Name: glycoprotein\|Gene |
| | GSRSRIFGSFSKPMLYQSSVSWHTLLQVAEITDLDKYQLDWLDTPYISRPGGSEC | Symbol: G Without N- |
| | PFGNYCPTVCWEGTYNDVYSLTPNNDLFVTVYLKSEQVAENPYFAIFSRDQILKE | terminal methionine |
| | FPLDAWISSARTTTISCFMFNNEIWCIAALEITRLNDDIIRPIYYSFWLPTDCRTPY | |
| | PHTGKMTRVPLRSTYNY | |
| 160 | ATNRDNTITSAEVSQEDKVKKYYGVETAEKVADSISGNKVFILMNTLLILTGAIITI | gb: NC_025352:8716- |
| | TLNITNLTAAKSQQNMLKIIQDDVNAKLEMFVNLDQLVKGEIKPKVSLINTAVSV | 11257\|Organism: Mojiang |
| | SIPGQISNLQTKFLQKYVYLEESITKQCTCNPLSGIFPTSGPTYPPTDKPDDDTTDD | virus\|Strain |
| | DKVDTTIKPIEYPKPDGCNRTGDHFTMEPGANFYTVPNLGPASSNSDECYTNPS | Name: Tongguan1\|Protein |
| | FSIGSSIYMFSQEIRKTDCTAGEILSIQIVLGRIVDKGQQGPQASPLLVWAVPNPKI | Name: attachment |
| | INSCAVAAGDEMGWVLCSVTLTAASGEPIPHMFDGFWLYKLEPDTEVVSYRITG | glycoprotein\|Gene |
| | YAYLLDKQYDSVFIGKGGGIQKGNDLYFQMYGLSRNRQSFKALCEHGSCLGTGG | Symbol: G Without N- |
| | GGYQVLCDRAVMSFGSEESLITNAYLKVNDLASGKPVIIGQTFPPSDSYKGSNGR | terminal methionine |
| | MYTIGDKYGLYLAPSSWNRYLRFGITPDISVRSTTWLKSQDPIMKILSTCTNTDR | |
| | DMCPEICNTRGYQDIFPLSEDSEYYTYIGITPNNGGTKNFVAVRDSDGHIASIDIL | |
| | QNYYSITSATISCFMYKDEIWCIAITEGKKQKDNPQRIYAHSYKIRQMCYNMKSA | |
| | TVTVGNAKNITIRRY | |
| 161 | DFDKLNKIGVVQGRVLNYKIKGDPMTKDLVLKFIPNIVNITECVREPLSRYNETVR | gb: JQ001776:6129- |
| | RLLLPIHNMLGLYLNNTNAKMTGLMIAGVIMGGIAIGIATAAQITAGFALYEAKK | 8166\|Organism: Cedar |
| | NTENIQKLTDSIMKTQDSIDKLTDSVGTSILILNKLQTYINNQLVPNLELLSCRQNK | virus\|Strain |
| | IEFDLMLTKYLVDLMTVIGPNINNPVNKDMTIQSLSLLFDGNYDIMMSELGYTP | Name: CG1a\|Protein |
| | QDFLDLIESKSITGQIIYVDMENLYVVIRTYLPTLIEVPDAQIYEFNKITMSSNGGEY | Name: fusion |
| | LSTIPNFILIRGNYMSNIDVATCYMTKASVICNQDYSLPMSQNLRSCYQGETEYC | glycoprotein\|Gene |
| | PVEAVIASHSPRFALTNGVIFANCINTICRCQDNGKTITQNINQFVSMIDNSTCN | Symbol: F (without signal |
| | DVMVDKFTIKVGKYMGRKDINNINIQIGPQIIIDKVDLSNEINKMNQSLKDSIFYL | sequence) |
| | REAKRILDSVNISLISPSVQLFLIIISVLSFIILLIIIVYLYCKSKHSYKYNKFIDDPDY | |
| | YNDYKRERINGKASKSNNIYYVGD | |
| 162 | SRALLRETDNYSNGLIVENLVRNCHHPSKNNLNYTKTQKRDSTIPYRVEERKGHY | gb: NC_025256:6865- |
| | PKIKHLIDKSYKHIKRGKRRNGHNGNIITIILLLILILKTQMSEGAIHYETLSKIGLIKGI | 8853\|Organism: Bat |
| | TREYKVKGTPSSKDIVIKLIPNVTGLNKCTNISMENYKEQLDKILIPINNIIELYANST | Paramyxovirus |
| | KSAPGNARFAGVIIAGVALGVAAAAQITAGIALHEARQNAERINLLKDSISATNN | Eid_hel/GH- |
| | AVAELQEATGGIVNVITGMQDYINTNLVPQIDKLQCSQIKTALDISLSQYYSEILT | M74a/GHA/2009\|Strain |
| | VFGPNLQNPVTTSMSIQAISQSFGGNIDLLLNLLGYTANDLLDLLESKSITGQITYI | Name: BatPV/Eid_hel/GH- |
| | NLEHYFMVIRVYYPIMTTISNAYVQELIKISFNVDGSEWVSLVPSYILIRNSYLSNID | M74a/GHA/2009\|Protein |
| | ISECLITKNSVICRHDFAMPMSYTLKECLTGDTEKCPREAVVTSYVPRFAISGGVIY | Name: fusion |
| | ANCLSTTCQCYQTGKVIAQDGSQTLMMIDNQTCSIVRIEEILISTGKYLGSQEYN | protein\|Gene Symbol: F |
| | TMHVSVGNPVFTDKLDITSQISNINQSIEQSKFYLDKSKAILDKINLNLIGSVPISILF | (without signal sequence) |
| | IIAILSLILSIITFVIVMIIVRRYNKYTPLINSDPSSRRSTIQDVYIIPNPGEHSIRSAA | |
| | RSIDRDRD | |
| 163 | ILHY EKLSKIGLVK GITRKYKIKS | Hendra virus F protein |
| | NPLTKDIVIK MIPNVSNVSK CTGTVMENYK SRLTGILSPI KGAIELYNNN | Uniprot O89342 (without |
| | THDLVGDVKL AGVVMAGIAI GIATAAQITA GVALYEAMKN ADNINKLKSS | signal sequence) |
| | IESTNEAVVK LQETAEKTVY VLTALQDYIN TNLVPTIDQI SCKQTELALD | |
| | LALSKYLSDL LFVFGPNLQD PVSNSMTIQA ISQAFGGNYE TLLRTLGYAT | |
| | EDFDDLLESD SIAGQIVYVD LSSYYIIVRV YFPILTEIQQ AYVQELLPVS | |
| | FNNDNSEWIS IVPNFVLIRN TLISNIEVKY CLITKKSVIC NQDYATPMTA | |
| | SVRECLTGST DKCPRELVVS SHVPRFALSG GVLFANCISV TCQCQTTGRA | |
| | ISQSGEQTLL MIDNTTCTTV VLGNIIISLG KYLGSINYNS ESIAVGPPVY | |
| | TDKVDISSQI SSMNQSLQQS KDYIKEAQKI LDTVNPSLIS MLSMIILYVL | |
| | SIAALCIGLI TFISFVIVEK KRGNYSRLDD RQVRPVSNGD LYYIGT | |
| 164 | IHYDSLSKVGVIKGLTYNYKIKGSPSTKLMVVKLIPNIDSVKNCTQKQYDEYKNLV | gb: NC_025352: 5950- |
| | RKALEPVKMAIDTMLNNVKSGNNKYRFAGAIMAGVALGVATAATVTAGIALH | 8712\|Organism: Mojiang |
| | RSNENAQAIANMKSAIQNTNEAVKQLQLANKQTLAVIDTIRGEINNNIIPVINQL | virus\|Strain |
| | SCDTIGLSVGIRLTQYYSEIITAFGPALQNPVNTRITIQAISSVFNGNFDELLKIMGY | Name: Tongguan1\|Protein |
| | TSGDLYEILHSELIRGNIIDVDVDAGYIALEIEFPNLTLVPNAVVQELMPISYNIDG | Name: fusion |
| | DEWVTLVPRFVLTRTTLLSNIDTSRCTITDSSVICDNDYALPMSHELIGCLQGDTS | protein\|Gene Symbol: F |
| | KCAREKVVSSYVPKFALSDGLVYANCLNTICRCMDTDTPISQSLGATVSLLDNKR | (without signal sequence) |
| | CSVYQVGDVLISVGSYLGDGEYNADNVELGPPIVIDKIDIGNQLAGINQTLQEAE | |
| | DYIEKSEEFLKGVNPSIITLGSMVVLYIFMILIAIVSVIALVLSIKLTVKGNVVRQQFT | |
| | YTQHVPSMENINYVSH | |
| 165 | (GGGS)n wherein n is 1 to 10 | Peptide linker |
| 32 | GGGGSGGGGSGGGGS | Peptide linker |
| 166 | TTAASGSSGGSSSGA | Peptide linker |
| 33 | GSTSGSGKPGSGEGSTKG | Peptide linker |

TABLE 23
Exemplary sequences of CD47

| SEQ | | |
|---|---|---|
| ID NO: | Sequence | Description |
| 167 | MWPLVAALLLGSACCGSAQLLFNKTKSVEFTFCNDTVVIPCFVTNMEAQNTTEVY | Amino acid |
| | VKWKFKGRDIYTFDGALNKSTVPTDFSSAKIEVSQLLKGDASLKMDKSDAVSHTG | sequence encoded |
| | NYTCEVTELTREGETIIELKYRVVSWFSPNENILIVIFPIFAILLFWGQFGIKTLKYRSG | by CDS of |
| | GMDEKTIALLVAGLVITVIVIVGAILFVPGEYSLKNATGLGLIVTSTGILILLHYYVFST | NM_001777.4 |
| | AIGLTSFVIAILVIQVIAYILAVVGLSLCIAACIPMHGPLLISGLSILALAQLLGLVYMKF | |
| | VASNQKTIQPPRKAVEEPLNAFKESKGMMNDE | |
| 168 | MWPLVAALLLGSACCGSAQLLFNKTKSVEFTFCNDTVVIPCFVTNMEAQNTTEVY | Amino acid |
| | VKWKFKGRDIYTFDGALNKSTVPTDFSSAKIEVSQLLKGDASLKMDKSDAVSHTG | sequence encoded |
| | NYTCEVTELTREGETIIELKYRVVSWFSPNENILIVIFPIFAILLFWGQFGIKTLKYRSG | by CDS of |
| | GMDEKTIALLVAGLVITVIVIVGAILFVPGEYSLKNATGLGLIVTSTGILILLHYYVFST | NM_198793.2 |
| | AIGLTSFVIAILVIQVIAYILAVVGLSLCIAACIPMHGPLLISGLSILALAQLLGLVYMKF | |
| | VASNQKTIQPPRNN | |
| 169 | atgtggcccctggtagcggcgctgttgctgggctcggcgtgctgcggatcagctcagctactat | Nucleotide |
| | ttaataaaacaaaatctgtagaattcacgttttgtaatgacactgtcgtcattccatgctttgtt | sequence of |
| | actaatatggaggcacaaaacactactgaagtatacgtaaagtggaaatttaaaggaagag | NM_001777.4 CDS |
| | atatttacacctttgatggagctctaaacaagtccactgtccccactgactttagtagtgcaaa | (nts 124-1095) |
| | aattgaagtctcacaattactaaaaggagatgcctctttgaagatggataagagtgatgctgt | |
| | ctcacacacaggaaactacacttgtgaagtaacagaattaaccagagaaggtgaaacgatc | |
| | atcgagctaaaatatcgtgttgtttcatggttttctccaaatgaaaatattcttattgttattttcc | |
| | caatttttgctatactcctgttctggggacagtttggtattaaaacacttaaatatagatccggt | |
| | ggtatggatgagaaaacaattgctttacttgttgctggactagtgatcactgtcattgtcattgt | |
| | tggagccattcttttcgtcccaggtgaatattcattaaagaatgctactggccttggtttaattgt | |
| | gacttctacagggatattaatattacttcactactatgtgtttagtacagcgattggattaacct | |
| | ccttcgtcattgccatattggttattcaggtgatagcctatatcctcgctgtggttggactgagtc | |
| | tctgtattgcggcgtgtataccaatgcatggccctcttctgatttcaggtttgagtatcttagctc | |
| | tagcacaattacttggactagtttatatgaaatttgtggcttccaatcagaagactatacaacc | |
| | tcctaggaaagctgtagaggaaccccttaatgcattcaaagaatcaaaaggaatgatgaatg | |
| | atgaataa | |
| 170 | atgtggcccctggtagcggcgctgttgctgggctcggcgtgctgcggatcagctcagctactatttaata | Nucleotide |
| | aaacaaaatctgtagaattcacgttttgtaatgacactgtcgtcattccatgctttgttactaatatggag | sequence of |
| | gcacaaaacactactgaagtatacgtaaagtggaaatttaaaggaagagatatttacacctttgatgg | NM_198793.2 CDS |
| | agctctaaacaagtccactgtccccactgactttagtagtgcaaaaattgaagtctcacaattactaaaa | (nts 181-1098) |
| | ggagatgcctctttgaagatggataagagtgatgctgtctcacacacaggaaactacacttgtgaagta | |
| | acagaattaaccagagaaggtgaaacgatcatcgagctaaaatatcgtgttgtttcatggttttctccaa | |
| | atgaaaatattcttattgttattttcccaatttttgctatactcctgttctggggacagtttggtattaaaac | |
| | acttaaatatagatccggtggtatggatgagaaaacaattgctttacttgttgctggactagtgatcact | |
| | gtcattgtcattgttggagccattcttttcgtcccaggtgaatattcattaaagaatgctactggccttggt | |
| | ttaattgtgacttctacagggatattaatattacttcactactatgtgtttagtacagcgattggattaacc | |
| | tccttcgtcattgccatattggttattcaggtgatagcctatatcctcgctgtggttggactgagtctctgta | |
| | ttgcggcgtgtataccaatgcatggccctcttctgatttcaggtttgagtatcttagctctagcacaattac | |
| | ttggactagtttatatgaaatttgtggcttccaatcagaagactatacaacctcctaggaataactga | |
| 171 | atgtggcccctggtcgccgccctgttgctgggctcggcatgctgcggatcagctcagctactgtttaataa | Codon-optimized |
| | aacaaaatctgtagaattcacgttttgtaacgacactgtcgtgatcccatgctttgttactaatatggagg | nucleotide |
| | cacaaaacaccactgaagtgtacgtgaagtggaaattcaaaggcagagacatttacacctttgacggc | sequence |
| | gccctcaacaagtccaccgtgcccactgactttagtagcgcaaaaattgaggtcagccaattactaaaa | encoding CD47 |
| | ggagatgcctctttgaagatggacaagagcgatgctgtcagccacacagggaactacacttgtgaagt | |
| | aacagagttaacccgcgaaggtgaaacgatcatcgagctgaagtatcgagtggtgtcctggttttctcc | |
| | gaacgagaatatccttatcgtaattttcccaattttcgctatcctcctgttctggggccagtttggtatcaa | |
| | gacactcaaatatcggtccggtgggatggatgagaagacaattgccctgcttgttgctggactcgtgatc | |
| | accgtcatcgtgattgttggggccatccttttcgtcccaggggagtacagcctgaagaatgctacgggcc | |
| | tgggattaattgtgacctctacagggatactcatcctgcttcactactatgtgttcagtaccgcgattgga | |
| | ctgacctccttcgtcattgccatattggtgattcaggtgatagcctacatcctcgccgtggttggcctgagt | |
| | ctctgtatcgcggcgtgcatacccatgcatggccctcttctgatttcagggttgagtatcctcgcactagc | |
| | acagttgctgggactggtttatatgaaatttgtggcctccaaccagaagactatacagcctcctaggaag | |
| | gctgtagaggagcccctgaatgcattcaaggaatcaaaaggcatgatgaatgatgaa | |

TABLE 24
Sequences of 2A peptides

| SEQ | | 2A |
|---|---|---|
| ID NO: | Amino Acid Sequence | Peptide |
| 172 | (GSG)EGRGSLLTCGDVEENPGP | T2A |
| 173 | (GSG)ATNFSLLKQAGDVEENPGP | P2A |
| 174 | (GSG)QCTNYALLKLAGDVESNPGP | E2A |
| 175 | (GSG)VKQTLNFDLLKLAGDVESNPGP | F2A |

TABLE 25
Sequences of furin sites

| SEQ | | Furin |
|---|---|---|
| ID NO: | Amino Acid Sequence | site |
| 176 | RRRR(GSG) | FC1 |
| 177 | RKRR(GSG) | FC2 |
| 178 | RKRR(GSG)TPDPW(GSG) | FC3 |

TABLE 26
Exemplary Cas nuclease variants and their PAM sequences

| CRISPR | | PAM Sequence |
|---|---|---|
| Nuclease | Source Organism | (5′-3′) |
| SpCas9 | Streptococcus pyogenes | ngg or nag |
| SaCas9 | Staphylococcus aureus | ngrrt or ngrn |
| NmeCas9 | Neisseria meningitidis | nnnngatt |
| CjCas9 | Campylobacter jejuni | nnnnryac |
| StCas9 | Streptococcus thermophilus | nnagaaw |
| TdCas9 | Treponema denticola | naaaac |
| LbCas12a | Lachnospiraceae bacterium | tttv |
| (Cpf1) | | |
| AsCas12a | Acidaminococcus sp. | tttv |
| (Cpf1) | | |
| AacCas12b | Alicyclobacillus acidiphilus | ttn |
| BhCas12bv4 | Bacillus hisashii | attn, tttn, or gttn |

r = a or g; y = c or t; w = a or t; v = a or c or g; n = any base

TABLE 27
Exemplary gRNA structure and sequence for CRISPR/Cas

| CRISPR | | |
|---|---|---|
| Nuclease | Sequence (5′-3′) | Description |
| | nnnnnnnnnnnnnnnnnnnn | Exemplary spCas9 1 |
| | | Complementary |
| | | region (spacer) |
| 180 | Guuuuagagcua | Exemplary spCas9 1 |
| | | crRNA repeat region |
| | gaaa | Exemplary spCas9 t |
| | | tetraloop |
| 182 | uagcaaguuaaaauaaggcuaguccguuaucaacuugaaaaaguggcaccgagucggugcuu | Exemplary spCas9 1 |
| | uuuu | tracrRNA |
| | nnnnnnnnnnnnnnnnnnnn | Exemplary spCas9 2 |
| | | Complementary |
| | | region (spacer) |
| 184 | guuusagagcuaugcug | Exemplary spCas9 2 |
| | | crRNA repeat region |
| | gaaa | Exemplary spCas9 2 |
| | | tetraloop |
| 186 | cagcauagcaaguusaaauaaggcuaguccguuaucaacuugaaaaaguggcaccgagucggu | Exemplary spCas9 2 |
| | gcuuuuuu | tracrRNA |
| | nnnnnnnnnnnnnnnnnnnn | Exemplary saCas9 |
| | | Complementary |
| | | region |
| 188 | guuuuaguacucug | Exemplary saCas9 |
| | | crRNA repeat region |
| | gaaa | Exemplary saCas9 |
| | | tetraloop |
| 190 | cagaaucuacuaaaacaaggcaaaaugccguguuuaucucgucaacuuguuggcgagauuuu | Exemplary saCas9 |
| | uu | tracrRNA |
| 191 | gucgucuauaggacggcgaggacaacgggaagugccaaugugcucuuuccaagagcaaacacc | Exemplary |
| | ccguuggcuucaagaugaccgcucg | AkCas12b tracrRNA |
| | aaaa | Exemplary |
| | | AkCas12b tetraloop |
| 193 | cgagcggucugagaaguggcacu | Exemplary |
| | | AkCas12b crDNA |
| | | repeat region |
| | nnnnnnnnnnnnnnnnnnnn | Exemplary |
| | | AkCas12b |
| | | Complementary |
| | | region (spacer) |

s = c or g; n = any base

TABLE 28
Experimental Groups for Production of GPRC5D CARs

| Experimental | | VH SEQ | VL SEQ |
|---|---|---|---|
| Group | Construct | ID NO: | ID NO: |
| Mock | GFP | — | — |
| Control | | — | — |
| scFv | CAR 1 (VL-VH) | 19 | 22 |
| | CAR 2 (VL-VH) | 20 | 23 |
| | CAR 3 (VL-VH) | 21 | 24 |

TABLE 29
Experimental Groups for in vivo tumor challenge

| | Mouse | Tumor | Tumor | | CAR-T cell | |
|---|---|---|---|---|---|---|
| Group | Strain | Challenge | cells/Animal | CAR-T cells | dose | Mice/Group |
| 1 | NSG | MM.1S:Wasabi- | 1.00E+07 | CAR1 | 5.00E+06 | 5 |
| | | ffLuc | | | | |
| 2 | NSG | MM.1S:Wasabi- | 1.00E+07 | CAR2 | 5.00E+06 | 5 |
| | | ffLuc | | | | |
| 3 | NSG | MM.1S:Wasabi- | 1.00E+07 | CAR3 | 5.00E+06 | 5 |
| | | ffLuc | | | | |
| 4 | NSG | MM.1S:Wasabi- | 1.00E+07 | CAR1 | 1.00E+06 | 5 |
| | | ffLuc | | | | |
| 5 | NSG | MM.1S:Wasabi- | 1.00E+07 | CAR2 | 1.00E+06 | 5 |
| | | ffLuc | | | | |
| 6 | NSG | MM.1S:Wasabi- | 1.00E+07 | CAR3 | 1.00E+06 | 5 |
| | | ffLuc | | | | |
| 7 | NSG | MM.1S:Wasabi- | 1.00E+07 | Benchmark | 5.00E+06 | 5 |
| | | ffLuc | | control | | |
| 8 | NSG | MM.1S:Wasabi- | 1.00E+07 | Mock | 1.00E+06 | 5 |
| | | ffLuc | | | | |
| 9 | NSG | MM.1S:Wasabi- | 1.00E+07 | Mock | 5.00E+06 | 5 |
| | | ffLuc | | | | |

TABLE 30
Exemplary CAR Constructs

| SEQ ID | | |
|---|---|---|
| NO: | Description | Sequence |
| 195 | CD8a-CD8TM-41BB-zeta | TTTPAPRPPTPAPTIASQPLSLRPEACRPAAGGAVHTRGLDFACD |
| | | IYIWAPLAGTCGVLLLSLVITLYCKRGRKKLLYIFKQPFMRPVQTT |
| | | QEEDGCSCRFPEEEEGGCELRVKFSRSADAPAYQQGQNQLYNEL |
| | | NLGRREEYDVLDKRRGRDPEMGGKPRRKNPQEGLYNELQKDK |
| | | MAEAYSEIGMKGERRRGKGHDGLYQGLSTATKDTYDALHMQA |
| | | LPPR |
| 196 | IgG4hinge-CD8TM-41BB-zeta | ESKYGPPCPPCPIYIWAPLAGTCGVLLLSLVITLYCKRGRKKLLYIFK |
| | | QPFMRPVQTTQEEDGCSCRFPEEEEGGCELRVKFSRSADAPAYQ |
| | | QGQNQLYNELNLGRREEYDVLDKRRGRDPEMGGKPRRKNPQE |
| | | GLYNELQKDKMAEAYSEIGMKGERRRGKGHDGLYQGLSTATKD |
| | | TYDALHMQALPPR |
| 197 | IgG4hinge-CD28TM-41BB- | ESKYGPPCPPCPFWVLVVVGGVLACYSLLVTVAFIIFWVKRGRKK |
| | zeta | LLYIFKQPFMRPVQTTQEEDGCSCRFPEEEEGGCELRVKFSRSAD |
| | | APAYQQGQNQLYNELNLGRREEYDVLDKRRGRDPEMGGKPRR |
| | | KNPQEGLYNELQKDKMAEAYSEIGMKGERRRGKGHDGLYQGLS |
| | | TATKDTYDALHMQALPPR |
| 198 | CD8a-CD28TM-41BB-zeta | TTTPAPRPPTPAPTIASQPLSLRPEACRPAAGGAVHTRGLDFACD |
| | | FWVLVVVGGVLACYSLLVTVAFIIFWVKRGRKKLLYIFKQPFMRP |
| | | VQTTQEEDGCSCRFPEEEEGGCELRVKFSRSADAPAYQQGQNQ |
| | | LYNELNLGRREEYDVLDKRRGRDPEMGGKPRRKNPQEGLYNEL |
| | | QKDKMAEAYSEIGMKGERRRGKGHDGLYQGLSTATKDTYDALH |
| | | MQALPPR |
| 199 | CD28ext-CD8TM-41BB-zeta | AAAIEVMYPPPYLDNEKSNGTIIHVKGKHLCPSPLFPGPSKPIYIW |
| | | APLAGTCGVLLLSLVITLYCKRGRKKLLYIFKQPFMRPVQTTQEED |
| | | GCSCRFPEEEEGGCELRVKFSRSADAPAYQQGQNQLYNELNLGR |
| | | REEYDVLDKRRGRDPEMGGKPRRKNPQEGLYNELQKDKMAEA |
| | | YSEIGMKGERRRGKGHDGLYQGLSTATKDTYDALHMQALPPR |
| 200 | CD28ext-CD28TM-41BB-zeta | AAAIEVMYPPPYLDNEKSNGTIIHVKGKHLCPSPLFPGPSKPFWV |
| | | LVVVGGVLACYSLLVTVAFIIFWVKRGRKKLLYIFKQPFMRPVQTT |
| | | QEEDGCSCRFPEEEEGGCELRVKFSRSADAPAYQQGQNQLYNEL |
| | | NLGRREEYDVLDKRRGRDPEMGGKPRRKNPQEGLYNELQKDK |
| | | MAEAYSEIGMKGERRRGKGHDGLYQGLSTATKDTYDALHMQA |
| | | LPPR |
| 201 | CD28Hinge-CD28TM-CD28Z | AAAIEVMYPPPYLDNEKSNGTIIHVKGKHLCPSPLFPGPSKPFWV |
| | | LVVVGGVLACYSLLVTVAFIIFWVRSKRSRLLHSDYMNMTPRRP |
| | | GPTRKHYQPYAPPRDFAAYRSRVKFSRSADAPAYQQGQNQLYN |
| | | ELNLGRREEYDVLDKRRGRDPEMGGKPRRKNPQEGLYNELQKD |
| | | KMAEAYSEIGMKGERRRGKGHDGLYQGLSTATKDTYDALHMQ |
| | | ALPPR |
| 202 | CD28ext-CD28TM-CD28z1XX | AAAIEVMYPPPYLDNEKSNGTIIHVKGKHLCPSPLFPGPSKPFWV |
| | | LVVVGGVLACYSLLVTVAFIIFWVRSKRSRLLHSDYMNMTPRRP |
| | | GPTRKHYQPYAPPRDFAAYRSRVKFSRSADAPAYQQGQNQLYN |
| | | ELNLGRREEYDVLDKRRGRDPEMGGKPRRKNPQEGLFNELQKD |
| | | KMAEAFSEIGMKGERRRGKGHDGLFQGLSTATKDTFDALHMQ |
| | | ALPPR |

1-6. (canceled)

7. An isolated polypeptide comprising an amino acid sequence selected from:
a) SEQ ID NOs: 1, 4, and 7;
b) SEQ ID NOs: 2, 5, and 8;
c) SEQ ID NOs: 3, 6, and 9;
d) SEQ ID NO: 10, GNK, and SEQ ID NO: 16;
e) SEQ ID NO: 11, GKN, and SEQ ID NO: 17; or
f) SEQ ID NO: 12, GKN, and SEQ ID NO: 18.

8-13. (canceled)

14. The isolated polypeptide of claim 7, wherein the polypeptide comprises a heavy chain variable region (VH) comprising an amino acid sequence selected from SEQ ID NOs: 19-21.

15. (canceled)

16. The isolated polypeptide of claim 7, wherein the polypeptide comprises a light chain variable region (VL) comprising an amino acid sequence selected from SEQ ID NOs: 22-24.

17-19. (canceled)

20. The isolated polypeptide of claim 7, wherein the polypeptide comprises:
a) a heavy chain variable region (VH) comprising the sequence of SEQ ID NO: 19, and a light chain variable region (VL) comprising the sequence of SEQ ID NO: 22;
b) a heavy chain variable region (VH) comprising the sequence of SEQ ID NO: 20, and a light chain variable region (VL) comprising the sequence of SEQ ID NO: 23;
c) a heavy chain variable region (VH) comprising the sequence of SEQ ID NO: 21, and a light chain variable region (VL) comprising the sequence of SEQ ID NO: 24
d) three heavy chain complementarity determining regions (HCDR1, HCDR2, and HCDR3) and three light chain complementarity determining regions (LCDR1, LCDR2, and LCDR3), wherein HCDR1, HCDR2, HCDR3, LCDR1, LCDR2, and LCDR3, respectively, comprise SEQ ID NOs: 1, 4, 7, and 10, GKN, and SEQ ID NO: 16;
e) three heavy chain complementarity determining regions (HCDR1, HCDR2, and HCDR3) and three light chain complementarity determining regions (LCDR1, LCDR2, and LCDR3), wherein HCDR1, HCDR2, HCDR3, LCDR1, LCDR2, and LCDR3, respectively, comprise SEQ ID NOs: 2, 5, 8, and 11, GKN, and SEQ ID NO: 17: or
f) three heavy chain complementarity determining regions (HCDR1, HCDR2, and HCDR3) and three light chain complementarity determining regions (LCDR1, LCDR2, and LCDR3), wherein HCDR1, HCDR2, HCDR3, LCDR1, LCDR2, and LCDR3, respectively, comprise SEQ ID NOs: 3, 6, 9, and 12, GKN, and SEQ ID NO: 18.

21-25. (canceled)

26. The isolated polypeptide of claim 20, wherein the polypeptide is an antibody or antigen binding fragment thereof, wherein the antibody or antigen binding fragment thereof is a bispecific antibody, Fab, Fab′, F(ab′)2, Fd, scFv, (scFv)2, scFv Fc, sdAb, VHH, or Fv fragment.

27-37. (canceled)

38. The isolated polypeptide of claim 26, wherein the bispecific antibody or antigen binding fragment thereof comprises an antibody or antigen binding fragment thereof that specifically binds at least one additional cell surface molecule, wherein the at least one additional cell surface molecule comprises CD3, CD19, BCMA, 4-IBB, IL-6, NKG2D, Fc-gamma-RIIIA (CD16), APRIL, CD38, TACI, Fc-gamma-RIIIA (CD16) and NKG2D, CD3and serum albumin, or CD47 and TACI.

39-42. (canceled)

43. An isolated polynucleotide encoding the isolated polypeptide of claim 7.

44. An isolated vector comprising the polynucleotide of claim 43.

45-50. (canceled)

51. An isolated host cell comprising the polynucleotide of claim 43.

52. A chimeric antigen receptor (CAR) comprising an extracellular binding domain that specifically binds G protein-coupled receptor class C group 5 member D (GPRC5D), wherein the extracellular binding domain comprises an antigen binding domain that comprises the polypeptide of claim 7.

53. (canceled)

54. The CAR of claim 52, wherein the CAR comprises one or more of a signal peptide, an extracellular binding domain, a hinge domain, a transmembrane domain, an intracellular costimulatory domain, and an intracellular signaling domain.

55. The CAR of any one of claim 54, wherein:
a) the signal peptide is selected from a CD8a signal peptide, a IgK signal peptide, a GMCSFR-a (CSF2RA) signal peptide, or Immunoglobulin heavy chain signal peptide;
b) the hinge domain is selected from a CD8a hinge domain, CD28 hinge domain, CD28 hinge domain, IqG4 hinge domain, or IqG4 hinge-CH2-CH3 domain;
c) the transmembrane domain is selected from a CD8a transmembrane domain or a CD28 transmembrane domain; and
d) the intracellular domain is selected from a CD137 (4-IBB) signaling domain, a CD28 signaling domain, or a CD3zeta signaling domain.

56-65. (canceled)

66. An isolated polynucleotide encoding the CAR of claim 52.

67-77. (canceled)

78. A viral vector targeting an immune cell, the viral vector comprising:
a. an antibody or antigen binding fragment thereof that binds to a cell surface molecule on the immune cell, wherein the antibody or antigen binding fragment thereof is attached to a membrane-bound protein in the viral vector envelope or to a fusogen on the outer surface of the viral vector; and
b. at least one polynucleotide encoding the chimeric antigen receptor (CAR) of claim 52.

79-81. (canceled)

82. The viral vector of claim 78, wherein the viral vector comprises a henipavirus envelope glycoprotein G (G protein) or a biologically active portion thereof, and/or a henipavirus F protein molecule or a biologically active portion thereof.

83. (canceled)

84. The viral vector of claim 82, wherein the viral vector comprises a henipavirus envelope glycoprotein G (G protein) or a biologically active portion thereof attached to the antibody or antigen binding fragment thereof.

85-143. (canceled)

144. An engineered cell comprising:
a. the CAR of claim 52; and
b. one or more modifications that (i) reduce or eliminate expression of one or more MHC class I molecules and/or one or more MHC class II molecules, and/or (ii) increase expression of one of more tolerogenic factors, wherein the reduced or eliminated expression of (i) and the increase expression of (ii) is relative to a cell of the same cell type that does not comprise the modifications.

145-149. (canceled)

150. The engineered cell of claim 144, wherein the one or more tolerogenic factors comprise one or more tolerogenic factor selected from A20/TNFAIP3, CI-Inhibitor, CCL21, CCL22, CD16, CD16 Fe receptor, CD24, CD27, CD35, CD39, CD46, CD47, CD52, CD55, CD59, CD200, CRI, CTLA4-Ig, DUX4, FasL, H2-M3, HLA-C, HLA-E, HLA-E heavy chain, HLA-F, HLA-G, 1DO1, IL-10, IL-15RF, IL-35, MANF, Mfge8, PD-L1, and Serpinb9.

151. (canceled)

152. A method for treating a disease in a subject comprising administering to the subject an effective amount of the engineered cell of claim 144.

153-158. (canceled)

159. The method of claim 152, wherein the disease is cancer.

160-164. (canceled)
